(19) United States
(12) Patent Application Publication
(10) Pub. No.: US 2004/0014173 A1
(43) Pub. Date: Jan. 22, 2004

(54) Novel polynucleotides, polypeptides encoded thereby and methods of use thereof

(76) Inventors: David W. Anderson, Plantsville, CT (US); Ferenc L. Boldog, North Haven, CT (US); Stacie J. Casman, North Haven, CT (US); Shlomit R. Edinger, New Haven, CT (US); Karen Ellerman, Branford, CT (US); Elma R. Fernandes, Branford, CT (US); Erik Gunther, Branford, CT (US); Martin D. Leach, Madison, CT (US); John R. MacDougall, Hamden, CT (US); Muralidhara Padigaru, Branford, CT (US); Richard A. Shimkets, Guilford, CT (US); Glennda Smithson, Guilford, CT (US); Kimberly A. Spytek, Ellington, CT (US)

Correspondence Address: MINTZ, LEVIN, COHN, FERRIS, GLOVSKY, AND POPEO, P.C., ONE FINANCIAL CENTER, BOSTON, MA, 02111, US

(21) Appl. No.: 10/384,974

(22) Filed: Mar. 10, 2003

Related U.S. Application Data
(63) Continuation of application No. 10/081,407, filed on Feb. 21, 2002, now abandoned.
Application No. 10/081,407 is a continuation-in-part of application No. 09/569,269, filed on May 11, 2000.
(60) Provisional application No. 60/134,315, filed on May 14, 1999.
(60) Provisional application No. 60/175,744, filed on Jan. 12, 2000.
(60) Provisional application No. 60/188,274, filed on Mar. 10, 2000.

(51) Int. Cl. C12Q 1/68; A61K 38/17; C07H 21/04; C12P 21/02; C12N 5/06; C07K 14/705; C07K 16/28

(52) U.S. Cl. 435/69.1; 435/6; 435/320.1; 435/325; 530/350; 530/388.22; 514/12; 536/23.5

(57) ABSTRACT

The present invention provides novel polypeptides, termed SECX polypeptides, as well as polynucleotides encoding SECX polypeptides and antibodies that immunospecifically bind to SECX or a derivative, variant, mutant, or fragment of the SECX polypeptide, polynucleotide or antibody. The invention additionally provides methods in which the SECX polypeptide, polynucleotide and antibody are used in detection and treatment of a broad range of pathological states, as well as to others uses.

### RELATED APPLICATIONS

This application is a Continuation of U.S. Ser. No. 10/081,407, which is a Continuation-in-Part of U.S. Ser. No. 09/569,269 filed May 11, 2000, which claims priority to U.S. Ser. No. 60/134,315 filed May 14, 1999; U.S. Ser. No. 60/175,744, filed Jan. 12, 2000; and U.S. Ser. No. 60/188,274, filed Mar. 10, 2000, all of which are hereby incorporated by reference.

### FIELD OF THE INVENTION

The invention relates to polynucleotides and polypeptides encoded by such polynucleotides, as well as vectors, host cells, antibodies and recombinant methods for producing the polypeptides and polynucleotides.

### BACKGROUND OF THE INVENTION

Eukaryotic cells are subdivided by membranes into multiple functionally distinct compartments that are referred to as organelles. Each organelle includes proteins essential for its proper function. These proteins can include sequence motifs often referred to as sorting signals. The sorting signals can aid in targeting the proteins to their appropriate cellular organelle. In addition, sorting signals can direct some proteins to be exported, or secreted, from the cell.

One type of sorting signal is a signal sequence, which is also referred to as a signal peptide or leader sequence. The signal sequence is present as an amino-terminal extension on a newly synthesized polypeptide chain A signal sequence can target proteins to an intracellular organelle called the endoplasmic reticulum (ER).

The signal sequence takes part in an array of protein-protein and protein-lipid interactions that result in translocation of a polypeptide containing the signal sequence through a channel in the ER. After translocation, a membrane-bound enzyme, named a signal peptidase, liberates the mature protein from the signal sequence.

The ER functions to separate membrane-bound proteins and secreted proteins from proteins that remain in the cytoplasm. Once targeted to the ER, both secreted and membrane-bound proteins can be further distributed to another cellular organelle called the Golgi apparatus. The Golgi directs the proteins to other cellular organelles such as vesicles, lysosomes, the plasma membrane, mitochondria and microbodies.

Secreted and membrane-bound proteins are involved in many biologically diverse activities. Examples of known secreted proteins include human insulin, interferon, interleukins, transforming growth factor-beta, human growth hormone, erythropoietin, and lymphokines. Only a limited number of genes encoding human membrane-bound and secreted proteins have been identified.

### SUMMARY OF THE INVENTION

The invention is based in part on the discovery of novel nucleic acids and secreted polypeptides encoded thereby. The nucleic acids and polypeptides are collectively referred to herein as “SECX”.

Accordingly, in one aspect, the invention provides an isolated nucleic acid molecule that includes the sequence of any of SEQ ID NO: 2n−1, wherein n is an integer between 1-20, that encodes a novel polypeptide, or a fragment, homolog, analog or derivative thereof. The nucleic acid can include, e.g., a nucleic acid sequence encoding a polypeptide at least 85% identical to a polypeptide comprising the amino acid sequences of SEQ ID NO: 2n, wherein n is an integer between 1-20. The nucleic acid can be, e.g., a genomic DNA fragment, or a cDNA molecule.

Also included in the invention is a vector containing one or more of the nucleic acids described herein, and a cell containing the vectors or nucleic acids described herein.

The invention is also directed to host cells transformed with a vector comprising any of the nucleic acid molecules described above.

In another aspect, the invention includes a pharmaceutical composition that includes an SECX nucleic acid and a pharmaceutically acceptable carrier or diluent.

In a further aspect, the invention includes a substantially purified SECX polypeptide, e.g., any of the SECX polypeptides encoded by an SECX nucleic acid, and fragments, homologs, analogs, and derivatives thereof. The invention also includes a pharmaceutical composition that includes a SECX polypeptide and a pharmaceutically acceptable carrier or diluent.

In a still a further aspect, the invention provides an antibody that binds specifically to an SECX polypeptide. The antibody can be, e.g., a monoclonal or polyclonal antibody, and fragments, homologs, analogs, and derivatives thereof. The invention also includes a pharmaceutical composition including SECX antibody and a pharmaceutically acceptable carrier or diluent. The invention is also directed to isolated antibodies that bind to an epitope on a polypeptide encoded by any of the nucleic acid molecules described above.

The invention also includes kits comprising any of the pharmaceutical compositions described above.

The invention further provides a method for producing an SECX polypeptide by providing a cell containing a SECX nucleic acid, e.g., a vector that includes a SECX nucleic acid, and culturing the cell under conditions sufficient to express the SECX polypeptide encoded by the nucleic acid. The expressed SECX polypeptide is then recovered from the cell. Preferably, the cell produces little or no endogenous SECX polypeptide. The cell can be, e.g., a prokaryotic cell or eukaryotic cell.

The invention is also directed to methods of identifying an SECX polypeptide or nucleic acids in a sample by contacting the sample with a compound that specifically binds to the polypeptide or nucleic acid, and detecting complex formation, if present.

The invention further provides methods of identifying a compound that modulates the activity of a SECX polypeptide by contacting SECX polypeptide with a compound and determining whether the SECX polypeptide activity is modified.

The invention is also directed to compounds that modulate SECX polypeptide activity identified by contacting a SECX polypeptide with the compound and determining whether the compound modifies activity of the SECX polypeptide, binds to the SECX polypeptide, or binds to a nucleic acid molecule encoding a SECX polypeptide.

In a another aspect, the invention provides a method of determining the presence of or predisposition of an SECX-associated disorder in a subject. The method includes providing a sample from the subject and measuring the amount of SECX polypeptide in the subject sample. The amount of SECX polypeptide in the subject sample is then compared to the amount of SECX polypeptide in a control sample. An alteration in the amount of SECX polypeptide in the subject protein sample relative to the amount of SECX polypeptide in the control protein sample indicates the subject has a tissue proliferation-associated condition. A control sample is preferably taken from a matched individual, i.e., an individual of similar age, sex, or other general condition but who is not suspected of having a tissue proliferation-associated condition. Alternatively, the control sample may be taken from the subject at a time when the subject is not suspected of having a tissue proliferation-associated disorder. In some embodiments, the SECX is detected using a SECX antibody.

In a further aspect, the invention provides a method of determining the presence of or predisposition of an SECX-associated disorder in a subject. The method includes providing a nucleic acid sample, e.g., RNA or DNA, or both, from the subject and measuring the amount of the SECX nucleic acid in the subject nucleic acid sample. The amount of SECX nucleic acid sample in the subject nucleic acid is then compared to the amount of an SECX nucleic acid in a control sample. An alteration in the amount of SECX nucleic acid in the sample relative to the amount of SECX in the control sample indicates the subject has a tissue proliferation-associated disorder.

In a still further aspect, the invention provides method of treating or preventing or delaying a SECX-associated disorder. The method includes administering to a subject in which such treatment or prevention or delay is desired a SECX nucleic acid, a SECX polypeptide, or an SECX antibody in an amount sufficient to treat, prevent, or delay a tissue proliferation-associated disorder in the subject.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and not intended to be limiting.

Other features and advantages of the invention will be apparent from the following detailed description and claims.

### BRIEF DESCRIPTION OF THE DRAWINGS

FIG. 1 is a representation of a Western blot analysis showing expression of FGF10AC0044 in embryonic kidney 293 cells.

FIG. 2 is a representation of a Western blot analysis showing expression of FGF10AC0044 in E. coli cells.

FIG. 3 is a representation of a Western blot analysis showing expression of h16399139 as a 55 kDa protein secreted by human embryonic kidney 293 cells.

FIG. 4 shows that h10326230 is expressed as a 50 kDa protein secreted by 293 cells.

### DETAILED DESCRIPTION OF THE INVENTION

The invention provides novel polypeptides and nucleotides encoded thereby. Included in the invention are ten novel nucleic acid sequences and their encoded polypeptides. The sequences are collectively referred to as “SECX nucleic acids” or “SECX polynucleotides” and the corresponding encoded polypeptide is referred to as a “SECX polypeptide” or “SECX protein”. For example, an SECX nucleic acid according to the invention is a nucleic acid including an SECX nucleic acid, and an SECX polypeptide according to the invention is a polypeptide that includes the amino acid sequence of an SECX polypeptide. Unless indicated otherwise, “SECX” is meant to refer to any of the novel sequences disclosed herein.

Table 1 provides a summary of the SECX nucleic acids and their encoded polypeptides.

Column 1 of Table 1, entitled “SECX No.”, denotes an SECX number assigned to a nucleic acid according to the invention.

Column 2 of Table 1, entitled “Clone Identification number” provides a second identification number for the indicated SECX.

Column 3 of Table 1, entitled “Tissue Expression”, indicates the tissue in which the indicated SECX nucleic acid is expressed.

Columns 4-9 of Table 1 describes structural information as indicated for the indicated SECX nucleic acids and polypeptides.

Column 10 of Table 1, entitled “Protein Similarity” lists previously described proteins that are related to polypeptides encoded by the indicated SECX. Genbank identifiers for the previously described proteins are provided.

Column 11 of Table 1, entitled “Signal Peptide Cleavage Site” indicates the putative nucleotide position where the signal peptide is cleaved as determined by SignalP.

Column 12 of Table 1, entitled “Cellular Localization” indicates the putative cellular localization of the indicated SECX polypeptides.

TABLE 1

| | | | | Open | | | | | | Signal | Cellu- |
|---|---|---|---|---|---|---|---|---|---|---|---|
| | Clone | | | Reading | Amino | Calculated | | Stop | | Peptide | lar |
| SECX | Identification | Tissue | Nucleotide | Frame | Acid | Molecular | | in 5′ | | Cleavage | Locali- |
| No. | Number | Expression | length | (nt) | length | Weight | Kozak | UTR | Protein Similarity | Site (nt) | zation |
| 1 | FGF10AC004449 | | 670 | 130-639 | 170 | 19662.4 | | | Swiss New-Acc: | 22 and 23: | extra- |
| | | | | | | | | | O15520 fibroblast | | cellular. |
| | | | | | | | | | growth factor-10 | | |
| | | | | | | | | | precursor (fgf-10) | | |
| | | | | | | | | | (keratinocyte | | |
| | | | | | | | | | growth factor | | |
| | | | | | | | | | 2) - homo sapiens | | |
| | | | | | | | | | (human), | | |
| | | | | | | | | | 208 aa. | | |
| 2 | 10326230.0.38 | Spleen | 1680 | 177-1655 | 493 | 55238.6 | yes | yes | Acc:O43354 | 27 and 28: | plasma |
| | | | | | | | | | BAC Clone | | mem- |
| | | | | | | | | | GS099h08, com- | | brane |
| | | | | | | | | | plete sequence - | | |
| | | | | | | | | | homo sapiens | | |
| | | | | | | | | | (human), 522 aa. | | |
| | | | | | | | | | with acc:o88279 | | |
| | | | | | | | | | megf4 - Rattus | | |
| | | | | | | | | | Norvegicus (rat), | | |
| | | | | | | | | | 1531 aa. | | |
| 3 | 16399139.0.7 | Thalamus | 1908 | 230-1669 | 480 | 53945. | yes | yes | Acc:BAA76820 | 18 and 19: | endo- |
| | | | | | | | | | KIAA0976 | | plasmic |
| | | | | | | | | | protein - homo | | reticu- |
| | | | | | | | | | sapiens (human), | | lum |
| | | | | | | | | | 364 aa. | | (mem- |
| | | | | | | | | | | | brane) |
| 4 | 3440544.0.81 | Prostate gland, | 1597 | 122-1039 | 306 | 34245.1 | yes | yes | Acc:AADL2839 | | plasma |
| | | Thyroid gland, | | | | | | | y25cla.7b | | mem- |
| | | Placenta | | | | | | | protein - | | brane |
| | | Lymphoid tissue., | | | | | | | Caenorhabditis | | |
| | | Adrenal gland, | | | | | | | elegans, 287 aa. | | |
| | | brain, fetal brain, | | | | | | | Acc:O14546 | | |
| | | liver, fetal liver, | | | | | | | Polyspecific | | |
| | | skeletal muscle, | | | | | | | Organic Cation | | |
| | | pancreas, kidney, | | | | | | | Transporter - | | |
| | | heart, lung | | | | | | | homo sapiens | | |
| | | Bone.,Bone | | | | | | | (human), 551 aa | | |
| | | marrow | | | | | | | | | |
| 5 | 3581980.0.30 | Pituitary | 1782 | 949-1332 | 128 | 13962.1 | yes | yes | Acc:BAA24860 | | cyto- |
| | | | | | | | | | KIAA0430 | | plasm |
| | | | | | | | | | protein - homo | | |
| | | | | | | | | | sapiens (human), | | |
| | | | | | | | | | 1056 aa | | |
| | | | | | | | | | (fragment). | | |
| 6 | 4418354.0.6 | Many | 1265 | 142-1236 | 365 | 49899.1 | yes | yes | Acc:Q62825 | | cyto- |
| | | | | | | | | | (rsec6) - Rattus | | plasm |
| | | | | | | | | | Norvegicus (rat), | | |
| | | | | | | | | | 755 aa | | |
| | | | | | | | | | (fragment). | | |
| 7 | 4418354.0.9 | Many | 2833 | 142-2082 | 647 | 72791.2 | yes | yes | Acc:Q62825 | | cyto- |
| | | | | | | | | | (rsec6) - Rattus | | plasm |
| | | | | | | | | | Norvegicus (rat), | | |
| | | | | | | | | | 755 aa | | |
| | | | | | | | | | (fragment). | | |
| | | | | | | | | | Acc:O60645 Sec6 | | |
| | | | | | | | | | homolog - homo | | |
| | | | | | | | | | sapiens (human), | | |
| | | | | | | | | | 471 aa | | |
| | | | | | | | | | (fragment). | | |
| 8 | 6779999.0.31 | Adrenal gland | 1213 nt | 762-1028 | 89 | 10598.7 | yes | yes | | 30 and 31 | outside |
| 9 | 8484782.0.5 | Placenta | 1755 nt | 890-1531 | 214 | 24449.8 | yes | yes | Acc:OL4667 | | nucleus |
| | | | | | | | | | Gamma-Heregu- | | |
| | | | | | | | | | lin - homo | | |
| | | | | | | | | | sapiens (human), | | |
| | | | | | | | | | 768 aa. | | |
| 10 | 16399139.S124A | Thalmus | 1908 nt | 144-1433 | 430 | 48548.6 | | | SPTREMBL- | 18 and 19 | mito- |
| | | | | | | | | | ACC:Q9Y212: | | condria |
| | | | | | | | | | K1AA0976 | | |

Table 2 provides a cross reference to the assigned SECX number, clone identification number and sequence identification numbers (SEQ ID NOs.).

TABLE 2

| | Clone | | |
|---|---|---|---|
| SECX | Identification | SEQ ID NO | SEQ ID NO |
| No. | Number | Nucleic Acid | Polypeptide |
| 1 | FGF10AC004449 | 1 | 2 |
| 2 | 10326230.0.38 | 3 | 4 |
| 3 | 16399139.0.7 | 5 | 6 |
| 4 | 3440544.0.81 | 7 | 8 |
| 5 | 3581980.0.30 | 9 | 10 |
| 6 | 4418354.0.6 | 11 | 12 |
| 7 | 4418354.0.9 | 13 | 14 |
| 8 | 6779999.0.31 | 15 | 16 |
| 9 | 8484782.0.5 | 17 | 18 |
| 10 | 16399139.S124S | 19 | 20 |

Nucleic acid sequences and polypeptide sequences for SECX nucleic acids and polypeptides according to the invention are provided in the following section of the specification, which is entitled “Disclosed Sequences of SECX Nucleic Acid and Polypeptide Sequences.”

A polypeptide or protein described herein includes the product of a naturally occurring polypeptide or precursor form or proprotein. The naturally occurring polypeptide, precursor or proprotein includes, e.g., the full length gene product, encoded by the corresponding gene. The naturally occurring polypeptide also includes the polypeptide, precursor or proprotein encoded by an open reading frame described herein. A “mature” form of a polypeptide or protein arises as a result of one or more naturally occurring processing steps as they may occur within the cell, including a host cell. The processing steps occur as the gene product arises, e.g., via cleavage of the amino-terminal methionine residue encoded by the initiation codon of an open reading frame, or the proteolytic cleavage of a signal peptide or leader sequence. Thus, a mature form arising from a precursor polypeptide or protein that has residues 1 to N, where residue 1 is the N-terminal methionine, would have residues 2 through N remaining. Alternatively, a mature form arising from a precursor polypeptide or protein having residues 1 to N, in which an amino-terminal signal sequence from residue 1 to residue M is cleaved, includes the residues from residue M+1 to residue N remaining. A “mature” form of a polypeptide or protein may also arise from non-proteolytic post-translational modification. Such non-proteolytic processes include, e.g., glycosylation, myristylation or phosphorylation. In general, a mature polypeptide or protein may result from the operation of only one of these processes, or the combination of any of them.

As used herein, “identical” residues correspond to those residues in a comparison between two sequences where the equivalent nucleotide base or amino acid residue in an alignment of two sequences is the same residue. Residues are alternatively described as “similar” or “positive” when the comparisons between two sequences in an alignment show that residues in an equivalent position in a comparison are either the same amino acid or a conserved amino acid as defined below.

SECX nucleic acids, and their encoded polypeptides, according to the invention are useful in a variety of applications and contexts. For example, various SECX nucleic acids and polypeptides according to the invention are useful, inter alia, as novel members of the protein families according to the presence of domains and sequence relatedness to previously described proteins

SECX nucleic acids and polypeptides according to the invention can also be used to identify cell types for an indicated SECX according to the invention. Examples of such cell types are listed in Table 1, column 3 for a SECX according to the invention. Additional utilities for SECX nucleic acids and polypeptides according to the invention are disclosed herein.

Disclosed Sequences of SECX Nucleic Acid and Polypeptide Sequences SEC1

A SEC1 nucleic acid and polypeptide according to the invention includes the nucleic acid and encoded polypeptide sequence of FGF10_AC004449. The predicted open reading frame codes for a 170 amino acid long secreted protein with 54% identity to the Human Fibroblast Growth Factor 10 precursor (SWISSNEW-Acc. No. O15520), which is also known as Keratinocyte Growth Factor 2 (see PCT publication WO 98/16642-A1).

The disclosed SEC1 polypeptide sequence is predicted by the PSORT program to localize extracellularly with a certainty of 0.5374. The program SignalP predicts that there is a signal peptide, with the most likely cleavage site between residues 22 and 23 in the sequence AAG-TP.

The fibroblast growth factor (FGF) family includes a number of structurally related polypeptide growth factors that are heparin-binding polypeptides. These molecules have been implicated in a variety of human neoplasms. Their expression is controlled at the levels of transcription, mRNA stability, and translation. The bioavailability of FGFs is further modulated by posttranslational processing and regulated protein trafficking. FGFs typically bind to receptor tyrosine kinases (FGFRs), heparan sulfate proteoglycans (HSPG), and a cysteine-rich FGF receptor (CFR).

FGFRs are required for most biological activities of FGFs. HSPGs alter FGF-FGFR interactions, and CFR participates in FGF intracellular transport. FGF signaling pathways are intricate and are intertwined with insulin-like growth factor, transforming growth factor-beta, bone morphogenetic protein, and vertebrate homologs of Drosophila wingless activated pathways. FGFs are major regulators of embryonic development: They influence the formation of the primary body axis, neural axis, limbs, and other structures. The activities of FGFs depend on their coordination of fundamental cellular functions, such as survival, replication, differentiation, adhesion, and motility, through effects on gene expression and the cytoskeleton.

FGF signaling is mediated by a dual-receptor system, consisting of four high-affinity tyrosine kinase receptors, termed fibroblast growth factor receptors (FGFRs), and of low-affinity heparan sulfate proteoglycan receptors that enhance ligand presentation to the FGFRs. Several FGFs, including FGF-1, -2, -3, -4, -5, -6, and -7, and several FGFR variants, among them the 2 immunoglobulin-like form and the IIIc splice variant of FGFR-1 and the keratinocyte growth factor receptor, a splice variant of FGFR-2, are expressed in human pancreatic cancer cell lines and are overexpressed in human pancreatic cancers or in the pancreas of chronic pancreatitis and, therefore, may play important roles in the pathobiology of these pancreatic diseases.

Additionally, SEC1 has high similarity to several segments from a human metalloprotease thrombospondin 1 (METH1) related EST (AC004449) of 38186 bp (see PCT publication WO 99/37660-A1). Metalloprotease thrombospondins are potent inhibitors of angiogenesis both in vitro and in vivo. Accordingly, SEC1 nucleic acids and polypeptides may be useful in treating cancer and other disorders related to angiogenesis including abnormal wound healing, inflammation, rheumatoid arthritis, psoriasis, endometrial bleeding disorders, diabetic retinopathy, some forms of macular degeneration, haemangiomas, and arterial-venous malformations.

The FGF10_AC004449 nucleic acid and encoded polypeptide has the following sequence:

| | | |
|---|---|---|
| 1 | CCATTGGCCGGCGTCCCCGCCCCAGCGAACCCGGCCCCGCCCCCG | (SEQ ID NO:1) |
| 46 | ACGCGCCCCATTGGCCCCGCCGCGCGAAGGCAGAGCCCCGGACGC | |
| 91 | CCGGGAGCGACGAGCGCGCAGCGAACCGGGTGCCGGGTCATGCGC | |
| | MetArg | (SEQ ID NO:2) |
| 136 | CGCCGCCTGTGGCTGGGCCTGGCCTGGCTGCTGCTGGCGCGGGCG | |
| | ArgArgLeuTrpLeuGlyLeuAlaTrpLeuLeuLeuAlaArgAla | |
| 181 | CCGGACCCCGCGGGAACCCCGAGCGCGTCGCGGGGACCGCGCAGC | |
| | ProAspAlaAlaGlyThrProSerAlaSerArgGlyProArgSer | |
| 226 | TACCCGCACCTGGAGGGCGACGTGCGCTGGCGCCGCCTCTTCTCC | |
| | TyrProHisLeuGluGlyAspValArgTrpArgArgLeuPheSer | |
| 271 | TCCACTCACTTCTTCCTGCGCGTGGATCCCGCCGGCCGCGTGCAG | |
| | SerThrHisPhePheLeuArgValAspProGlyGlyArgValGln | |
| 316 | GGCACCCGCTGGCGCCACGGCCACGACAGCATCCTGGAGATCCGC | |
| | GlyThrArgTrpArgHisGlyGlnAspSerIleLeuGluIleArg | |
| 361 | TCTGTACACGTGGGCGTCGTGCTCATCAAAGCAGTGTCCTCACGC | |
| | SerValHisValGlyValValValIleLysAlaValSerSerGly | |
| 406 | TTCTACGTGGCCATGAACCGCCGCGGCCGCCTCTACGGGTCGCGA | |
| | PheTyrValAlaMetAsnArgArgGlyArgLeuTyrGlySerArg | |
| 451 | CTCTACACCGTGGACTGCAGGTTCCGGGAGCGCATCGAAGAGAAC | |
| | LeuTyrThrValAspCysArgPheArgGluArgIleGluGluAsn | |
| 496 | GGCCACAACACCTACGCCTCACACCGCTGGCGCCCCCGCGGCCAG | |
| | GlyHisAsnThrTyrAlaSerGlnArgTrpArgArgArgGlyGln | |
| 541 | CCCATGTTCCTGGCGCTGGACAGGAGGGGGGGGCCCCGGCCAGGC | |
| | ProMetPheLeuAlaLeuAspArgArgGlyGlyProArgProGly | |
| 586 | GGCCGGACGCGGCGGTACCACCTGTCCGCCCACTTCCTGCCCGTC | |
| | GlyArgThrArgArgTyrHisLeuSerAlaHisPheLeuProVal | |
| 631 | CTGGTCTCCTGAGGCCCTGAGAGGCCGGCGGCTCCCCAAG | |
| | LeuValSer | |

SEC2

A SEC2 nucleic acid and polypeptide according to the invention includes the nucleic acid and encoded polypeptide sequence of 10326230.0.38.

The disclosed SEC2 polypeptide is predicted by the PSORT program to localize in the plasma membrane with a certainty of 0.4600. The program SignalP predicts that there is a signal peptide, with the most likely cleavage site between residues 27 and 28, in the sequence ARA-GR.

The disclosed SEC2 polypeptide has 143 of 392 amino acid residues (36%) identical to, and 220 of 392 residues (56%) positive with, the 522 residue protein encoded in human BAC CLONE GS099H08 (GenBank Acc. No.:O43354).

The SEC2 nucleic acid is highly epxressed in brain tissue. Expression information describing the amount of RNA homologous to SEC2 and various other SECX nucleic acids according to the invention is provided in the Examples, including Table 5.

The 10326230 nucleic acid and encoded polypeptide has the following sequence:

| | | |
|---|---|---|
| 1 | TGACAGGCCGGCCGGTGAGGCGCCGCCGGGAGAGGCCGCGACGGA | (SEQ ID NO:3) |
| 46 | GCTCCCAGACCGGCCATGGGCTGAGACACGTCCTCGCCGAGCAGT | |
| 91 | GACCCTTCCGTACCCCACCAGAACATGCCCGGGTGACCTCCTCCC | |
| 136 | AGATCTTCCTTGTGGCCTTCCTCGCCCACTCCAGTGACACTATGC | |
| | MetH | (SEQ ID NO:4) |
| 181 | ACCCCCACCGTGACCCGAGAGGCCTCTGGCTCCTGCTGCCGTCCT | |
| | isProHisArgAspProArgGlyLeuTrpLeuLeuLeuProSerL | |
| 226 | TGTCCCTGCTGCTTTTTGACGTGGCCAGAGCTGGCCGAGCCGTGG | |
| | euSerLeuLeuLeuPheGluValAlaArgAlaGlyArgAlaValV | |
| 271 | TTAGCTGTCCTGCCGCCTGCTTGTGCGCCAGCAACATCCTCAGCT | |
| | alSerCysProAlaAlaCysLeuCysAlaSerAsnIleLeuSerC | |
| 316 | GCTCCAAGCAGCAGCTGCCCAATGTGCCCCATTCCTTGCCCAGTT | |
| | ysSerLysGlnGlnLeuProAsnValProHisSerLeuProSerT | |
| 361 | ACACAGCACTACTGGACCTCAGTCACAACAACCTGAGCCGCCTGC | |
| | yrThrAlaLeuLeuAspLeuSerHisAsnAsnLeuSerArgLeuA | |
| 406 | GGGCCGAGTGGACCCCCACGCGCCTGACCCAACTGCACTCCCTGC | |
| | rgAlaGluTrpThrProThrArgLeuThrGlnLeuHisSerLeuL | |
| 451 | TGCTGAGCCACAACCACCTGAACTTCATCTCCTCTGAGGCCTTTT | |
| | euLeuSerHisAsnHisbeuAsnPheIleSerSerGluAlaPheS | |
| 496 | CCCCGGTACCCAACCTGCGCTACCTGGACCTCTCCTCCAACCAGC | |
| | erProValProAsnLeuArgTyrLeuAspLeuSerSerAsnGlnL | |
| 541 | TGCGTACACTGGATGAGTTCCTGTTCAGTGACCTGCAAGTACTGG | |
| | euArgThrLeuAspGluPheLeuPheSerAspLeuGlnValLeuG | |
| 586 | AGGTGCTGCTGCTCTACAATAACCACATCATGGCGGTGGACCGGT | |
| | luValLeuLeuLeuTyrAsnAsnHisIleMetAlaValAspArgC | |
| 631 | GCGCCTTCGATGACATGGCCCAGCTGCAGAAACTCTACTTGAGCC | |
| | ysAlaPheAspAspMetAlaGlnLeuGlnLysLeuTyrLeuSerG | |
| 676 | AGAACCAGATCTCTCGCTTCCCTCTGGAACTGGTCAAGGAAGGAG | |
| | lnAsnGlnIleSerArgPheProLeuGluLeuValLysGluGlyA | |
| 721 | CCAAGCTACCCAAACTAACGCTCCTGGATCTCTCTTCTAACAAGC | |
| | laLysLeuProLysLeuThrLeuLeuAspLeuSerSerAsnLysL | |
| 766 | TGAAGAACTTGCCATTGCCTGACCTGCAGAAGCTGCCGGCCTGGA | |
| | euLysAsnLeuProLeuProAspbeuGlnLysLeuProAlaTrpI | |
| 811 | TCAAGAATGGGCTGTACCTACATAACAACCCCCTGAACTGCCACT | |
| | leLysAsnGlyLeuTyrLeuHisAsnAsnProLeuAsnCysAspC | |
| 856 | GTGAGCTCTACCAGCTCTTTTCACACTGGCAGTATCGGCAGCTGA | |
| | ysGluLeuTyrGlnLeuPheSerHisTrpGlnTyrArgGlnLeuS | |
| 901 | GCTCCGTGATGGACTTTCAAGAGGATCTGTACTCCATGAACTCCA | |
| | erSerValMetAspPheGlnGluAspLeuTyrCysMetAsnSerL | |
| 946 | AGAAGCTGCACAATGTCTTCAACCTGAGTTTCCTCAACTGTGGCG | |
| | ysLysLeuHisAsnValPheAsnLeuSerPheLeuAsnCysGlyG | |
| 991 | AGTACAAGGAGCGTGCCTGGGAGGCCCACCTGGGTGACACCTTGA | |
| | luTyrLysGluArgAlaTrpGluAlaHisLeuGlyAspThrLeuI | |
| 1036 | TCATCAAGTGTGACACCAAGCAGCAAGGGATGACCAAGGTGTGCG | |
| | leIleLysCysAspThrLysGlnGlnGlyMetThrLysValTrpV | |
| 1081 | TGACACCAAGTAATGAACGGGTGCTAGATGAGGTGACCAATGGCA | |
| | alThrProSerAsnGluArgValLeuAspGluValThrAsnGlyT | |
| 1126 | CAGTGAGTGTGTCTAAGGATGGCAGTCTTCTTTTCCACCAGGTGC | |
| | hrValSerValSerLysAspGlySerLeuLeuPheGlnGlnValG | |
| 1171 | AGGTCGAGGACGGTGGTGTGTATACCTGCTATGCCATGGGAGAGA | |
| | lnValGluAspGlyGlyValTyrThrCysTyrAlaMetGlyGluT | |
| 1216 | CTTTCAATGAGACACTGTCTGTGGAATTGAAAGTGCACAATTTCA | |
| | hrPheAsnGluThrLeuSerValGluLeuLysValHisAsnPheT | |
| 1261 | CCTTGCACGGACACCATGACACCCTCAACACAGCCTATACCAGCC | |
| | hrPheHisGlyHisHisAspThrLeuAsnThrAlaTyrThrThrL | |
| 1306 | TAGTGGGCTGTATCCTTAGTGTGGTCCTGGTCCTCATATACCTAT | |
| | euValGlyCysIleLeuSerValValLeuValLeuIleTyrLeuT | |
| 1351 | ACCTCACCCCTTGCCGCTGCTGGTGCCGGGGTGTAGAGAAGCCTT | |
| | yrLeuThrProCysArgCysTrpCysArgGlyValGluLysProS | |
| 1396 | CCAGCCATCAAGGAGACAGCCTCAGCTCTTCCATGCTTAGTACCA | |
| | erSerHisGlnGlyAspSerLeuSerSerSerMetLeuSerThrT | |
| 1441 | CACCCAACCATGATCCTATGGCTGGTGGGGACAAAGATGATGGTT | |
| | hrProAsnHisAspProMetAlaGlyGlyAspLysAspAspGlyP | |
| 1486 | TTGACCGGCGGGTGGCTTTCCTGGAACCTGCTGGACCTGGGCAGG | |
| | heAspArgArgValAlaPheLeuGluProAlaGlyProGlyGlnG | |
| 1531 | GTCAAAACGGCAAGCTCAAGCCAGGCAACACCCTGCCAGTGCCTG | |
| | lyGlnAsnGlyLysLeuLysProGlyAsnThrLeuProValProG | |
| 1576 | AGGCCACAGGCAAGGGCCAACGGAGGATGTCGGATCCAGAATCAG | |
| | luAlaThrGlyLysGlyGlnArqArgMetSerAspProGluSerV | |
| 1621 | TCAGCTCGGTCTTCTCTGATACGCCCATTGTGGTGTGAGCAGGAT | |
| | alSerSerValPheSerAspThrProIleValVal | |
| 1666 | GGGTTGGTGGGGAGA | |

Construction of the mammalian expression vector pCEP4/Sec. The oligonucleotide primers, pSec-V5-His Forward (CTCGTC CTCGAG GGT AAG CCT ATC CCT AAC) (SEQ ID NO: 52) and the pSec-V5-His Reverse (CTCGTC GGGCCC CTGATCAGCGGGTTIAAAC) (SEQ ID NO: 53), were designed to amplify a fragment from the pcDNA3.1-V5His (Invitrogen, Carlsbad, Calif.) expression vector. The PCR product was digested with XhoI and ApaI and ligated into the XhoI/ApaI digested pSecTag2 B vector (Invitrogen, Carlsbad Calif.). The correct structure of the resulting vector, pSecV5His, was verified by DNA sequence analysis. The vector pSecV5His was digested with PmeI and NheI, and the PmeI-NheI fragment was ligated into the BamHI/Klenow and NheI treated vector pCEP4 (Invitrogen, Carlsbad, Calif.). The resulting vector was named as pCEP4/Sec.

Expression of h10326230 in human embryonic kidney 293 cells. A 0.4 kb BamHI-XhoI fragment containing the h10326230 sequence was isolated from pCR2.1-10326230 and subcloned into BamHI-XhoI digested pCEP4/Sec to generate expression vector pCEP4/Sec-10326230. The pCEP4/Sec-10326230 vector was transfected into 293 cells using the LipofectaminePlus reagent following the manufacturer's instructions (Gibco/BRL). The cell pellet and supernatant were harvested 72 hours after transfection and examined for h10326230 expression by Western blotting (reducing conditions) with an anti-V5 antibody. FIG. 4 shows that h10326230 is expressed as a 50 kDa protein secreted by 293 cells.

SEC3

A SEC3 nucleic acid and polypeptide according to the invention includes the nucleic acid and encoded polypeptide sequence of 16399139. The disclosed SEC3 is predicted by the PSORT program to localize to the membrane of the endoplasmic reticulum with a certainty of 0.6400. The program SignalP predicts that there is a signal peptide, with the most likely cleavage site between residues 18 and 19, in the sequence VSS-VM.

The disclosed SEC3 polypeptide has 362 of 363 residues (99%) identical to, and 100.0% of 363 residues positive with, the 364 residue protein encoded by the human sequence KIAA0976 (GenBank Accession No: BAA76820).

Tissue expression analysis shows SEC3 to be singularly expressed in colon cancer, renal cancer and liver cancer cells. (Table5)

The 16399139 nucleic acid and encoded polypeptide has the following sequence:

| | | |
|---|---|---|
| 1 | GGCTTCCACCAAAGTCCTCAATATACCTGAATACGCACAATATCT | (SEQ ID NO:5) |
| 46 | TAACTCTTCATATTTGGTTTTGCGATCTGCTTTGAGGTCCCATCT | |
| 91 | TCATTTAAAAAAAAATACAGAGACCTACCTACCCCTACGCATACA | |
| 136 | TACATATGTGTATATATATGTAAACTAGACAAAGATCGCAGATCA | |
| 181 | TAAAGCAAGCTCTGCTTTAGTTTCCAAGAAGATTACAAAGAATTT | |
| 226 | AGAGATGTATTTGTCAAGATTCCTGTCGATTCATGCCCTTTGGGT | |
| | MetTyrLeuSerArgPheLeuSerIleHisAlaLeuTrpVa | (SEQ ID NO:6) |
| 271 | TACGGTGTCCTCAGTGATGCAGCCCTACCCTTTGGTTTGGGGACA | |
| | lThrValSerSerValMetClnProTyrProLeuValTrpGlyHi | |
| 316 | TTATGATTTGTGTAAGACTCAGATTTACACGGAAGAAGGGAAAGT | |
| | sTyrAspLeuCysLysThrGlnIleTyrThrGluGluGlyLysVa | |
| 361 | TTGGGATTACATGGCCTGCCAGCCGGAATCCACGGACATGACAAA | |
| | lTrpAspTyrMetAlaCysGlnProGluSerThrAspMetThrLy | |
| 406 | ATATCTGAAACTGAAACTCGATCCTCCGCATATTACCTGTGGAGA | |
| | sTyrLeuLysValLysLeuAspProProAspIleThrCysGlyAs | |
| 451 | CCCTCCTGAGACGTTCTGTGCAATGCGCAATCCCTACATGTCCAA | |
| | pProProGluThrPheCysAlaMetGlyAsnProTyrMetCysAs | |
| 496 | TAATGAGTGTGATGCGAGTACCCCTGAGCTGGCACACCCCCCTGA | |
| | nAsnGluCysAspAlaSerThrProGluLeuAlaHisProProGl | |
| 541 | GCTGATGTTTGATTTTGAAGGAAGACATCCCTCCACATTTTGCCA | |
| | uLeuMetPheAspPheGluGlyArgHisProSerThrPheTrpGl | |
| 586 | GTCTGCCACTTGGAACGAGTATCCCAAGCCTCTCCAGGTTAACAT | |
| | nSerAlaThrTrpLysGluTyrProLySProLeuGlnValAsnIl | |
| 631 | CACTCTGTCTTGGAGCAAAACCATTGAGCTAACAGACAACATAGT | |
| | eThrLeuSerTrpSerLysThrIleGluLeuThrAspAsnIleVa | |
| 676 | TATTACCTTTGAATCTGGGCGTCCAGACCAAATGATCCTGGAGAA | |
| | lIleThrPheGluSerGlyArgProAspGlnMetIleLeuGluLy | |
| 721 | GTCTCTCGATTATGGACGAACATGGCAGCCCTATCAGTATTATGC | |
| | sSerLeuAspTyrGlyArgThrTrpGlnProTyrGlnTyrTyrAl | |
| 766 | CACAGACTGCTTAGATGCTTTTCACATGGATCCTAAATCCGTGAA | |
| | aThrAspCysLeuAspAlaPheHisMetAspProLysSerValLy | |
| 811 | GGATTTATCACACCATACGGTCTTAGAAATCATTTGCACAGAAGA | |
| | sAspLeuSerGlnHisThrValLeuGluIleIleCysThrGluGl | |
| 856 | GTACTCAACAGGGTATACAACAAATAGCAAAATAATCCACTTTGA | |
| | uTyrSerThrGlyTyrThrThrAsnSerLysIleIleHisPheGl | |
| 901 | AATCAAAGACAGGTTCGCGTTTTTTGCTGGACCTCGCCTACGCAA | |
| | uIleLysAspArgPheAlaPhePheAlaGlyProArgLeuArgAs | |
| 946 | TATGGCTTCCCTCTACGGACAGCTGGATACAACCAAGAAACTCAG | |
| | nMetAlaSerLeuTyrGlyGlnLeuAspThrThrLysLysLeuAr | |
| 991 | AGATTTCTTTACAGTCACAGACCTGAGGATAAGGCTGTTAAGACC | |
| | gAspPhePheThrValThrAspLeuArgIleArgLeuLeuArgPr | |
| 1036 | AGCCGTTGGGGAAATATTTGTAGATGAGCTACACTTGGCACGCTA | |
| | oAlaValGlyGluIlePheValAspGluLeuHisLeuAlaArgTy | |
| 1081 | CTTTTACGCGATCTCAGACATAAAGGTCCGAGGAAGGTGCAAGTG | |
| | rPheTyrAlaIleSerAspIleLysValArgGlyArgCysLysCy | |
| 1126 | TAATCTCCATGCCACTGTATGTGTGTATGACAACAGCAAATTGAC | |
| | sAsnLeuHisAlaThrValCysValTyrAspAsnSerLysLeuTh | |
| 1171 | ATGCGAATGTGAGCACAACACTACAGGTCCAGACTGTGGGAAATG | |
| | rCysGluCysGluHisAsnThrThrGlyProAspCysGlyLysCy | |
| 1216 | CAAGAAGAATTATCAGGGCCGACCTTGGAGTCCAGCCTCCTATCT | |
| | sLysLysAsnTyrGlnGlyArgProTrpSerProGlySerTyrLe | |
| 1261 | CCCCATCCCCAAAGGCACTGCAAATACCTGTATCCCCAGTATTTC | |
| | uProIleProLysGlyThrAlaAsnThrCysIleProSerIleSe | |
| 1306 | CAGTATTGGTAATCCTCCAAAGTTTAATAGGATATGCCCGAATAT | |
| | rSerIleGlyAsnProProLysPheAsnArgIleTrpProAsnIl | |
| 1351 | TTCTTCCCTTGAGGTTTCTAACCCAAAACAAGTTGCTCCCAAATT | |
| | eSerSerLeuGluValSerAsnProLysGlnValAlaProLysLe | |
| 1396 | AGCTTTGTCAACAGTTTCTTCTGTTCAAGTTGCAAACCACAAGAG | |
| | uAlaLeuSerThrValSerSerValGlnValAlaAsnHisLysAr | |
| 1441 | AGCGAATGTCTGCGACAACGAGCTCCTGCACTGCCAGAACGGAGG | |
| | gAlaAsnValCysAspAsnGluLeuLeuHisCysGlnAsnGlyGl | |
| 1486 | GACGTGCCACAACAACGTGCGCTGCCTGTGCCCGGCCGCATACAC | |
| | yThrCysHisAsnAsnValArgCysLeuCysProAlaAlaTyrTh | |
| 1531 | GGGCATCCTCTGCGAGAAGCTGCGCTGCGAGGAGGCTGGCAGCTG | |
| | rGlyIleLeuCysGluLysLeuArgCysGluGluAlaGlySerCy | |
| 1576 | CGGCTCCGACTCTGGCCAGGGCGCGCCCCCGCACGGCTCCCCAGC | |
| | sGlySerAspSerGlyGlnGlyAlaProProHisGlySerProAl | |
| 1621 | GCTGCTGCTCCTGACCACGCTGCTGGGAACCGCCAGCCCCCTGGT | |
| | aLeuLeuLeuLeuThrThrLeuLeuGlyThrAlaSerProLeuVa | |
| 1666 | GTTCTAGGTCTCACCTCCAGCCACACCGGACGGGCCTGTGCCGTG | |
| | lPhe | |
| 1711 | GGGAAGCAGACACAACCCAAACATTTGCTACTAACATACGAAACA | |
| 1756 | CACACATACAGACACCCCCACTCAGACAGTGTACAAACTAAGAAG | |
| 1801 | GCCTAACTGAACTAAGCCATATTTATCACCCGTGCACAGCACATC | |
| 1846 | CGAGTCAGGACTGTTAATTTCTGACTCCAGAGGAGTTGGCAGCTG | |
| 1891 | TTCATATTATCACTGCAA | |

Construction of the mammalian expression vector pCEP4/Sec. The oligonucleotide primers, pSec-V5-His Forward (CTCGTC CTCGAG GGT AAG CCT ATC CCT AAC) (SEQ ID NO: 52) and the pSec-V5-His Reverse (CTCGTC GGGCCC CTGATCAGCGGGTTTAAAC) (SEQ ID NO: 53), were designed to amplify a fragment from the pcDNA3.1-V5His (Invitrogen, Carlsbad, Calif.) expression vector. The PCR product was digested with XhoI and ApaI and ligated into the XhoI/ApaI digested pSecTag2 B vector (Invitrogen, Carlsbad Calif.). The correct structure of the resulting vector, pSecVS5His, was verified by DNA sequence analysis. The vector pSecV5His was digested with PmeI and NheI, and the PmeI-NheI fragment was ligated into the BamHI/Klenow and NheI treated vector pCEP4 (Invitrogen, Carlsbad, Calif.). The resulting vector was named as pCEP4/Sec.

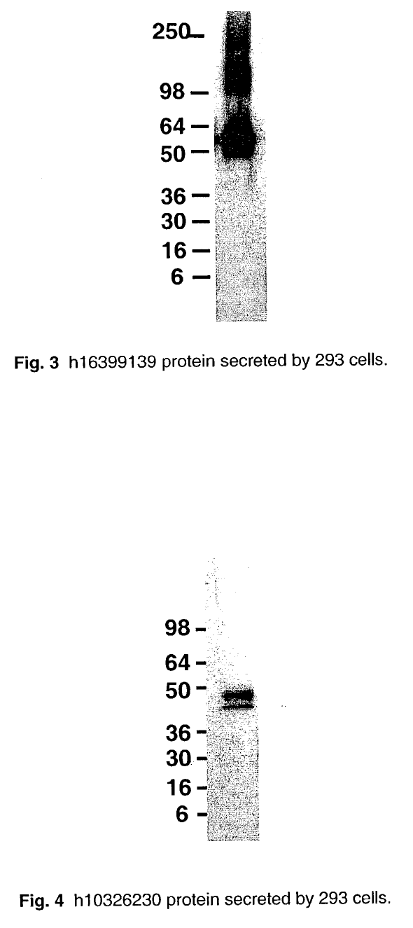

Expression of h16399139 in human embryonic kidney 293 cells. A 1.5 kb BamHI-XhoI fragment containing the h16399139 sequence was isolated from pCR2.1-16399139 and subcloned into BamHI-XhoI digested pCEP4/Sec to generate expression vector pCEP4/Sec-16399139. The pCEP4/Sec-16399139 vector was transfected into 293 cells using the LipofectaminePlus reagent following the manufacturer's instructions (Gibco/BRL). The cell pellet and supernatant were harvested 72 hours after transfection and examined for h16399139 expression by Western blotting (reducing conditions) with an anti-V5 antibody. FIG. 3 shows that h16399139 is expressed as a 55 kDa protein secreted by 293 cells. Several higher molecular bands were also visible, which may represent non-reduced proteins.

SEC4

A SEC4 nucleic acid and polypeptide according to the invention includes the nucleic acid and encoded polypeptide sequence of 3440544.0.81.

The disclosed SEC 4 polypeptide is predicted by the PSORT program to localize in the plasma membrane with a certainty of 0.6000.

The disclosed SEC4 polypeptide has 95 of 225 residues (42%) identical to, and 95 of 225 residues positive with, the 287 residue Y25C1A 7B protein from Caenorhabditis elegans (GenBank Accession No: AAD12839). In addition, the SEC4 polypeptide has 44 of 174 residues (25%), identical to, and 82 of 174 residues (47%) positive with, the 551 residue human polyspecific oraganic cation transporter (GenBank Accession No: O14546).

The 3440544.0.81 nucleic acid and corresponding polypeptide has the following sequence:

| | | |
|---|---|---|
| 1 | CTGGACCGAAACCGGCGCGGANAACTGAGGCCCGAGCCTTCTGGG | (SEQ ID NO:7) |
| 46 | GACCCGGGGGACGCCTAACCCCGCGAGACCCCTGCAAATTTTTTT | |
| 91 | CCTCATAATTGGGAGAAGACTCACTGGCCGAATGGCAGCAGTAGA | |
| | MetAlaAlaValAs | (SEQ ID NO:8) |
| 136 | TGATTTGCAATTTGAAGAATTTGGCAATGCAGCCACTTCTCTGAC | |
| | pAspLeuGlnPheGluGluPheGlyAsnAlaAlaThrSerLeuTh | |
| 181 | AGCAAACCCAGATCCCACCACAGTAAACATTGAGGTTCCTGGTGA | |
| | rAlaAsnProAspAlaThrThrValAsnIleGluValProGlyGl | |
| 226 | AACCCCAAAACATCAGCCAGGTTCCCCAAGAGGCTCAGGAAGAGA | |
| | uThrProLysHisGlnProGlySerProArgGlySerGlyArgGl | |
| 271 | AGAAGATGATGAGTTACTGGGAAATGATGACTCTGACAAAACTGA | |
| | uGluAspAspGluLeuLeuGlyAsnAspAspSerAspLysThrGl | |
| 316 | GTTACTTGCTGGACAGAAGAAAAGCTCCCCCTTTTGGACATTTGA | |
| | uLeuLeuAlaGlyGlnLysLysSerSerProPheTrpThrPheGl | |
| 361 | ATACTACCAAACATTCTTTGATGTGGACACCTACCTGGTCTTTGA | |
| | uTyrTyrGlnThrPhePheAspValAspThrTyrLeuValPheAs | |
| 406 | CAGAATTAAAGGATCTCTTTTGCCAATACCCGGGAAAAACTTTGT | |
| | pArgIleLysGlySerLeuLeuProIleProGlyLysAsnPheVa | |
| 451 | GAGGTTATATATCCGCAGCAATCCAGATCTCTATGGCCCCTTTTG | |
| | lArgLeuTyrIleArgSerAsnProAspLeuTyrGlyProPheTr | |
| 496 | GATATGTGCCACGTTGGTCTTTGCCATAGCAATTAGTGGGAATCT | |
| | pIleCysAlaThrLeuValPheAlaIleAlaIleSerGlyAsnLe | |
| 541 | TTCCAACTTCTTGATCCATCTGGGAGAGAAGACGTACCATTATGT | |
| | uSerAsnPheLeuIleHisLeuGlyGluLysThrTyrHisTyrVa | |
| 586 | GCCCGAATTCCGAAAAGTGTCCATAGCAGCTACCATCATCTATGC | |
| | lProGluPheArgLysValSerIleAlaAlaThrIleIleTyrAl | |
| 631 | CTATGCCTCGCTGGTTCCTCTTGCACTCTGGGGTTTCCTCATGTG | |
| | aTyrAlaTrpLeuValProLeuAlaLeuTrpGlyPheLeuMetTr | |
| 676 | GAGAAACAGCAAAGTTATGAACATCGTCTCCTATTCATTTCTGGA | |
| | pArgAsnSerLysValMetAsnIleValSerTyrSerPheLeuGl | |
| 721 | GATTGTGTGTGTCTATGGATATTCCCTCTTCATTTATATCCCCAC | |
| | uIleValCysValTyrGlyTyrSerLeuPheIleTyrIleProTh | |
| 766 | CGCAATACTGTGGATTATCCCCCAGAAAGCTGTTCGTTGGATTCT | |
| | rAlaIleLeuTrpIleIleProGlnLysAlaValArgTrpIleLe | |
| 811 | AGTCATGATTGCCCTGGGCATCTCAGGATCTCTCTTGGCAATGAC | |
| | uValMetIleAlaLeuGlyIleSerGlySerLeuLeuAlaMetTh | |
| 856 | ATTTTGGCCAGCTGTTCGTGAGGATAACCGACGCGTTGCATTGGC | |
| | rPheTrpProAlaValArgGluAspAsnArgArgValAlaLeuAl | |
| 901 | CACAATTGTGACAATTGTGTTGCTCCATATGCTGCTTTCTGTGGG | |
| | aThrIleValThrIleValLeuLeuHisMetLeuLeuSerValGl | |
| 946 | CTGCTTGGCATACTTTTTTGATGCACCAGAGATGGACCATCTCCC | |
| | yCysLeuAlaTyrPhePheAspAlaProGluMetAspHisLeuPr | |
| 991 | AACAACTACAGCTACTCCAAACCAAACAGTTGCTGCAGCCAAGTC | |
| | oThrThrThrAlaThrProAsnGlnThrValAlaAlaAlaLysSe | |
| 1036 | CAGCTAATGAGGAAATTCTCTTTTGTTTTTTGGAGCATGGTTCTT | |
| | rSer | |
| 1081 | TGGGAAGTGGCATCCACTGCAGGAAAGCAGAATGAGCAGAGCCAG | |
| 1126 | CAGAACTGATGGAGTGGCACAAATTCCCAGTGTCTGGATGGTGCC | |
| 1171 | ACACTGGCGCCTAATCACCCGTTTAACAAGCAGAAATTAAATGTT | |
| 1216 | GCTCAGCACATGTGTCTTTCAGCTCTTCCTTTTCACCCATGGATG | |
| 1261 | ATCATTGCGAGCATGCGCTGATTGGACTGAAATGCCGGGGAATAG | |
| 1306 | GTTAGGCATGCTCAGTGCCGTCCCTTTGCCACCACAGTCAAATGA | |
| 1351 | CATGCTTCACTGTGGTACCTTAATACCTGAAATAGAACCATGCAA | |
| 1396 | AATTCTGATGTCCTCTCTCTGAATTATGTACAGACTACCTGGGGG | |
| 1441 | ATCCTCTTCTCTCCAAATGTTAGCCATCCTGAACTAGCCGAACAG | |
| 1486 | TAGAAACTTTGGTGGGGATTAACCGGGAGCTTGAAAATTTGTCTT | |
| 1531 | TGGTAACCTGATACTGGACAGCTGAACTGAATGGCTGCAAAATAA | |
| 1576 | ATACCTCACATGAAAAAAAAAA | |

SEC5

A SEC5 nucleic acid and polypeptide according to the invention includes the nucleic acid and encoded polypeptide sequence of 3581980.0.30.

The disclosed SEC5 polypeptide is predicted by the PSORT program to localize in the cytoplasm with a certainty of 0.4500. The disclosed SEC5 polypeptide has 27 of 90 residues (30%) identical to, and 38 of 90 residues (42%) positive with, the 1056 residue human KIAA0430 protein.

SEC5 nucleic acids are primarily expressed in adipose, brain and bladder tissue.

The 3581980.0.30 nucleic acid and encoded polypeptide according to the invention has the following sequence:

| | | |
|---|---|---|
| 1 | CAGAATATCAGGAAGCTCTTGAGATCAGGAGGAAGCCCCATTTCC | (SEQ ID NO:9) |
| 46 | TGATGTATAATTATCGGCAACAAAGCTGGCATCTACGAGACCCCA | |
| 91 | TCTAACTGTTGTGCTATTTCTTAATTGCTTTACAACCCAGAGGCA | |
| 136 | AAGGGACTGTGATTAATGCCCTTCTCAAAAACTCCAGCACCTGGC | |
| 181 | ACTTAGTGGATGCTAAATAAATATTCATTGAGTTGATTTTGTTGA | |
| 226 | GTCATGGCCCAGGAATGGGATGGTCAATCTGGAAAGTAGTGAGAT | |
| 271 | CCCCATCAAAGAGAGAAAAAACAGAGAGTGACGACTACATGACAG | |
| 316 | AAAGGCTACAGAGGCAATTTCAATATGAGTTCTGAATTGCATTAG | |
| 361 | ATGTCTTTTAAAATATGTGCCAGACTTGAGGTTTTACAGTCACGT | |
| 406 | GGCTCAGGAGACACTATAGTAAATCTAAAACTATTTTATTAACAA | |
| 451 | CAACAACAACAACAACAACAACAAAAACTAAGGGCCTTGGAATTC | |
| 496 | TGGAAGTTGAGTCACTTGCCCAGGGAGGACCAAAAACATCTTGAA | |
| 541 | GATGATCATCCCTTTCTAAATGAGCCAGAGAATACCATGCTACTC | |
| 586 | ACCCACTCAGACCATGTGGCATTAGATTCATTTGACATAAAACAA | |
| 631 | AAAATAATGCCCCTATCTTAGCTTGGGCTTCCCCAAAAGCACAAC | |
| 676 | CCAAGAAAAGGGCTGGGAGTGGTTCCTTTGGAAGGTAATTCAGCG | |
| 721 | AAGCAAGAGTGAAGAAGTGAGCCGGTAGAGGAAGACAGGCAGACA | |
| 766 | AGTGCGTCAATGTGAGGGTGTGCTGTGGAGAACAGGGGCTCGATT | |
| 811 | CTCCTGAGACCACATGAGATACTGAAAAATCTTCCATAATTGTCT | |
| 856 | GCACGAAAGGCAAAAGACTGGCACATTTATCCATGTCTCCTCAGA | |
| 901 | CAATGATTGTGCTGGCACCAGGGTCGCTCTCTGCCCTGCACTTGT | |
| 946 | GGAATGAGTTTCCCTGCACACAGTGATGTGAGGTCAGTCTGCAAG | |
| | MetSerLeuProAlaHisSerAspValArgSerValCysLys | (SEQ ID NO:10) |
| 991 | TCTGAGCTGCCCCAGCCAGTCCTAGCCAAAAGGAGATATGGGATG | |
| | SerGluLeuProGlnProValLeuAlaLysArgArgTyrGlyMet | |
| 1036 | AGCGCAGGAGACATCGGCACCACAGGCAGCTGCAGCCCTAAACTC | |
| | SerAlaGlyAspMetGlyThrThrGlySerCysSerProLysLeu | |
| 1081 | ATCACTCCATGTCCTCCAGATCTCCAAAGTCACAGCCCTGTGTGC | |
| | IleThrProCysArgProAspLeuGlnSerHisSerProValCys | |
| 1126 | CAGTCTCCAAGCTGTTGCTTCTGTGATCTTCCTGAGACTGTCTTC | |
| | GlnSerProSerCysCysPheCysAspLeuProGluThrValPhe | |
| 1171 | CTTCCTCAAAACCCACAGGACTACAAGACAAGTCTAAAACCCTTC | |
| | LeuAlaGlnAsnProGlnAspTyrLysThrSerLeuLysProPhe | |
| 1216 | TCCATGGGATCCCCCACTCCACTGGTCCATCTCAACCTATGGCTG | |
| | SerMetGlySerProThrProLeuValHisLeuAsnLeuTrpLeu | |
| 1261 | CTCCTCCTCCAATCAGAACCTTCCCCTTGCACTCCAATGAGTCAC | |
| | LeuLeuLeuGlnSerGluProSerProCysThrProMetSerHis | |
| 1306 | CTGCCATTCCTTACTCATGTCCTTCCCTAAAGGCCTTTGTGCTCT | |
| | LeuProPheLeuThrHisValLeuPro | |
| 1351 | GGCGCAAAGAGCTCTGTCTGGAACACCATTTAGTTTCATTCCCAT | |
| 1396 | CCATCAAACTCCATCCCCTCCTCGACAGCCCAGCTGAAACATTTC | |
| 1441 | TTCCAGGGAATTTGCTCCCTTGTGAGTATACTTACTGAGTTGCAT | |
| 1486 | TGTAATTTGTGTAAGTGTTGGTGTCCTCACAAAAAAGGAGCTTCT | |
| 1531 | TTAAGGTCAGGGATAAAGTTGTAATCTAACTTCAGGGCCATCCAT | |
| 1576 | AAAGGAGATATTCAGTGAAAGGTGGCTGAGTAAATGAATGGATGA | |
| 1621 | CTCCAGAAAACTTCTCCCTTCAAGGCCTCAGCTTCTTCCACTTTA | |
| 1666 | GAATGAAGAAGTGGCAGGAGCTGAATTAGAGTTTCCTGCAGCATT | |
| 1711 | TTCTCAGAAGTCCTAGCACTGCCAGATGCCTCTAAAGAACATATT | |
| 1756 | CTGAGGCCTAATGGGTTTGAGAGATGC | |

SEC6

A SEC6 nucleic acid and polypeptide according to the invention includes the nucleic acid and encoded polypeptide sequence of 4418354.0.6.

The polypeptide of SEC6 protein is predicted by the PSORT program to localize in the cytoplasm with a certainty of 0.6500. The disclosed SEC6 polypeptide has 289 of 364 residues (79%) identical to, and 301 of 364 residues (82%) positive with, a region of the 755 residue rat protein of GenBank Accession No: Q62825. Also there is a 100% identity over 74 residues to the 471 human homologue (SPTREMBL:O60645).

In addition, the nucleotide sequence and polypeptide sequence of the disclosed SEC6 has similarity to a vesicle transport protein disclosed in U.S. Pat. No. 5,989,818 (“the '818 patent”). The disclosed SEC6 nucleic acid sequence has 934 of 985 bp (94%) identical to a 2464 bp cDNA disclosed in the '818 patent. The disclosed SEC6 polypeptide has 301 of 364 residues (82%) identical to, and 306 of 364 residues (84%) positive with, the corresponding polypeptide disclosed in the '818 patent.

Based on homology to a vesicle transport protein, a SEC6 polypeptide of the invention is expected to exhibit cytostatic, immunomodulatory and neuroprotective activity. The SEC6 polynucleotides and the protein encoded therein can be used for the treatment of cancer, neurodegenerative and immune disorders.

SEC6 nuclic acid is expressed in most tissue, particaularly hig expression is found in certain cancers, e.g. colon cancer, large cell and squamous lung cancer, breast cancer and melenoma.

The 4418354.0.6 nucleic acid and corresponding polypeptide according to the invention has the following sequence:

| | | |
|---|---|---|
| 1 | AAAAAAAAAAAAAAAAAAAAAAGCGGCCGCTCAATTCTAGGCGGC | (SEQ ID NO:11) |
| 46 | GGCGGCGGCGGCGGCGCCCGCGGCGGCGTAGCCGTAGAGGTGCAC | |
| 91 | AGAGAACACCCCTAGCATGAACAGTGTCAGGATTCCACCAGCTTT | |
| 136 | TTCACCATGAAGGAGACAGACCGGGAGGCCGTTGCGACAGCAGGT | |
| | MetLysGluThrAspArgGluAlaValAlaThrAlaGly | (SEQ ID NO:12) |
| 181 | GCAAAGGGTTCCTGCGATGCTCCAGCGCCCGGACCAGCTGGACAA | |
| | AlaLysGlyCysTrpAspAlaProAlaProGlyProAlaGlyGln | |
| 226 | GGTGGAGCAGTATCGCAGGACAGAAGCGCGGAAGAAGGCCTCCGT | |
| | GlyGlyAlaValSerGlnGluArgSerAlaGluGluGlyLeuArg | |
| 271 | GGAGGCCANGAATTTGAAGAGAGCGGATCTGAAAGCTCAGGTGCC | |
| | GlyGly---GluPheGluGluSerGlySerGluSerSerGlyAla | |
| 316 | CGATTCTGTCCTGTGGGTCAGCCGTCCTGGGGCCAAGTTGTGGTG | |
| | ArgPheCysProValGlyGlnProSerTrpGlyGlnValValVal | |
| 361 | CTGCGCACAGGCCTCAGCCAGCTCCACAACGCCCTGAATGACGTC | |
| | LeuArgThrGlyLeuSerGlnLeuHisAsnAlaLeuAsnAspVal | |
| 406 | AAAGACATCCAGCAGTCGCTGGCAGACGTCACCAAGGACTGGAGG | |
| | LysAspIleGlnGlnSerLeuAlaAspValSerLysAspTrpArg | |
| 451 | CAGAGCATCAACACCATTGAGAGCCTCAAGGACGTCAAAGACGCC | |
| | GlnSerIleAsnThrIleGluSerLeuLysAspValLysAspAla | |
| 496 | GTGGTGCAGCACAGCCAGCTCGCCGCAGCCGTGGAGAACCTCAAG | |
| | ValValGlnHisSerGlnLeuAlaAlaAlaValGluAsnLeuLys | |
| 541 | AACATCTTCTCAGTGCCTGAGATTNTGAGGGAGACCCAGGACCTA | |
| | AsnIlePheSerVaiProGluIle---ArgGluThrGlnAspLeu | |
| 586 | ATTGAACAAGGGGCACTCCTGCAAGCCCACCGGGAAGCTGATGGA | |
| | IleGluGlnGlyAlaLeuLeuGlnAlaHisArgGluAlaAspGly | |
| 631 | CCTGGAGTGCTCCCGGGACGGCTGATGTACGAGCAGTACCGCATG | |
| | ProGlyValLeuProGlyArgLeuMetTyrGluGlnTyrArgMet | |
| 676 | GACAGTGGGAACACGCGTGACATGACCCTCATCCATGGCTACTTT | |
| | AspSerGlyAsnThrArgAspMetThrLeuIleHisGlyTyrPhe | |
| 721 | GGCAGCACGCAGGGGCTCTCTGATGAGCTGGCTAAGCAGCTGTGG | |
| | GlySerThrGlnGlyLeuSerAspGluLeuAlaLysGlnLeuTrp | |
| 766 | ATGGTGCTGCAGAGGTCACTGGTCACTGTCCGCCGTGACCCCACC | |
| | MetValLeuGlnArgSerLeuValThrValArgArgAspProThr | |
| 811 | TTGCTGGTCTCAGTTGTCAGGATCATTGAAAGGGAAGAGAAAATT | |
| | LeuLeuValSerValValArgIleIleGluArgGluGluLysIle | |
| 856 | GACAGGCGCATACTTGACCGGAAAAAGCAAACTGGCTTTGTTCCT | |
| | AspArgArgIleLeuAspArgLysLysGlnThrGlyPheValPro | |
| 901 | CCTGGGACGCCCAAGAATTGGAAGGAGAAAATGTTCACCATCTTG | |
| | ProGlyArgProLysAsnTrpLysGluLysMetPheThrIleLeu | |
| 946 | GAGAGGACTGTGACCACCAGAATTGAGGGCACACAGGCAGATACC | |
| | GluArgThrValThrThrArgIleGluGlyThrGlnAlaAspThr | |
| 991 | AGAGAGTCTGACAAGATGTGGCTTGTCCGCCACCTGGAAATTATA | |
| | ArgGluSerAspLysMetTrpLeuValArgHisLeuGluIleIle | |
| 1036 | AGGAAGTACGTCCTGGATGACCTCATTGTCGCCAAAAACCTGATG | |
| | ArgLysTyrValLeuAspAspLeuIleValAlaLysAsnLeuMet | |
| 1081 | GTTCAGTGCTTTCCTCCCCACTATGAGATCTTTAAGAACCTCCTG | |
| | ValGlnCysPheProProHisTyrGluIlePheLysAsnLeuLeu | |
| 1126 | AACATGTACCACCAAGCCCTGAGCACGCGGATGCAGGACCTCGCA | |
| | AsnMetTyrHisGlnAlaLeuSerThrArgMetGlnAspLeuAla | |
| 1171 | TCGGAAGACCTGGAAGCCAATGAGATCGTGAGCCTCTTGACGTGG | |
| | SerGluAspLeuGluAlaAsnGluIleValSerLeuLeuThrTrp | |
| 1216 | GTCTTAAACACCTACACAAGGTAAAGCTAACCTGGCGCCTGTGTT | |
| | ValLeuAsnThrTyrThrArg | |
| 1261 | GGCTC | |

SEC7

A SEC7 nucleic acid nucleic acid and polypeptide according to the invention includes the nucleic acid and encoded polypeptide sequence of 4418354.0.9. SEC7 is identical at its 5′ end to SEC6 (see above), but is considerably extended at the 3′ end. The SEC7 polypeptide is predicted by the PSORT program to localize in the cytoplasm with a certainty of 0.6500.

The polypeptide encoded by clone 4418354.0.9 has 528 of 620 residues (85%) identical to, and 546 of 620 residues (88%) positive with, a fragment of the 755 residue rat protein (ACC:Q62825). It also has a 100% identity to 330 residues in the 471 residue a human homolog (SPTREMBL:060645). The protein of clone 4418354.0.9 also shows 555 of 620 residues (89%) identical to, and 560 of 620 residues (90%) positive with the human protein vesicle transport protein having 754 amino acid residues disclosed in U.S. Pat. No. 5,989,818. Based on this homology, a SEC7 according to the invention is expected to exhibit cytostatic, immunomodulatory and neuroprotective activity. The polynucleotides and the protein encoded therein can be used for the treatment of cancer, neurodegenerative and immune disorders.

The 4418354.0.9 nucleic acid and encoded polypeptide have the following sequences:

| | | |
|---|---|---|
| 1 | AAAAAAAAAAAAAAAAAAAAAAGCGGCCGCTGAATTCTACGCGGC | (SEQ ID NO:13) |
| 46 | GGCGGCGGCGGCGGCGGCGGCGGCGGCGTAGCCGTAGACGTGCAC | |
| 91 | AGAGAACACCCCTAGCATGAACAGTGTGAGCATTCCACCAGCTTT | |
| 136 | TTCACCATGAAGGAGACAGACCGCGAGGCCGTTGCGACAGCAGGT | |
| | MetLysGluThrAspArgGluAlaValAlaThrAlaGly | (SEQ ID NO:14) |
| 181 | GCAAAGGGTTGCTGCGATGCTCCAGCGCCCGGACCAGCTGGACAA | |
| | AlaLysGlyCysTrpAspAlaProAlaProGlyProAlaGlyGln | |
| 226 | GGTGGAGCAGTATCGCAGGAGAGAAGCGCGGAAGAAGGCCTCCGT | |
| | GlyGlyAlaValSerGlnGluArgSerAlaGluGluGlyLeuArg | |
| 271 | GGAGGCCANGAATTTGAAGAGAGCGGATCTGAAAGCTCAGGTGCC | |
| | GlyGly---GluPheGluGluSerGlySerGluSerSerGlyAla | |
| 316 | GGATTCTGTCCTGTGGGTCAGCCGTCCTGGGGCCAAGTTGTGGTG | |
| | ArgPheCysProValGlyGlnProSerTrpGlyGlnValValVal | |
| 361 | CTGCGCACAGGCCTCAGCCAGCTCCACAACGCCCTGAATGACGTC | |
| | LeuArgThrGlyLeuSerGlnLeuHisAsnAlaLeuAsnAspVal | |
| 406 | AAAGACATCCAGCAGTCGCTGGCAGACGTCAGCAAGGACTGGAGG | |
| | LysAspIleGlnGlnSerLeuAlaAspValSerLysAspTrpArg | |
| 451 | CAGAGCATCAACACCATTGAGAGCCTCAAGGACGTCAAAGACGCC | |
| | GlnSerIleAsnThrIleGluSerLeuLysAspValLysAspAla | |
| 496 | GTGGTGCAGCACAGCCAGCTCGCCGCAGCCGTGGAGAACCTCAAG | |
| | ValValGlnHisSerGlnLeuAlaAlaAlaValGluAsnLeuLys | |
| 541 | AACATCTTCTCAGTGCCTGAGATTNTGAGGGAGACCCAGCACCTA | |
| | AsnIlePheSerValProGluIle---ArgGluThrGlnAspLeu | |
| 586 | ATTGAACAAGGGGCACTCCTGCAAGCCCACCGGGAAGCTGATGGA | |
| | IleGluGlnGlyAlaLeuLeuGlnAlaHisArgGluAlaAspGly | |
| 631 | CCTGGAGTGCTCCCGGGACGGCTGATGTACGAGCAGTACCGCATG | |
| | ProGlyValLeuProGlyArgLeuMetTyrGluGlnTyrArgMet | |
| 676 | GACAGTGGGAACACGCGTGACATGACCCTCATCCATGGCTACTTT | |
| | AspSerGlyAsnThrArgAspMetThrLeuIleHisGlyTyrPhe | |
| 721 | GGCAGCACGCAGGGGCTCTCTGATGAGCTGGCTAAGCAGCTGTGG | |
| | GlySerThrGlnGlyLeuSerAspGluLeuAlaLysGlnLeuTrp | |
| 766 | ATGGTGCTGCAGAGGTCACTGGTCACTGTCCGCCGTGACCCCACC | |
| | MetValLeuGlnArgSerLeuValThrValArgArgAspProThr | |
| 811 | TTGCTGGTCTCAGTTGTCAGGATCATTGAAAGGGAAGAGAAAATT | |
| | LeuLeuValSerValValArgIleIleGluArgGluGluLysIle | |
| 856 | GACAGGCGCATACTTGACCGGAAAAAGCAAACTGGCTTTGTTCCT | |
| | AspArgArgIleLeuAspArgLysLysGlnThrGlyPheValPro | |
| 901 | CCTGGGAGGCCCAAGAATTGGAAGGAGAAAATGTTCACCATCTTG | |
| | ProGlyArgProLysAsnTrpLysGluLysMetPheThrIleLeu | |
| 946 | GAGAGGACTGTGACCACCAGAATTGAGGGCACACAGGCAGATACC | |
| | GluArgThrValThrThrArgIleGluGlyThrGlnAlaAspThr | |
| 991 | AGAGAGTCTGACAAGATGTGGCTTGTCCGCCACCTGGAAATTATA | |
| | ArgGluSerAspLysMetTrpLeuValArgHisLeuGluIleIle | |
| 1036 | AGGAAGTACGTCCTGGATGACCTCATTGTCGCCAAAAACCTGATG | |
| | ArgLysTyrValLeuAspAspLeuIleValAlaLysAsnLeuMet | |
| 1081 | GTTCAGTGCTTTCCTCCCCACTATGAGATCTTTAAGAACCTCCTG | |
| | ValGlnCysPheProProHisTyrGluIlePheLysAsnLeuLeu | |
| 1126 | AACATGTACCACCAAGCCCTGAGCACGCGGATGCAGGACCTCGCA | |
| | AsnMetTyrHisGlnAlaLeuSerThrArgMetGlnAspLeuAla | |
| 1171 | TCGGAAGACCTGGAAGCCAATGAGATCGTGAGCCTCTTGACCTGG | |
| | SerGluAspLeuGluAlaAsnGluIleValSerLeuLeuThrTrp | |
| 1216 | GTCTTAAACACCTACACAAGTACTGAGATGATGAGGAACGTGGAG | |
| | ValLeuAsnThrTyrThrSerThrGluMetMetArgAsnValGlu | |
| 1261 | CTGGCCCCGGAAGTGGATGTCGGCACCCTGGAGCCATTGCTTTCT | |
| | LeuAlaProGluValAspValGlyThrLeuGluProLeuLeuSer | |
| 1306 | CCACACGTGGTCTCTGAGCTGCTTGACACGTACATGTCCACGCTC | |
| | ProHisValValSerGluLeuLeuAspThrTyrMetSerThrLeu | |
| 1351 | ACTTCAAACATCATCGCCTGGCTGCGGAAAGCGCTGGAGACAGAC | |
| | ThrSerAsnIleIleAlaTrpLeuArgLysAlaLeuGluThrAsp | |
| 1396 | AACAAAGACTGGGTCAAAGAGACAGAGCCAGAAGCCGACCAGGAC | |
| | LysLysAspTrpValLysGluThrGluProGluAlaAspGlnAsp | |
| 1441 | GGGTACTACCAGACCACACTCCCTGCCATTGTCTTCCAGATGTTT | |
| | GlyTyrTyrGlnThrThrLeuProAlaIleValPheGlnMetPhe | |
| 1486 | GAACAGAATCTTCAAGTTGCTGCTCAGATAAGTGAAGATTTGAAA | |
| | GluGlnAsnLeuGlnValAlaAlaGlnIleSerGluAspLeuLys | |
| 1531 | ACAAAGGTACTAGTTTTATGTCTTCAGCAGATGAATTCTTTCCTA | |
| | ThrLysValLeuValLeuCysLeuGlnGlnMetAsnSerPheLeu | |
| 1576 | AGCAGATATAAAGATGAAGCGCAGCTGTATAAAGAAGAGCACCTG | |
| | SerArgTyrLysAspGluAlaGlnLeuTyrLysGluGluHisLeu | |
| 1621 | AGGAATCGGCAGCACCCTCACTGCTACGTTCAGTACATGATCGCC | |
| | ArgAsnArgGlnHisProHisCysTyrValGlnTyrMetIleAla | |
| 1666 | ATCATCAACAACTGCCAGACCTTCAAGGAATCCATAGTCAGTTTA | |
| | IleIleAsnAsnCysGlnThrPheLysGluSerIleValSerLeu | |
| 1711 | AAAAGAAAGTATTTAAAGAATGAAGTGGAAGAGGGTGTGTCTCCG | |
| | LysArgLysTyrLeuLysAsnGluValGluGluGlyValSerPro | |
| 1756 | AGCCAGCCCAGCATGGACGGGATTTTAGACGCCATCGCGAAGGAG | |
| | SerGlnProSerMetAspGlyIleLeuAspAlaIleAlaLysGlu | |
| 1801 | GGCTGCAGCCGTTTGCTCGAGGAGGTCTTCCTGGACCTGGAGCAA | |
| | GlyCysSerGlyLeuLeuGluGluValPheLeuAspLeuGluGln | |
| 1846 | CATCTGAATGAATTGATGACGAAGAAGTGGCTATTAGGGTCAAAC | |
| | HisLeuAsnGluLeuMetThrLysLysTrpLeuLeuGlySerAsn | |
| 1891 | GCTGTAGACATTATCTGTGTCACCGTGGAAGACTATTTCAACGAT | |
| | AlaValAspIleIleCysValThrValGluAspTyrPheAsnAsp | |
| 1936 | TTTGCCAAAATTAAAAAGCCGTATAAGAAGAGGATGACGGCCGAG | |
| | PheAlaLysIleLysLysProTyrLysLysArgMetThrAlaGlu | |
| 1981 | GCGCACCGGCGCGTGGTGGTTGGAGTACCTGCCGGCGGTCATGCA | |
| | AlaHisArgArgValValValGlyValProAlaGlyGlyHisAla | |
| 2026 | GAAGCGCATTTCCTTCCGGAGCCCGGAGGAGCGCAAGGAGGGTGC | |
| | GluAlaHisPheLeuProGluProGlyGlyAlaGlnGlyGlyCys | |
| 2071 | CGAGAAGATGGTTAGGGAGGCAGAGCAGCGGCGCTTCCTGTTCCG | |
| | ArgGluAspGly | |
| 2116 | GAAGCTGGCGTCCGGTTTCGGGGAAGACGTGGACGGATACTGCGA | |
| 2161 | CACCATCGTGGCTGTGGCCGAAGTGATCAAGCTGACAGACCCTTC | |
| 2206 | TCTGCTCTACCTGGAGGTCTCCACTCTGGTCAGCAAGTATCCAGA | |
| 2251 | CATCAGGGATGACCACATCGGTGCGCTGCTGGCTGTGCGTGGGGA | |
| 2296 | CGCCAGCCGTGACATGAAGCAGACCATCATGGAGACCCTGGAGCA | |
| 2341 | GGGCCCAGCACAGGCCAGCCCCAGCTACGTGCCCCTCTTCAAGCA | |
| 2386 | CATTGTCGTGCCCAGCCTGAACGTGGCCAAGCTGCTCAAGTAGCC | |
| 2431 | TCCGCCGGCCTGCCCTGCTCGCCCCTCCACAGCCTCGGTCCCTGC | |
| 2476 | CTTTAGAAACGCGGGACAGCTGATTGCTCTCCTTGGCCACACGTG | |
| 2521 | CTCCTTTTAGCTGCACGGCCTGTCTTTAGGTGCCAGTGTGATGCA | |
| 2566 | CCGGGTGTGCGTCGAGTGAGCGTCCCGAGGCCACGTGCGGAGGCC | |
| 2611 | CCTCACTGTGCTCTCAAAGGCCTGTGGGTGCACGGCTCTGCCGCA | |
| 2656 | CAGCCTCTCTTGGGTGCTTGTTTGTTGCAGTGGTTGAAAGTGTGT | |
| 2701 | GGGGCACAGAGGACGTCCACCTCCCTGCCCTCCTCCTCCCTGGGC | |
| 2746 | CTTCACCGCACCCCATCTGCTTAAGTGCTCGGAACCCCGTCACCT | |
| 2791 | AATTAAAGTTTCTCGGCTTCCTCAGAAAAAAAAAAAAAAAAAA | |

SEC8

A SEC8 nucleic acid nucleic acid and polypeptide according to the invention includes the nucleic acid and encoded polypeptide sequence of 6779999.0.31.

The disclosed SEC8 polypeptide is predicted by the program SignalP to have a signal peptide, with the most likely cleavage site between residues 30 and 31, in the sequence ARC-LV. The disclosed SEC8 nucleic acid is expressed in cerebellum and testicular tissue.

The 6779999.0.31 nucleic acid and corresponding polypeptide according to the invention has the following sequence:

| | | |
|---|---|---|
| 1 | CTATTTTTGTATGGCCCTACCACTACAAGTATTTCTTACATTCTT | (SEQ ID NO:15) |
| 46 | AAAGGGTAATGGGGAAAAACACAAATAAGAATATATGCTAGACAC | |
| 91 | TGTAAGTGGGACACAAAGCCTCAACTATTTGCCATCTGTCCTGTT | |
| 136 | ACATAATTAATCCACTATTACCTATGTTTATTAGATTAATTATAC | |
| 181 | TTCTAGAAGTCTGTCAGAGGCAAATAATCAGATATGGGCGGACTA | |
| 226 | AAGACTGATGAAATGGACAGACATACTCAGCAAGAACTTAGAGTG | |
| 271 | AACTTATATTTCTAACAGTAATGGAGTGGACAGTCATAAGACATT | |
| 316 | CATACAGTAAAACTATTTTCTAGAAATAATGAAATAGAGAAATGT | |
| 361 | TCCTAATGAAGTATAAGATGTAAAACTGTATATGGAATATACTGT | |
| 406 | ACATCAAGGAAAGACTCCAAGGAGATAAATATTCAAGTGCTTACT | |
| 451 | CTGAATGTTAGACTTATAGGTGATTTTTTAATTTTTTAATGCTTT | |
| 496 | TTCATGTTATCTCAGCTTCCTAGTTTTGATCTTATAATCAAAGAA | |
| 541 | AAAAACATATCTTTGCTCCTTCTGTTATGGCCACTAAAACAATAT | |
| 586 | GAAGAAAGCTGCGTGTGGTGTTGCATGCCTGTAGTCCCAGCTATT | |
| 631 | TGGGAGACTGAGGCAAGAGGATTGCTTGAGCCCAGGAATTCTAAT | |
| 676 | CCAGCTTGGGTAATATAACAAGACACTGTCTCTAAAAAAAAAGTT | |
| 721 | AAATAATTAAAAATTAAAAAAGAAAAAAAGAACGAAGAGACATGA | |
| | MetA | (SEQ ID NO:16) |
| 766 | GAGTTGAGAAAATAAAGAACCCTTTGAGGAATGTGTCTCTGTTAT | |
| | rgValGluLysIleLysAsnProLeuArgAsnValSerLeuLeuP | |
| 811 | TCATCTTCATATATATCCAGTCCCAGACATTAGCTAGGTGCTTGG | |
| | heIlePheIleTyrIleGlnCysGlnThrLeuAlaArgCysLeuV | |
| 856 | TAAACATTTGTTTAAAGAATGGGCAACTAGGTCGTGAATATGAAA | |
| | alAsnIleCysLeuLysAsnGlyGlnLeuGlyArgGluTyrGluL | |
| 901 | AACTGCTCAGCCTCAAAGAGATGCAAATTCAAATTATATATAATT | |
| | ysLeuLeuSerLeuLysGluMetGlnIleGlnIleIleTyrAsnP | |
| 946 | TTCCCCATATCAAATTAGCAAATATTTTGTTTAATAAAAATTCTT | |
| | heProHisIleLysLeuAlaAsnIleLeuPheAsnLysAsnSerC | |
| 991 | GTTGTGTTTTTTTTTTAAGTTGGATTTTTTTGGAGATATAATTGA | |
| | ysCysValPhePheLeuSerTrpIlePheLeuGluIle | |
| 1036 | CATATAATAAAATTCACCCTTTTTACAAATGTACAGTTTGATGCA | |
| 1081 | TTTTGAAAACTGGATAATTGTGTAACCATGGCCACTATCAACACA | |
| 1126 | GGGAATCTTCCCATTTCCATCACCCCAAAATGTCCCCTTGTACTC | |
| 1171 | CATTCTCTCCTCTTACTCCTAATACCATGCTGTCACTACTTTG | |

SEC9

A SEC9 nucleic acid nucleic acid and polypeptide according to the invention includes the nucleic acid and encoded polypeptide sequence of 8484782.0.5.

The polypeptide of SEC9 is predicted by the PSORT program to localize to the nucleus with a certainty of 0.7600.

The disclosed SEC9 polypeptide has 109 of 172 residues (63%) identical to, and 132 of 172 residues (76%) positive with the 768 residue human gamma-heregulin (SPTREMBL-ACC:O14667). The polypeptide of SEC9 was found to have 109 of 172 residues (63%) identical to, and 132 of 172 residues (76%) positive with, the 768 residue human gamma-heregulin (PCT publication WO 98/02541).

The disclosed SEC9 nucleic acid is highly expressed in the adrenal gland, and moderately expressed in brain tissues. The 8484782.0.5 nucleic acid and encoded polypeptide has the following sequence:

| | | |
|---|---|---|
| 1 | GAGAAAGGAGATTAAAAATAACCTCTGGATATTCCTCTCATGTGA | (SEQ ID NO:17) |
| 46 | TCTTTATTCTGGATGAAGCATTAGGACAGCTAATAGCCGTGTGTC | |
| 91 | ACTGTGTGATTTCTTCCCTAAGACTAAGGACCCATCATTTTAGTG | |
| 136 | CAACCTTCTTCATTTAAATGGAGAGTTGTAATTGCCAATGCTCAC | |
| 181 | AGCTACTCCTGCTCCGGCAATTTGCTGCCAGAAGTGTGTTTTCCT | |
| 226 | TTTTAAAAGGCAGTAAATTCAAGATGTTGTGGTGGATGTAGATTT | |
| 271 | TTGCTGCAAGGAAATAACAGCTGGTGATGGAATTTCATTCTTTTG | |
| 316 | ACTTCTAGATTGCCTGTGAAGAGCTGCTTCCTCGGAAGAGCACCC | |
| 361 | TAAGGCTGGGTGGCCACTATCCTTTGCCTTGGCAGAGCCAGCCAG | |
| 406 | AAGGCCTAGGCACAACCCGCTGTGTTTGCTGACAGCCAACCTACC | |
| 451 | CTGGAGTTCCGGAGCGGCTTCCTACGAACACTGGGGAGCGGTAGA | |
| 496 | AAAATGGCTCTGCTGAGATGAGCTCTTAATTAATGCACTGAGAGC | |
| 541 | CTCCAAGTCCCACCTCTCAACAGGAATGATTGACGTCCAAGGATA | |
| 586 | CATAAATTACACTAACTGAGCTCTGCCTCTATATAAGCTTTCCAC | |
| 631 | ATCCAACTCATCAGAGAAGCTAGGCTTGTACCATAACCAATACCC | |
| 676 | CTGCTTGGCAACTCTAATGAGCAAACTGCCGCAAAATTGAGAGAG | |
| 721 | AACACACCTTTTTGATTTCCTGCTCTTCTAAGACACAGTGATTTA | |
| 766 | GAATTTCTGTTCAAGCAAGAGAACTAAAGACTTCTTTAAAGAAGA | |
| 811 | GAAGAGAGGCCAATGAGACTTGAACCCTCAGCCTAAGTTGTCACC | |
| 856 | AGCAGGACTGATGTGCACACAGAAGGAATGAAGTATGGATGTGAA | |
| | MetAspValLy | (SEQ ID NO:18) |
| 901 | AGAACGCAGGCCTTACTGCTCCCTGACCAAGAGCACACGAGAGAA | |
| | sGluArgArgProTyrCysSerLeuThrLysSerArgArgGluLy | |
| 946 | GGAACGGCGCTACACAAATTCCTCCGCAGACAATGAGGAGTGCCG | |
| | sGluArgArgTyrThrAsnSerSerAlaAspAsnGluGluCysAr | |
| 991 | GGTACCCACACACAACTCCTACAGTTCCAGCGAGACATTGAAAGC | |
| | gValProThrHisAsnSerTyrSerSerSerGluThrLeuLysAl | |
| 1036 | TTTTGATCATGATTCCTCGCGGCTGCTTTACGGCAGAGAGTGAA | |
| | aPheAspHisAspSerSerArgLeuLeuTyrGlyAsnArgValLy | |
| 1081 | GGATTTGGTTCACAGAGAAGCAGACGAGTTCACTAGACAAGGACA | |
| | sAspLeuVaiHisArgGluAlaAspGluPheThrArgGlnGlyGl | |
| 1126 | GAATTTTACCCTAAGGCAGTTAGGAGTTTGTGAACCAGCAACTCG | |
| | nAsnPheThrLeuArgGlnLeuGlyValCysGluProAlaThrAr | |
| 1171 | AAGAGGACTGGCATTTTGTGCGGAAATGGGGCTCCCTCACAGAGG | |
| | gArgGlyLeuAlaPheCysAlaGluMetGlyLeuProHisArgGl | |
| 1216 | TTACTCTATCAGTGCAGGGTCAGATGCTGATACTGAAATGAAGC | |
| | yTyrSerIleSerAlaClySerAspAlaAspThrGluAsnGluAl | |
| 1261 | AGTGATGTCCCCAGAGCATGCCATGAGACTTTGGCGCAGGGGGTT | |
| | aValMetSerProGluHisAlaMetArgLeuTrpGlyArgGlyPh | |
| 1306 | CAAATCAGGCCGCAGCTCCTGCCTGTCAAGTCGGTCCAACTCAGC | |
| | eLysSerGlyArgSerSerCysLeuSerSerArgSerAsnSerAl | |
| 1351 | CCTCACCCTGACAGATACGGAGCACGAAACAAGTCCGACAGTGA | |
| | aLeuThrLeuThrAspThrGluHisGluAsnLysSerAspSerGl | |
| 1396 | GAATGGAGGGTCAAGCAGTTGGTTCGGTTTTCATTCGAATTTTTA | |
| | uAsnGlyGlySerSerSerTrpPheGlyPheHisTrpAsnPheTy | |
| 1441 | TGTGAGTAAAGCTTCCTGTTTGCTGCGCTTGCCTAGGATTTTCTT | |
| | rValSerLysAlaSerCysLeuLeuArgLeuProArgIlePheLe | |
| 1486 | ATCCCACAACTACAATGTGAACAAAGAGATGAGAGAGAAATTATG | |
| | uSerHisAsnTyrAsnValAsnLysGluMetArgGluLysLeuCy | |
| 1531 | CTAATGCATTTTGGTGGATCAAATGAGTCTTTCATGAGACAACTC | |
| | s | |
| 1576 | AAATTTTTGTTAGCTATATGGTGTTGGAATATAATTTCAAAGACA | |
| 1621 | ACTAAGCCCTAAAATAGGAGATTTATTTAAAACATAACTTTTCCT | |
| 1666 | TGAATGAAAGGATGTTTTTGTTCTTTCTCTGACAAATATGATTTG | |
| 1711 | AGAATAAAAGACCTGCCCGGGCAGCCGCTCGAGCCCTATAGTGAG | |

SEC10

A SEC 10 nucleic acid nucleic acid and polypeptide according to the invention includes the nucleic acid and encoded polypeptide sequence of 16399139.S124A. The disclosed SEC10 polypeptide is predicted by the PSORT program to localize to the mitochondrial matrix space with a certainty of 0.8044. The program SignalP predicts that there is a signal peptide, with a putative cleavage site between residues 18 and 19, in the sequence VSS-VM.

The SEC10 polypeptide has 361 of 363 residues (99%) identical to, and 362 of 363 residues (99%) positive with, the 364 residue protein encoded by the human sequence KIAA0976 (SPTREMBL-ACC:Q9Y212).

The 16399139.S124A nucleic acid and encoded polypeptide has the following sequence:

| (SEQ ID NO:19) | |
|---|---|
| 1 | GTGATGGTGATGATGACCCGTACGCGTACAATCGAGACCGACGAGAGGGTTAGGGATACGCTTACCTTCGAACCGCGGGC |
| 81 | CCTCTAGACTCGAGCGGCCGCCACTGTGCTGGATATCTGCAGAATTGCCCTTAGATCTCCACCATGTATTTGTCAAGATT |
| 161 | CCTGTCGATTCATGCCCTTTGGGCTACGGTGTCCTCAGTGATGCAGCCCTACCCTTTGGTTTGGGGACATTATGATTTGT |
| 241 | GTAAGACTCAGATTTACACGGAAGAAGGGAAAGTTTGGGATTACATGGCCTGCCAGCCGGAATCCACGGACATGACAAAA |
| 321 | TATCTGAAAGTGAAACTCGATCCTCCGGATATTACCTGTGGAGACCCTCCTGAGACGTTCTGTGCAATGGGCAATCCCTA |
| 401 | CATGTGCAATAATGAGTGTGATGCGAGTACCCCTGAGCTGGCACACCCCCCTCAGCTGATGTTTGATTTTGAAQGAAGAC |
| 481 | ATCCCTCCACATTTTGCCAGTCTGCCACTTGCAAGGAGTATCCCAAGCCTCTCCAGGTTAACATCACTCTGTCTTGGAGC |
| 561 | AAAACCATTGAGCTAACAGACAACATAGTTATTACCTTTGAATCTCGGCGPCCAGACCAAATGATCCTGGAGAAGTCTCT |
| 641 | CGATTATGGACGAACATGGCAGCCCTATCAGTATTATGCCACAGACTCCTTACATGCTTTTCACATCCATCCTAAATCCG |
| 721 | TGAAGGATTTATCACAGCATACGGTCTTACAAATCATTTGCACAGAAGAGTACTCAACAGGGTATACAACAAATAGCAAA |
| 801 | ATAATCCACTTTGAAATCAAAGACAGCTTCGCGTTTTTTGCTGGACCTCGCCTACGCAATATGGCTTCCCTCTACGGACA |
| 881 | GCTGGATACAACCAAGAAACTCAGAGATTTCTTTACAGTCACAGACCTGAGGATAAGGCTGTTAAGACCAGCCGTTGGGG |
| 961 | AAATATTTGTACATGAGCTACACTTGGCACGCTACTTTTACCCGATCTCAGACATAAAGGTGCGAGGAAGGTGCAAGTGT |
| 1041 | AATCTCCATGCCACTGTATGTGTGTATGACPACAGCAAATTGACATGCGAATGTGAGCACAACACTACAGGTCCAGACTG |
| 1121 | TGGGAAATCCAAGAAGAATTATCAGGGCCGACCTTGGAGTCCAGGCTCCTATCTCCCCATCCCCAAAGGCACTGCAAATA |
| 1201 | CCTGTATCCCCAGTATTTCCAGTATTGCTACGAATGTCTGCGACAACGAGCTCCTGCACTGCCAGAACGGAGGGACGTGC |
| 1281 | CACAACAACCTGCGCTGCCTGTGCCCGGCCCCATACACCCCCATCCTCTGCGAGAAGCTGCGGTGCGAGGAGGCTGGCAG |
| 1361 | CTGCGGCTCCGACTCTGGCCAGGGCGCGCCCCCGCACGGCTCCCTCGAGAAGGGCAATTCCACCACACTGGACTAGTGGA |
| 1441 | TCCGAGCTCGGTACCAAGCTTAACTAGCCAGCTTGGGTCTCCCTATAGTGAGTCGTATTAATTTCGATAAGCCAGTAAGC |
| 1521 | AGTGGGTTCTCTAGTTAGCCAGAGAGCTCTGCTTATATAGACCTCCCACCGTACACGCCTACAA |
| (SEQ ID NO:20) | |
| 1 | MYLSRFLSIHALWATVSSVMQPYPLVWGHYDLCKTQIYTEEGKVWDYMACQPESTDNTKYLKVKLDPPDITCGDPFETFC |
| 81 | AMGNPYMCNNECDASTPELAHPPELMFDFEGRHPSTFWQSATWKEYPKPLQVNITLSWSKTIELTDNIVITFESGRPDQH |
| 161 | ILEKSLDYGRTWQPYQYYATDCLDAPEMDPKSVKDLSQHTVLEIICTEEYSTGYTTNSRIIHFEIKDRFAFFAGPRLRNM |
| 241 | ASLYGQLDTTKKLRDFFTVTDLRIRLLRPAVGEIFVDELHLARYFYAISDIKVRGRCKCNIHATVCVYDNSKLTCECEHN |
| 321 | TTGPDCCKCKKNYQGRPWSPGSYLPIPKGTANTCIPSISSIGTNVCDNELLHCQNGGTCHNNVRCLCPAAYTGILCEKLR |
| 403 | CEEAGSCGSDSGQGAPPHGSLEKGNSTTLD |

SECX Nucleic Acids

The novel nucleic acids of the invention include those that encode an SECX or SECX-like protein, or biologically active portions thereof. The nucleic acids include nucleic acids encoding polypeptides that include the amino acid sequence of one or more of SEQ ID NO: 2n, wherein n=1 to 20. The encoded polypeptides can thus include, e.g., the amino acid sequences of SEQ ID NO: 2, 4, . . . ,16, and/or 20.

In some embodiments, a nucleic acid encoding a polypeptide having the amino acid sequence of one or more of SEQ ID NO: 2n (wherein n=1 to 20) includes the nucleic acid sequence of any of SEQ ID NO: 2n−1 (wherein n=1 to 20), or a fragment thereof. Additionally, the invention includes mutant or variant nucleic acids of any of SEQ ID NO: 2n−1 (wherein n=1 to 20), or a fragment thereof, any of whose bases may be changed from the disclosed sequence while still encoding a protein that maintains its SECX-like activities and physiological functions. The invention further includes the complement of the nucleic acid sequence of any of SEQ ID NO: 2n−1 (wherein n=1 to 20), including fragments, derivatives, analogs and homolog thereof. The invention additionally includes nucleic acids or nucleic acid fragments, or complements thereto, whose structures include chemical modifications.

Also included are nucleic acid fragments sufficient for use as hybridization probes to identify SECX-encoding nucleic acids (e.g., SECX mRNA) and fragments for use as polymerase chain reaction (PCR) primers for the amplification or mutation of SECX nucleic acid molecules. As used herein, the term “nucleic acid molecule” is intended to include DNA molecules (e.g., cDNA or genomic DNA), RNA molecules (e.g., mRNA), analogs of the DNA or RNA generated using nucleotide analogs, and derivatives, fragments and homologs thereof. The nucleic acid molecule can be single-stranded or double-stranded, but preferably is double-stranded DNA.

“Probes” refer to nucleic acid sequences of variable length, preferably between at least about 10 nucleotides (nt), 100 nt, or as many as about, e.g., 6,000 nt, depending on use. Probes are used in the detection of identical, similar, or complementary nucleic acid sequences. Longer length probes are usually obtained from a natural or recombinant source, are highly specific and much slower to hybridize than oligomers. Probes may be single- or double-stranded and designed to have specificity in PCR, membrane-based hybridization technologies, or ELISA-like technologies.

An “isolated” nucleic acid molecule is one that is separated from other nucleic acid molecules that are present in the natural source of the nucleic acid. Examples of isolated nucleic acid molecules include, but are not limited to, recombinant DNA molecules contained in a vector, recombinant DNA molecules maintained in a heterologous host cell, partially or substantially purified nucleic acid molecules, and synthetic DNA or RNA molecules. Preferably, an “isolated” nucleic acid is free of sequences which naturally flank the nucleic acid (i.e., sequences located at the 5′ and 3′ ends of the nucleic acid) in the genomic DNA of the organism from which the nucleic acid is derived. For example, in various embodiments, the isolated SECX nucleic acid molecule can contain less than about 50 kb, 25 kb, 5 kb, 4 kb, 3 kb, 2 kb, 1 kb, 0.5 kb or 0.1 kb of nucleotide sequences which naturally flank the nucleic acid molecule in genomic DNA of the cell from which the nucleic acid is derived. Moreover, an “isolated” nucleic acid molecule, such as a cDNA molecule, can be substantially free of other cellular material or culture medium when produced by recombinant techniques, or of chemical precursors or other chemicals when chemically synthesized.

A nucleic acid molecule of the present invention, e.g., a nucleic acid molecule having the nucleotide sequence of SEQ ID NO: 2n−1 (wherein n=1 to 20), or a complement of any of this nucleotide sequence, can be isolated using standard molecular biology techniques and the sequence information provided herein. Using all or a portion of the nucleic acid sequence of any of SEQ ID NO: 2n−1 (wherein n=1 to 20) as a hybridization probe, SECX nucleic acid sequences can be isolated using standard hybridization and cloning techniques (e.g., as described in Sambrook et al., eds., MOLECULAR CLONING: A LABORATORY MANUAL 2^{nd} Ed., Cold Spring Harbor Laboratory Press, Cold Spring Harbor, N.Y., 1989; and Ausubel, et al., eds., CURRENT PROTOCOLS IN MOLECULAR BIOLOGY, John Wiley & Sons, New York, N.Y., 1993.)

A nucleic acid of the invention can be amplified using cDNA, mRNA or alternatively, genomic DNA, as a template and appropriate oligonucleotide primers according to standard PCR amplification techniques. The nucleic acid so amplified can be cloned into an appropriate vector and characterized by DNA sequence analysis. Furthermore, oligonucleotides corresponding to SECX nucleotide sequences can be prepared by standard synthetic techniques, e.g., using an automated DNA synthesizer.

As used herein, the term “oligonucleotide” refers to a series of linked nucleotide residues, which oligonucleotide has a sufficient number of nucleotide bases to be used in a PCR reaction. A short oligonucleotide sequence may be based on, or designed from, a genomic or cDNA sequence and is used to amplify, confirm, or reveal the presence of an identical, similar or complementary DNA or RNA in a particular cell or tissue. Oligonucleotides comprise portions of a nucleic acid sequence having about 10 nt, 50 nt, or 100 nt in length, preferably about 15 nt to 30 nt in length. In one embodiment, an oligonucleotide comprising a nucleic acid molecule less than 100 nt in length would further comprise at lease 6 contiguous nucleotides of any of SEQ ID NO: 2n−1 (wherein n=1 to 20), or a complement thereof. Oligonucleotides may be chemically synthesized and may be used as probes.

In another embodiment, an isolated nucleic acid molecule of the invention comprises a nucleic acid molecule that is a complement of the nucleotide sequence shown in any of SEQ ID NO: 2n−1 (wherein n=1 to 20). In another embodiment, an isolated nucleic acid molecule of the invention comprises a nucleic acid molecule that is a complement of the nucleotide sequence shown in any of SEQ ID NO: 2n−1 (wherein n=1 to 20), or a portion of this nucleotide sequence. A nucleic acid molecule that is complementary to the nucleotide sequence shown in is one that is sufficiently complementary to the nucleotide sequence shown in of any of SEQ ID NO: 2n−1 (wherein n=1 to 20) that it can hydrogen bond with little or no mismatches to the nucleotide sequence shown in of any of SEQ ID NO: 2n−1 (wherein n=1 to 20), thereby forming a stable duplex.

As used herein, the term “complementary” refers to Watson-Crick or Hoogsteen base pairing between nucleotides units of a nucleic acid molecule, and the term “binding” means the physical or chemical interaction between two polypeptides or compounds or associated polypeptides or compounds or combinations thereof. Binding includes ionic, non-ionic, Von der Waals, hydrophobic interactions, etc. A physical interaction can be either direct or indirect. Indirect interactions may be through or due to the effects of another polypeptide or compound. Direct binding refers to interactions that do not take place through, or due to, the effect of another polypeptide or compound, but instead are without other substantial chemical intermediates.

Moreover, the nucleic acid molecule of the invention can comprise only a portion of the nucleic acid sequence of any of SEQ ID NO: 2n−1 (wherein n=1 to 20), e.g., a fragment that can be used as a probe or primer, or a fragment encoding a biologically active portion of SECX. Fragments provided herein are defined as sequences of at least 6 (contiguous) nucleic acids or at least 4 (contiguous) amino acids, a length sufficient to allow for specific hybridization in the case of nucleic acids or for specific recognition of an epitope in the case of amino acids, respectively, and are at most some portion less than a full length sequence. Fragments may be derived from any contiguous portion of a nucleic acid or amino acid sequence of choice. Derivatives are nucleic acid sequences or amino acid sequences formed from the native compounds either directly or by modification or partial substitution. Analogs are nucleic acid sequences or amino acid sequences that have a structure similar to, but not identical to, the native compound but differs from it in respect to certain components or side chains. Analogs may be synthetic or from a different evolutionary origin and may have a similar or opposite metabolic activity compared to wild type.

Derivatives and analogs may be full length or other than full length, if the derivative or analog contains a modified nucleic acid or amino acid, as described below. Derivatives or analogs of the nucleic acids or proteins of the invention include, but are not limited to, molecules comprising regions that are substantially homologous to the nucleic acids or proteins of the invention, in various embodiments, by at least about 70%, 80%, 85%, 90%, 95%, 98%, or even 99% identity (with a preferred identity of 80-99%) over a nucleic acid or amino acid sequence of identical size or when compared to an aligned sequence in which the alignment is done by a computer homology program known in the art, or whose encoding nucleic acid is capable of hybridizing to the complement of a sequence encoding the aforementioned proteins under stringent, moderately stringent, or low stringent conditions. See e.g. Ausubel, et al., CURRENT PROTOCOLS IN MOLECULAR BIOLOGY, John Wiley & Sons, New York, N.Y., 1993, and below. An exemplary program is the Gap program (Wisconsin Sequence Analysis Package, Version 8 for UNIX, Genetics Computer Group, University Research Park, Madison, Wis.) using the default settings, which uses the algorithm of Smith and Waterman (Adv. Appl. Math., 1981, 2: 482-489, which is incorporated herein by reference in its entirety).

A “homologous nucleic acid sequence” or “homologous amino acid sequence,” or variations thereof, refer to sequences characterized by a homology at the nucleotide level or amino acid level as discussed above. Homologous nucleotide sequences encode those sequences coding for isoforms of SECX polypeptide. Isoforms can be expressed in different tissues of the same organism as a result of, for example, alternative splicing of RNA. Alternatively, isoforms can be encoded by different genes. In the present invention, homologous nucleotide sequences include nucleotide sequences encoding for a SECX polypeptide of species other than humans, including, but not limited to, mammals, and thus can include, e.g., mouse, rat, rabbit, dog, cat cow, horse, and other organisms. Homologous nucleotide sequences also include, but are not limited to, naturally occurring allelic variations and mutations of the nucleotide sequences set forth herein. A homologous nucleotide sequence does not, however, include the nucleotide sequence encoding human SECX protein. Homologous nucleic acid sequences include those nucleic acid sequences that encode conservative amino acid substitutions (see below) in any of SEQ ID NO: 2n (wherein n=1 to 20) as well as a polypeptide having SECX activity. Biological activities of the SECX proteins are described below. A homologous amino acid sequence does not encode the amino acid sequence of a human SECX polypeptide.

The nucleotide sequence determined from the cloning of the human SECX gene allows for the generation of probes and primers designed for use in identifying the cell types disclosed and/or cloning SECX homologues in other cell types, e.g., from other tissues, as well as SECX homologues from other mammals. The probe/primer typically comprises a substantially purified oligonucleotide. The oligonucleotide typically comprises a region of nucleotide sequence that hybridizes under stringent conditions to at least about 12, 25, 50, 100, 150, 200, 250, 300, 350 or 400 or more consecutive sense strand nucleotide sequence of SEQ ID NO: 2n−1 (wherein n=1 to 20); or an anti-sense strand nucleotide sequence of SEQ ID NO: 2n−1 (wherein n=1 to 20); or of a naturally occurring mutant of SEQ ID NO: 2n−1 (wherein n=1 to 20).

Probes based on the human SECX nucleotide sequence can be used to detect transcripts or genomic sequences encoding the same or homologous proteins. In various embodiments, the probe further comprises a label group attached thereto, e.g., the label group can be a radioisotope, a fluorescent compound, an enzyme, or an enzyme co-factor. Such probes can be used as a part of a diagnostic test kit for identifying cells or tissue which misexpress a SECX protein, such as by measuring a level of a SECX-encoding nucleic acid in a sample of cells from a subject e.g., detecting SECX mRNA levels or determining whether a genomic SECX gene has been mutated or deleted.

“A polypeptide having a biologically active portion of SECX” refers to polypeptides exhibiting activity similar, but not necessarily identical to, an activity of a polypeptide of the present invention, including mature forms, as measured in a particular biological assay, with or without dose dependency. A nucleic acid fragment encoding a “biologically active portion of SECX” can be prepared by isolating a portion of SEQ ID NO: 2n−1 (wherein n=1 to 20), that encodes a polypeptide having a SECX biological activity (biological activities of the SECX proteins are summarized in Table 1), expressing the encoded portion of SECX protein (e.g., by recombinant expression in vitro) and assessing the activity of the encoded portion of SECX.

SECX Variants

The invention further encompasses nucleic acid molecules that differ from the disclosed SECX nucleotide sequences due to degeneracy of the genetic code. These nucleic acids thus encode the same SECX protein as that encoded by the nucleotide sequence shown in SEQ ID NO: 2n−1 (wherein n=1 to 20). In another embodiment, an isolated nucleic acid molecule of the invention has a nucleotide sequence encoding a protein having an amino acid sequence shown in any of SEQ ID NO: 2n (wherein n=1 to 20).

In addition to the human SECX nucleotide sequence shown in any of SEQ ID NO: 2n−1 (wherein n=1 to 20), it will be appreciated by those skilled in the art that DNA sequence polymorphisms that lead to changes in the amino acid sequences of SECX may exist within a population (e.g., the human population). Such genetic polymorphism in the SECX gene may exist among individuals within a population due to natural allelic variation. As used herein, the terms “gene” and “recombinant gene” refer to nucleic acid molecules comprising an open reading frame encoding a SECX protein, preferably a mammalian SECX protein. Such natural allelic variations can typically result in 1-5% variance in the nucleotide sequence of the SECX gene. Any and all such nucleotide variations and resulting amino acid polymorphisms in SECX that are the result of natural allelic variation and that do not alter the functional activity of SECX are intended to be within the scope of the invention.

Moreover, nucleic acid molecules encoding SECX proteins from other species, and thus that have a nucleotide sequence that differs from the human sequence of any of SEQ ID NO: 2n−1 (wherein n=1 to 20), are intended to be within the scope of the invention. Nucleic acid molecules corresponding to natural allelic variants and homologues of the SECX cDNAs of the invention can be isolated based on their homology to the human SECX nucleic acids disclosed herein using the human cDNAs, or a portion thereof, as a hybridization probe according to standard hybridization techniques under stringent hybridization conditions.

In another embodiment, an isolated nucleic acid molecule of the invention is at least 6 nucleotides in length and hybridizes under stringent conditions to the nucleic acid molecule comprising the nucleotide sequence of any of SEQ ID NO: 2n−1 (wherein n=1 to 20). In another embodiment, the nucleic acid is at least 10, 25, 50, 100, 250, 500 or 750 nucleotides in length. In another embodiment, an isolated nucleic acid molecule of the invention hybridizes to the coding region. As used herein, the term “hybridizes under stringent conditions” is intended to describe conditions for hybridization and washing under which nucleotide sequences at least 60% homologous to each other typically remain hybridized to each other.

Homologs (i.e., nucleic acids encoding SECX proteins derived from species other than human) or other related sequences (e.g., paralogs) can be obtained by low, moderate or high stringency hybridization with all or a portion of the particular human sequence as a probe using methods well known in the art for nucleic acid hybridization and cloning. As used herein, the phrase “stringent hybridization conditions” refers to conditions under which a probe, primer or oligonucleotide will hybridize to its target sequence, but to no other sequences. Stringent conditions are sequence-dependent and will be different in different circumstances. Longer sequences hybridize specifically at higher temperatures than shorter sequences. Generally, stringent conditions are selected to be about 5° C. lower than the thermal melting point (Tm) for the specific sequence at a defined ionic strength and pH. The Tm is the temperature (under defined ionic strength, pH and nucleic acid concentration) at which 50% of the probes complementary to the target sequence hybridize to the target sequence at equilibrium. Since the target sequences are generally present at excess, at Tm, 50% of the probes are occupied at equilibrium. Typically, stringent conditions will be those in which the salt concentration is less than about 1.0 M sodium ion, typically about 0.01 to 1.0 M sodium ion (or other salts) at pH 7.0 to 8.3 and the temperature is at least about 30° C. for short probes, primers or oligonucleotides (e.g., 10 nt to 50 nt) and at least about 60° C. for longer probes, primers and oligonucleotides. Stringent conditions may also be achieved with the addition of destabilizing agents, such as formamide.

Stringent conditions are known to those skilled in the art and can be found in CURRENT PROTOCOLS IN MOLECULAR BIOLOGY, John Wiley & Sons, N.Y. (1989), 6.3.1-6.3.6. Preferably, the conditions are such that sequences at least about 65%, 70%, 75%, 85%, 90%, 95%, 98%, or 99% homologous to each other typically remain hybridized to each other. A non-limiting example of stringent hybridization conditions is hybridization in a high salt buffer comprising 6×SSC, 50 mM Tris-HCI (pH 7.5), 1 mM EDTA, 0.02% PVP, 0.02% Ficoll, 0.02% BSA, and 500 mg/ml denatured salmon sperm DNA at 65° C. This hybridization is followed by one or more washes in 0.2×SSC, 0.01% BSA at 50° C. An isolated nucleic acid molecule of the invention that hybridizes under stringent conditions to the sequence of any of SEQ ID NO: 2n−1 (wherein n=1 to 20) corresponds to a naturally occurring nucleic acid molecule. As used herein, a “naturally-occurring” nucleic acid molecule refers to an RNA or DNA molecule having a nucleotide sequence that occurs in nature (e.g., encodes a natural protein).

In a second embodiment, a nucleic acid sequence that is hybridizable to the nucleic acid molecule comprising the nucleotide sequence of any of SEQ ID NO: 2n−1 (wherein n=1 to 20), or fragments, analogs or derivatives thereof, under conditions of moderate stringency is provided. A non-limiting example of moderate stringency hybridization conditions are hybridization in 6×SSC, 5× Denhardt's solution, 0.5% SDS and 100 mg/ml denatured salmon sperm DNA at 55° C., followed by one or more washes in 1×SSC, 0.1% SDS at 37° C. Other conditions of moderate stringency that may be used are well known in the art. See, e.g., Ausubel et al. (eds.), 1993, CURRENT PROTOCOLS IN MOLECULAR BIOLOGY, John Wiley & Sons, NY, and Kriegler, 1990, GENE TRANSFER AND EXPRESSION, A LABORATORY MANUAL, Stockton Press, NY.

In a third embodiment, a nucleic acid that is hybridizable to the nucleic acid molecule comprising the nucleotide sequence of any of SEQ ID NO: 2n−1 (wherein n=1 to 20), or fragments, analogs or derivatives thereof, under conditions of low stringency, is provided. A non-limiting example of low stringency hybridization conditions are hybridization in 35% formamide, 5×SSC, 50 mM Tris-HCl (pH 7.5), 5 mM EDTA, 0.02% PVP, 0.02% Ficoll, 0.2% BSA, 100 mg/ml denatured salmon sperm DNA, 10% (wt/vol) dextran sulfate at 40° C., followed by one or more washes in 2×SSC, 25 mM Tris-HCl (pH 7.4), 5 mM EDTA, and 0.1% SDS at 50° C. Other conditions of low stringency that may be used are well known in the art (e.g., as employed for cross-species hybridizations). See, e.g., Ausubel et al. (eds.), 1993, CURRENT PROTOCOLS IN MOLECULAR BIOLOGY, John Wiley & Sons, NY, and Kriegler, 1990, GENE TRANSFER AND EXPRESSION, A LABORATORY MANUAL, Stockton Press, NY; Shilo and Weinberg, 1981, Proc Natl Acad Sci USA 78: 6789-6792.

Conservative Mutations

In addition to naturally-occurring allelic variants of the SECX sequence that may exist in the population, the skilled artisan will further appreciate that changes can be introduced by mutation into the nucleotide sequence of any of SEQ ID NO: 2n−1 (wherein n=1 to 20), thereby leading to changes in the amino acid sequence of the encoded SECX protein, without altering the functional ability of the SECX protein. For example, nucleotide substitutions leading to amino acid substitutions at “non-essential” amino acid residues can be made in the sequence of any of SEQ ID NO: 2n−1 (wherein n=1 to 20).

A “non-essential” amino acid residue is a residue that can be altered from the wild-type sequence of SECX without altering the biological activity, whereas an “essential” amino acid residue is required for biological activity. For example, amino acid residues that are conserved among the SECX proteins of the present invention, are predicted to be particularly unamenable to alteration.

Amino acid residues that are conserved among members of an SECX family members are predicted to be less amenable to alteration. For example, an SECX protein according to the present invention can contain at least one domain (e.g., as shown in Table 1) that is a typically conserved region in an SECX family member. As such, these conserved domains are not likely to be amenable to mutation. Other amino acid residues, however, (e.g., those that are not conserved or only semi-conserved among members of the SECX family) may not be as essential for activity and thus are more likely to be amenable to alteration.

Another aspect of the invention pertains to nucleic acid molecules encoding SECX proteins that contain changes in amino acid residues that are not essential for activity. Such SECX proteins differ in amino acid sequence from any of any of SEQ ID NO: 2n (wherein n=1 to 20), yet retain biological activity. In one embodiment, the isolated nucleic acid molecule comprises a nucleotide sequence encoding a protein, wherein the protein comprises an amino acid sequence at least about 75% homologous to the amino acid sequence of any of SEQ ID NO: 2n (wherein n=1 to 20). Preferably, the protein encoded by the nucleic acid is at least about 80% homologous to any of SEQ ID NO: 2n (wherein n=1 to 20), more preferably at least about 90%, 95%, 98%, and most preferably at least about 99% homologous to SEQ ID NO: 2.

An isolated nucleic acid molecule encoding a SECX protein homologous to the protein of any of SEQ ID NO: 2n (wherein n=1 to 20) can be created by introducing one or more nucleotide substitutions, additions or deletions into the corresponding nucleotide sequence, i.e. SEQ ID NO: 2n−1 for the corresponding n, such that one or more amino acid substitutions, additions or deletions are introduced into the encoded protein. Mutations can be introduced into SEQ ID NO: 2n−1 (wherein n=1 to 20) by standard techniques, such as site-directed mutagenesis and PCR-mediated mutagenesis.

Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted nonessential amino acid residue in SECX is replaced with another amino acid residue from the same side chain family. Alternatively, in another embodiment, mutations can be introduced randomly along all or part of a SECX coding sequence, such as by saturation mutagenesis, and the resultant mutants can be screened for SECX biological activity to identify mutants that retain activity. Following mutagenesis of SEQ ID NO: 2n−1 (wherein n=1 to 20). the encoded protein can be expressed by any recombinant technology known in the art and the activity of the protein can be determined.

In one embodiment, a mutant SECX protein can be assayed for (1) the ability to form protein:protein interactions with other SECX proteins, other cell-surface proteins, or biologically active portions thereof, (2) complex formation between a mutant SECX protein and a SECX receptor; (3) the ability of a mutant SECX protein to bind to an intracellular target protein or biologically active portion thereof; (e.g., avidin proteins); (4) the ability to bind BRA protein; or (5) the ability to specifically bind an anti-SECX protein antibody.

Antisense

Another aspect of the invention pertains to isolated antisense nucleic acid molecules that are hybridizable to or complementary to the nucleic acid molecule comprising the nucleotide sequence of SEQ ID NO: 2n−1 (wherein n=1 to 20), or fragments, analogs or derivatives thereof. An “antisense” nucleic acid comprises a nucleotide sequence that is complementary to a “sense” nucleic acid encoding a protein, e.g., complementary to the coding strand of a double-stranded cDNA molecule or complementary to an mRNA sequence. In specific aspects, antisense nucleic acid molecules are provided that comprise a sequence complementary to at least about 10, 25, 50, 100, 250 or 500 nucleotides or an entire SECX coding strand, or to only a portion thereof. Nucleic acid molecules encoding fragments, homologs, derivatives and analogs of a SECX protein of any of SEQ ID NO: 2n (wherein n=1 to 20) or antisense nucleic acids complementary to a SECX nucleic acid sequence of SEQ ID NO: 2n−1 (wherein n=1 to 20) are additionally provided.

In one embodiment, an antisense nucleic acid molecule is antisense to a “coding region” of the coding strand of a nucleotide sequence encoding SECX. The term “coding region” refers to the region of the nucleotide sequence comprising codons which are translated into amino acid residues (e.g., the protein coding region of a human SECX that corresponds to any of SEQ ID NO: 2n (wherein n=1 to 20)). In another embodiment, the antisense nucleic acid molecule is antisense to a “noncoding region” of the coding strand of a nucleotide sequence encoding SECX. The term “noncoding region” refers to 5′ and 3′ sequences which flank the coding region that are not translated into amino acids (i.e., also referred to as 5′ and 3′ untranslated regions).

Given the coding strand sequences encoding SECX disclosed herein (e.g., SEQ ID NO: 2n−1 (wherein n=1 to 20) ), antisense nucleic acids of the invention can be designed according to the rules of Watson and Crick or Hoogsteen base pairing. The antisense nucleic acid molecule can be complementary to the entire coding region of SECX mRNA, but more preferably is an oligonucleotide that is antisense to only a portion of the coding or noncoding region of SECX mRNA. For example, the antisense oligonucleotide can be complementary to the region surrounding the translation start site of SECX mRNA. An antisense oligonucleotide can be, for example, about 5, 10, 15, 20, 25, 30, 35, 40, 45 or 50 nucleotides in length. An antisense nucleic acid of the invention can be constructed using chemical synthesis or enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used.

Examples of modified nucleotides that can be used to generate the antisense nucleic acid include: 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding a SECX protein to thereby inhibit expression of the protein, e.g., by inhibiting transcription and/or translation. The hybridization can be by conventional nucleotide complementarity to form a stable duplex, or, for example, in the case of an antisense nucleic acid molecule that binds to DNA duplexes, through specific interactions in the major groove of the double helix. An example of a route of administration of antisense nucleic acid molecules of the invention includes direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface, e.g., by linking the antisense nucleic acid molecules to peptides or antibodies that bind to cell surface receptors or antigens. The antisense nucleic acid molecules can also be delivered to cells using the vectors described herein. To achieve sufficient intracellular concentrations of antisense molecules, vector constructs in which the antisense nucleic acid molecule is placed under the control of a strong pol II or pol III promoter are preferred.

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual β-units, the strands run parallel to each other (Gaultier et al. (1987) Nucleic Acids Res 15: 6625-6641). The antisense nucleic acid molecule can also comprise a 2′-o-methylribonucleotide (Inoue et al. (1987) Nucleic Acids Res 15: 6131-6148) or a chimeric RNA-DNA analogue (Inoue et al. (1987) FEBS Lett 215: 327-330).

Ribozymes and PNA Moieties

Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject.

In still another embodiment, an antisense nucleic acid of the invention is a ribozyme. Ribozymes are catalytic RNA molecules with ribonuclease activity that are capable of cleaving a single-stranded nucleic acid, such as an mRNA, to which they have a complementary region. Thus, ribozymes (e.g., hammerhead ribozymes (described in Haselhoff and Gerlach (1988) Nature 334:585-591)) can be used to catalytically cleave SECX mRNA transcripts to thereby inhibit translation of SECX mRNA. A ribozyme having specificity for a SECX-encoding nucleic acid can be designed based upon the nucleotide sequence of a SECX DNA disclosed herein (i.e., SEQ ID NO: 2n−1 (wherein n=1 to 20)). For example, a derivative of a Tetrahymena L-19 IVS RNA can be constructed in which the nucleotide sequence of the active site is complementary to the nucleotide sequence to be cleaved in a SECX-encoding mRNA. See, e.g., Cech et al. U.S. Pat. No. 4,987,071; and Cech et al. U.S. Pat. No. 5,116,742. Alternatively, SECX mRNA can be used to select a catalytic RNA having a specific ribonuclease activity from a pool of RNA molecules. See, e.g., Bartel et al., (1993) Science 261:1411-1418.

Alternatively, SECX gene expression can be inhibited by targeting nucleotide sequences complementary to the regulatory region of the SECX (e.g., the SECX promoter and/or enhancers) to form triple helical structures that prevent transcription of the SECX gene in target cells. See generally, Helene. (1991) Anticancer Drug Des. 6: 569-84; Helene. et al. (1992) Ann. N.Y. Acad. Sci. 660:27-36; and Maher (1992) Bioassays 14: 807-15.

In various embodiments, the nucleic acids of SECX can be modified at the base moiety, sugar moiety or phosphate backbone to improve, e.g., the stability, hybridization, or solubility of the molecule. For example, the deoxyribose phosphate backbone of the nucleic acids can be modified to generate peptide nucleic acids (see Hyrup et al. (1996) Bioorg Med Chem 4: 5-23). As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup et al. (1996) above; Perry-O'Keefe et al. (1996) PNAS 93: 14670-675.

PNAs of SECX can be used in therapeutic and diagnostic applications. For example, PNAs can be used as antisense or antigene agents for sequence-specific modulation of gene expression by, e.g., inducing transcription or translation arrest or inhibiting replication. PNAs of SECX can also be used, e.g., in the analysis of single base pair mutations in a gene by, e.g., PNA directed PCR clamping; as artificial restriction enzymes when used in combination with other enzymes, e.g., S1 nucleases (Hyrup B. (1996) above); or as probes or primers for DNA sequence and hybridization (Hyrup et al. (1996), above; Perry-O'Keefe (1996), above).

In another embodiment, PNAs of SECX can be modified, e.g., to enhance their stability or cellular uptake, by attaching lipophilic or other helper groups to PNA, by the formation of PNA-DNA chimeras, or by the use of liposomes or other techniques of drug delivery known in the art. For example, PNA-DNA chimeras of SECX can be generated that may combine the advantageous properties of PNA and DNA. Such chimeras allow DNA recognition enzymes, e.g., RNase H and DNA polymerases, to interact with the DNA portion while the PNA portion would provide high binding affinity and specificity. PNA-DNA chimeras can be linked using linkers of appropriate lengths selected in terms of base stacking, number of bonds between the nucleobases, and orientation (Hyrup (1996) above). The synthesis of PNA-DNA chimeras can be performed as described in Hyrup (1996) above and Finn et al. (1996) Nucl Acids Res 24: 3357-63. For example, a DNA chain can be synthesized on a solid support using standard phosphoramidite coupling chemistry, and modified nucleoside analogs, e.g., 5′-(4-methoxytrityl)amino-5′-deoxy-thymidine phosphoramidite, can be used between the PNA and the 5′ end of DNA (Mag et al. (1989) Nucl Acid Res 17: 5973-88). PNA monomers are then coupled in a stepwise manner to produce a chimeric molecule with a 5′ PNA segment and a 3′ DNA segment (Finn et al. (1996) above). Alternatively, chimeric molecules can be synthesized with a 5′ DNA segment and a 3′ PNA segment. See, Petersen et al. (1975) Bioorg Med Chem Lett 5: 1119-11124.

In other embodiments, the oligonucleotide may include other appended groups such as peptides (e.g., for targeting host cell receptors in vivo), or agents facilitating transport across the cell membrane (see, e.g., Letsinger et al., 1989, Proc. Natl. Acad. Sci. U.S.A. 86:6553-6556; Lemaitre et al., 1987, Proc. Natl. Acad. Sci. 84:648-652; PCT Publication No. W088/09810) or the blood-brain barrier (see, e.g., PCT Publication No. W089/10134). In addition, oligonucleotides can be modified with hybridization triggered cleavage agents (See, e.g., Krol et al., 1988, BioTechniques 6:958-976) or intercalating agents. (See, e.g., Zon, 1988, Pharm. Res. 5: 539-549). To this end, the oligonucleotide may be conjugated to another molecule, e.g., a peptide, a hybridization triggered cross-linking agent, a transport agent, a hybridization-triggered cleavage agent, etc.

SECX Polypeptides

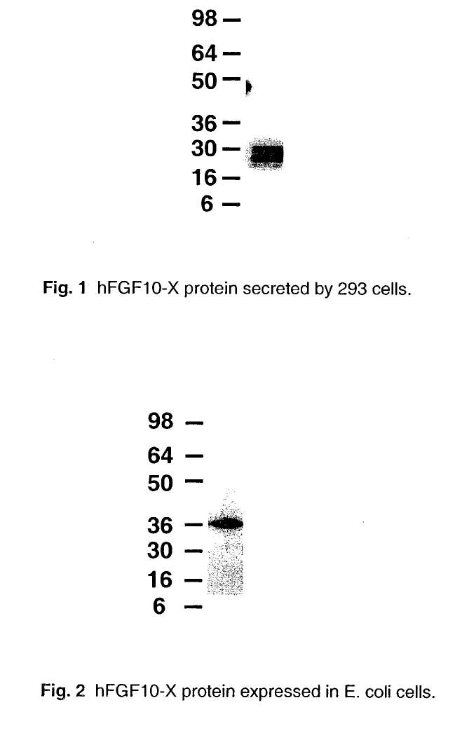

The novel protein of the invention includes the SECX-like protein whose sequence is provided in any of SEQ ID NO: 2n (wherein n=1 to 20). The invention also includes a mutant or variant protein any of whose residues may be changed from the corresponding residue shown in FIG. 1 while still encoding a protein that maintains its SECX-like activities and physiological functions, or a functional fragment thereof. For example, the invention includes the polypeptides encoded by the variant SECX nucleic acids described above. In the mutant or variant protein, up to 20% or more of the residues may be so changed.

In general, an SECX-like variant that preserves SECX-like function includes any variant in which residues at a particular position in the sequence have been substituted by other amino acids, and further include the possibility of inserting an additional residue or residues between two residues of the parent protein as well as the possibility of deleting one or more residues from the parent sequence. Any amino acid substitution, insertion, or deletion is encompassed by the invention. In favorable circumstances, the substitution is a conservative substitution as defined above. Furthermore, without limiting the scope of the invention, positions of any of SEQ ID NO: 2n (wherein n=1 to 20) may be substitute such that a mutant or variant protein may include one or more substitutions

The invention also includes isolated SECX proteins, and biologically active portions thereof, or derivatives, fragments, analogs or homologs thereof. Also provided are polypeptide fragments suitable for use as immunogens to raise anti-SECX antibodies.

In one embodiment, native SECX proteins can be isolated from cells or tissue sources by an appropriate purification scheme using standard protein purification techniques. In another embodiment, SECX proteins are produced by recombinant DNA techniques. Alternative to recombinant expression, a SECX protein or polypeptide can be synthesized chemically using standard peptide synthesis techniques.

An “isolated” or “purified” protein or biologically active portion thereof is substantially free of cellular material or other contaminating proteins from the cell or tissue source from which the SECX protein is derived, or substantially free from chemical precursors or other chemicals when chemically synthesized. The language “substantially free of cellular material” includes preparations of SECX protein in which the protein is separated from cellular components of the cells from which it is isolated or recombinantly produced. In one embodiment, the language “substantially free of cellular material” includes preparations of SECX protein having less than about 30% (by dry weight) of non-SECX protein (also referred to herein as a “contaminating protein”), more preferably less than about 20% of non-SECX protein, still more preferably less than about 10% of non-SECX protein, and most preferably less than about 5% non-SECX protein. When the SECX protein or biologically active portion thereof is recombinantly produced, it is also preferably substantially free of culture medium, i.e., culture medium represents less than about 20%, more preferably less than about 10%, and most preferably less than about 5% of the volume of the protein preparation.

The language “substantially free of chemical precursors or other chemicals” includes preparations of SECX protein in which the protein is separated from chemical precursors or other chemicals that are involved in the synthesis of the protein. In one embodiment, the language “substantially free of chemical precursors or other chemicals” includes preparations of SECX protein having less than about 30% (by dry weight) of chemical precursors or non-SECX chemicals, more preferably less than about 20% chemical precursors or non-SECX chemicals, still more preferably less than about 10% chemical precursors or non-SECX chemicals, and most preferably less than about 5% chemical precursors or non-SECX chemicals.

Biologically active portions of a SECX protein include peptides comprising amino acid sequences sufficiently homologous to or derived from the amino acid sequence of the SECX protein, e.g., the amino acid sequence shown in SEQ ID NO: 2 that include fewer amino acids than the full length SECX proteins, and exhibit at least one activity of a SECX protein. Typically, biologically active portions comprise a domain or motif with at least one activity of the SECX protein. A biologically active portion of a SECX protein can be a polypeptide which is, for example, 10, 25, 50, 100 or more amino acids in length.

A biologically active portion of a SECX protein of the present invention may contain at least one of the above-identified domains conserved between the FGF family of proteins. Moreover, other biologically active portions, in which other regions of the protein are deleted, can be prepared by recombinant techniques and evaluated for one or more of the functional activities of a native SECX protein.

In an embodiment, the SECX protein has an amino acid sequence shown in any of SEQ ID NO: 2n (wherein n=1 to 20). In other embodiments, the SECX protein is substantially homologous to any of SEQ ID NO: 2n (wherein n=1 to 20) and retains the functional activity of the protein of any of SEQ ID NO: 2n (wherein n=1 to 20), yet differs in amino acid sequence due to natural allelic variation or mutagenesis, as described in detail below. Accordingly, in another embodiment, the SECX protein is a protein that comprises an amino acid sequence at least about 45% homologous, and more preferably about 55, 65, 70, 75, 80, 85, 90, 95, 98 or even 99% homologous to the amino acid sequence of any of SEQ ID NO: 2n (wherein n=1 to 20) and retains the functional activity of the SECX proteins of the corresponding polypeptide having the sequence of SEQ ID NO: 2n (wherein n=1 to 20).

Determining Homology Between Two or More Sequences

To determine the percent homology of two amino acid sequences or of two nucleic acids, the sequences are aligned for optimal comparison purposes (e.g., gaps can be introduced in either of the sequences being compared for optimal alignment between the sequences). The amino acid residues or nucleotides at corresponding amino acid positions or nucleotide positions are then compared. When a position in the first sequence is occupied by the same amino acid residue or nucleotide as the corresponding position in the second sequence, then the molecules are homologous at that position (i.e., as used herein amino acid or nucleic acid “homology” is equivalent to amino acid or nucleic acid “identity”).

The nucleic acid sequence homology may be determined as the degree of identity between two sequences. The homology may be determined using computer programs known in the art, such as GAP software provided in the GCG program package. See, Needleman and Wunsch 1970 J Mol Biol 48: 443-453. Using GCG GAP software with the following settings for nucleic acid sequence comparison: GAP creation penalty of 5.0 and GAP extension penalty of 0.3, the coding region of the analogous nucleic acid sequences referred to above exhibits a degree of identity preferably of at least 70%, 75%, 80%, 85%, 90%, 95%, 98%, or 99%, with the CDS (encoding) part of the DNA sequence shown in SEQ ID NO: 2n−1 (wherein n=1 to 20).

The term “sequence identity” refers to the degree to which two polynucleotide or polypeptide sequences are identical on a residue-by-residue basis over a particular region of comparison. The term “percentage of sequence identity” is calculated by comparing two optimally aligned sequences over that region of comparison, determining the number of positions at which the identical nucleic acid base (e.g., A, T, C, G, U, or I, in the case of nucleic acids) occurs in both sequences to yield the number of matched positions, dividing the number of matched positions by the total number of positions in the region of comparison (i.e., the window size), and multiplying the result by 100 to yield the percentage of sequence identity. The term “substantial identity” as used herein denotes a characteristic of a polynucleotide sequence, wherein the polynucleotide comprises a sequence that has at least 80 percent sequence identity, preferably at least 85 percent identity and often 90 to 95 percent sequence identity, more usually at least 99 percent sequence identity as compared to a reference sequence over a comparison region. The term “percentage of positive residues” is calculated by comparing two optimally aligned sequences over that region of comparison, determining the number of positions at which the identical and conservative amino acid substitutions, as defined above, occur in both sequences to yield the number of matched positions, dividing the number of matched positions by the total number of positions in the region of comparison (i.e., the window size), and multiplying the result by 100 to yield the percentage of positive residues.

Chimeric and Fusion Proteins

The invention also provides SECX chimeric or fusion proteins. As used herein, a SECX “chimeric protein” or “fusion protein” includes a SECX polypeptide operatively linked to a non-SECX polypeptide. A “SECX polypeptide” refers to a polypeptide having an amino acid sequence corresponding to SECX, whereas a “non-SECX polypeptide” refers to a polypeptide having an amino acid sequence corresponding to a protein that is not substantially homologous to the SECX protein, e.g., a protein that is different from the SECX protein and that is derived from the same or a different organism. Within a SECX fusion protein the SECX polypeptide can correspond to all or a portion of a SECX protein. In one embodiment, a SECX fusion protein comprises at least one biologically active portion of a SECX protein. In another embodiment, a SECX fusion protein comprises at least two biologically active portions of a SECX protein. Within the fusion protein, the term “operatively linked” is intended to indicate that the SECX polypeptide and the non-SECX polypeptide are fused in-frame to each other. The non-SECX polypeptide can be fused to the N-terminus or C-terminus of the SECX polypeptide.

For example, in one embodiment a SECX fusion protein comprises a SECX polypeptide operably linked to the extracellular domain of a second protein. Such fusion proteins can be further utilized in screening assays for compounds that modulate SECX activity (such assays are described in detail below).

In another embodiment, the fusion protein is a GST-SECX fusion protein in which the SECX sequences are fused to the C-terminus of the GST (i.e., glutathione S-transferase) sequences. Such fusion proteins can facilitate the purification of recombinant SECX.

In yet another embodiment, the fusion protein is a SECX protein containing a heterologous signal sequence at its N-terminus. For example, the native SECX signal sequence can be removed and replaced with a signal sequence from another protein. In certain host cells (e.g., mammalian host cells), expression and/or secretion of SECX can be increased through use of a heterologous signal sequence.

In another embodiment, the fusion protein is a SECX-immunoglobulin fusion protein in which the SECX sequences comprising one or more domains are fused to sequences derived from a member of the immunoglobulin protein family. The SECX-immunoglobulin fusion proteins of the invention can be incorporated into pharmaceutical compositions and administered to a subject to inhibit an interaction between a SECX ligand and a SECX protein on the surface of a cell, to thereby suppress SECX-mediated signal transduction in vivo. In one nonlimiting example, a contemplated SECX ligand of the invention is an SECX receptor. The SECX-immunoglobulin fusion proteins can be used to modulate the bioavailability of a SECX cognate ligand.

Inhibition of the SECX ligand/SECX interaction may be useful therapeutically for both the treatment of proliferative and differentiative disorders, as well as modulating (e.g., promoting or inhibiting) cell survival. Moreover, the SECX-immunoglobulin fusion proteins of the invention can be used as immunogens to produce anti-SECX antibodies in a subject, to purify SECX ligands, and in screening assays to identify molecules that inhibit the interaction of SECX with a SECX ligand.

A SECX chimeric or fusion protein of the invention can be produced by standard recombinant DNA techniques. For example, DNA fragments coding for the different polypeptide sequences are ligated together in-frame in accordance with conventional techniques, e.g., by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers that give rise to complementary overhangs between two consecutive gene fragments that can subsequently be annealed and reamplified to generate a chimeric gene sequence (see, for example, Ausubel et al. (eds.) CURRENT PROTOCOLS IN MOLECULAR BIOLOGY, John Wiley & Sons, 1992). Moreover, many expression vectors are commercially available that already encode a fusion moiety (e.g., a GST polypeptide). A SECX-encoding nucleic acid can be cloned into such an expression vector such that the fusion moiety is linked in-frame to the SECX protein.

SECX Agonists and Antagonists

The present invention also pertains to variants of the SECX proteins that function as either SECX agonists (mimetics) or as SECX antagonists. Variants of the SECX protein can be generated by mutagenesis, e.g., discrete point mutation or truncation of the SECX protein. An agonist of the SECX protein can retain substantially the same, or a subset of, the biological activities of the naturally occurring form of the SECX protein. An antagonist of the SECX protein can inhibit one or more of the activities of the naturally occurring form of the SECX protein by, for example, competitively binding to a downstream or upstream member of a cellular signaling cascade which includes the SECX protein. Thus, specific biological effects can be elicited by treatment with a variant of limited function. In one embodiment, treatment of a subject with a variant having a subset of the biological activities of the naturally occurring form of the protein has fewer side effects in a subject relative to treatment with the naturally occurring form of the SECX proteins.

Variants of the SECX protein that function as either SECX agonists (mimetics) or as SECX antagonists can be identified by screening combinatorial libraries of mutants, e.g., truncation mutants, of the SECX protein for SECX protein agonist or antagonist activity. In one embodiment, a variegated library of SECX variants is generated by combinatorial mutagenesis at the nucleic acid level and is encoded by a variegated gene library. A variegated library of SECX variants can be produced by, for example, enzymatically ligating a mixture of synthetic oligonucleotides into gene sequences such that a degenerate set of potential SECX sequences is expressible as individual polypeptides, or alternatively, as a set of larger fusion proteins (e.g., for phage display) containing the set of SECX sequences therein. There are a variety of methods which can be used to produce libraries of potential SECX variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential SECX sequences. Methods for synthesizing degenerate oligonucleotides are known in the art (see, e.g., Narang (1983) Tetrahedron 39:3; Itakura et al. (1984) Annu Rev Biochem 53:323; Itakura et al. (1984) Science 198:1056; Ike et al. (1983) Nucl Acid Res 11:477.

Polypeptide Libraries

In addition, libraries of fragments of the SECX protein coding sequence can be used to generate a variegated population of SECX fragments for screening and subsequent selection of variants of a SECX protein. In one embodiment, a library of coding sequence fragments can be generated by treating a double stranded PCR fragment of a SECX coding sequence with a nuclease under conditions wherein nicking occurs only about once per molecule, denaturing the double stranded DNA, renaturing the DNA to form double stranded DNA that can include sense/antisense pairs from different nicked products, removing single stranded portions from reformed duplexes by treatment with S1 nuclease, and ligating the resulting fragment library into an expression vector. By this method, an expression library can be derived which encodes N-terminal and internal fragments of various sizes of the SECX protein.

Several techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of SECX proteins. The most widely used techniques, which are amenable to high throughput analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recrusive ensemble mutagenesis (REM), a new technique that enhances the frequency of functional mutants in the libraries, can be used in combination with the screening assays to identify SECX variants (Arkin and Yourvan (1992) PNAS 89:7811-7815; Delgrave et al. (1993) Protein Engineering 6:327-331).

Anti-SECX Antibodies

The invention further encompasses antibodies and antibody fragments, such as F_{ab} or (Fab)₂, that bind immunospecifically to any of the proteins of the invention.

An isolated SECX protein, or a portion or fragment thereof, can be used as an immunogen to generate antibodies that bind SECX using standard techniques for polyclonal and monoclonal antibody preparation. Full-length SECX protein can be used. Alternatively, the invention provides antigenic peptide fragments of SECX for use as immunogens. The antigenic peptide of SECX comprises at least 4 amino acid residues of the amino acid sequence shown in any of SEQ ID NO: 2n (wherein n=1 to 20). The antigenic peptide encompasses an epitope of SECX such that an antibody raised against the peptide forms a specific immune complex with SECX. The antigenic peptide may comprise at least 6 aa residues, at least 8 aa residues, at least 10 aa residues, at least 15 aa residues, at least 20 aa residues, or at least 30 aa residues. In one embodiment of the invention, the antigenic peptide comprises a polypeptide comprising at least 6 contiguous amino acids of any of SEQ ID NO: 2n (wherein n=1 to 20).

In an embodiment of the invention, epitopes encompassed by the antigenic peptide are regions of SECX that are located on the surface of the protein, e.g., hydrophilic regions. As a means for targeting antibody production, hydropathy plots showing regions of hydrophilicity and hydrophobicity may be generated by any method well known in the art, including, for example, the Kyte Doolittle or the Hopp Woods methods, either with or without Fourier transformation. See, e.g., Hopp and Woods, 1981, Proc. Nat. Acad. Sci. USA 78: 3824-3828; Kyte and Doolittle 1982, J. Mol. Biol. 157: 105-142, each incorporated herein by reference in their entirety.

As disclosed herein, an SECX protein sequence of any of SEQ ID NO: 2n (wherein n=1 to 20), or derivatives, fragments, analogs or homologs thereof, may be utilized as immunogens in the generation of antibodies that immunospecifically-bind these protein components. The term “antibody” as used herein refers to immunoglobulin molecules and immunologically active portions of immunoglobulin molecules, i.e., molecules that contain an antigen binding site that specifically binds (immunoreacts with) an antigen, such as SECX. Such antibodies include, but are not limited to, polyclonal, monoclonal, chimeric, single chain, F_{ab} and F_{(ab′)2} fragments, and an F_{ab} expression library. In a specific embodiment, antibodies to human SECX proteins are disclosed. Various procedures known within the art may be used for the production of polyclonal or monoclonal antibodies to a SECX protein sequence of any of SEQ ID NO: 2n (wherein n=1 to 20) or derivative, fragment, analog or homolog thereof. Some of these proteins are discussed below.

For the production of polyclonal antibodies, various suitable host animals (e.g., rabbit, goat, mouse or other mammal) may be immunized by injection with the native protein, or a synthetic variant thereof, or a derivative of the foregoing. An appropriate immunogenic preparation can contain, for example, recombinantly expressed SECX protein or a chemically synthesized SECX polypeptide. The preparation can further include an adjuvant. Various adjuvants used to increase the immunological response include, but are not limited to, Freund's (complete and incomplete), mineral gels (e.g., aluminum hydroxide), surface active substances (e.g., lysolecithin, pluronic polyols, polyanions, peptides, oil emulsions, dinitrophenol, etc.), human adjuvants such as Bacille Calmette-Guerin and Corynebacterium parvum, or similar immunostimulatory agents. If desired, the antibody molecules directed against SECX can be isolated from the mammal (e.g., from the blood) and further purified by well known techniques, such as protein A chromatography to obtain the IgG fraction.

The term “monoclonal antibody” or “monoclonal antibody composition”, as used herein, refers to a population of antibody molecules that contain only one species of an antigen binding site capable of immunoreacting with a particular epitope of SECX. A monoclonal antibody composition thus typically displays a single binding affinity for a particular SECX protein with which it immunoreacts. For preparation of monoclonal antibodies directed towards a particular SECX protein, or derivatives, fragments, analogs or homologs thereof, any technique that provides for the production of antibody molecules by continuous cell line culture may be utilized. Such techniques include, but are not limited to, the hybridoma technique (see Kohler & Milstein, 1975 Nature 256: 495-497); the trioma technique; the human B-cell hybridoma technique (see Kozbor, et al., 1983 Immunol Today 4: 72) and the EBV hybridoma technique to produce human monoclonal antibodies (see Cole, et al., 1985 In: MONOCLONAL ANTIBODIES AND CANCER THERAPY, Alan R. Liss, Inc., pp. 77-96). Human monoclonal antibodies may be utilized in the practice of the present invention and may be produced by using human hybridomas (see Cote, et al., 1983. Proc Natl Acad Sci USA 80: 2026-2030) or by transforming human B-cells with Epstein Barr Virus in vitro (see Cole, et al., 1985 In: MONOCLONAL ANTIBODIES AND CANCER THERAPY, Alan R. Liss, Inc., pp. 77-96). Each of the above citations are incorporated herein by reference in their entirety.

According to the invention, techniques can be adapted for the production of single-chain antibodies specific to a SECX protein (see e.g., U.S. Pat. No. 4,946,778). In addition, methods can be adapted for the construction of F_{ab} expression libraries (see e.g., Huse, et al., 1989 Science 246: 1275-1281) to allow rapid and effective identification of monoclonal F_{ab} fragments with the desired specificity for a SECX protein or derivatives, fragments, analogs or homologs thereof. Non-human antibodies can be “humanized” by techniques well known in the art. See e.g., U.S. Pat. No. 5,225,539. Each of the above citations are incorporated herein by reference. Antibody fragments that contain the idiotypes to a SECX protein may be produced by techniques known in the art including, but not limited to: (i) an F_{(ab′)2} fragment produced by pepsin digestion of an antibody molecule; (ii) an F_{ab} fragment generated by reducing the disulfide bridges of an F_{(ab′)2} fragment; (iii) an F_{ab} fragment generated by the treatment of the antibody molecule with papain and a reducing agent and (iv) Fᵥ fragments.

Additionally, recombinant anti-SECX antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art, for example using methods described in PCT International Application No. PCT/US86/02269; European Patent Application No. 184,187; European Patent Application No. 171,496; European Patent Application No. 173,494; PCT International Publication No. WO 86/01533; U.S. Pat. No. 4,816,567; European Patent Application No. 125,023; Better et al.(1988) Science 240:1041-1043; Liu et al. (1987) PNAS 84:3439-3443; Liu et al. (1987) J Immunol. 139:3521-3526; Sun et al. (1987) PNAS 84:214-218; Nishimura et al. (1987) Cancer Res 47:999-1005; Wood et al. (1985) Nature 314:446-449; Shaw et al. (1988), J. Natl Cancer Inst 80:1553-1559); Morrison(1985) Science 229:1202-1207; Oi et al. (1986) BioTechniques 4:214; U.S. Pat. No. 5,225,539; Jones et al. (1986) Nature 321:552-525; Verhoeyan et al. (1988) Science 239:1534; and Beidler et al. (1988) J Immunol 141:4053-4060. Each of the above citations are incorporated herein by reference.

In one embodiment, methods for the screening of antibodies that possess the desired specificity include, but are not limited to, enzyme-linked immunosorbent assay (ELISA) and other immunologically-mediated techniques known within the art. In a specific embodiment, selection of antibodies that are specific to a particular domain of a SECX protein is facilitated by generation of hybridomas that bind to the fragment of a SECX protein possessing such a domain. Antibodies that are specific for one or more domains within a SECX protein, e.g., the domain spanning the first fifty amino-terminal residues specific to SECX when compared to FGF-9, or derivatives, fragments, analogs or homologs thereof, are also provided herein.

Anti-SECX antibodies may be used in methods known within the art relating to the localization and/or quantitation of a SECX protein (e.g., for use in measuring levels of the SECX protein within appropriate physiological samples, for use in diagnostic methods, for use in imaging the protein, and the like). In a given embodiment, antibodies for SECX proteins, or derivatives, fragments, analogs or homologs thereof, that contain the antibody derived binding domain, are utilized as pharmacologically-active compounds [hereinafter “Therapeutics”].

An anti-SECX antibody (e.g., monoclonal antibody) can be used to isolate SECX by standard techniques, such as affinity chromatography or immunoprecipitation. An anti-SECX antibody can facilitate the purification of natural SECX from cells and of recombinantly produced SECX expressed in host cells. Moreover, an anti-SECX antibody can be used to detect SECX protein (e.g., in a cellular lysate or cell supernatant) in order to evaluate the abundance and pattern of expression of the SECX protein.

Anti-SECX antibodies can be used diagnostically to monitor protein levels in tissue as part of a clinical testing procedure, e.g., to, for example, determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

SECX Recombinant Vectors and Host Cells

Another aspect of the invention pertains to vectors, preferably expression vectors, containing a nucleic acid encoding SECX protein, or derivatives, fragments, analogs or homologs thereof. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, that is operatively linked to the nucleic acid sequence to be expressed. Within a recombinant expression vector, “operably linked” is intended to mean that the nucleotide sequence of interest is linked to the regulatory sequence(s) in a manner that allows for expression of the nucleotide sequence (e.g., in an in vitro transcription/translation system or in a host cell when the vector is introduced into the host cell). The term “regulatory sequence” is intended to includes promoters, enhancers and other expression control elements (e.g., polyadenylation signals). Such regulatory sequences are described, for example, in Goeddel; GENE EXPRESSION TECHNOLOGY: METHODS IN ENZYMOLOGY 185, Academic Press, San Diego, Calif. (1990). Regulatory sequences include those that direct constitutive expression of a nucleotide sequence in many types of host cell and those that direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein (e.g., SECX proteins, mutant forms of SECX, fusion proteins, etc.).

The recombinant expression vectors of the invention can be designed for expression of SECX in prokaryotic or eukaryotic cells. For example, SECX can be expressed in bacterial cells such as E. coli, insect cells (using baculovirus expression vectors) yeast cells or mammalian cells. Suitable host cells are discussed further in Goeddel, GENE EXPRESSION TECHNOLOGY: METHODS IN ENZYMOLOGY 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: (1) to increase expression of recombinant protein; (2) to increase the solubility of the recombinant protein; and (3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith and Johnson (1988) Gene 67:31-40), pMAL (New England Biolabs, Beverly, Mass.) and pRIT5 (Pharmacia, Piscataway, N.J.) that fuse glutathione S-transferase (GST), maltose E binding protein, or protein A, respectively, to the target recombinant protein. Examples of suitable inducible non-fusion E. coli expression vectors include pTrc (Amrann et al., (1988) Gene 69:301-315) and pET 11d (Studier et al., GENE EXPRESSION TECHNOLOGY: METHODS IN ENZYMOLOGY 185, Academic Press, San Diego, Calif. (1990) 60-89).

One strategy to maximize recombinant protein expression in E. coli is to express the protein in a host bacteria with an impaired capacity to proteolytically cleave the recombinant protein. See, Gottesman, GENE EXPRESSION TECHNOLOGY: METHODS IN ENZYMOLOGY 185, Academic Press, San Diego, Calif. (1990) 119-128. Another strategy is to alter the nucleic acid sequence of the nucleic acid to be inserted into an expression vector so that the individual codons for each amino acid are those preferentially utilized in E. coli (Wada et al., (1992) Nucleic Acids Res. 20:2111-2118). Such alteration of nucleic acid sequences of the invention can be carried out by standard DNA synthesis techniques.

In another embodiment, the SECX expression vector is a yeast expression vector. Examples of vectors for expression in yeast S. cerivisae include pYepSec1 (Baldari, et al., (1987) EMBO J 6:229-234), pMFa (Kurjan and Herskowitz, (1982) Cell 30:933-943), pJRY88 (Schultz et al., (1987) Gene 54:113-123), pYES2 (Invitrogen Corporation, San Diego, Calif.), and picZ (InVitrogen Corp, San Diego, Calif.).

Alternatively, SECX can be expressed in insect cells using baculovirus expression vectors. Baculovirus vectors available for expression of proteins in cultured insect cells (e.g., SF9 cells) include the pAc series (Smith et al. (1983) Mol Cell Biol 3:2156-2165) and the pVL series (Lucklow and Summers (1989) Virology 170:31-39).

In yet another embodiment, a nucleic acid of the invention is expressed in mammalian cells using a mammalian expression vector. Examples of mammalian expression vectors include pCDM8 (Seed (1987) Nature 329:840) and pMT2PC (Kaufman et al. (1987) EMBO J 6: 187-195). When used in mammalian cells, the expression vector's control functions are often provided by viral regulatory elements. For example, commonly used promoters are derived from polyoma, Adenovirus 2, cytomegalovirus and Simian Virus 40. For other suitable expression systems for both prokaryotic and eukaryotic cells. See, e.g., Chapters 16 and 17 of Sambrook et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert et al. (1987) Genes Dev 1:268-277), lymphoid-specific promoters (Calame and Eaton (1988) Adv Immunol 43:235-275), in particular promoters of T cell receptors (Winoto and Baltimore (1989) EMBO J 8:729-733) and immunoglobulins (Baneiji et al. (1983) Cell 33:729-740; Queen and Baltimore (1983) Cell 33:741-748), neuron-specific promoters (e.g., the neurofilament promoter; Byrne and Ruddle (1989) PNAS 86:5473-5477), pancreas-specific promoters (Edlund et al. (1985) Science 230:912-916), and mammary gland-specific promoters (e.g., milk whey promoter; U.S. Pat. No. 4,873,316 and European Application Publication No. 264,166). Developmentally-regulated promoters are also encompassed, e.g., the murine hox promoters (Kessel and Gruss (1990) Science 249:374-379) and the α-fetoprotein promoter (Campes and Tilghman (1989) Genes Dev 3:537-546).

The invention further provides a recombinant expression vector comprising a DNA molecule of the invention cloned into the expression vector in an antisense orientation. That is, the DNA molecule is operatively linked to a regulatory sequence in a manner that allows for expression (by transcription of the DNA molecule) of an RNA molecule that is antisense to SECX mRNA. Regulatory sequences operatively linked to a nucleic acid cloned in the antisense orientation can be chosen that direct the continuous expression of the antisense RNA molecule in a variety of cell types, for instance viral promoters and/or enhancers, or regulatory sequences can be chosen that direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see Weintraub et al., “Antisense RNA as a molecular tool for genetic analysis,” Reviews—Trends in Genetics, Vol. 1(1) 1986.

Another aspect of the invention pertains to host cells into which a recombinant expression vector of the invention has been introduced. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, SECX protein can be expressed in bacterial cells such as E. coli, insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (MOLECULAR CLONING: A LABORATORY MANUAL. 2nd ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Various selectable markers include those that confer resistance to drugs, such as G418, hygromycin and methotrexate. Nucleic acid encoding a selectable marker can be introduced into a host cell on the same vector as that encoding SECX or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) SECX protein. Accordingly, the invention further provides methods for producing SECX protein using the host cells of the invention. In one embodiment, the method comprises culturing the host cell of invention (into which a recombinant expression vector encoding SECX has been introduced) in a suitable medium such that SECX protein is produced. In another embodiment, the method further comprises isolating SECX from the medium or the host cell.

Transgenic Animals

The host cells of the invention can also be used to produce nonhuman transgenic animals. For example, in one embodiment, a host cell of the invention is a fertilized oocyte or an embryonic stem cell into which SECX-coding sequences have been introduced. Such host cells can then be used to create non-human transgenic animals in which exogenous SECX sequences have been introduced into their genome or homologous recombinant animals in which endogenous SECX sequences have been altered. Such animals are useful for studying the function and/or activity of SECX and for identifying and/or evaluating modulators of SECX activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, etc. A transgene is exogenous DNA that is integrated into the genome of a cell from which a transgenic animal develops and that remains in the genome of the mature animal, thereby directing the expression of an encoded gene product in one or more cell types or tissues of the transgenic animal. As used herein, a “homologous recombinant animal” is a non-human animal, preferably a mammal, more preferably a mouse, in which an endogenous SECX gene has been altered by homologous recombination between the endogenous gene and an exogenous DNA molecule introduced into a cell of the animal, e.g., an embryonic cell of the animal, prior to development of the animal.

A transgenic animal of the invention can be created by introducing SECX-encoding nucleic acid into the male pronuclei of a fertilized oocyte, e.g., by microinjection, retroviral infection, and allowing the oocyte to develop in a pseudopregnant female foster animal. The human SECX DNA sequence of SEQ ID NO: 2n−1 (wherein n=1 to 20) can be introduced as a transgene into the genome of a non-human animal. Alternatively, a nonhuman homologue of the human SECX gene, such as a mouse SECX gene, can be isolated based on hybridization to the human SECX cDNA (described further above) and used as a transgene. Intronic sequences and polyadenylation signals can also be included in the transgene to increase the efficiency of expression of the transgene. A tissue-specific regulatory sequence(s) can be operably linked to the SECX transgene to direct expression of SECX protein to particular cells. Methods for generating transgenic animals via embryo manipulation and microinjection, particularly animals such as mice, have become conventional in the art and are described, for example, in U.S. Pat. Nos. 4,736,866; 4,870,009; and 4,873,191; and Hogan 1986, In: MANIPULATING THE MOUSE EMBRYO, Cold Spring Harbor Laboratory Press, Cold Spring Harbor, N.Y. Similar methods are used for production of other transgenic animals. A transgenic founder animal can be identified based upon the presence of the SECX transgene in its genome and/or expression of SECX mRNA in tissues or cells of the animals. A transgenic founder animal can then be used to breed additional animals carrying the transgene. Moreover, transgenic animals carrying a transgene encoding SECX can further be bred to other transgenic animals carrying other transgenes.

To create a homologous recombinant animal, a vector is prepared which contains at least a portion of a SECX gene into which a deletion, addition or substitution has been introduced to thereby alter, e.g., functionally disrupt, the SECX gene. The SECX gene can be a human gene (e.g., SEQ ID NO: 2n−1 (wherein n=1 to 20)), but more preferably, is a non-human homologue of a human SECX gene. For example, a mouse homologue of human SECX gene of SEQ ID NO: 2n−1 (wherein n=1 to 20) can be used to construct a homologous recombination vector suitable for altering an endogenous SECX gene in the mouse genome. In one embodiment, the vector is designed such that, upon homologous recombination, the endogenous SECX gene is functionally disrupted (i.e., no longer encodes a functional protein; also referred to as a “knock out” vector).

Alternatively, the vector can be designed such that, upon homologous recombination, the endogenous SECX gene is mutated or otherwise altered but still encodes functional protein (e.g., the upstream regulatory region can be altered to thereby alter the expression of the endogenous SECX protein). In the homologous recombination vector, the altered portion of the SECX gene is flanked at its 5′ and 3′ ends by additional nucleic acid of the SECX gene to allow for homologous recombination to occur between the exogenous SECX gene carried by the vector and an endogenous SECX gene in an embryonic stem cell. The additional flanking SECX nucleic acid is of sufficient length for successful homologous recombination with the endogenous gene. Typically, several kilobases of flanking DNA (both at the 5′ and 3′ ends) are included in the vector. See e.g., Thomas et al. (1987) Cell 51:503 for a description of homologous recombination vectors. The vector is introduced into an embryonic stem cell line (e.g., by electroporation) and cells in which the introduced SECX gene has homologously recombined with the endogenous SECX gene are selected (see e.g., Li et al. (1992) Cell 69:915).

The selected cells are then injected into a blastocyst of an animal (e.g., a mouse) to form aggregation chimeras. See e.g., Bradley 1987, In: TERATOCARCINOMAS AND EMBRYONIC STEM CELLS: A PRACTICAL APPROACH, Robertson, ed. IRL, Oxford, pp. 113-152. A chimeric embryo can then be implanted into a suitable pseudopregnant female foster animal and the embryo brought to term. Progeny harboring the homologously recombined DNA in their germ cells can be used to breed animals in which all cells of the animal contain the homologously recombined DNA by germline transmission of the transgene. Methods for constructing homologous recombination vectors and homologous recombinant animals are described further in Bradley (1991) Curr Opin Biotechnol 2:823-829; PCT International Publication Nos.: WO 90/1184; WO 91/01140; WO 92/0968; and WO 93/04169.

In another embodiment, transgenic non-humans animals can be produced that contain selected systems that allow for regulated expression of the transgene. One example of such a system is the cre/loxP recombinase system of bacteriophage P1. For a description of the cre/loxP recombinase system, see, e.g., Lakso et al. (1992) PNAS 89:6232-6236. Another example of a recombinase system is the FLP recombinase system of Saccharomyces cerevisiae (O'Gorman et al. (1991) Science 251:181-185. If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut et al. (1997) Nature 385:810-813. In brief, a cell, e.g., a somatic cell, from the transgenic animal can be isolated and induced to exit the growth cycle and enter G₀ phase. The quiescent cell can then be fused, e.g., through the use of electrical pulses, to an enucleated oocyte from an animal of the same species from which the quiescent cell is isolated. The reconstructed oocyte is then cultured such that it develops to morula or blastocyte and then transferred to pseudopregnant female foster animal. The offspring borne of this female foster animal will be a clone of the animal from which the cell, e.g., the somatic cell, is isolated.

Pharmaceutical Compositions

The SECX nucleic acid molecules, SECX proteins, and anti-SECX antibodies (also referred to herein as “active compounds”) of the invention, and derivatives, fragments, analogs and homologs thereof, can be incorporated into pharmaceutical compositions suitable for administration. Such compositions typically comprise the nucleic acid molecule, protein, or antibody and a pharmaceutically acceptable carrier. As used herein, “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, finger's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., a SECX protein or anti-SECX antibody) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring. For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved.

The nucleic acid molecules of the invention can be inserted into vectors and used as gene therapy vectors. Gene therapy vectors can be delivered to a subject by any of a number of routes, e.g., as described in U.S. Pat. No. 5,703,055. Delivery can thus also include, e.g., intravenous injection, local administration (see U.S. Pat. No. 5,328,470) or stereotactic injection (see e.g., Chen et al. (1994) PNAS 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells that produce the gene delivery system.

The pharmaceutical compositions can be included in a kit, e.g., in a container, pack, or dispenser together with instructions for administration.

Also within the invention is the use of a therapeutic in the manufacture of a medicament for treating a syndrome associated with a human disease, the disease selected from a SECX-associated disorder, wherein said therapeutic is selected from the group consisting of a SECX polypeptide, a SECX nucleic acid, and a SECX antibody.

Additional Uses and Methods of the Invention

The nucleic acid molecules, proteins, protein homologues, and antibodies described herein can be used in one or more of the following methods: (a) screening assays; (b) detection assays (e.g., chromosomal mapping, cell and tissue typing, forensic biology), (c) predictive medicine (e.g., diagnostic assays, prognostic assays, monitoring clinical trials, and pharmacogenomics); and (d) methods of treatment (e.g., therapeutic and prophylactic).

The isolated nucleic acid molecules of the invention can be used to express SECX protein (e.g., via a recombinant expression vector in a host cell in gene therapy applications), to detect SECX mRNA (e.g., in a biological sample) or a genetic lesion in a SECX gene, and to modulate SECX activity, as described further below. In addition, the SECX proteins can be used to screen drugs or compounds that modulate the SECX activity or expression as well as to treat disorders characterized by insufficient or excessive production of SECX protein, for example proliferative or differentiative disorders, or production of SECX protein forms that have decreased or aberrant activity compared to SECX wild type protein. In addition, the anti-SECX antibodies of the invention can be used to detect and isolate SECX proteins and modulate SECX activity.

This invention further pertains to novel agents identified by the above described screening assays and uses thereof for treatments as described herein.

Screening Assays

The invention provides a method (also referred to herein as a “screening assay”) for identifying modulators, i.e., candidate or test compounds or agents (e.g., peptides, peptidomimetics, small molecules or other drugs) that bind to SECX proteins or have a stimulatory or inhibitory effect on, for example, SECX expression or SECX activity.

In one embodiment, the invention provides assays for screening candidate or test compounds which bind to or modulate the activity of a SECX protein or polypeptide or biologically active portion thereof. The test compounds of the present invention can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds (Lam (1997) Anticancer Drug Des 12:145).

Examples of methods for the synthesis of molecular libraries can be found in the art, for example in: DeWitt et al. (1993) Proc Natl Acad Sci U.S.A. 90:6909; Erb et al. (1994) Proc Natl Acad Sci U.S.A. 91:11422; Zuckermann et al. (1994) J Med Chem 37:2678; Cho et al. (1993) Science 261:1303; Carrell et al. (1994) Angew Chem Int Ed Engl 33:2059; Carell et al. (1994) Angew Chem Int Ed Engl 33:2061; and Gallop et al. (1994) J Med Chem 37:1233.

Libraries of compounds may be presented in solution (e.g., Houghten (1992) Biotechniques 13:412-421), or on beads (Lam (1991) Nature 354:82-84), on chips (Fodor (1993) Nature 364:555-556), bacteria (Ladner U.S. Pat. No. 5,223,409), spores (Ladner U.S. Pat. No. '409), plasmids (Cull et al. (1992) Proc Natl Acad Sci USA 89:1865-1869) or on phage (Scott and Smith (1990) Science 249:386-390; Devlin (1990) Science 249:404-406; Cwirla et al. (1990) Proc Natl Acad Sci U.S.A. 87:6378-6382; Felici (1991) J Mol Biol 222:301-310; Ladner above.).

In one embodiment, an assay is a cell-based assay in which a cell which expresses a membrane-bound form of SECX protein, or a biologically active portion thereof, on the cell surface is contacted with a test compound and the ability of the test compound to bind to a SECX protein determined. The cell, for example, can of mammalian origin or a yeast cell. Determining the ability of the test compound to bind to the SECX protein can be accomplished, for example, by coupling the test compound with a radioisotope or enzymatic label such that binding of the test compound to the SECX protein or biologically active portion thereof can be determined by detecting the labeled compound in a complex. For example, test compounds can be labeled with ¹²⁵I, ³⁵S, ¹⁴C, or ³H, either directly or indirectly, and the radioisotope detected by direct counting of radioemission or by scintillation counting. Alternatively, test compounds can be enzymatically labeled with, for example, horseradish peroxidase, alkaline phosphatase, or luciferase, and the enzymatic label detected by determination of conversion of an appropriate substrate to product. In one embodiment, the assay comprises contacting a cell which expresses a membrane-bound form of SECX protein, or a biologically active portion thereof, on the cell surface with a known compound which binds SECX to form an assay mixture, contacting the assay mixture with a test compound, and determining the ability of the test compound to interact with a SECX protein, wherein determining the ability of the test compound to interact with a SECX protein comprises determining the ability of the test compound to preferentially bind to SECX or a biologically active portion thereof as compared to the known compound.

In another embodiment, an assay is a cell-based assay comprising contacting a cell expressing a membrane-bound form of SECX protein, or a biologically active portion thereof, on the cell surface with a test compound and determining the ability of the test compound to modulate (e.g., stimulate or inhibit) the activity of the SECX protein or biologically active portion thereof. Determining the ability of the test compound to modulate the activity of SECX or a biologically active portion thereof can be accomplished, for example, by determining the ability of the SECX protein to bind to or interact with a SECX target molecule. As used herein, a “target molecule” is a molecule with which a SECX protein binds or interacts in nature, for example, a molecule on the surface of a cell which expresses a SECX interacting protein, a molecule on the surface of a second cell, a molecule in the extracellular milieu, a molecule associated with the internal surface of a cell membrane or a cytoplasmic molecule. A SECX target molecule can be a non-SECX molecule or a SECX protein or polypeptide of the present invention. In one embodiment, a SECX target molecule is a component of a signal transduction pathway that facilitates transduction of an extracellular signal (e.g., a signal generated by binding of a compound to a membrane-bound SECX molecule) through the cell membrane and into the cell. The target, for example, can be a second intercellular protein that has catalytic activity or a protein that facilitates the association of downstream signaling molecules with SECX.

Determining the ability of the SECX protein to bind to or interact with a SECX target molecule can be accomplished by one of the methods described above for determining direct binding. In one embodiment, determining the ability of the SECX protein to bind to or interact with a SECX target molecule can be accomplished by determining the activity of the target molecule. For example, the activity of the target molecule can be determined by detecting induction of a cellular second messenger of the target (i.e. intracellular Ca²⁺, diacylglycerol, IP₃, etc.), detecting catalytic/enzymatic activity of the target an appropriate substrate, detecting the induction of a reporter gene (comprising a SECX-responsive regulatory element operatively linked to a nucleic acid encoding a detectable marker, e.g., luciferase), or detecting a cellular response, for example, cell survival, cellular differentiation, or cell proliferation.

In yet another embodiment, an assay of the present invention is a cell-free assay comprising contacting a SECX protein or biologically active portion thereof with a test compound and determining the ability of the test compound to bind to the SECX protein or biologically active portion thereof. Binding of the test compound to the SECX protein can be determined either directly or indirectly as described above. In one embodiment, the assay comprises contacting the SECX protein or biologically active portion thereof with a known compound which binds SECX to form an assay mixture, contacting the assay mixture with a test compound, and determining the ability of the test compound to interact with a SECX protein, wherein determining the ability of the test compound to interact with a SECX protein comprises determining the ability of the test compound to preferentially bind to SECX or biologically active portion thereof as compared to the known compound.

In another embodiment, an assay is a cell-free assay comprising contacting SECX protein or biologically active portion thereof with a test compound and determining the ability of the test compound to modulate (e.g., stimulate or inhibit) the activity of the SECX protein or biologically active portion thereof. Determining the ability of the test compound to modulate the activity of SECX can be accomplished, for example, by determining the ability of the SECX protein to bind to a SECX target molecule by one of the methods described above for determining direct binding. In an alternative embodiment, determining the ability of the test compound to modulate the activity of SECX can be accomplished by determining the ability of the SECX protein further modulate a SECX target molecule. For example, the catalytic/enzymatic activity of the target molecule on an appropriate substrate can be determined as previously described.

In yet another embodiment, the cell-free assay comprises contacting the SECX protein or biologically active portion thereof with a known compound which binds SECX to form an assay mixture, contacting the assay mixture with a test compound, and determining the ability of the test compound to interact with a SECX protein, wherein determining the ability of the test compound to interact with a SECX protein comprises determining the ability of the SECX protein to preferentially bind to or modulate the activity of a SECX target molecule.

The cell-free assays of the present invention are amenable to use of both the soluble form or the membrane-bound form of SECX. In the case of cell-free assays comprising the membrane-bound form of SECX, it may be desirable to utilize a solubilizing agent such that the membrane-bound form of SECX is maintained in solution. Examples of such solubilizing agents include non-ionic detergents such as n-octylglucoside, n-dodecylglucoside, n-dodecylmaltoside, octanoyl-N-methylglucamide, decanoyl-N-methylglucamide, Triton® X-100, Triton® X-114, Thesit®, Isotridecypoly(ethylene glycol ether)ₙ, N-dodecyl—N,N-dimethyl-3-ammonio-1-propane sulfonate, 3-(3-cholamidopropyl)dimethylamminiol-1-propane sulfonate (CHAPS), or 3-(3-cholamidopropyl)dimethylamminiol-2-hydroxy-1-propane sulfonate (CHAPSO).

In more than one embodiment of the above assay methods of the present invention, it may be desirable to immobilize either SECX or its target molecule to facilitate separation of complexed from uncomplexed forms of one or both of the proteins, as well as to accommodate automation of the assay. Binding of a test compound to SECX, or interaction of SECX with a target molecule in the presence and absence of a candidate compound, can be accomplished in any vessel suitable for containing the reactants. Examples of such vessels include microtiter plates, test tubes, and micro-centrifuge tubes. In one embodiment, a fusion protein can be provided that adds a domain that allows one or both of the proteins to be bound to a matrix. For example, GST-SECX fusion proteins or GST-target fusion proteins can be adsorbed onto glutathione sepharose beads (Sigma Chemical, St. Louis, Mo.) or glutathione derivatized microtiter plates, that are then combined with the test compound or the test compound and either the non-adsorbed target protein or SECX protein, and the mixture is incubated under conditions conducive to complex formation (e.g., at physiological conditions for salt and pH). Following incubation, the beads or microtiter plate wells are washed to remove any unbound components, the matrix immobilized in the case of beads, complex determined either directly or indirectly, for example, as described above. Alternatively, the complexes can be dissociated from the matrix, and the level of SECX binding or activity determined using standard techniques.

Other techniques for immobilizing proteins on matrices can also be used in the screening assays of the invention. For example, either SECX or its target molecule can be immobilized utilizing conjugation of biotin and streptavidin. Biotinylated SECX or target molecules can be prepared from biotin-NHS (N-hydroxy-succinimide) using techniques well known in the art (e.g., biotinylation kit, Pierce Chemicals, Rockford, Ill.), and immobilized in the wells of streptavidin-coated 96 well plates (Pierce Chemical). Alternatively, antibodies reactive with SECX or target molecules, but which do not interfere with binding of the SECX protein to its target molecule, can be derivatized to the wells of the plate, and unbound target or SECX trapped in the wells by antibody conjugation. Methods for detecting such complexes, in addition to those described above for the GST-immobilized complexes, include immunodetection of complexes using antibodies reactive with the SECX or target molecule, as well as enzyme-linked assays that rely on detecting an enzymatic activity associated with the SECX or target molecule.

In another embodiment, modulators of SECX expression are identified in a method wherein a cell is contacted with a candidate compound and the expression of SECX mRNA or protein in the cell is determined. The level of expression of SECX mRNA or protein in the presence of the candidate compound is compared to the level of expression of SECX mRNA or protein in the absence of the candidate compound. The candidate compound can then be identified as a modulator of SECX expression based on this comparison. For example, when expression of SECX mRNA or protein is greater (statistically significantly greater) in the presence of the candidate compound than in its absence, the candidate compound is identified as a stimulator of SECX mRNA or protein expression. Alternatively, when expression of SECX mRNA or protein is less (statistically significantly less) in the presence of the candidate compound than in its absence, the candidate compound is identified as an inhibitor of SECX mRNA or protein expression. The level of SECX mRNA or protein expression in the cells can be determined by methods described herein for detecting SECX mRNA or protein.

In yet another aspect of the invention, the SECX proteins can be used as “bait proteins” in a two-hybrid assay or three hybrid assay (see, e.g., U.S. Pat. No. 5,283,317; Zervos et al. (1993) Cell 72:223-232; Madura et al. (1993) J Biol Chem 268:12046-12054; Bartel et al. (1993) Biotechniques 14:920-924; Iwabuchi et al. (1993) Oncogene 8:1693-1696; and Brent WO94/10300), to identify other proteins that bind to or interact with SECX (“SECX-binding proteins” or “SECX-bp”) and modulate SECX activity. Such SECX-binding proteins are also likely to be involved in the propagation of signals by the SECX proteins as, for example, upstream or downstream elements of the SECX pathway.

The two-hybrid system is based on the modular nature of most transcription factors, which consist of separable DNA-binding and activation domains. Briefly, the assay utilizes two different DNA constructs. In one construct, the gene that codes for SECX is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming a SECX-dependent complex, the DNA-binding and activation domains of the transcription factor are brought into close proximity. This proximity allows transcription of a reporter gene (e.g., LacZ) that is operably linked to a transcriptional regulatory site responsive to the transcription factor. Expression of the reporter gene can be detected and cell colonies containing the functional transcription factor can be isolated and used to obtain the cloned gene that encodes the protein which interacts with SECX.

Screening can also be performed in vivo. For example, in one embodiment, the invention includes a method for screening for a modulator of activity or of latency or predisposition to a SECX-associated disorder by administering a test compound or to a test animal at increased risk for a SECX-associated disorder. In some embodiments, the test animal recombinantly expresses a SECX polypeptide. Activity of the polypeptide in the test animal after administering the compound is measured, and the activity of the protein in the test animal is compared to the activity of the polypeptide in a control animal not administered said polypeptide. A change in the activity of said polypeptide in said test animal relative to the control animal indicates the test compound is a modulator of latency of or predisposition to a SECX-associated disorder.

In some embodiments, the test animal is a recombinant test animal that expresses a test protein transgene or expresses the transgene under the control of a promoter at an increased level relative to a wild-type test animal. Preferably, the promoter is not the native gene promoter of the transgene.

This invention further pertains to novel agents identified by the above-described screening assays and uses thereof for treatments as described herein.

Detection Assays

Portions or fragments of the cDNA sequences identified herein (and the corresponding complete gene sequences) can be used in numerous ways as polynucleotide reagents. For example, these sequences can be used to: (i) map their respective genes on a chromosome; and, thus, locate gene regions associated with genetic disease; (ii) identify an individual from a minute biological sample (tissue typing); and (iii) aid in forensic identification of a biological sample.

The SECX sequences of the present invention can also be used to identify individuals from minute biological samples. In this technique, an individual's genomic DNA is digested with one or more restriction enzymes, and probed on a Southern blot to yield unique bands for identification. The sequences of the present invention are useful as additional DNA markers for RFLP (“restriction fragment length polymorphisms,” described in U.S. Pat. No. 5,272,057).

Furthermore, the sequences of the present invention can be used to provide an alternative technique that determines the actual base-by-base DNA sequence of selected portions of an individual's genome. Thus, the SECX sequences described herein can be used to prepare two PCR primers from the 5′ and 3′ ends of the sequences. These primers can then be used to amplify an individual's DNA and subsequently sequence it.

Panels of corresponding DNA sequences from individuals, prepared in this manner, can provide unique individual identifications, as each individual will have a unique set of such DNA sequences due to allelic differences. The sequences of the present invention can be used to obtain such identification sequences from individuals and from tissue. The SECX sequences of the invention uniquely represent portions of the human genome. Allelic variation occurs to some degree in the coding regions of these sequences, and to a greater degree in the noncoding regions. It is estimated that allelic variation between individual humans occurs with a frequency of about once per each 500 bases. Much of the allelic variation is due to single nucleotide polymorphisms (SNPs), which include restriction fragment length polymorphisms (RFLPs).

Each of the sequences described herein can, to some degree, be used as a standard against which DNA from an individual can be compared for identification purposes. Because greater numbers of polymorphisms occur in the noncoding regions, fewer sequences are necessary to differentiate individuals. The noncoding sequences of SEQ ID NO: 2n−1 (wherein n=1 to 20), as described above, can comfortably provide positive individual identification with a panel of perhaps 10 to 1,000 primers that each yield a noncoding amplified sequence of 100 bases. If predicted coding sequences are used, a more appropriate number of primers for positive individual identification would be 500-2,000.

Predictive Medicine

The present invention also pertains to the field of predictive medicine in which diagnostic assays, prognostic assays, pharmacogenomics, and monitoring clinical trials are used for prognostic (predictive) purposes to thereby treat an individual prophylactically. Accordingly, one aspect of the present invention relates to diagnostic assays for determining SECX protein and/or nucleic acid expression as well as SECX activity, in the context of a biological sample (e.g., blood, serum, cells, tissue) to thereby determine whether an individual is afflicted with a disease or disorder, or is at risk of developing a disorder, associated with aberrant SECX expression or activity. The invention also provides for prognostic (or predictive) assays for determining whether an individual is at risk of developing a disorder associated with SECX protein, nucleic acid expression or activity. For example, mutations in a SECX gene can be assayed in a biological sample. Such assays can be used for prognostic or predictive purpose to thereby prophylactically treat an individual prior to the onset of a disorder characterized by or associated with SECX protein, nucleic acid expression or activity.

Another aspect of the invention provides methods for determining SECX protein, nucleic acid expression or SECX activity in an individual to thereby select appropriate therapeutic or prophylactic agents for that individual (referred to herein as “pharmacogenomics”). Pharmacogenomics allows for the selection of agents (e.g., drugs) for therapeutic or prophylactic treatment of an individual based on the genotype of the individual (e.g., the genotype of the individual examined to determine the ability of the individual to respond to a particular agent.)

Yet another aspect of the invention pertains to monitoring the influence of agents (e.g., drugs, compounds) on the expression or activity of SECX in clinical trials.

Use of Partial SECX Sequences in Forensic Biology

DNA-based identification techniques can also be used in forensic biology. Forensic biology is a scientific field employing genetic typing of biological evidence found at a crime scene as a means for positively identifying, for example, a perpetrator of a crime. To make such an identification, PCR technology can be used to amplify DNA sequences taken from very small biological samples such as tissues, e.g., hair or skin, or body fluids, e.g., blood, saliva, or semen found at a crime scene. The amplified sequence can then be compared to a standard, thereby allowing identification of the origin of the biological sample.

The sequences of the present invention can be used to provide polynucleotide reagents, e.g., PCR primers, targeted to specific loci in the human genome, that can enhance the reliability of DNA-based forensic identifications by, for example, providing another “identification marker” (i.e. another DNA sequence that is unique to a particular individual). As mentioned above, actual base sequence information can be used for identification as an accurate alternative to patterns formed by restriction enzyme generated fragments. Sequences targeted to noncoding regions of SEQ ID NO: 2n−1 (where n=1 to 20) are particularly appropriate for this use as greater numbers of polymorphisms occur in the noncoding regions, making it easier to differentiate individuals using this technique. Examples of polynucleotide reagents include the SECX sequences or portions thereof, e.g., fragments derived from the noncoding regions of one or more of SEQ ID NO: 2n−1 (where n=1 to 20), having a length of at least 20 bases, preferably at least 30 bases.

The SECX sequences described herein can further be used to provide polynucleotide reagents, e.g., labeled or label-able probes that can be used, for example, in an in situ hybridization technique, to identify a specific tissue, e.g., brain tissue, etc. This can be very useful in cases where a forensic pathologist is presented with a tissue of unknown origin. Panels of such SECX probes can be used to identify tissue by species and/or by organ type.

In a similar fashion, these reagents, e.g., SECX primers or probes can be used to screen tissue culture for contamination (i.e. screen for the presence of a mixture of different types of cells in a culture).

Predictive Medicine

The present invention also pertains to the field of predictive medicine in which diagnostic assays, prognostic assays, pharmacogenomics, and monitoring clinical trials are used for prognostic (predictive) purposes to thereby treat an individual prophylactically. Accordingly, one aspect of the present invention relates to diagnostic assays for determining SECX protein and/or nucleic acid expression as well as SECX activity, in the context of a biological sample (e.g., blood, serum, cells, tissue) to thereby determine whether an individual is afflicted with a disease or disorder, or is at risk of developing a disorder, associated with aberrant SECX expression or activity. The invention also provides for prognostic (or predictive) assays for determining whether an individual is at risk of developing a disorder associated with SECX protein, nucleic acid expression or activity. For example, mutations in a SECX gene can be assayed in a biological sample. Such assays can be used for prognostic or predictive purpose to thereby prophylactically treat an individual prior to the onset of a disorder characterized by or associated with SECX protein, nucleic acid expression or activity.

Another aspect of the invention provides methods for determining SECX protein, nucleic acid expression or SECX activity in an individual to thereby select appropriate therapeutic or prophylactic agents for that individual (referred to herein as “pharmacogenomics”). Pharmacogenomics allows for the selection of agents (e.g., drugs) for therapeutic or prophylactic treatment of an individual based on the genotype of the individual (e.g., the genotype of the individual examined to determine the ability of the individual to respond to a particular agent.) Yet another aspect of the invention pertains to monitoring the influence of agents (e.g., drugs, compounds) on the expression or activity of SECX in clinical trials. These and other agents are described in further detail in the following sections.

Diagnostic Assays

Other conditions in which proliferation of cells plays a role include tumors, restenosis, psoriasis, Dupuytren's contracture, diabetic complications, Kaposi's sarcoma and rheumatoid arthritis.

An SECX polypeptide may be used to identify an interacting polypeptide a sample or tissue. The method comprises contacting the sample or tissue with SECX, allowing formation of a complex between the SECX polypeptide and the interacting polypeptide, and detecting the complex, if present.

The proteins of the invention may be used to stimulate production of antibodies specifically binding the proteins. Such antibodies may be used in immunodiagnostic procedures to detect the occurrence of the protein in a sample. The proteins of the invention may be used to stimulate cell growth and cell proliferation in conditions in which such growth would be favorable. An example would be to counteract toxic side effects of chemotherapeutic agents on, for example, hematopoiesis and platelet formation, linings of the gastrointestinal tract, and hair follicles. They may also be used to stimulate new cell growth in neurological disorders including, for example, Alzheimer's disease. Alternatively, antagonistic treatments may be administered in which an antibody specifically binding the SECX -like proteins of the invention would abrogate the specific growth-inducing effects of the proteins. Such antibodies may be useful, for example, in the treatment of proliferative disorders including various tumors and benign hyperplasias.

Polynucleotides or oligonucleotides corresponding to any one portion of the SECX nucleic acids of SEQ ID NO: 2n−1 (wherein n=1 to 20) may be used to detect DNA containing a corresponding ORF gene, or detect the expression of a corresponding SECX gene, or SECX-like gene. For example, an SECX nucleic acid expressed in a particular cell or tissue, as noted in Table 2, can be used to identify the presence of that particular cell type.

An exemplary method for detecting the presence or absence of SECX in a biological sample involves obtaining a biological sample from a test subject and contacting the biological sample with a compound or an agent capable of detecting SECX protein or nucleic acid (e.g., mRNA, genomic DNA) that encodes SECX protein such that the presence of SECX is detected in the biological sample. An agent for detecting SECX mRNA or genomic DNA is a labeled nucleic acid probe capable of hybridizing to SECX mRNA or genomic DNA. The nucleic acid probe can be, for example, a full-length SECX nucleic acid, such as the nucleic acid of SEQ ID NO: 2n−1 (wherein n=1 to 20), or a portion thereof, such as an oligonucleotide of at least 15, 30, 50, 100, 250 or 500 nucleotides in length and sufficient to specifically hybridize under stringent conditions to SECX mRNA or genomic DNA, as described above. Other suitable probes for use in the diagnostic assays of the invention are described herein. An agent for detecting SECX protein is an antibody capable of binding to SECX protein, preferably an antibody with a detectable label. Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin. The term “biological sample” is intended to include tissues, cells and biological fluids isolated from a subject, as well as tissues, cells and fluids present within a subject. That is, the detection method of the invention can be used to detect SECX mRNA, protein, or genomic DNA in a biological sample in vitro as well as in vivo. For example, in vitro techniques for detection of SECX mRNA include Northern hybridizations and in situ hybridizations. In vitro techniques for detection of SECX protein include enzyme linked immunosorbent assays (ELISAs), Western blots, immunoprecipitations and immunofluorescence. In vitro techniques for detection of SECX genomic DNA include Southern hybridizations. Furthermore, in vivo techniques for detection of SECX protein include introducing into a subject a labeled anti-SECX antibody. For example, the antibody can be labeled with a radioactive marker whose presence and location in a subject can be detected by standard imaging techniques.

In one embodiment, the biological sample contains protein molecules from the test subject. Alternatively, the biological sample can contain mRNA molecules from the test subject or genomic DNA molecules from the test subject. A preferred biological sample is a peripheral blood leukocyte sample isolated by conventional means from a subject.

In another embodiment, the methods further involve obtaining a control biological sample from a control subject, contacting the control sample with a compound or agent capable of detecting SECX protein, mRNA, or genomic DNA, such that the presence of SECX protein, mRNA or genomic DNA is detected in the biological sample, and comparing the presence of SECX protein, mRNA or genomic DNA in the control sample with the presence of SECX protein, mRNA or genomic DNA in the test sample.

The invention also encompasses kits for detecting the presence of SECX in a biological sample. For example, the kit can comprise: a labeled compound or agent capable of detecting SECX protein or mRNA in a biological sample; means for determining the amount of SECX in the sample; and means for comparing the amount of SECX in the sample with a standard. The compound or agent can be packaged in a suitable container. The kit can further comprise instructions for using the kit to detect SECX protein or nucleic acid.

Prognostic Assays

The diagnostic methods described herein can furthermore be utilized to identify subjects having or at risk of developing a disease or disorder associated with aberrant SECX expression or activity. For example, the assays described herein, such as the preceding diagnostic assays or the following assays, can be utilized to identify a subject having or at risk of developing a disorder associated with SECX protein, nucleic acid expression or activity in, e.g., proliferative or differentiative disorders such as hyperplasias, tumors, restenosis, psoriasis, Dupuytren's contracture, diabetic complications, or rheumatoid arthritis, etc.; and glia-associated disorders such as cerebral lesions, diabetic neuropathies, cerebral edema, senile dementia, Alzheimer's disease, etc. Alternatively, the prognostic assays can be utilized to identify a subject having or at risk for developing a disease or disorder. Thus, the present invention provides a method for identifying a disease or disorder associated with aberrant SECX expression or activity in which a test sample is obtained from a subject and SECX protein or nucleic acid (e.g., mRNA, genomic DNA) is detected, wherein the presence of SECX protein or nucleic acid is diagnostic for a subject having or at risk of developing a disease or disorder associated with aberrant SECX expression or activity. As used herein, a “test sample” refers to a biological sample obtained from a subject of interest. For example, a test sample can be a biological fluid (e.g., serum), cell sample, or tissue.

Furthermore, the prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant SECX expression or activity. For example, such methods can be used to determine whether a subject can be effectively treated with an agent for a disorder, such as a proliferative disorder, differentiative disorder, glia-associated disorders, etc. Thus, the present invention provides methods for determining whether a subject can be effectively treated with an agent for a disorder associated with aberrant SECX expression or activity in which a test sample is obtained and SECX protein or nucleic acid is detected (e.g., wherein the presence of SECX protein or nucleic acid is diagnostic for a subject that can be administered the agent to treat a disorder associated with aberrant SECX expression or activity.)

The methods of the invention can also be used to detect genetic lesions in a SECX gene, thereby determining if a subject with the lesioned gene is at risk for, or suffers from, a proliferative disorder, differentiative disorder, glia-associated disorder, etc. In various embodiments, the methods include detecting, in a sample of cells from the subject, the presence or absence of a genetic lesion characterized by at least one of an alteration affecting the integrity of a gene encoding a SECX-protein, or the mis-expression of the SECX gene. For example, such genetic lesions can be detected by ascertaining the existence of at least one of (1) a deletion of one or more nucleotides from a SECX gene; (2) an addition of one or more nucleotides to a SECX gene; (3) a substitution of one or more nucleotides of a SECX gene, (4) a chromosomal rearrangement of a SECX gene; (5) an alteration in the level of a messenger RNA transcript of a SECX gene, (6) aberrant modification of a SECX gene, such as of the methylation pattern of the genomic DNA, (7) the presence of a non-wild type splicing pattern of a messenger RNA transcript of a SECX gene, (8) a non-wild type level of a SECX-protein, (9) allelic loss of a SECX gene, and (10) inappropriate post-translational modification of a SECX-protein. As described herein, there are a large number of assay techniques known in the art which can be used for detecting lesions in a SECX gene. A preferred biological sample is a peripheral blood leukocyte sample isolated by conventional means from a subject. However, any biological sample containing nucleated cells may be used, including, for example, buccal mucosal cells.

In certain embodiments, detection of the lesion involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran et al. (1988) Science 241:1077-1080; and Nakazawa et al. (1994) PNAS 91:360-364), the latter of which can be particularly useful for detecting point mutations in the SECX-gene (see Abravaya et al. (1995) Nucl Acids Res 23:675-682). This method can include the steps of collecting a sample of cells from a patient, isolating nucleic acid (e.g., genomic, mRNA or both) from the cells of the sample, contacting the nucleic acid sample with one or more primers that specifically hybridize to a SECX gene under conditions such that hybridization and amplification of the SECX gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (Guatelli et al., 1990, Proc Natl Acad Sci USA 87:1874-1878), transcriptional amplification system (Kwoh, et al., 1989, Proc Natl Acad Sci USA 86:1173-1177), Q-Beta Replicase (Lizardi et al, 1988, BioTechnology 6:1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in a SECX gene from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, for example, U.S. Pat. No. 5,493,531) can be used to score for the presence of specific mutations by development or loss of a ribozyme cleavage site.

In other embodiments, genetic mutations in SECX can be identified by hybridizing a sample and control nucleic acids, e.g., DNA or RNA, to high density arrays containing hundreds or thousands of oligonucleotides probes (Cronin et al. (1996) Human Mutation 7: 244-255; Kozal et al. (1996) Nature Medicine 2: 753-759). For example, genetic mutations in SECX can be identified in two dimensional arrays containing light-generated DNA probes as described in Cronin et al. above. Briefly, a first hybridization array of probes can be used to scan through long stretches of DNA in a sample and control to identify base changes between the sequences by making linear arrays of sequential overlapping probes. This step allows the identification of point mutations. This step is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the SECX gene and detect mutations by comparing the sequence of the sample SECX with the corresponding wild-type (control) sequence. Examples of sequencing reactions include those based on techniques developed by Maxim and Gilbert (1977) PNAS 74:560 or Sanger (1977) PNAS 74:5463. It is also contemplated that any of a variety of automated sequencing procedures can be utilized when performing the diagnostic assays (Naeve et al., (1995) Biotechniques 19:448), including sequencing by mass spectrometry (see, e.g., PCT International Pub]. No. WO 94/16101; Cohen et al. (1996) Adv Chromatogr 36:127-162; and Griffin et al. (1993) Appl Biochem Biotechnol 38:147-159).

Other methods for detecting mutations in the SECX gene include methods in which protection from cleavage agents is used to detect mismatched bases in RNA/RNA or RNA/DNA heteroduplexes (Myers et al. (1985) Science 230:1242). In general, the art technique of “mismatch cleavage” starts by providing heteroduplexes of formed by hybridizing (labeled) RNA or DNA containing the wild-type SECX sequence with potentially mutant RNA or DNA obtained from a tissue sample. The double-stranded duplexes are treated with an agent that cleaves single-stranded regions of the duplex such as which will exist due to basepair mismatches between the control and sample strands. For instance, RNA/DNA duplexes can be treated with RNase and DNA/DNA hybrids treated with S1 nuclease to enzymatically digesting the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, for example, Cotton et al (1988) Proc Natl Acad Sci USA 85:4397; Saleeba et al (1992) Methods Enzymol 217:286-295. In an embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in SECX cDNAs obtained from samples of cells. For example, the mutY enzyme of E. coli cleaves A at G/A mismatches and the thymidine DNA glycosylase from HeLa cells cleaves T at G/T mismatches (Hsu et al. (1994) Carcinogenesis 15:1657-1662). According to an exemplary embodiment, a probe based on a SECX sequence, e.g., a wild-type SECX sequence, is hybridized to a cDNA or other DNA product from a test cell(s). The duplex is treated with a DNA mismatch repair enzyme, and the cleavage products, if any, can be detected from electrophoresis protocols or the like. See, for example, U.S. Pat. No. 5,459,039.

In other embodiments, alterations in electrophoretic mobility will be used to identify mutations in SECX genes. For example, single strand conformation polymorphism (SSCP) may be used to detect differences in electrophoretic mobility between mutant and wild type nucleic acids (Orita et al. (1989) Proc Natl Acad Sci USA: 86:2766, see also Cotton (1993) Mutat Res 285:125-144; Hayashi (1992) Genet Anal Tech Appl 9:73-79). Single-stranded DNA fragments of sample and control SECX nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA, rather than DNA, in which the secondary structure is more sensitive to a change in sequence. In one embodiment, the subject method utilizes heteroduplex analysis to separate double stranded heteroduplex molecules on the basis of changes in electrophoretic mobility. See, e.g., Keen et al. (1991) Trends Genet 7:5.

In yet another embodiment the movement of mutant or wild-type fragments in polyacrylamide gels containing a gradient of denaturant is assayed using denaturing gradient gel electrophoresis (DGGE). See, e.g., Myers et al (1985) Nature 313:495. When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA. See, e.g., Rosenbaum and Reissner (1987) Biophys Chem 265:12753.

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions that permit hybridization only if a perfect match is found. See, e.g., Saiki et al. (1986) Nature 324:163); Saiki et al. (1989) Proc Natl Acad. Sci USA 86:6230. Such allele specific oligonucleotides are hybridized to PCR amplified target DNA or a number of different mutations when the oligonucleotides are attached to the hybridizing membrane and hybridized with labeled target DNA.

Alternatively, allele specific amplification technology that depends on selective PCR amplification may be used in conjunction with the instant invention. Oligonucleotides used as primers for specific amplification may carry the mutation of interest in the center of the molecule (so that amplification depends on differential hybridization) (Gibbs et al. (1989) Nucleic Acids Res 17:2437-2448) or at the extreme 3′ end of one primer where, under appropriate conditions, mismatch can prevent, or reduce polymerase extension (Prossner (1993) Tibtech 11:238). In addition it may be desirable to introduce a novel restriction site in the region of the mutation to create cleavage-based detection. See, e.g., Gasparini et al (1992) Mol Cell Probes 6:1. It is anticipated that in certain embodiments amplification may also be performed using Taq ligase for amplification. See, e.g., Barany (1991) Proc Natl Acad Sci USA 88:189. In such cases, ligation will occur only if there is a perfect match at the 3′ end of the 5′ sequence, making it possible to detect the presence of a known mutation at a specific site by looking for the presence or absence of amplification.

The methods described herein may be performed, for example, by utilizing pre-packaged diagnostic kits comprising at least one probe nucleic acid or antibody reagent described herein, which may be conveniently used, e.g., in clinical settings to diagnose patients exhibiting symptoms or family history of a disease or illness involving a SECX gene.

Furthermore, any cell type or tissue, preferably peripheral blood leukocytes, in which SECX is expressed may be utilized in the prognostic assays described herein. However, any biological sample containing nucleated cells may be used, including, for example, buccal mucosal cells.

Pharmacogenomics

Agents, or modulators that have a stimulatory or inhibitory effect on SECX activity (e.g., SECX gene expression), as identified by a screening assay described herein can be administered to individuals to treat (prophylactically or therapeutically) disorders (e.g., neurological, cancer-related or gestational disorders) associated with aberrant SECX activity. In conjunction with such treatment, the pharmacogenomics (i.e., the study of the relationship between an individual's genotype and that individual's response to a foreign compound or drug) of the individual may be considered. Differences in metabolism of therapeutics can lead to severe toxicity or therapeutic failure by altering the relation between dose and blood concentration of the pharmacologically active drug. Thus, the pharmacogenomics of the individual permits the selection of effective agents (e.g., drugs) for prophylactic or therapeutic treatments based on a consideration of the individual's genotype. Such pharmacogenomics can further be used to determine appropriate dosages and therapeutic regimens. Accordingly, the activity of SECX protein, expression of SECX nucleic acid, or mutation content of SECX genes in an individual can be determined to thereby select appropriate agent(s) for therapeutic or prophylactic treatment of the individual.

Pharmacogenomics deals with clinically significant hereditary variations in the response to drugs due to altered drug disposition and abnormal action in affected persons. See e.g., Eichelbaum, 1996, Clin Exp Pharmacol Physiol, 23:983-985 and Linder, 1997, Clin Chem, 43:254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare defects or as polymorphisms. For example, glucose-6-phosphate dehydrogenase (G6PD) deficiency is a common inherited enzymopathy in which the main clinical complication is haemolysis after ingestion of oxidant drugs (anti-malarials, sulfonamides, analgesics, nitrofurans) and consumption of fava beans.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome P450 enzymes CYP2D6 and CYP2C19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C19 quite frequently experience exaggerated drug response and side effects when they receive standard doses. If a metabolite is the active therapeutic moiety, PM show no therapeutic response, as demonstrated for the analgesic effect of codeine mediated by its CYP2D6-formed metabolite morphine. The other extreme are the so called ultra-rapid metabolizers who do not respond to standard doses. Recently, the molecular basis of ultra-rapid metabolism has been identified to be due to CYP2D6 gene amplification.

Thus, the activity of SECX protein, expression of SECX nucleic acid, or mutation content of SECX genes in an individual can be determined to thereby select appropriate agent(s) for therapeutic or prophylactic treatment of the individual. In addition, pharmacogenetic studies can be used to apply genotyping of polymorphic alleles encoding drug-metabolizing enzymes to the identification of an individual's drug responsiveness phenotype. This knowledge, when applied to dosing or drug selection, can avoid adverse reactions or therapeutic failure and thus enhance therapeutic or prophylactic efficiency when treating a subject with a SECX modulator, such as a modulator identified by one of the exemplary screening assays described herein.

Monitoring Clinical Efficacy

Monitoring the influence of agents (e.g., drugs, compounds) on the expression or activity of SECX (e.g., the ability to modulate aberrant cell proliferation and/or differentiation) can be applied in basic drug screening and in clinical trials. For example, the effectiveness of an agent determined by a screening assay as described herein to increase SECX gene expression, protein levels, or upregulate SECX activity, can be monitored in clinical trials of subjects exhibiting decreased SECX gene expression, protein levels, or downregulated SECX activity. Alternatively, the effectiveness of an agent determined by a screening assay to decrease SECX gene expression, protein levels, or downregulate SECX activity, can be monitored in clinical trials of subjects exhibiting increased SECX gene expression, protein levels, or upregulated SECX activity. In such clinical trials, the expression or activity of SECX and, preferably, other genes that have been implicated in, for example, a proliferative or neurological disorder, can be used as a “read out” or marker of the responsiveness of a particular cell.

For example, genes, including SECX, that are modulated in cells by treatment with an agent (e.g., compound, drug or small molecule) that modulates SECX activity (e.g., identified in a screening assay as described herein) can be identified. Thus, to study the effect of agents on cellular proliferation disorders, for example, in a clinical trial, cells can be isolated and RNA prepared and analyzed for the levels of expression of SECX and other genes implicated in the disorder. The levels of gene expression (i.e., a gene expression pattern) can be quantified by Northern blot analysis or RT-PCR, as described herein, or alternatively by measuring the amount of protein produced, by one of the methods as described herein, or by measuring the levels of activity of SECX or other genes. In this way, the gene expression pattern can serve as a marker, indicative of the physiological response of the cells to the agent. Accordingly, this response state may be determined before, and at various points during, treatment of the individual with the agent.

In one embodiment, the invention provides a method for monitoring the effectiveness of treatment of a subject with an agent (e.g., an agonist, antagonist, protein, peptide, nucleic acid, peptidomimetic, small molecule, or other drug candidate identified by the screening assays described herein) comprising the steps of (i) obtaining a pre-administration sample from a subject prior to administration of the agent; (ii) detecting the level of expression of a SECX protein, mRNA, or genomic DNA in the preadministration sample; (iii) obtaining one or more post-administration samples from the subject; (iv) detecting the level of expression or activity of the SECX protein, mRNA, or genomic DNA in the post-administration samples; (v) comparing the level of expression or activity of the SECX protein, mRNA, or genomic DNA in the pre-administration sample with the SECX protein, mRNA, or genomic DNA in the post administration sample or samples; and (vi) altering the administration of the agent to the subject accordingly. For example, increased administration of the agent may be desirable to increase the expression or activity of SECX to higher levels than detected, i.e., to increase the effectiveness of the agent. Alternatively, decreased administration of the agent may be desirable to decrease expression or activity of SECX to lower levels than detected, i.e., to decrease the effectiveness of the agent.

Methods of Treatment

The present invention provides for both prophylactic and therapeutic methods of treating a subject at risk of (or susceptible to) a disorder or having a disorder associated with aberrant SECX expression or activity.

Diseases and disorders that are characterized by increased (relative to a subject not suffering from the disease or disorder) levels or biological activity may be treated with Therapeutics that antagonize (i.e., reduce or inhibit) activity. Therapeutics that antagonize activity may be administered in a therapeutic or prophylactic manner. Therapeutics that may be utilized include, but are not limited to, (i) a SECX polypeptide, or analogs, derivatives, fragments or homologs thereof; (ii) antibodies to a SECX peptide; (iii) nucleic acids encoding a SECX peptide; (iv) administration of antisense nucleic acid and nucleic acids that are “dysfunctional” (i.e., due to a heterologous insertion within the coding sequences of coding sequences to a SECX peptide) that are utilized to “knockout” endogenous function of a SECX peptide by homologous recombination (see, e.g., Capecchi, 1989, Science 244: 1288-1292); or (v) modulators (i.e., inhibitors, agonists and antagonists, including additional peptide mimetic of the invention or antibodies specific to a peptide of the invention) that alter the interaction between a SECX peptide and its binding partner.

Diseases and disorders that are characterized by decreased (relative to a subject not suffering from the disease or disorder) levels or biological activity may be treated with Therapeutics that increase (i.e., are agonists to) activity. Therapeutics that upregulate activity may be administered in a therapeutic or prophylactic manner. Therapeutics that may be utilized include, but are not limited to, a SECX peptide, or analogs, derivatives, fragments or homologs thereof; or an agonist that increases bioavailability.

Increased or decreased levels can be readily detected by quantifying peptide and/or RNA, by obtaining a patient tissue sample (e.g., from biopsy tissue) and assaying it in vitro for RNA or peptide levels, structure and/or activity of the expressed peptides (or mRNAs of a SECX peptide). Methods that are well-known within the art include, but are not limited to, immunoassays (eg., by Western blot analysis, immunoprecipitation followed by sodium dodecyl sulfate (SDS) polyacrylamide gel electrophoresis, immunocytochemistry, etc.) and/or hybridization assays to detect expression of mRNAs (e.g., Northern assays, dot blots, in situ hybridization, etc.).

In one aspect, the invention provides a method for preventing, in a subject, a disease or condition associated with an aberrant SECX expression or activity, by administering to the subject an agent that modulates SECX expression or at least one SECX activity. Subjects at risk for a disease that is caused or contributed to by aberrant SECX expression or activity can be identified by, for example, any or a combination of diagnostic or prognostic assays as described herein. Administration of a prophylactic agent can occur prior to the manifestation of symptoms characteristic of the SECX aberrancy, such that a disease or disorder is prevented or, alternatively, delayed in its progression. Depending on the type of SECX aberrancy, for example, a SECX agonist or SECX antagonist agent can be used for treating the subject. The appropriate agent can be determined based on screening assays described herein.

Another aspect of the invention pertains to methods of modulating SECX expression or activity for therapeutic purposes. The modulatory method of the invention involves contacting a cell with an agent that modulates one or more of the activities of SECX protein activity associated with the cell. An agent that modulates SECX protein activity can be an agent as described herein, such as a nucleic acid or a protein, a naturally-occurring cognate ligand of a SECX protein, a peptide, a SECX peptidomimetic, or other small molecule. In one embodiment, the agent stimulates one or more SECX protein activity. Examples of such stimulatory agents include active SECX protein and a nucleic acid molecule encoding SECX that has been introduced into the cell. In another embodiment, the agent inhibits one or more SECX protein activity. Examples of such inhibitory agents include antisense SECX nucleic acid molecules and anti-SECX antibodies. These modulatory methods can be performed ii vitro (e.g., by culturing the cell with the agent) or, alternatively, in vivo (e.g., by administering the agent to a subject). As such, the present invention provides methods of treating an individual afflicted with a disease or disorder characterized by aberrant expression or activity of a SECX protein or nucleic acid molecule. In one embodiment, the method involves administering an agent (e.g., an agent identified by a screening assay described herein), or combination of agents that modulates (e.g., upregulates or downregulates) SECX expression or activity. In another embodiment, the method involves administering a SECX protein or nucleic acid molecule as therapy to compensate for reduced or aberrant SECX expression or activity.

Determination of the Biological Effect of a Therapeutic

In various embodiments of the present invention, suitable in vitro or in vivo assays are utilized to determine the effect of a specific Therapeutic and whether its administration is indicated for treatment of the affected tissue.

In various specific embodiments, in vitro assays may be performed with representative cells of the type(s) involved in the patient's disorder, to determine if a given Therapeutic exerts the desired effect upon the cell type(s). Compounds for use in therapy may be tested in suitable animal model systems including, but not limited to rats, mice, chicken, cows, monkeys, rabbits, and the like, prior to testing in human subjects. Similarly, for in vivo testing, any of the animal model system known in the art may be used prior to administration to human subjects.

Malignancies

Some SECX polypeptides are expressed in cancerous cells (see, e.g., Tables 1 and 2). Accordingly, the corresponding ORF protein is involved in the regulation of cell proliferation. Accordingly, Therapeutics of the present invention may be useful in the therapeutic or prophylactic treatment of diseases or disorders that are associated with cell hyperproliferation and/or loss of control of cell proliferation (e.g., cancers, malignancies and tumors). For a review of such hyperproliferation disorders, see e.g., Fishman, et al., 1985. MEDICINE, 2nd ed., J. B. Lippincott Co., Philadelphia, Pa.

Therapeutics of the present invention may be assayed by any method known within the art for efficacy in treating or preventing malignancies and related disorders. Such assays include, but are not limited to, in vitro assays utilizing transformed cells or cells derived from the patient's tumor, as well as in vivo assays using animal models of cancer or malignancies. Potentially effective Therapeutics are those that, for example, inhibit the proliferation of tumor-derived or transformed cells in culture or cause a regression of tumors in animal models, in comparison to the controls.

In the practice of the present invention, once a malignancy or cancer has been shown to be amenable to treatment by modulating (i.e., inhibiting, antagonizing or agonizing) activity, that cancer or malignancy may subsequently be treated or prevented by the administration of a Therapeutic that serves to modulate protein function.

Premalignant Conditions

The Therapeutics of the present invention that are effective in the therapeutic or prophylactic treatment of cancer or malignancies may also be administered for the treatment of pre-malignant conditions and/or to prevent the progression of a pre-malignancy to a neoplastic or malignant state. Such prophylactic or therapeutic use is indicated in conditions known or suspected of preceding progression to neoplasia or cancer, in particular, where non-neoplastic cell growth consisting of hyperplasia, metaplasia or, most particularly, dysplasia has occurred. For a review of such abnormal cell growth see e.g., Robbins & Angell, 1976. BASIC PATHOLOGY, 2nd ed., W. B. Saunders Co., Philadelphia, Pa.

Hyperplasia is a form of controlled cell proliferation involving an increase in cell number in a tissue or organ, without significant alteration in its structure or function. For example, it has been demonstrated that endometrial hyperplasia often precedes endometrial cancer. Metaplasia is a form of controlled cell growth in which one type of mature or fully differentiated cell substitutes for another type of mature cell. Metaplasia may occur in epithelial or connective tissue cells. Dysplasia is generally considered a precursor of cancer, and is found mainly in the epithelia. Dysplasia is the most disorderly form of non-neoplastic cell growth, and involves a loss in individual cell uniformity and in the architectural orientation of cells. Dysplasia characteristically occurs where there exists chronic irritation or inflammation, and is often found in the cervix, respiratory passages, oral cavity, and gall bladder.

Alternatively, or in addition to the presence of abnormal cell growth characterized as hyperplasia, metaplasia, or dysplasia, the presence of one or more characteristics of a transformed or malignant phenotype displayed either in vivo or in vitro within a cell sample derived from a patient, is indicative of the desirability of prophylactic/therapeutic administration of a Therapeutic that possesses the ability to modulate activity of An aforementioned protein. Characteristics of a transformed phenotype include, but are not limited to: (i) morphological changes; (ii) looser substratum attachment; (iii) loss of cell-to-cell contact inhibition; (iv) loss of anchorage dependence; (v) protease release; (vi) increased sugar transport; (vii) decreased serum requirement; (viii) expression of fetal antigens, (ix) disappearance of the 250 kDal cell-surface protein, and the like. See e.g., Richards, et al., 1986. MOLECULAR PATHOLOGY, W. B. Saunders Co., Philadelphia, Pa.

In a specific embodiment of the present invention, a patient that exhibits one or more of the following predisposing factors for malignancy is treated by administration of an effective amount of a Therapeutic: (i) a chromosomal translocation associated with a malignancy (e.g., the Philadelphia chromosome (bcr/abl) for chronic myclogenous leukemia and t(14;20) for follicular lymphoma, etc.); (ii) familial polyposis or Gardner's syndrome (possible forerunners of colon cancer); (iii) monoclonal gammopathy of undetermined significance (a possible precursor of multiple myeloma) and (iv) a first degree kinship with persons having a cancer or pre-cancerous disease showing a Mendelian (genetic) inheritance pattern (e.g., familial polyposis of the colon, Gardner's syndrome, hereditary exostosis, polyendocrine adenomatosis, Peutz-Jeghers syndrome, neurofibromatosis of Von Recklinghausen, medullary thyroid carcinoma with amyloid production and pheochromocytoma, retinoblastoma, carotid body tumor, cutaneous melanocarcinoma, intraocular melanocarcinoma, xeroderma pigmentosum, ataxia telangiectasia, Chediak-Higashi syndrome, albinism, Fanconi's aplastic anemia and Bloom's syndrome).

In another embodiment, a Therapeutic of the present invention is administered to a human patient to prevent the progression to breast, colon, lung, pancreatic, or uterine cancer, or melanoma or sarcoma.

Hyperproliferative and Dysproliferative Disorders

In one embodiment of the present invention, a Therapeutic is administered in the therapeutic or prophylactic treatment of hyperproliferative or benign dysproliferative disorders. The efficacy in treating or preventing hyperproliferative diseases or disorders of a Therapeutic of the present invention may be assayed by any method known within the art. Such assays include in vitro cell proliferation assays, in vitro or in vivo assays using animal models of hyperproliferative diseases or disorders, or the like. Potentially effective Therapeutics may, for example, promote cell proliferation in culture or cause growth or cell proliferation in animal models in comparison to controls.

Specific embodiments of the present invention are directed to the treatment or prevention of cirrhosis of the liver (a condition in which scarring has overtaken normal liver regeneration processes); treatment of keloid (hypertrophic scar) formation causing disfiguring of the skin in which the scarring process interferes with normal renewal; psoriasis (a common skin condition characterized by excessive proliferation of the skin and delay in proper cell fate determination); benign tumors; fibrocystic conditions and tissue hypertrophy (e.g., benign prostatic hypertrophy).

Neurodegenerative Disorders

Some SECX proteins are found in cell types have been implicated in the deregulation of cellular maturation and apoptosis, which are both characteristic of neurodegenerative disease. Accordingly, Therapeutics of the invention, particularly but not limited to those that modulate (or supply) activity of an aforementioned protein, may be effective in treating or preventing neurodegenerative disease. Therapeutics of the present invention that modulate the activity of an aforementioned protein involved in neurodegenerative disorders can be assayed by any method known in the art for efficacy in treating or preventing such neurodegenerative diseases and disorders. Such assays include in vitro assays for regulated cell maturation or inhibition of apoptosis or in vivo assays using animal models of neurodegenerative diseases or disorders, or any of the assays described below. Potentially effective Therapeutics, for example but not by way of limitation, promote regulated cell maturation and prevent cell apoptosis in culture, or reduce neurodegeneration in animal models in comparison to controls.

Once a neurodegenerative disease or disorder has been shown to be amenable to treatment by modulation activity, that neurodegenerative disease or disorder can be treated or prevented by administration of a Therapeutic that modulates activity. Such diseases include all degenerative disorders involved with aging, especially osteoarthritis and neurodegenerative disorders.

Disorders Related to Organ Transplantation

Some SECX can be associated with disorders related to organ transplantation, in particular but not limited to organ rejection. Therapeutics of the invention, particularly those that modulate (or supply) activity, may be effective in treating or preventing diseases or disorders related to organ transplantation. Therapeutics of the invention (particularly Therapeutics that modulate the levels or activity of an aforementioned protein) can be assayed by any method known in the art for efficacy in treating or preventing such diseases and disorders related to organ transplantation. Such assays include in vitro assays for using cell culture models as described below, or in vivo assays using animal models of diseases and disorders related to organ transplantation, see e.g., below. Potentially effective Therapeutics, for example but not by way of limitation, reduce immune rejection responses in animal models in comparison to controls.

Accordingly, once diseases and disorders related to organ transplantation are shown to be amenable to treatment by modulation of activity, such diseases or disorders can be treated or prevented by administration of a Therapeutic that modulates activity.

Cardiovascular Disease

SECX has been implicated in cardiovascular disorders, including in atherosclerotic plaque formation. Diseases such as cardiovascular disease, including cerebral thrombosis or hemorrhage, ischemic heart or renal disease, peripheral vascular disease, or thrombosis of other major vessel, and other diseases, including diabetes mellitus, hypertension, hypothyroidism, cholesterol ester storage disease, systemic lupus erythematosus, homocysteinemia, and familial protein or lipid processing diseases, and the like, are either directly or indirectly associated with atherosclerosis. Accordingly, Therapeutics of the invention, particularly those that modulate (or supply) activity or formation may be effective in treating or preventing atherosclerosis-associated diseases or disorders. Therapeutics of the invention (particularly Therapeutics that modulate the levels or activity) can be assayed by any method known in the art, including those described below, for efficacy in treating or preventing such diseases and disorders.

A vast array of animal and cell culture models exist for processes involved in atherosclerosis. A limited and non-exclusive list of animal models includes knockout mice for premature atherosclerosis (Kurabayashi and Yazaki, 1996, Int. Angiol. 15: 187-194), transgenic mouse models of atherosclerosis (Kappel et al., 1994, FASEB J. 8: 583-592), antisense oligonucleotide treatment of animal models (Callow, 1995, Curr. Opin. Cardiol. 10: 569-576), transgenic rabbit models for atherosclerosis (Taylor, 1997, Ann. N.Y. Acad. Sci 811: 146-152), hypercholesterolemic animal models (Rosenfeld, 1996, Diabetes Res. Clin. Pract. 30 Suppl.: 1-11), hyperlipidemic mice (Paigen et al., 1994, Curr. Opin. Lipidol. 5: 258-264), and inhibition of lipoxygenase in animals (Sigal et al., 1994, Ann. N.Y. Acad. Sci. 714: 211-224). In addition, in vitro cell models include but are not limited to monocytes exposed to low density lipoprotein (Frostegard et al., 1996, Atherosclerosis 121: 93-103), cloned vascular smooth muscle cells (Suttles et al., 1995, Exp. Cell Res. 218: 331-338), endothelial cell-derived chemoattractant exposed T cells (Katz et al., 1994, J. Leukoc. Biol. 55: 567-573), cultured human aortic endothelial cells (Farber et al., 1992, Am. J. Physiol. 262: H1088-1085), and foam cell cultures (Libby et al., 1996, Curr Opin Lipidol 7: 330-335). Potentially effective Therapeutics, for example but not by way of limitation, reduce foam cell formation in cell culture models, or reduce atherosclerotic plaque formation in hypercholesterolemic mouse models of atherosclerosis in comparison to controls.

Accordingly, once an atherosclerosis-associated disease or disorder has been shown to be amenable to treatment by modulation of activity or formation, that disease or disorder can be treated or prevented by administration of a Therapeutic that modulates activity.

Cytokine and Cell Proliferation/Differentiation Activity

A SECX protein of the present invention may exhibit cytokine, cell proliferation (either inducing or inhibiting) or cell differentiation (either inducing or inhibiting) activity or may induce production of other cytokines in certain cell populations. Many protein factors discovered to date, including all known cytokines, have exhibited activity in one or more factor dependent cell proliferation assays, and hence the assays serve as a convenient confirmation of cytokine activity. The activity of a protein of the present invention is evidenced by any one of a number of routine factor dependent cell proliferation assays for cell lines including, without limitation, 32D, DA2, DA1G, T10, B9, B9/11, BaF3, MC9/G, M+(preB M+), 2E8, RB5, DA1, 123, T1165, HT2, CTLL2, TF-1, Mo7e and CMK.

The activity of a protein of the invention may, among other means, be measured by the following methods: Assays for T-cell or thymocyte proliferation include without limitation those described in: CURRENT PROTOCOLS IN IMMUNOLOGY, Ed by Coligan et al., Greene Publishing Associates and Wiley-Interscience (Chapter 3 and Chapter 7); Takai et al., J Immunol 137:3494-3500, 1986; Bertagnoili et al., J Immunol 145:1706-1712, 1990; Bertagnolli et al., Cell Immunol 133:327-341, 1991; Bertagnolli, et al., J Immunol 149:3778-3783, 1992; Bowman et al., J Immunol 152:1756-1761, 1994. Assays for cytokine production and/or proliferation of spleen cells, lymph node cells or thymocytes include, without limitation, those described by Kruisbeek and Shevach, In: CURRENT PROTOCOLS IN IMMUNOLOGY. Coligan et al., eds. Vol 1, pp. 3.12.1-14, John Wiley and Sons, Toronto 1994; and by Schreiber, In: CURRENT PROTOCOLS IN IMMUNOLOGY. Coligan eds. Vol 1 pp. 6.8.1-8, John Wiley and Sons, Toronto 1994. Assays for proliferation and differentiation of hematopoietic and lymphopoietic cells include, without limitation, those described by Bottomly et al., In: CURRENT PROTOCOLS IN IMMUNOLOGY. Coligan et al., eds. Vol 1 pp. 6.3.1-6.3.12, John Wiley and Sons, Toronto 1991; deVries et al., J Exp Med 173:1205-1211, 1991; Moreau et al., Nature 336:690-692, 1988; Greenberger et al., Proc Natl Acad Sci U.S.A. 80:2931-2938, 1983; Nordan, In: CURRENT PROTOCOLS IN IMMUNOLOGY. Coligan et al., eds. Vol 1 pp. 6.6.1-5, John Wiley and Sons, Toronto 1991; Smith et al., Proc Natl Acad Sci U.S.A. 83:1857-1861, 1986; Measurement of human Interleukin 11-Bennett, et al. In: CURRENT PROTOCOLS IN IMMUNOLOGY. Coligan et al., eds. Vol 1 pp. 6.15.1 John Wiley and Sons, Toronto 1991; Ciarletta, et al., In: CURRENT PROTOCOLS IN IMMUNOLOGY. Coligan et al., eds. Vol 1 pp. 6.13.1, John Wiley and Sons, Toronto 1991.

Assays for T-cell clone responses to antigens (which will identify, among others, proteins that affect APC-T cell interactions as well as direct T-cell effects by measuring proliferation and cytokine production) include, without limitation, those described In: CURRENT PROTOCOLS IN IMMUNOLOGY. Coligan et al., eds., Greene Publishing Associates and Wiley-Interscience (Chapter 3Chapter 6, Chapter 7); Weinberger et al., Proc Natl Acad Sci USA 77:6091-6095, 1980; Weinberger et al., Eur J Immun 11:405-411, 1981; Takai et al., J Immunol 137:3494-3500, 1986; Takai et al., J Immunol 140:508-512, 1988.

Immune Stimulating or Suppressing Activity

A SECX protein of the present invention may also exhibit immune stimulating or immune suppressing activity, including without limitation the activities for which assays are described herein. A protein may be useful in the treatment of various immune deficiencies and disorders (including severe combined immunodeficiency (SCID)), e.g., in regulating (up or down) growth and proliferation of T and/or B lymphocytes, as well as effecting the cytolytic activity of NK cells and other cell populations. These immune deficiencies may be genetic or be caused by vital (e.g., 1HV) as well as bacterial or fungal infections, or may result from autoimmune disorders. More specifically, infectious diseases causes by vital, bacterial, fungal or other infection may be treatable using a protein of the present invention, including infections by HIV, hepatitis viruses, herpesviruses, mycobacteria, Leishmania species., malaria species. and various fungal infections such as candidiasis. Of course, in this regard, a protein of the present invention may also be useful where a boost to the immune system generally may be desirable, i.e., in the treatment of cancer.

Autoimmune disorders which may be treated using a protein of the present invention include, for example, connective tissue disease, multiple sclerosis, systemic lupus erythematosus, rheumatoid arthritis, autoimmune pulmonary inflammation, Guillain-Barre syndrome, autoimmune thyroiditis, insulin dependent diabetes mellitus, myasthenia gravis, graft-versus-host disease and autoimmune inflammatory eye disease. Such a protein of the present invention may also to be useful in the treatment of allergic reactions and conditions, such as asthma (particularly allergic asthma) or other respiratory problems. Other conditions, in which immune suppression is desired (including, for example, organ transplantation), may also be treatable using a protein of the present invention.

Using the proteins of the invention it may also be possible to immune responses, in a number of ways. Down regulation may be in the form of inhibiting or blocking an immune response already in progress or may involve preventing the induction of an immune response. The functions of activated T cells may be inhibited by suppressing T cell responses or by inducing specific tolerance in T cells, or both. Immunosuppression of T cell responses is generally an active, non-antigen-specific, process which requires continuous exposure of the T cells to the suppressive agent. Tolerance, which involves inducing non-responsiveness or energy in T cells, is distinguishable from immunosuppression in that it is generally antigen-specific and persists after exposure to the tolerizing agent has ceased. Operationally, tolerance can be demonstrated by the lack of a T cell response upon re-exposure to specific antigen in the absence of the tolerizing agent.

Down regulating or preventing one or more antigen functions (including without limitation B lymphocyte antigen functions (such as, for example, B7), e.g., preventing high level lymphokine synthesis by activated T cells, will be useful in situations of tissue, skin and organ transplantation and in graft-versus-host disease (GVHD). For example, blockage of T cell function should result in reduced tissue destruction in tissue transplantation. Typically, in tissue transplants, rejection of the transplant is initiated through its recognition as foreign by T cells, followed by an immune reaction that destroys the transplant. The administration of a molecule which inhibits or blocks interaction of a B7 lymphocyte antigen with its natural ligand(s) on immune cells (such as a soluble, monomeric form of a peptide having B7-2 activity alone or in conjunction with a monomeric form of a peptide having an activity of another B lymphocyte antigen (e.g., B7-1, B7-3) or blocking antibody), prior to transplantation can lead to the binding of the molecule to the natural ligand(s) on the immune cells without transmitting the corresponding costimulatory signal. Blocking B lymphocyte antigen function in this matter prevents cytokine synthesis by immune cells, such as T cells, and thus acts as an immunosuppressant. Moreover, the lack of costimulation may also be sufficient to energize the T cells, thereby inducing tolerance in a subject. Induction of long-term tolerance by B lymphocyte antigen-blocking reagents may avoid the necessity of repeated administration of these blocking reagents. To achieve sufficient immunosuppression or tolerance in a subject, it may also be necessary to block the function of B lymphocyte antigens.

The efficacy of particular blocking reagents in preventing organ transplant rejection or GVHD can be assessed using animal models that are predictive of efficacy in humans. Examples of appropriate systems which can be used include allogeneic cardiac grafts in rats and xenogeneic pancreatic islet cell grafts in mice, both of which have been used to examine the immunosuppressive effects of CTLA4Ig fusion proteins in vivo as described in Lenschow et al., Science 257:789-792 (1992) and Turka et al., Proc Natl Acad Sci USA, 89:11102-11105 (1992). In addition, murine models of GVHD (see Paul ed., FUNDAMENTAL IMMUNOLOGY, Raven Press, New York, 1989, pp. 846-847) can be used to determine the effect of blocking B lymphocyte antigen function in vivo on the development of that disease.

Blocking antigen function may also be therapeutically useful for treating autoimmune diseases. Many autoimmune disorders are the result of inappropriate activation of T cells that are reactive against self tissue and which promote the production of cytokines and auto-antibodies involved in the pathology of the diseases. Preventing the activation of autoreactive T cells may reduce or eliminate disease symptoms. Administration of reagents which block costimulation of T cells by disrupting receptor:ligand interactions of B lymphocyte antigens can be used to inhibit T cell activation and prevent production of auto-antibodies or T cell-derived cytokines which may be involved in the disease process. Additionally, blocking reagents may induce antigen-specific tolerance of autoreactive T cells which could lead to long-term relief from the disease. The efficacy of blocking reagents in preventing or alleviating autoimmune disorders can be determined using a number of well-characterized animal models of human autoimmune diseases. Examples include murine experimental autoimmune encephalitis, systemic lupus erythematosis in MRL/lpr/lpr mice or NZB hybrid mice, murine autoimmune collagen arthritis, diabetes mellitus in NOD mice and BB rats, and murine experimental myasthenia gravis (see Paul ed., FUNDAMENTAL IMMUNOLOGY, Raven Press, New York, 1989, pp. 840-856).

Upregulation of an antigen function (preferably a B lymphocyte antigen function), as a means of up regulating immune responses, may also be useful in therapy. Upregulation of immune responses may be in the form of enhancing an existing immune response or eliciting an initial immune response. For example, enhancing an immune response through stimulating B lymphocyte antigen function may be useful in cases of viral infection. In addition, systemic vital diseases such as influenza, the common cold, and encephalitis might be alleviated by the administration of stimulatory forms of B lymphocyte antigens systemically.

Alternatively, anti-viral immune responses may be enhanced in an infected patient by removing T cells from the patient, costimulating the T cells in vitro with viral antigen-pulsed APCs either expressing a peptide of the present invention or together with a stimulatory form of a soluble peptide of the present invention and reintroducing the in vitro activated T cells into the patient. Another method of enhancing anti-vital immune responses would be to isolate infected cells from a patient, transfect them with a nucleic acid encoding a protein of the present invention as described herein such that the cells express all or a portion of the protein on their surface, and reintroduce the transfected cells into the patient. The infected cells would now be capable of delivering a costimulatory signal to, and thereby activate, T cells in vivo.

In another application, up regulation or enhancement of antigen function (preferably B lymphocyte antigen function) may be useful in the induction of tumor immunity. Tumor cells (e.g., sarcoma, melanoma, lymphoma, leukemia, neuroblastoma, carcinoma) transfected with a nucleic acid encoding at least one peptide of the present invention can be administered to a subject to overcome tumor-specific tolerance in the subject. If desired, the tumor cell can be transfected to express a combination of peptides. For example, tumor cells obtained from a patient can be transfected ex vivo with an expression vector directing the expression of a peptide having B7-2-like activity alone, or in conjunction with a peptide having B7-1-like activity and/or B7-3-like activity. The transfected tumor cells are returned to the patient to result in expression of the peptides on the surface of the transfected cell. Alternatively, gene therapy techniques can be used to target a tumor cell for transfection in vivo.

The presence of the peptide of the present invention having the activity of a B lymphocyte antigen(s) on the surface of the tumor cell provides the necessary costimulation signal to T cells to induce a T cell mediated immune response against the transfected tumor cells. In addition, tumor cells which lack MHC class I or MHC class II molecules, or which fail to reexpress sufficient amounts of MHC class I or MHC class II molecules, can be transfected with nucleic acid encoding all or a portion of (e.g., a cytoplasmic-domain truncated portion) of an MHC class Iα chain protein and β₂ microglobulin protein or an MHC class II a chain protein and an MHC class II , chain protein to thereby express MHC class I or MHC class II proteins on the cell surface. Expression of the appropriate class I or class II MHC in conjunction with a peptide having the activity of a B lymphocyte antigen (e.g., B7-1, B7-2, B7-3) induces a T cell mediated immune response against the transfected tumor cell. Optionally, a gene encoding an antisense construct which blocks expression of an MHC class II associated protein, such as the invariant chain, can also be cotransfected with a DNA encoding a peptide having the activity of a B lymphocyte antigen to promote presentation of tumor associated antigens and induce tumor specific immunity. Thus, the induction of a T cell mediated immune response in a human subject may be sufficient to overcome tumor-specific tolerance in the subject.

The activity of a protein of the invention may, among other means, be measured by the following methods: Suitable assays for thymocyte or splenocyte cytotoxicity include, without limitation, those described In: CURRENT PROTOCOLS IN IMMUNOLOGY. Coligan et al., eds. Greene Publishing Associates and Wiley-Interscience (Chapter 3, Chapter 7); Herrmann et al., Proc Natl Acad Sci USA 78:2488-2492, 1981; Herrmann et al., J Immunol 128:1968-1974, 1982; Handa et al., J Immunol 20:1564-1572, 1985; Takai et al., J Immunol 137:3494-3500, 1986; Takai et al., J Immunol 140:508-512, 1988; Herrmann et al., Proc Natl Acad Sci USA 78:2488-2492, 1981; Herrmann et al., J Immunol 128:1968-1974, 1982; Handa et al., J Immunol 18:1564-1572, 1985; Takai et al., J Immunol 137:3494-3500, 1986; Bowman et al., J Virology 61:1992-1998; Takai et al., J Immunol 140:508-512, 1988; Bertagnolli et al., Cell Immunol 133:327-341, 1991; Brown et al., J Immunol 153:3079-3092, 1994.

Assays for T-cell-dependent immunoglobulin responses and isotype switching (which will identify, among others, proteins that modulate T-cell dependent antibody responses and that affect Th1/Th2 profiles) include, without limitation, those described in: Maliszewski, J Immunol 144:3028-3033, 1990; and Mond and Brunswick In: CURRENT PROTOCOLS IN IMMUNOLOGY. Coligan et al., (eds.) Vol 1 pp. 3.8.1-3.8.16, John Wiley and Sons, Toronto 1994.

Mixed lymphocyte reaction (MLR) assays (which will identify, among others, proteins that generate predominantly Th1 and CTL responses) include, without limitation, those described In: CURRENT PROTOCOLS IN IMMUNOLOGY. Coligan et al., eds. Greene Publishing Associates and Wiley-Interscience (Chapter 3, Chapter 7); Takai et al., J Immunol 137:3494-3500, 1986; Takai et al., J Immunol 140:508-512, 1988; Bertagnolli et al., J Immunol 149:3778-3783, 1992.

Dendritic cell-dependent assays (which will identify, among others, proteins expressed by dendritic cells that activate naive T-cells) include, without limitation, those described in: Guery et al., J Immunol 134:536-544, 1995; Inaba et al., J Exp Med 173:549-559, 1991; Macatonia et al., J Immunol 154:5071-5079, 1995; Porgador et al., J Exp Med 182:255-260, 1995; Nair et al., J Virol 67:4062-4069, 1993; Huang et al., Science 264:961-965, 1994; Macatonia et al., J Exp Med 169:1255-1264, 1989; Bhardwaj et al., J Clin Investig 94:797-807, 1994; and Inaba et al., J Exp Med 172:631-640, 1990. Assays for lymphocyte survival/apoptosis (which will identify, among others, proteins that prevent apoptosis after superantigen induction and proteins that regulate lymphocyte homeostasis) include, without limitation, those described in: Darzynkiewicz et al., Cytometry 13:795-808, 1992; Gorczyca et al., Leukemia 7:659-670, 1993; Gorczyca et al., Cancer Res 53:1945-1951, 1993; Itoh et al., Cell 66:233-243, 1991; Zacharchuk, J Immunol 145:4037-4045, 1990; Zamai et al., Cytometry 14:891-897, 1993; Gorczyca et al., Internat J Oncol 1:639-648, 1992.

Assays for proteins that influence early steps of T-cell commitment and development include, without limitation, those described in: Antica et al., Blood 84:111-117, 1994; Fine et al., Cell Immunol 155: 111-122, 1994; Galy et al., Blood 85:2770-2778, 1995; Toki et al., Proc Nat Acad Sci USA 88:7548-7551, 1991.

Hematopoiesis Regulating Activity

A SECX protein of the present invention may be useful in regulation of hematopoiesis and, consequently, in the treatment of myeloid or lymphoid cell deficiencies. Even marginal biological activity in support of colony forming cells or of factor-dependent cell lines indicates involvement in regulating hematopoiesis, e.g. in supporting the growth and proliferation of erythroid progenitor cells alone or in combination with other cytokines, thereby indicating utility, for example, in treating various anemias or for use in conjunction with irradiation/chemotherapy to stimulate the production of erythroid precursors and/or erythroid cells; in supporting the growth and proliferation of myeloid cells such as granulocytes and monocytes/macrophages (i.e., traditional CSF activity) useful, for example, in conjunction with chemotherapy to prevent or treat consequent myelo-suppression; in supporting the growth and proliferation of megakaryocytes and consequently of platelets thereby allowing prevention or treatment of various platelet disorders such as thrombocytopenia, and generally for use in place of or complimentary to platelet transfusions; and/or in supporting the growth and proliferation of hematopoietic stem cells which are capable of maturing to any and all of the above-mentioned hematopoietic cells and therefore find therapeutic utility in various stem cell disorders (such as those usually treated with transplantation, including, without limitation, aplastic anemia and paroxysmal nocturnal hemoglobinuria), as well as in repopulating the stem cell compartment post irradiation/chemotherapy, either in-vivo or ex-vivo (i.e., in conjunction with bone marrow transplantation or with peripheral progenitor cell transplantation (homologous or heterologous)) as normal cells or genetically manipulated for gene therapy.

The activity of a protein of the invention may, among other means, be measured by the following methods:

Suitable assays for proliferation and differentiation of various hematopoietic lines are cited above.

Assays for embryonic stem cell differentiation (which will identify, among others, proteins that influence embryonic differentiation hematopoiesis) include, without limitation, those described in: Johansson et al. Cellular Biology 15:141-151, 1995; Keller et al., Mol. Cell. Biol. 13:473-486, 1993; McClanahan et al., Blood 81:2903-2915, 1993. Assays for stem cell survival and differentiation (which will identify, among others, proteins that regulate lympho-hematopoiesis) include, without limitation, those described in: Methylcellulose colony forming assays, Freshney, In: CULTURE OF HEMATOPOIETIC CELLS. Freshney, et al. (eds.) Vol pp. 265-268, Wiley-Liss, Inc., New York, N.Y. 1994; Hirayama et al., Proc Natl Acad Sci USA 89:5907-5911, 1992; McNiece and Briddeli, In: CULTURE OF HEMATOPOETIC CELLS. Freshney, et al. (eds.) Vol pp. 23-39, Wiley-Liss, Inc., New York, N.Y. 1994; Neben et al., Exp Hematol 22:353-359, 1994; Ploemacher, In: CULTURE OF HEMATOPOETIC CELLS. Freshney, et al. eds. Vol pp. 1-21, Wiley-Liss, Inc., New York, N.Y. 1994; Spoonceret al., In: CULTURE OF HEMATOPOIETIC CELLS. Freshhey, et al., (eds.) Vol pp. 163-179, Wiley-Liss, Inc., New York, N.Y. 1994; Sutherland, In: CULTURE OF HEMATOPOIETIC CELLS. Freshney, et al., (eds.) Vol pp. 139-162, Wiley-Liss, Inc., New York, N.Y. 1994.

Tissue Growth Activity

A SECX protein of the present invention also may have utility in compositions used for bone, cartilage, tendon, ligament and/or nerve tissue growth or regeneration, as well as for wound healing and tissue repair and replacement, and in the treatment of burns, incisions and ulcers.

A protein of the present invention, which induces cartilage and/or bone growth in circumstances where bone is not normally formed, has application in the healing of bone fractures and cartilage damage or defects in humans and other animals. Such a preparation employing a protein of the invention may have prophylactic use in closed as well as open fracture reduction and also in the improved fixation of artificial joints. De novo bone formation induced by an osteogenic agent contributes to the repair of congenital, trauma induced, or oncologic resection induced craniofacial defects, and also is useful in cosmetic plastic surgery.

A protein of this invention may also be used in the treatment of periodontal disease, and in other tooth repair processes. Such agents may provide an environment to attract bone-forming cells, stimulate growth of bone-forming cells or induce differentiation of progenitors of bone-forming cells. A protein of the invention may also be useful in the treatment of osteoporosis or osteoarthritis, such as through stimulation of bone and/or cartilage repair or by blocking inflammation or processes of tissue destruction (collagenase activity, osteoclast activity, etc.) mediated by inflammatory processes.

Another category of tissue regeneration activity that may be attributable to the protein of the present invention is tendon/ligament formation. A protein of the present invention, which induces tendon/ligament-like tissue or other tissue formation in circumstances where such tissue is not normally formed, has application in the healing of tendon or ligament tears, deformities and other tendon or ligament defects in humans and other animals. Such a preparation employing a tendon/ligament-like tissue inducing protein may have prophylactic use in preventing damage to tendon or ligament tissue, as well as use in the improved fixation of tendon or ligament to bone or other tissues, and in repairing defects to tendon or ligament tissue. De novo tendon/ligament-like tissue formation induced by a composition of the present invention contributes to the repair of congenital, trauma induced, or other tendon or ligament defects of other origin, and is also useful in cosmetic plastic surgery for attachment or repair of tendons or ligaments. The compositions of the present invention may provide an environment to attract tendon- or ligament-forming cells, stimulate growth of tendon- or ligament-forming cells, induce differentiation of progenitors of tendon- or ligament-forming cells, or induce growth of tendon/ligament cells or progenitors ex vivo for return in vivo to effect tissue repair. The compositions of the invention may also be useful in the treatment of tendonitis, carpal tunnel syndrome and other tendon or ligament defects. The compositions may also include an appropriate matrix and/or sequestering agent as a career as is well known in the art.

The protein of the present invention may also be useful for proliferation of neural cells and for regeneration of nerve and brain tissue, i.e. for the treatment of central and peripheral nervous system diseases and neuropathies, as well as mechanical and traumatic disorders, which involve degeneration, death or trauma to neural cells or nerve tissue. More specifically, a protein may be used in the treatment of diseases of the peripheral nervous system, such as peripheral nerve injuries, peripheral neuropathy and localized neuropathies, and central nervous system diseases, such as Alzheimer's, Parkinson's disease, Huntington's disease, amyotrophic lateral sclerosis, and Shy-Drager syndrome. Further conditions which may be treated in accordance with the present invention include mechanical and traumatic disorders, such as spinal cord disorders, head trauma and cerebrovascular diseases such as stroke. Peripheral neuropathies resulting from chemotherapy or other medical therapies may also be treatable using a protein of the invention.

Proteins of the invention may also be useful to promote better or faster closure of non-healing wounds, including without limitation pressure ulcers, ulcers associated with vascular insufficiency, surgical and traumatic wounds, and the like. It is expected that a protein of the present invention may also exhibit activity for generation or regeneration of other tissues, such as organs (including, for example, pancreas, liver, intestine, kidney, skin, endothelium), muscle (smooth, skeletal or cardiac) and vascular (including vascular endothelium) tissue, or for promoting the growth of cells comprising such tissues. Part of the desired effects may be by inhibition or modulation of fibrotic scarring to allow normal tissue to regenerate. A protein of the invention may also exhibit angiogenic activity.

A protein of the present invention may also be useful for gut protection or regeneration and treatment of lung or liver fibrosis, reperfusion injury in various tissues, and conditions resulting from systemic cytokine damage.

A protein of the present invention may also be useful for promoting or inhibiting differentiation of tissues described above from precursor tissues or cells; or for inhibiting the growth of tissues described above.

The activity of a protein of the invention may, among other means, be measured by the following methods:

Assays for tissue generation activity include, without limitation, those described in: International Patent Publication No. WO95/16035 (bone, cartilage, tendon); International Patent Publication No. WO95/05846 (nerve, neuronal); International Patent Publication No. WO91/07491 (skin, endothelium).

Assays for wound healing activity include, without limitation, those described in: Winter, EPIDERMAL WOUND HEALING, pp. 71-112 (Maibach and Rovee, eds.), Year Book Medical Publishers, Inc., Chicago, as modified by Eaglstein and Menz, J. Invest. Dermatol 71:382-84 (1978).

Activin/Inhibin Activity

A SECX protein of the present invention may also exhibit activin- or inhibin-related activities. Inhibins are characterized by their ability to inhibit the release of follicle stimulating hormone (FSH), while activins and are characterized by their ability to stimulate the release of follicle stimulating hormone (FSH). Thus, a protein of the present invention, alone or in heterodimers with a member of the inhibin a family, may be useful as a contraceptive based on the ability of inhibins to decrease fertility in female mammals and decrease spermatogenesis in male mammals. Administration of sufficient amounts of other inhibins can induce infertility in these mammals. Alternatively, the protein of the invention, as a homodimer or as a heterodimer with other protein subunits of the inhibin-b group, may be useful as a fertility inducing therapeutic, based upon the ability of activin molecules in stimulating FSH release from cells of the anterior pituitary. See, for example, U.S. Pat. No. 4,798,885. A protein of the invention may also be useful for advancement of the onset of fertility in sexually immature mammals, so as to increase the lifetime reproductive performance of domestic animals such as cows, sheep and pigs.

The activity of a protein of the invention may, among other means, be measured by the following methods:

Assays for activin/inhibin activity include, without limitation, those described in: Vale et al., Endocrinology 91:562-572, 1972; Ling et al., Nature 321:779-782, 1986; Vale et al., Nature 321:776-779, 1986; Mason et al., Nature 318:659-663, 1985; Forage et al., Proc Natl Acad Sci USA 83:3091-3095, 1986.

Chemotactic/Chemokinetic Activity

A protein of the present invention may have chemotactic or chemokinetic activity (e.g., act as a chemokine) for mammalian cells, including, for example, monocytes, fibroblasts, neutrophils, T-cells, mast cells, eosinophils, epithelial and/or endothelial cells. Chemotactic and chemokinetic proteins can be used to mobilize or attract a desired cell population to a desired site of action. Chemotactic or chemokinetic proteins provide particular advantages in treatment of wounds and other trauma to tissues, as well as in treatment of localized infections. For example, attraction of lymphocytes, monocytes or neutrophils to tumors or sites of infection may result in improved immune responses against the tumor or infecting agent.

A protein or peptide has chemotactic activity for a particular cell population if it can stimulate, directly or indirectly, the directed orientation or movement of such cell population. Preferably, the protein or peptide has the ability to directly stimulate directed movement of cells. Whether a particular protein has chemotactic activity for a population of cells can be readily determined by employing such protein or peptide in any known assay for cell chemotaxis.

The activity of a protein of the invention may, among other means, be measured by following methods:

Assays for chemotactic activity (which will identify proteins that induce or prevent chemotaxis) consist of assays that measure the ability of a protein to induce the migration of cells across a membrane as well as the ability of a protein to induce the adhesion of one cell population to another cell population. Suitable assays for movement and adhesion include, without limitation, those described in: CURRENT PROTOCOLS IN IMMUNOLOGY, Coligan et al., eds. (Chapter 6.12, MEASUREMENT OF ALPHA AND BETA CHEMOKINES 6.12.1-6.12.28); Taub et al. J Clin Invest 95:1370-1376, 1995; Lind et al. APMIS 103:140-146, 1995; Muller et al., Eur J Immunol 25: 1744-1748; Gruberet al. J Immunol 152:5860-5867, 1994; Johnston et al., J Immunol 153: 1762-1768, 1994.

Hemostatic and Thrombolytic Activity

A protein of the invention may also exhibit hemostatic or thrombolytic activity. As a result, such a protein is expected to be useful in treatment of various coagulation disorders (including hereditary disorders, such as hemophilias) or to enhance coagulation and other hemostatic events in treating wounds resulting from trauma, surgery or other causes. A protein of the invention may also be useful for dissolving or inhibiting formation of thromboses and for treatment and prevention of conditions resulting therefrom (such as, for example, infarction of cardiac and central nervous system vessels (e.g., stroke).

The activity of a protein of the invention may, among other means, be measured by the following methods:

Assay for hemostatic and thrombolytic activity include, without limitation, those described in: Linet et al., J. Clin. Pharmacol. 26:131-140, 1986; Burdick et al., Thrombosis Res. 45:413-419, 1987; Humphrey et al., Fibrinolysis 5:71-79 (1991); Schaub, Prostaglandins 35:467-474, 1988.

Receptor/Ligand Activity

A protein of the present invention may also demonstrate activity as receptors, receptor ligands or inhibitors or agonists of receptor/ligand interactions. Examples of such receptors and ligands include, without limitation, cytokine receptors and their ligands, receptor kinases and their ligands, receptor phosphatases and their ligands, receptors involved in cell—cell interactions and their ligands (including without limitation, cellular adhesion molecules (such as selectins, integrins and their ligands) and receptor/ligand pairs involved in antigen presentation, antigen recognition and development of cellular and humoral immune responses). Receptors and ligands are also useful for screening of potential peptide or small molecule inhibitors of the relevant receptor/ligand interaction. A protein of the present invention (including, without limitation, fragments of receptors and ligands) may themselves be useful as inhibitors of receptor/ligand interactions.

The activity of a protein of the invention may, among other means, be measured by the following methods:

Suitable assays for receptor-ligand activity include without limitation those described in: CURRENT PROTOCOLS IN IMMUNOLOGY, Ed by Coligan, et al., Greene Publishing Associates and Wiley-Interscience (Chapter 7.28, Measurement of Cellular Adhesion under static conditions 7.28.1-7.28.22), Takai et al., Proc Natl Acad Sci USA 84:6864-6868, 1987; Bierer et al., J. Exp. Med. 168:1145-1156, 1988; Rosenstein et al., J. Exp. Med. 169:149-160 1989; Stoltenborg et al., J Immunol Methods 175:59-68, 1994; Stitt et al., Cell 80:661-670, 1995.

Anti-Inflammatory Activity

Proteins of the present invention may also exhibit anti-inflammatory activity. The anti-inflammatory activity may be achieved by providing a stimulus to cells involved in the inflammatory response, by inhibiting or promoting cell-cell interactions (such as, for example, cell adhesion), by inhibiting or promoting chemotaxis of cells involved in the inflammatory process, inhibiting or promoting cell extravasation, or by stimulating or suppressing production of other factors which more directly inhibit or promote an inflammatory response. Proteins exhibiting such activities can be used to treat inflammatory conditions including chronic or acute conditions), including without limitation inflammation associated with infection (such as septic shock, sepsis or systemic inflammatory response syndrome (SIRS)), ischemia-reperfusion injury, endotoxin lethality, arthritis, complement-mediated hyperacute rejection, nephritis, cytokine or chemokine-induced lung injury, inflammatory bowel disease, Crohn's disease or resulting from over production of cytokines such as TNF or IL-1. Proteins of the invention may also be useful to treat anaphylaxis and hypersensitivity to an antigenic substance or material.

Tumor Inhibition Activity

In addition to the activities described above for immunological treatment or prevention of tumors, a protein of the invention may exhibit other anti-tumor activities. A protein may inhibit tumor growth directly or indirectly (such as, for example, via ADCC). A protein may exhibit its tumor inhibitory activity by acting on tumor tissue or tumor precursor tissue, by inhibiting formation of tissues necessary to support tumor growth (such as, for example, by inhibiting angiogenesis), by causing production of other factors, agents or cell types which inhibit tumor growth, or by suppressing, eliminating or inhibiting factors, agents or cell types which promote tumor growth.

Other Activities

A protein of the invention may also exhibit one or more of the following additional activities or effects: inhibiting the growth, infection or function of, or killing, infectious agents, including, without limitation, bacteria, viruses, fungi and other parasites; effecting (suppressing or enhancing) bodily characteristics, including, without limitation, height, weight, hair color, eye color, skin, fat to lean ratio or other tissue pigmentation, or organ or body part size or shape (such as, for example, breast augmentation or diminution, change in bone form or shape); effecting biorhythms or circadian cycles or rhythms; effecting the fertility of male or female subjects; effecting the metabolism, catabolism, anabolism, processing, utilization, storage or elimination of dietary fat, lipid, protein, carbohydrate, vitamins, minerals, cofactors or other nutritional factors or component(s); effecting behavioral characteristics, including, without limitation, appetite, libido, stress, cognition (including cognitive disorders), depression (including depressive disorders) and violent behaviors; providing analgesic effects or other pain reducing effects; promoting differentiation and growth of embryonic stem cells in lineages other than hematopoietic lineages; hormonal or endocrine activity; in the case of enzymes, correcting deficiencies of the enzyme and treating deficiency-related diseases; treatment of hyperproliferative disorders (such as, for example, psoriasis); immunoglobulin-like activity (such as, for example, the ability to bind antigens or complement); and the ability to act as an antigen in a vaccine composition to raise an immune response against such protein or another material or entity which is cross-reactive with such protein.

Neural disorders in general include Parkinson's disease, Alzheimer's disease, Huntington's disease, multiple sclerosis, amyotrophic lateral sclerosis (ALS), peripheral neuropathy, tumors of the nervous system, exposure to neurotoxins, acute brain injury, peripheral nerve trauma or injury, and other neuropathies, epilepsy, and/or tremors.

### EXAMPLES

### Example 1

Chromosomal Localization of SECX Nucleic Acid Sequences

Radiation hybrid mapping using human chromosome markers was performed to determine the chromosomal location of various SECX nucleic acids of the invention. Mapping was performed generally as described in Steen, R G et al. (A High-Density Integrated Genetic Linkage and Radiation Hybrid Map of the Laboratory Rat, Genome Research 1999 (Published Online on May 21, 1999)Vol. 9, AP1-AP8, 1999). A panel of 93 cell clones containing randomized radiation-induced human chromosomal fragments was screened in 96 well plates using PCR primers designed to specifically identify SECX nucleic acids of the invention. The chromsomes to which various SECX nucleic acids, along with first marker, second marker, and origin marker genes, are shown Table 3.

TABLE 3

| | | | 1^{st} Marker | | |
|---|---|---|---|---|---|
| SECX | Clone | Chr. | Gene | 2^{nd} Marker Gene | Origin Marker |
| SEC2 | 10326230 | 1 | AFMB014ZB9 | GCT8C07 | NIB1364 |
| SEC3 | 16399139 | 1 | AFMB014ZB9 | GCT8C07 | NIB1364 |
| SEC4 | 3440544.0.81 | 1 | D1S417 | AFMA230VH5 | NIB1364 |
| SEC5 | 3581980.0.30 | 8 | AFMA053XF1 | CHLC.GATA50D10 | WI-6641 |
| SEC6 | 4418354.0.6 | 5 | — | WI-9907 | WI-9907 |
| SEC7 | 4418354.0.9 | 5 | — | WI-9907 | WI-9907 |
| SEC8 | 6779999.0.31 | 9 | WI-3309 | CHLC.GATA28C02 | CHLC.GCT3G05 |
| SEC9 | 8484782.0.5 | 4 | AFM312WG1 | WI-4886 | WI-6657 |

### Example 2

Molecular Cloning of the Full Length FGF10-AC004449

In this example, cloning is described for the full length FGF10-AC004449 clone. Olignucleotide primers were designed to PCR amplify the full length FGF10-AC004449 (SEQ ID NO: 1) sequence. The forward primers include an in-frame BglII restriction site: 4301999 TOPO 5′: AGATCT CCACC ATG CGC CGC CGC CTG TGG CTG GGC CTG-3′ (SEQ ID NO: 21 ), and 4301999 Forward: 5′-CTCGTC AGATCT CCACC ATG CGC CGC CGC CTG TGG CTG GGC CTG-3′ (SEQ ID NO: 22). The forward primers also include a consensus Kozak sequence (CCACC) upstream to the ATG Start codon. The reverse primers contains an in-frame XhoI restriction site: 4301999 TOPO: 5′-CTCGAG GGA GAC CAG GAC GGG CAG GAA GTG GGC GGA-3′ (SEQ ID NO: 23) and 4301999 Reverse: 5′-CTCGTC CTCGAG GGA GAC CAG GAC GGG CAG GAA GTG GGC GGA-3′ (SEQ ID NO: 24).

Independent PCR reactions were performed using 5 ng human fetal brain cDNA template and corresponding primer pairs. The reaction mixtures contained 1 μM of each of the 4301999 TOPO Forward and 4301999 TOPO Reverse or 4301999 Forward and 4301999 Reverse primers, 5 micromoles dNTP (Clontech Laboratories, Palo Alto Calif.) and 1 microliter of 50× Advantage-HF 2 polymerase (Clontech Laboratories, Palo Alto Calif.) in 50 microliter volume. The following reaction conditions were used:

| | | |
|---|---|---|
| a) | 96° C. | 3 minutes |
| b) | 96° C. | 30 seconds denaturation |
| c) | 70° C. | 30 seconds, primer annealing. This temperature was |
| | | gradually decreased by 1° C./cycle |
| d) | 72° C. | 1 minute extension. |

Repeat steps b-d 10 times

| | | |
|---|---|---|
| e) | 96° C. | 30 seconds denaturation |
| f) | 60° C. | 30 seconds annealing |
| g) | 72° C. | 1 minute extension |

Repeat steps e-g 25 times

| | | |
|---|---|---|
| h) | 72° C. | 5 minutes final extension |

The expected 510 bp amplified product was detected by agarose gel electrophoresis in both samples. The fragments were purified from agarose gel. The fragment derived from the 4301999 TOPO Forward and 4301999 TOPO Reverse primed reaction was cloned into the pCDNA3.1-TOPO-V5-His vector (Invitrogen, Carlsbad, Calif.). The fragment, derived from the 4301999 Forward and 4301999 Reverse primed reaction was cloned into the pBIgHis vector (CuraGen Corp.) The cloned inserts were sequenced and verified as an open reading frame coding for the predicted full length FGF10-AC004449. The cloned sequence was determined to be 100% identical to the predicted sequence.

### Example 3

Molecular Cloning of the Mature Form of FGF10-AC004449

In this example, cloning is described for the mature form of the FGF10-AC004449 clone. Using the verified FGF10-AC004449 insert from the pCDNA3.1-TOPO-V5-His construct, as template, oliglonucleotide primers were designed to PCR amplify the mature form of FGF10-AC004449 PCR reaction was set up to amplify the mature form of FGF10-AC004449. The forward primer, FGF10-AC004449 C forward:5′-AGATCT ACC CCG AGC GCG TCG CGG GGA CCG-3′ (SEQ ID NO: 25). The reverse primer, 4301999 Reverse:5′-CTCGTC CTCGAG GGA GAC CAG GAC GGG CAG GAA GTG GGC GGA-3′ (SEQ ID NO: 26).

The PCR reactions were set up using 0.1 ng pCDNA3.1-TOPO-V5-His-FGF10-AC004449 plasmid DNA template representing the full length FGF10-AC004449, 1 μM of each of the corresponding primer pairs, 5 micromoles dNTP (Clontech Laboratories, Palo Alto Calif.) and 1 microliter of 50 × Advantage-HF 2 polymerase (Clontech Laboratories, Palo Alto Calif.) in 50 microliter volume. The following reaction conditions were used:

| a) | 96° C. | 3 minutes denaturation |
|---|---|---|
| b) | 96° C. | 30 seconds denaturation |
| c) | 60° C. | 30 seconds primer annealing |
| d) | 72° C. | 1 minute extension |
| repeat steps b-d 15 times | | |
| e) | 72° C. | 5 minutes final extension |

The expected 450 bp amplified product was detected by agrose gel electrophoresis. The fragments were purified from the agarose gel and ligated to pCR2.1 vector (Invitrogen, Carlsbad, Calif.). The cloned inserts were sequenced and the inserts were verified as open reading frames coding for the predicted mature form of FGF10-AC004449.

### Example 4

Preparation of the Mammalian Expression Vector pCEP4/Sec.

An expression vector, named pCEP4/Sec, was constructed for examining expression of SECX nucleic acid sequences. pCEP4/Sec is an expression vector that allows heterologous protein expression and secretion by fusing any protein to the Ig Kappa chain signal peptide. Detection and purification of the expressed protein are aided by the presence of the V5 epitope tag and 6× His tag at the C-terminus (Invitrogen, Carlsbad, Calif.).

To construct pCEP4/SEC, theoligonucleotide primers, pSec-V5-His Forward: 5′-CTCGTCCTCGAGGGTAAGCCTATCCCTAAC-3′ (SEQ ID NO: 27) and 5′-pSec-V5-His Reverse:CTCGTCGGGCCCCTGATCAGCGGGTTTAAAC-3′ (SEQ ID NO: 28), were designed to amplify a fragment from the pcDNA3.1-V5His (Invitrogen, Carlsbad, Calif.) expression vector that includes V5 and His6. The PCR product was digested with XhoI and ApaI and ligated into the XhoI/ApaI digested pSecTag2 B vector harboring an Ig kappa leader sequence (Invitrogen, Carlsbad Calif.). The correct structure of the resulting vector, pSecV5His, including an in-frame Ig-kappa leader and V5-His6 was verified by DNA sequence analysis. The vector pSecV5His was digested with PmeI and NheI to provide a fragment retaining the above elements in the correct frame. The PmeI-NheI fragment was ligated into the BamHI/Klenow and NheI treated vector pCEP4 (Invitrogen, Carlsbad, Calif.). The resulting vector was named pCEP4/Sec and includes an in-frame Ig kappa leader, a site for insertion of a clone of interest, V5 and His6 under control of the PCMV and/or the mT7 promoter.

### Example 5

Expression of FGF10AC0044 in Human Embryonic Kidney 293 Cells

A 0.5 kb BglII-XhoI fragment containing the FGF10AC0044 sequence was isolated from pCR2.1-FGF10-X and subcloned into BamHI-XhoI digested pCEP4/Sec to generate expression vector pCEP4/Sec-FGF10-X. The pCEP4/Sec-FGF10-X vector was transfected into human embryonic kidney 293 cells using the LipofectaminePlus reagent following the manufacturer's instructions (Gibco/BRL). The cell pellet and supernatant were harvested 72 hours after transfection and examined for FGF10AC0044 expression by Western blotting under reducing conditions with an anti-V5 antibody. As shown in FIG. 1, FGF10AC0044 is expressed as a 33 kDa protein secreted by human embryonic kidney 293 cells.

### Example 6

Expression of FGF10AC0044 in Recombinant E. coli.

The vector pRSETA (InVitrogen Inc., Carlsbad, Calif.) was digested with XhoI and NcoI restriction enzymes. Oligonucleotide linkers CATGGTCAGCCTAC (SEQ ID NO: 178) and TCGAGTAGGCTGAC (SEQ ID NO: 179) were annealed at 37° C. and ligated into the XhoI-NcoI treated pRSETA. The resulting vector was confirmed by restriction analysis and sequencing and was named pETMY. The BamHI-XhoI fragment (see above) was ligated into the pETMY that was digested with BamHI and XhoI restriction enzymes. The expression vector was named pETMY-FGF10-X. In this vector, hFGF10-X was fused to the 6× His tag and T7 epitope at its N-terminus. The plasmid pETMY-FGF11-X was then transformed into the E. coli expression host BL21(DE3, pLys) (Novagen, Madison, Wis.) and the expression induction of protein FGF10-X was carried out according to the manufacturer's instructions. After induction, total cells were harvested, and proteins were analyzed by Western blotting using anti-HisGly antibody (Invitrogen, Carlsbad, Calif.). FIG. 2 demonstrates that FGF10AC0044 was expressed as a 29 kDa protein in E. coli cells.

### Example 7

Molecular Cloning of 16399139.S124A

In this example, cloning is described for the full length 16399139.S124A clone. Olignucleotide primers were designed to PCR amplify the full length sequence. The forward primer included 16399139C-Forward: 5′-CTCGTCAGATCTGTGATGCAGCCCTACCCTTTGGTTTG-3′ (SEQ ID NO: 29). The reverse primwe included 16399139 F-TOPO-Reverse: 5′-CTCGAGGGAGCCGTGCGGGGGCGCGCCCTGGCCAGA-3′ (SEQ ID NO: 30).

Independent PCR reactions were performed using 5 ng human fetal brain cDNA template, with the corresponding primer pairs. The reaction mixtures contained 1 μM of each of the 16399139C-Forward and 16399139 F-TOPO-Reverse primers, 5 micromoles dNTP (Clontech Laboratories, Palo Alto Calif.) and 1 microliter of 50× Advantage-BF 2 polymerase (Clontech Laboratories, Palo Alto Calif.) in 50 microliter volume. The following reaction conditions were used:

| | | |
|---|---|---|
| a) | 96° C. | 3 minutes |
| b) | 96° C. | 30 seconds denaturation |
| c) | 70° C. | 30 seconds, primer annealing. This temperature was |
| | | gradually decreased by 1° C./cycle |
| d) | 72° C. | 1 minute extension. |

Repeat steps b-d 10 times

| | | |
|---|---|---|
| e) | 96° C. | 30 seconds denaturation |
| f) | 60° C. | 30 seconds annealing |
| g) | 72° C. | 1 minute extension |

Repeat steps e-g 25 times

| | | |
|---|---|---|
| h) | 72° C. | 5 minutes final extension |

The PCR product was cloned into the pCR2.1 vector (Invitrogen, Carlsbad Calif.) and sequenced using vector specific primers and the following gene specific primers:

| | | |
|---|---|---|
| 16399139 S1: | AATGAGTGTGATGCGAGT; | (SEQ ID NO:31) |
| 16399139 S2: | CAGCATACGGTCTTAGAA; | (SEQ ID NO:32) |
| | and | |
| 16399139 S3: | ACATGCGAATGTGAGCAC. | (SEQ ID NO:33) |

### Example 8

Tissue Expression Analysis of SECX Nucleic Acids

The quantitative tissue expression of various clones was assessed in 41 normal and 55 tumor samples by real time quantitative PCR (TAQMAN®) performed on a Perkin-Elmer Biosystems ABI PRISM® 7700 Sequence Detection System 96 RNA samples were normalized to β-actin and GAPDH. cDNA was produced from RNA (˜50 ng total or ˜1 ng polyA+) using the TAQMAN® Reverse Transcription Reagents Kit (PE Biosystems, Foster City, Calif.; cat #N808-0234) and random hexamers according to the manufacturer's protocol. Reactions were performed in 20 ul and incubated for 30 min. at 48° C. cDNA (5 ul) was then transferred to a separate plate for the TAQMAN® reaction using β-actin and GAPDH TAQMAN® Assay Reagents (PE Biosystems; cat. #'s 4310881E and 4310884E, respectively) and TAQMAN® universal PCR Master Mix (PE Biosystems; cat #4304447) according to the manufacturer's protocol. Reactions were performed in 25 ul using the following parameters: 2 min. at 50° C.; 10 min. at 95° C.; 15 sec. at 95° C./1 min. at 60° C. (40 cycles). Results were recorded as CT values (cycle at which a given sample crosses a threshold level of fluorescence) using a log scale, with the difference in RNA concentration between two samples being represented as 2 to the power of delta CT. The percent relative expression is then obtained by taking the reciprocal of this RNA difference and multiplying by 100.The average CT values obtained for β-actin and GAPDH were used to normalize RNA samples. The RNA sample generating the highest CT value required no further diluting, while all other samples were diluted relative to this sample according to their β-actin/GAPDH average CT values.

Normalized RNA (5 ul) was converted to cDNA and analyzed via TAQMAN® using One Step RT-PCR Master Mix Reagents (PE Biosystems; cat. #4309169) and gene-specific primers according to the manufacturer's instructions. Probes and primers were designed for each assay according to Perkin Elmer Biosystem's Primer Express Software package (version I for Apple Computer's Macintosh Power PC) using the nucleic acid sequences of the invention as input. A summary of the specific probes and primers constricted is shown in Table 4. Default settings were used for reaction conditions and the following parameters were set before selecting primers: primer concentration=250 nM, primer melting temperature (Tₘ) range=58°-60° C., primer optimal Tm=59° C., maximum primer difference=2° C., probe does not have 5′ G, probe Tₘ must be 10° C. greater than primer Tₘ, amplicon size 75 bp to 100 bp. The probes and primers selected (see below) were synthesized by Synthegen (Houston, Tex., USA). Probes were double purified by HPLC to remove uncoupled dye and evaluated by mass spectroscopy to verify coupling of reporter and quencher dyes to the 5′ and 3′ ends of the probe. Final concentrations were for the forward and reverse primers were 900 nM. Final concentration for the probes were 200 nM.

PCR was performed as follows, normalized RNA from each tissue and each cell line was spotted in each well of a 96 well PCR plate (Perkin Elmer Biosystems). PCR cocktails including two probes (SECX-specific and another gene-specific probe multiplexed with the SECX probe) were set up using 1× TaqMan™ PCR Master Mix for the PE Biosystems 7700, with 5 mM MgCl2, dNTPs (dA, G, C, U at 1:1:1:2 ratios), 0.25 U/ml AmpliTaq Gold™ (PE Biosystems), and 0.4 U/μl RNase inhibitor, and 0.25 U/μl reverse transcriptase. Reverse transcription was performed at 48° C. for 30 minutes followed by amplification/PCR cycles as follows: 95° C. 10 min, then 40 cycles of 95° C. for 15 seconds, 60° C. for 1 minute.

A summary of the expression results is presented in Table 5. Expression in the indicated cell or tissue for the given SECX sequence is presented as a percentage of expression relative to the reference transcript.

TABLE 4

| | | | | |
|---|---|---|---|---|
| | | Target | | |
| | Clone | Sequence | | SEQ ID |
| SECX | Identification | Position | Primers/Probes | NO |

| | | | | |
|---|---|---|---|---|
| 10326230.0.38 | 588-663 | Ag 2 (F): | 5′-GTGCTGCTGCTCTACAATAACCA-3′ | 34 |
| | | Ag 2 (R): | 5′-GTTTCTGCAGCTGGGCCAT-3′ | 35 |
| | | Ag 2 (P): | -FAM-5′-TGGACCGGTGCGCCTTCGAT-3′-TAMRA | 36 |
| 16399139.0.7 | 418-495 | Ag 40 (F): | 5′-GGCACGTCCCTCCGTTCT-3′ | 37 |
| | | Ag 40 (R): | 5′-CTGTTCAAGTTGCAAACCACAAG-3′ | 38 |
| | | Ag 40 (P): | FAM-5′-CTGCGACAACGAGCTCCTGCACTG-3′-TAMRA | 39 |
| 3581980.0.30 | 5-80 | Ag 151 (F): | 5′-CCCATGTGACAGTGACGAAGTC-3′ | 40 |
| | | Ag 151 (R): | 5′-AGTGCTGATTGCCGGGTTTAC-3′ | 41 |
| | | Ag 151 (P): | FAM-5′-CTGTTTTCTCTCGCGTCTCTCTGTTTCTGG-3′-TAMRA | 42 |
| 4418354 | 495-570 | Ag 156 (F): | 5′-AGCACCATCCACAGCTGCTT-3 | 43 |
| | | Ag 156 (R): | 5′-TGACCCTCATCCATGGCTACT-3′ | 44 |
| | | Ag 156 (P): | TET-5′-CTCATCAGAGAGCCCCTGCGTGC-3′-TAMRA | 45 |
| 6779999.0.31 | 610-688 | Ag 108 (F): | 5′-GCATGCCTGTAGTCCCAGCTA-3′ | 46 |
| | | Ag 108 (R): | 5′-ACCCAAGCTGGATTAGAATTCCT-3′ | 47 |
| | | Ag 108 (P): | FAM-5′-AAGCAATCCTCTTGCCTCAGTCTCCCAA-3′-TAMRA | 48 |
| 8484782.0.5 | 354-432 | Ag18 (F): | 5′-ACCCGCTGTGTTTGCTGAC-3′ | 49 |
| | | Ag18 (R): | 5′-TTTTCTACCGCTCCCCAGTCT-3′ | 50 |
| | | Ag18 (P): | FAM-5′-AACCTACCCTGGAGTTCCGGAGCG-TAMRA | 51 |

TABLE 5

| | Relative Expression (%) | | | | | |
|---|---|---|---|---|---|---|
| | SEC2 | SEC3 | SEC5 | SEC6 | SEC8 | SEC9 |
| Endothelial cells | 0.46 | 0.00 | 0.00 | 8.42 | 0.45 | 0.00 |
| Endothelial cells (treated) | 0.08 | 0.00 | 0.00 | 9.47 | 0.07 | 0.00 |
| Pancreas | 4.87 | 0.03 | 0.16 | 28.32 | 0.39 | 0.00 |
| Adipose | 14.87 | 0.19 | 28.52 | 50.35 | 0.25 | 0.00 |
| Adrenal gland | 25.35 | 0.06 | 0.47 | 46.65 | 1.10 | 100.00 |
| Thyroid | 8.19 | 0.00 | 0.00 | 34.39 | 0.20 | 0.00 |
| Salavary gland | 7.38 | 0.02 | 0.05 | 41.47 | 3.40 | 0.00 |
| Pituitary gland | 3.00 | 0.00 | 0.74 | 22.38 | 0.05 | 0.00 |
| Brain (fetal) | 12.24 | 4.04 | 26.24 | 12.41 | 4.18 | 4.42 |
| Brain (whole) | 78.46 | 17.31 | 42.93 | 20.17 | 17.80 | 31.43 |
| Brain (amygdala) | 26.98 | 11.34 | 17.56 | 48.63 | 0.78 | 1.69 |
| Brain (cerebellum) | 100.00 | 10.37 | 100.00 | 54.34 | 85.86 | 23.33 |
| Brain (hippocampus) | 87.06 | 20.31 | 50.00 | 55.86 | 3.74 | 5.75 |
| Brain (hypothalamus) | 21.61 | 0.11 | 0.02 | 50.35 | 3.77 | 0.54 |
| Brain (substantia nigra) | 28.92 | 6.34 | 4.18 | 68.30 | 2.94 | 0.31 |
| Brain (thalamus) | 29.12 | 100.00 | 4.18 | 68.78 | 0.51 | 0.13 |
| Spinal cord | 4.94 | 0.44 | 0.00 | 30.15 | 0.02 | 0.00 |
| Heart | 0.00 | 0.00 | 4.54 | 52.49 | 0.30 | 0.00 |
| Skeletal muscle | 15.18 | 0.00 | 0.00 | 100.00 | 0.23 | 0.00 |
| Bone marrow | 1.30 | 0.00 | 1.08 | 44.75 | 1.01 | 0.00 |
| Thymus | 7.23 | 0.03 | 0.03 | 56.25 | 13.97 | 0.00 |
| Spleen | 5.29 | 0.00 | 0.05 | 40.61 | 1.28 | 0.00 |
| Lymph node | 11.19 | 0.08 | 0.03 | 18.82 | 6.75 | 0.00 |
| Colon (ascending) | 0.00 | 0.00 | 4.15 | 43.53 | 7.23 | 0.00 |
| Stomach | 10.51 | 0.05 | 5.75 | 22.69 | 5.11 | 0.00 |
| Small intestine | 3.47 | 0.02 | 0.45 | 29.52 | 7.08 | 0.00 |
| Bladder | 9.67 | 0.03 | 30.35 | 81.23 | 1.88 | 0.00 |
| Trachea | 5.95 | 0.00 | 3.49 | 24.83 | 5.11 | 0.00 |
| Kidney | 8.66 | 0.06 | 0.06 | 75.26 | 1.96 | 0.00 |
| Kidney (fetal) | 6.52 | 0.10 | 0.85 | 43.83 | 3.24 | 0.01 |
| Liver | 3.85 | 0.00 | 0.47 | 24.49 | 9.02 | 0.00 |
| Liver (fetal) | 0.95 | 0.00 | 1.29 | 34.63 | 0.26 | 0.00 |
| Lung | 8.90 | 0.03 | 1.76 | 22.07 | 0.00 | 0.00 |
| Lung (fetal) | 1.63 | 0.00 | 0.00 | 9.54 | 2.68 | 0.00 |
| Mammary gland | 13.97 | 0.00 | 4.74 | 44.44 | 1.99 | 0.00 |
| Ovary | 8.54 | 0.08 | 0.00 | 50.70 | 0.00 | 0.00 |
| Myometrium | 2.80 | 0.00 | 5.59 | 27.74 | 1.31 | 0.00 |
| Uterus | 5.87 | 0.13 | 0.08 | 46.33 | 2.70 | 0.00 |
| Plancenta | 4.45 | 0.00 | 1.41 | 43.83 | 0.00 | 0.00 |
| Prostate | 6.21 | 0.03 | 3.82 | 38.96 | 1.35 | 0.00 |
| Testis | 13.40 | 0.00 | 4.74 | 92.66 | 100.00 | 0.00 |
| Breast ca.* (pl. effusion) MCF-7 | 42.34 | 0.00 | 0.00 | 100.00 | 32.09 | 0.00 |
| Breast ca.* (pl.ef) MDA-MB-231 | 10.08 | 0.00 | 0.00 | 16.72 | 4.45 | 0.00 |
| Breast ca. BT-549 | 37.37 | 0.00 | 0.22 | 18.95 | 0.00 | 0.00 |
| Breast ca.* (pl. effusion) T47D | 28.13 | 0.00 | 1.98 | 51.76 | 100.00 | 0.00 |
| Breast ca. MDA-N | 11.99 | 0.00 | 0.32 | 41.18 | 42.04 | 0.00 |
| Ovarian ca. OVCAR-3 | 12.59 | 0.00 | 0.00 | 35.85 | 6.70 | 0.00 |
| Ovarian ca.* (ascites) SK-OV-3 | 21.32 | 0.00 | 0.00 | 7.91 | 24.15 | 0.00 |
| Ovarian ca. OVCAR-4 | 5.40 | 0.00 | 0.00 | 6.56 | 0.48 | 0.00 |
| Ovarian ca. OVCAR-5 | 19.21 | 0.00 | 13.30 | 35.11 | 34.39 | 0.00 |
| Ovarian ca. IGROV-1 | 7.13 | 0.00 | 0.27 | 21.46 | 16.61 | 0.00 |
| Ovarian ca. OVCAR-8 | 52.49 | 0.00 | 0.87 | 55.86 | 64.17 | 0.00 |
| CNS ca. (glio/astro) U87-MG | 4.51 | 0.00 | 0.00 | 23.98 | 17.92 | 0.00 |
| CNS ca (astro) SW 1783 | 2.90 | 0.00 | 0.33 | 12.07 | 8.30 | 0.00 |
| CNS ca. (glio/astro) U-118-MG | 0.29 | 0.00 | 0.00 | 13.77 | 25.88 | 0.00 |
| CNS ca.* (neuro; met) SK-N-AS | 9.15 | 0.00 | 0.34 | 32.31 | 5.48 | 0.00 |
| CNS ca. (astro) SF-539 | 0.40 | 0.00 | 0.00 | 14.36 | 14.97 | 0.00 |
| CNS ca. (astro) SNB-75 | 3.08 | 0.00 | 37.11 | 15.07 | 32.09 | 0.00 |
| CNS ca. (glio) SNB-19 | 29.32 | 0.00 | 29.73 | 25.35 | 35.11 | 100.00 |
| CNS ca. (glio) U251 | 12.07 | 0.00 | 0.54 | 10.73 | 10.01 | 0.00 |
| CNS ca. (glio) SF-295 | 8.78 | 0.00 | 0.00 | 21.02 | 6.70 | 0.00 |
| Colon ca. SW480 | 2.42 | 0.00 | 2.68 | 14.26 | 3.93 | 0.00 |
| Colon ca.* (SW480 met)SW620 | 3.08 | 86.45 | 0.00 | 15.28 | 22.69 | 0.00 |
| Colon ca. HT29 | 4.84 | 72.70 | 0.00 | 32.53 | 29.12 | 0.00 |
| Colon ca HCT-116 | 1.77 | 0.00 | 0.53 | 16.96 | 0.00 | 0.00 |
| Colon ca. CaCo-2 | 4.90 | 0.00 | 9.67 | 15.18 | 5.56 | 0.00 |
| Gastric ca.* (liver met) NCI-N87 | 44.44 | 0.00 | 0.00 | 51.76 | 96.59 | 0.00 |
| Colon ca. HCT-15 | 25.70 | 0.00 | 0.00 | 42.04 | 20.45 | 0.00 |
| Colon ca. HCC-2998 | 9.09 | 2.29 | 0.00 | 64.62 | 38.42 | 0.00 |
| Renal ca. 786-0 | 0.73 | 0.00 | 15.93 | 19.89 | 35.85 | 0.00 |
| Renal ca. A498 | 0.13 | 0.00 | 40.05 | 24.49 | 5.08 | 0.00 |
| Renal ca. RXF 393 | 0.68 | 0.00 | 19.34 | 3.56 | 5.26 | 0.00 |
| Renal ca. ACHN | 7.97 | 0.00 | 0.00 | 10.66 | 3.67 | 0.00 |
| Renal ca. UO-31 | 4.77 | 0.00 | 0.00 | 17.68 | 7.97 | 0.00 |
| Renal ca. TK-10 | 12.16 | 83.51 | 0.00 | 23.16 | 86.45 | 0.00 |
| Liver ca. (hepatoblast) HepG2 | 5.75 | 100.00 | 6.84 | 34.87 | 28.13 | 0.00 |
| Lung ca. (small cell) LX-1 | 1.86 | 0.00 | 18.17 | 20.45 | 28.92 | 0.00 |
| Lung ca. (small cell) NCI-H69 | 1.77 | 0.00 | 0.71 | 13.12 | 25.00 | 1.30 |
| Lung ca (s.cell var.) SHP-77 | 82.36 | 0.00 | 100.00 | 15.71 | 0.00 | 0.00 |
| Lung ca. (non-sm. cell) A549 | 5.59 | 0.00 | 0.00 | 33.92 | 42.63 | 0.00 |
| Lung ca. (squam.) SW 900 | 11.42 | 0.00 | 0.00 | 59.87 | 22.07 | 0.00 |
| Lung ca. (squam.) NCI-H596 | 3.02 | 0.00 | 0.00 | 16.61 | 28.72 | 60.71 |
| Lung ca. (non-s.cell) NCI-H23 | 20.88 | 0.00 | 1.01 | 32.31 | 27.93 | 0.00 |
| Lung ca. (large cell) NCI-H460 | 61.13 | 0.00 | 14.56 | 54.71 | 0.00 | 0.00 |
| Lung ca (non-s.cell) HOP-62 | 2.88 | 0.00 | 0.63 | 40.05 | 3.19 | 0.00 |
| Lung ca. (non-s.cl) NCI-H522 | 37.37 | 0.00 | 0.00 | 18.56 | 33.68 | 0.00 |
| Pancreatic Ca. CAPAN 2 | 0.02 | 0.00 | 4.12 | 17.08 | 12.76 | 0.00 |
| Prostate ca.* (bone met)PC-3 | 100.00 | 0.00 | 1.02 | 53.22 | 0.00 | 0.00 |
| Melanoma Hs688(A).T | 1.94 | 0.00 | 0.00 | 8.36 | 0.11 | 0.00 |
| Melanoma* (met) Hs688(B).T | 2.05 | 0.00 | 0.68 | 12.85 | 4.07 | 0.00 |
| Melanoma UACC-62 | 3.04 | 0.00 | 0.00 | 26.24 | 0.00 | 0.00 |
| Melanoma M14 | 17.19 | 0.00 | 39.50 | 16.49 | 22.22 | 0.00 |
| Melanoma LOX IMVI | 8.78 | 0.00 | 0.00 | 9.02 | 3.54 | 0.00 |
| Melanoma* (met) SK-MEL-5 | 5.01 | 0.00 | 25.53 | 33.22 | 19.48 | 0.00 |
| Melanoma SK-MEL-28 | 9.15 | 0.00 | 0.32 | 100.00 | 14.66 | 0.00 |
| Melanoma UACC-257 | 1.72 | 0.00 | 0.37 | 100.00 | 15.93 | 0.00 |

ca. = carcinoma
met = metastasis
non-s = non-sm = non-small
pl. eff = pleural effusion
astro = astrocytoma
*= established from metastasis
s cell var = small cell variant,
squam = squamous,
glio = glioma,
neutro = neuroblastoma.

### Example 9

CG51051-07: Netrin/Laminin Like-Proteins

Assembly: 228549724

TABLE 6
Molecular Cloning of CG51051-07: FL_480

(SEQ ID NO:54)

>CG51051-07
CAAGCTCTGCTTTAGTTTCCAAGAAGATTACAAAGAATTTAGAGATGTATTTGTCAAGATTCCTGTCGATTCATGCCCTT
TGGGTTACGGTGTCCTCAGTGATGCAGCCCTACCCTTTGGTTTGGGGACATTATGATTTGTGTAAGACTCAGATTTACAC
GGAAGAAGGGAAAGTTTGGGATTACATGGCCTGCCAGCCGGAATCCACGGACATGACAAAATATCTGAAAGTGAAACT
CGATCCTCCGGATATTACCTGTGGAGACCCTCCTGAGACGTTCTGTGCAATGGGCAATCCCTACATGTGCAATAATGAG
TGTGATGCGAGTACCCCTGAGCTGGCACACCCCCCTGAGCTGATGTTTGATTTTGAAGGAAGACATCCCTCCACATTTT
GGCAGTCTGCCACTTGGAAGGAGTATCCCAAGCCTCTCCAGGTTAACATCACTCTGTCTTGGAGCAAAACCATTGAGCT
AACAGACAACATAGTTATTACCTTTGAATCTGGGCGTCCAGACCAAATGATCCTGGAGAAGTCTCTCGATTATGGACGA
ACATGGCAGCCCTATCAGTATTATGCCACAGACTGCTTAGATGCTTTTCACATGGATCCTAAATCCGTGAAGGATTTAT
CACAGCATACGGTCTTAGAAATCATTTGCACAGAAGAGTACTCAACAGGGTATACAACAAATAGCAAAATAATCCACT
TTGAAATCAAAGACAGGTTCGCGTTTTTTGCTGGACCTCGCCTACGCAATATGGCTTCCCTCTACGGACAGCTGGATAC
AACCAAGAAACTCAGAGATTTCTTTACAGTCACAGACCTGAGGATAAGGCTGTTAAGACCAGCCGTTGGGGAAATATT
TGTAGATGAGCTACACTTGGCACGCTACTTTTACGCGATCTCAGACATAAAGGTGCGAGGAAGGTGCAAGTGTAATCTC
CATGCCACTGTATGTGTGTATGACAACAGCAAATTGACATGCGAATGTGAGCACAACACTACAGGTCCAGACTGTGGG
AAATGCAAGAAGAATTATCAGGGCCGACCTTGGAGTCCAGGCTCCTATCTCCCCATCCCCAAAGGCACTGCAAATACC
TGTATCCCCAGTATTTCCAGTATTGGTAATCCTCCAAAGTTTAATAGGATATGGCCGAATATTTCTTCCCTTGAGGTTTC
TAACCCAAAACAAGTTGCTCCCAAATTAGCTTTGTCAACAGTTTCTTCTGTTCAAGTTGCAAACCACAAGAGAGCGAAT
GTCTGCGACAACGAGCTCCTGCACTGCCAGAACGGAGGGACGTGCCACAACAACGTGCGCTGCCTGTGCCCGGCCGCA
TACACGGGCATCCTCTGCGAGAAGCTGCGGTGCGAGGAGGCTGGCAGCTGCGGCTCCGACTCTGGCCAGGGCGCGCCC
CCGCACGGCTCCCCAGCGCTGCTGCTGCTGACCACGCTGCTGGGAACCGCCAGCCCCCTGGTGTTCTAGGTGTCAC

The cDNA coding for the mature form of CG51051-07 from residue 20 to 480 was targeted for “in-frame” cloning by PCR. The PCR template is based on the previously identified plasmid.

Two PCR reactions were set up using a total of 1-5 ng of the plasmid that contains the insert for CG51051-07.

The reaction mixtures contained 2 microliters of each of the primers (original concentration: 5 pmol/ul), 1 microliter of 10 mM DNTP (Clontech Laboratories, Palo Alto Calif.) and 1 microliter of 50× Advantage-HF 2 polymerase (Clontech Laboratories) in 50 microliter-reaction volume. The following reaction conditions were used:

PCR condition 1:

| | | |
|---|---|---|
| a) | 96° C. | 3 minutes |
| b) | 96° C. | 30 seconds denaturation |
| c) | 60° C. | 30 seconds, primer annealing |
| d) | 72° C. | 6 minutes extension |

Repeat steps b-d 15 times

| | | |
|---|---|---|
| e) | 96° C. | 15 seconds denaturation |
| f) | 60° C. | 30 seconds, primer annealing |
| g) | 72° C. | 6 minutes extension |

Repeat steps e-g 29 times

| | | |
|---|---|---|
| e) | 72° C. | 10 minutes final extension |

PCR condition 2:

| | | |
|---|---|---|
| a) | 96° C. | 3 minutes |
| b) | 96° C. | 15 seconds denaturation |
| c) | 76° C. | 30 seconds, reducing the temperature by 1° C. per cycle |
| d) | 72° C. | 4 minutes extension |

Repeat steps b-d 34 times

| | | |
|---|---|---|
| e) | 72° C. | 10 minutes final extension. |

An amplified product was detected by agarose gel electrophoresis. The fragment was gel-purified and ligated into the pCR2.1 vector (Invitrogen, Carlsbad, Calif.) following the manufacturer's recommendation. Twelve clones per PCR reaction were picked and sequenced. The inserts were sequenced using vector-specific M13 Forward and M13.

TABLE 7

Reverse primers and the following gene-specific
primers:

| | | |
|---|---|---|
| SF1: | CCCCCTGAGCTGATGTTTGATTTT | (SEQ ID NO:58) |
| SF2: | GTATTATGCCACAGACTGCTTTAGATGC | (SEQ ID NO:59) |
| SF3: | TCAGAGATTTCTTTACAGTCACAGACC | (SEQ ID NO:60) |
| SF4: | GCAAGAAGAATTATCAGGGCCGA | (SEQ ID NO:61) |
| SF5: | AGTTGCTCCCAAATTAGCTTTGTCA | (SEQ ID NO:62) |
| SR1: | CTCAGGGGTACTCGCATCACACT | (SEQ ID NO:63) |
| SR2: | GATAGGGCTGCCATGTTCGTCCATAAT | (SEQ ID NO:64) |
| SR3: | GTTTCTTGGTTGTATCCAGCTGTCC | (SEQ ID NO:65) |
| SR4: | TTTCCCACAGTCTGGACCTGTAGTGTT | (SEQ ID NO:66) |
| SR5: | CTTGTTTTGGGTTAGAAACCTCAAGGG | (SEQ ID NO:67) |

Results:

The insert assembly 228549724 was found to encode an open reading frame between residues 20 and 480 of the target sequence CG51051-07. The cloned insert is 100% identical to the original sequence. The alignment with CG51051-07 is displayed in a ClustalW below. Note that differing amino acids have a white or grey background, and deleted/inserted amino acids can be detected by a dashed line in the sequence that does not code at that position.

TABLE 8
Nucleotide Sequence for Assembly No. 228549724:

(SEQ ID NO:56)

>228549724
AGGCTCCGCGGCCGCCCCCTTCACCGTGATGCAGCCCTACCCTITGGTTTGGGGACATTATGATTTGTGTAAGACTCAG
ATTTACACGGAAGAAGGGAAAGTTTGGGATTACATGGCCTGCCAGCCGGAATCCACGGACATGACAAAATATCTGAAA
GTGAAACTCGATCCTCCGGATATTACCTGTGGAGACCCTCCTGAGACGTTCTGTGCAATGGGCAATCCCTACATGTGCA
ATAATGAGTGTGATGCGAGTACCCCTGAGCTGGCACACCCCCCTGAGCTGATGTTTGATTTTGAAGGAAGACATCCCTC
CACATTTGGCAGTCTGCCACTTGGAAGGAGTATCCCAAGCCTCTCCAGGTTAACATCACTCTGTCTTGGAGCAAAACC
ATTGAGCTAACAGACAACATAGTTATTACCTTTGAATCTGGGCGTCCAGACCAAATGATCCTGGAGAAGTCTCTCGATT
ATGGACGAACATGGCAGCCCTATCAGTA1TATGCCACAGACTGCTTAGATGCTTTTCACATGGATCCTAAATCCGTGAA
GGATTTATCACAGCATACGGTCTTAGAAATCATTTGCACAGAAGAGTACTCAACAGGGTATACAACAAATAGCAAAAT
AATCCACTTTGAAATCAAAGACAGGTTCGCGTTTTTTGCTGGACCTCGCCTACGCAATATGGCTTCCCTCTACGGACAG
CTGGATACAACCAAGAAACTCAGAGATTTCTTTACAGTCACAGACCTGAGGATAAGGCTGTTAAGACCAGCCGTTGGG
GAAATATTTGTAGATGAGCTACACTTGGCACGCTACTTTTACGCGATCTCAGACATAAAGGTGCGAGGAAGGTGCAAG
TGTAATCTCCATGCCACTGTATGTGTGTATGACAACAGCAAATTGACATGCGAATGTGAGCACAACACTACAGGTCCAG
ACTGTGGGAAATGCAAGAAGAATTATCAGGGCCGACC1TGGAGTCCAGGCTCCTATCTCCCCATCCCCAAAGGCACTG
CAAATACCTGTATCCCCAGTATTCCAGTATGGTAATCCTCCAAAGTTTAATAGGATATGGCCGAATATTTCTTCCCTTT
GAGGTTTCTAACCCAAAACAAGTTGCTCCCAAATTAGCTTTGTCAACAGTTTCTTCTGTTCAATTGCAAACCACAAGA
GAGCGAATGTCTGCGACAACGAGCTCCTGCACTGCCAGAACGGAGGGACGTGCCACAACAACGTGCGCTGCCTGTGCC
CGGCCGCATACACGGGCATCCTCTGCGAGAAGCTGCGGTGCGAGGAGGCTGGCAGCTGCGGCTCCGACTCTGGCCAGG
GCGCGCCCCCGCACGGCTCCCCAGCGCTGCTGCTGCTGACCACGCTGCTGGGAACCGCCAGCCCCGTGGTGTTCAAGG
GTGGGCGCGCC

[image]

### Example 10

CG51051-07: Netrin/Laminin-Like Proteins

Assembly: 228506045

The cDNA coding for the full length form of CG51051-07 from residue 1 to 480 was targeted for “in-frame” cloning by PCR. The PCR template is based on the previously identified plasmid.

TABLE 10

DNA Sequence Analysis for Assembly No. 228549724
(DNA SEQ ID NO.56; Protein SEQ ID NO:57)
View DNA Sequence Analysis of 228549724

Translated Protein-Frame: 2-Nucleotide 2 to 1426
Printed 80 characters to a line.

| | |
|---|---|
| 1 | AGGCTCCGCGGCCGCCCCCTTCACCGTGATGCAGCCCTACCCTTTGGTTTGGGGACATTATGATTTGTGTAAGACTCAGA |
| | G S A A A P F T V M Q P Y P L V W G H Y D L C K T Q I |
| 81 | TTTACACGGAAGAAGGGAAAGTTTGGGATTACATGGCCTGCCAGCCGGAATCCACGGACATGACAAAATATCTGAAAGTG |
| | Y T E E G K V W D Y M A C Q P E S T D M T K Y L K V |
| 161 | AAACTCGATCCTCCGGATATTACCTGTGGAGACCCTCCTGAGACGTTCTGTGCAATGGGCAATCCCTACATGTGCAATAA |
| | K L D P P D I T C G D P P E T F C A M G M P Y M C N N |
| 241 | TGAGTGTGATGCGAGTACCCCTGAGCTGGCACACCCCCCTGAGCTGATGTTTGATTTTGAAGGAAGACATCCCTCCACAT |
| | E C D A S T P E L A H P P E L M F D F E G R H P S T F |
| 321 | TTTGGCAGTCTGCCACTTGGAAGGAGTATCCCAAGCCTCTCCAGGTTAACATCACTCTGTCTTGGAGCAAAACCATTGAG |
| | W Q S A T W K E Y P K P L Q V N I T L S W S K T I E |
| 401 | CTAACAGACAACATAGTTATTACCTTTGAATCTGGGCGTCCAGACCAAATGATCCTGGAGAAGTCTCTCGATTATGGACG |
| | L T D N I V I T F E S G RP D Q M I L E K S L D Y G R |
| 481 | AACATGGCAGCCCTATCAGTATTATGCCACAGACTGCTTAGATGCTTTTCACATGGATCCTAAATCCGTGAAGGATTTAT |
| | T W Q P V Q Y Y A T D C L D A F H M D P K S V K D L S |
| 561 | CACAGCATACGGTCTTAGAAATCATTTGCACAGAAGAGTACTCAACAGGGTATACAACAAATAGCAAAATAATCCACTTT |
| | Q H T V L E I I C T E E Y S T G Y T T N S K I I H F |
| 641 | GAAATCAAAGACAGGTTCGCGTTTTTTGCTGGACCTCGCCTACGCAATATGGCTTCCCTCTACGGACAGCTGGATACAAC |
| | E I K D R F A F F A G P R L R N M A S L Y G Q L D T T |
| 721 | CAAGAAACTCAGAGATTTCTTTACAGTCACAGACCTGAGGATAAGGCTGTTAAGACCAGCCGTTGGGGAAATATTTGTAG |
| | K K L R D F F T V T D L R I R L R P A V G E I F V D |
| 801 | ACTGTATGTGTGTATGACAACAGCAAATTGACATGCGAATGTGAGCACAACACTACAGGTCCAGACTGTGGGAAATGCAA |
| | E L H L A R Y F Y A I S D I K V R G R C K C N L H A |
| 881 | ACTGTATGTGTGTATGACAACAGCAAATTGACATGCGAATGTGAGCACAACACTACAGGTCCAGACTGTGGGAAATGCAA |
| | T V C V Y D N S K L T C E C E H N T T G P D C G K C K |
| 961 | GAAGAATTATCAGGGCCGACCTTGGAGTCCAGGCTCCTATCTCCCCATCCCCAAAGGCACTGCAAATACCTGTATCCCCA |
| | K N Y Q G R P W S P G S Y L P I P K G T A N T C I P S |
| 1041 | GTATTTCCAGTATTGGTAATCCTCCAAAGTTTAATAGGATATGGCCGAATATTTCTTCCCTTGAGGTTTCTAACCCAAAA |
| | I S S I G N P P K F N R I W P N I S S L E V S N P K |
| 1121 | CAAGTTGCTCCCAAATTAGCTTTGTCAACAGTTTCTTCTGTTCAAGTTGCAAACCACAAGAGAGCGAATGTCTGCGACAA |
| | Q V A P K L A L S T V S S V Q V A N H K R A N V C D N |
| 1201 | CGAGCTCCTGCACTGCCAGAACGGAGGGACGTGCCACAACAACGTGCGCTGCCTGTGCCCGGCCGCATACACGGGCATCC |
| | E L L H C Q N G G T C H N N V R C L C P A A Y T G I L |
| 1281 | TCTGCGAGAAGCTGCGGTGCGAGGAGGCTGGCAGCTGCGGCTCCGACTCTGGCCAGGGCGCGCCCCCGCACGGCTCCCCA |
| | C E K L R C E E A G S C G S D S G Q G A P P H G S P |
| 1361 | GCGCTGCTGCTGCTGACCACGCTGCTGGGAACCGCCAGCCCCCTGGTGTTCAAGGGTGGGCGCGCC |
| | A L L L L T T L L G T A S P L V F K G G R A |

TABLE 11

Oligonucleotide Primers Used
to Clone the target cDNA sequence:

(SEQ ID NO:68)

| | |
|---|---|
| F1 | 5′-AGATCT ATGTATTTGTCAAGATTCCTGTCGATTCATGC-3′ |

(SEQ ID NO:69)

| | |
|---|---|
| R1 | 5′-CTCGAG GAACACCAGGGGGCTGGCGGTTCCCAGCAGC-3′ |

For downstream cloning purposes, the forward primer includes an in-frame BglII restriction site and the reverse primer contains an in-frame XhoI restriction site.

Two PCR reactions were set up using a total of 1-5 ng of the plasmid that contains the insert for CG51051-07.

The reaction mixtures contained 2 microliters of each of the primers (original concentration: 5 pmol/ul), 1 microliter of 10 mM dNTP (Clontech Laboratories, Palo Alto Calif.) and 1 microliter of 50× Advantage-HF 2 polymerase (Clontech Laboratories) in 50 microliter-reaction volume. The following reaction conditions were used:

PCR condition 1:

| | | |
|---|---|---|
| a) | 96° C. | 3 minutes |
| b) | 96° C. | 30 seconds denaturation |
| c) | 60° C. | 30 seconds, primer annealing |
| d) | 72° C. | 6 minutes extension |

Repeat steps b-d 15 times

| | | |
|---|---|---|
| e) | 96° C. | 15 seconds denaturation |
| f) | 60° C. | 30 seconds, primer annealing |
| g) | 72° C. | 6 minutes extension |

Repeat steps e-g 29 times

| | | |
|---|---|---|
| e) | 72° C. | 10 minutes final extension |

PCR condition 2:

| | | |
|---|---|---|
| a) | 96° C. | 3 minutes |
| b) | 96° C. | 15 seconds denaturation |
| c) | 76° C. | 30 seconds, reducing the temperature by 1° C. per cycle |
| d) | 72° C. | 4 minutes extension |

Repeat steps b-d 34 times

| | | |
|---|---|---|
| e) | 72° C. | 10 minutes final extension. |

An amplified product was detected by agarose gel electrophoresis. The fragment was gel-purified and ligated into the pCR2.1 vector (Invitrogen, Carlsbad, Calif.) following the manufacturer's recommendation. Twelve clones per PCR reaction were picked and sequenced. The inserts were sequenced using vector-specific M13 Forward and M13.

TABLE 12

Reverse primers and the following gene-specific
primers:

| | | |
|---|---|---|
| SF1: | CCCTACATGTGCAATAATGAGTGTGA | (SEQ ID NO:70) |
| SF2: | GTCTCTCGATTATGGACGAACATGGC | (SEQ ID NO:71) |
| SF3: | TACGGACAGCTGGATACAACCAAGA | (SEQ ID NO:72) |
| SF4: | GTCCAGACTGTGGGAAATGCAAG | (SEQ ID NO:73) |
| SF5: | AGTTGCTCCCAAATTAGCTTTGTCA | (SEQ ID NO:74) |
| SR1: | ATTGCACAGAACGTCTCAGGAGG | (SEQ ID NO:75) |
| SR2: | AGACTTCTCCAGGATCATTTGGTCT | (SEQ ID NO:76) |
| SR3: | AGAGGGAAGCCATATTGCGTAGG | (SEQ ID NO:77) |
| SR4: | ACCTGTAGTGTTGTGCTCACATTCG | (SEQ ID NO:78) |
| SR5: | CTTGTTTTGGGTTAGAAACCTCAAGGG | (SEQ ID NO:79) |

Results:

The insert assembly 228506045 was found to encode an open reading frame between residues 1 and 480 of the target sequence CG51051-07. The cloned insert is 100% identical to the original sequence. The alignment with CG51051-07 is displayed in a ClustalW below. Note that differing amino acids have a white or grey background, and deleted/inserted amino acids can be detected by a dashed line in the sequence that does not code at that position.

[image]

TABLE 14
Nucleotide Sequence for Assembly No. 228506045

(SEQ ID NO:80)

>228506045
AGGCTCCGCGGCCGCCCCCTTCACCATGTATGTCAAGATTCCTGTCGATFCATGCCCTITGGGTTACGGTGTCCTCAG
TGATGCAGCCCTACCCTTTGGTTTGGGGACATTATGATTTGTGTAAGACTCAGATTTACACGGAAGAAGGGAAAGTTTG
GGATTACATGGCCTGCCAGCCGGAATCCACGGACATGACAAAATATCTGAAAGTGAAACTCGATCCTCCGGATATTAC
CTGTGGAGACCCTCCTGAGACGTTCTGTGCAATGGGCAATCCCTACATGTGCAATAATGAGTGTGATGCGAGTACCCCT
GAGCTGGCACACCCCCCTGAGCTGATGTTTGATTTTGAAGGAAGACATCCCTCCACATTTTGGCAGTCTGCCACTTGGA
AGGAGTATCCCAAGCCTCTCCAGGTTAACATCACTCTGTCTTGGAGCAAAACCATTGAGCTAACAGACAACATAGTTAT
TACCTTTGAATCTGGGCGTCCAGACCAAATGATCCTGGAGAAGTCTCTCGATTATGGACGAACATGGCAGCCCTATCAG
TATTATGCCACAGACTGCTTAGATGCTTTTCACATGGATCCTAAATCCGTGAAGGATTTATCACAGCATACGGTCTTAG
AAATCATTTGCACAGAAGAGTACTCAACAGGGTATACAACAAATAGCAAAATAATCCACTTTGAAATCAAAGACAGGT
TCGCGTTTTTTGCTGGACCTCGCCTACGCAATATGGCTTCCCTCTACGGACAGCTGGATACAACCAAGAAACTCAGAGA
TTTCTTTACAGTCACAGACCTGAGGATAAGGCTGTTAAGACCAGCCGTTGGGGAAATATTTGTAGATGAGCTACACTTG
GCACGCTACTTTTACGCGATCTCAGACATAAAGGTGCGAGGAAGGTGCAAGTGTAATCTCCATGCCACTGTATGTGTGT
ATGACAACAGCAAATTGACATGCGAATGTGAGCACAACACTACAGGTCCAGACTGTGGGAAATGCAAGAAGAATTATC
AGGGCCGACCTTGGAGTCCAGGCTCCTATCTCCCCATCCCCAAAGGCACTGCAAATACCTGTATCCCCAGTATTTCCAG
TATTGGTAATCCTCCAAAGTTTAATAGGATATGGCCGAATATTTCTTCCCTTGAGGTTTCTAACCCAAAACAAGTTGCTC
CCAAATTAGCTTTGTCAACAGTTTCTTCTGTTCAAGTTGCAAACCACAAGAGAGCGAATGTCTGCGACAACGAGCTCCT
GCACTGCCAGAACGGAGGGACGTGCCACAACAACGTGCGCTGCCTGTGCCCGGCCGCATACACGGGCATCCTCTGCGA
GAAGCTGCGGTGCGAGGAGGCTGGCAGCTGCGGCTCCGACTCTGGCCAGGGCGCGCCCCCGCACGGCTCCCCAGCGCT
GCTGCTGCTGACCACGCTGCTGGGAACCGCCAGCCCCCTGGTGTTCAAGGGTGGGCGCGCC

TABLE 15

DNA Sequence Analysis for Assembly No. 228506045:
(SEQ ID NO:80—Nucleotide Sequence; SEQ ID NO:81—Protein Sequence)

View DNA Sequence Analysis of 228504045
Translated Protein - Frame: 2 - Nucleotide 2 to 1480
Printed 80 characters to a line.

| | |
|---|---|
| 1 | AGGCTCCGCGGCCGCCCCCTTCACCATGTATTTGTCAAGATTCCTGTCGATTCATGCCCTTTGGGTTACGGTGTCCTCAG |
| | G S A A A P F T M Y L S R F L S I H A C W V T V S S V |
| 81 | TGATGCAGCCCTACCCTTTGGTTTGGGGACATTATGATTTGTGTAAGACTCAGATTTACACGGAAGAAGGGAAAGTTTGG |
| | M Q P Y P L V W G H Y D L C K T Q I Y T E E G K V W |
| 161 | GATTACATGGCCTGCCAGCCGGAATCCACGGACATGACAAAATATCTGAAAGTGAAACTCGATCCTCCGGATATTACCTG |
| | D Y M A C Q P E S T D M T K Y L K V K LD P P D I T C |
| 241 | TGGAGACCCTCCTGAGACGTTCTGTGCAATGGGCAATCCCTACATGTGCAATAATGAGTGTGATGCGAGTACCCCTGAGC |
| | G D P P E T F C AM G N P Y M C N N E C D A S T P E L |
| 321 | TGGCACACCCCCCTGAGCTGATGTTTGATTTTGAAGGAAGACATCCCTCCACATTTTGGCAGTCTGCCACTTGGAAGGAG |
| | A H P P E L M F D F E G R H P S T F W Q S A T W K E |
| 401 | TATCCCAAGCCTCTCCAGGTTAACATCACTCTGTCTTGGAGCAAAACCATTGAGCTAACAGACAACATAGTTATTACCTT |
| | Y P K P L Q V N I T L S W S K T I E L T D N I V I T F |
| 481 | TGAATCTGGGCGTCCAGACCAAATGATCCTGGAGAAGTCTCTCGATTATGGACGAACATGGCAGCCCTATCAGTATTATG |
| | E S G R P D Q M I L E K S L D Y G R T W Q P Y Q Y Y A |
| 561 | CCACAGACTGCTTAGATGCTTTTCACATGGATCCTAAATCCGTGAAGGATTTATCACAGCATACGGTCTTAGAAATCATT |
| | T D C L D A F H M D P K S V K D L S Q H T V L E I I |
| 641 | TGCACAGAAGAGTACTCAACAGGGTATACAACAAATAGCAAAATAATCCACTTTGAAATCAAAGACAGGTTCGCGTTTTT |
| | C T E E Y S T G Y T T N S K I I H F E I K D R F A F F |
| 721 | TGCTGGACCTCGCCTACGCAATATGGCTTCCCTGTACGGACAGCTGGATACAACCAAGAAACTCAGAGATTTCTTTACAG |
| | A G P R L R N M A S L Y G Q L D T T K K L R D E E T V |
| 801 | TCACAGACCTGAGGATAAGGCTGTTAAGACCAGCCGTTGGGGAAATATTTGTAGATGAGCTACACTTGGCACGCTACTTT |
| | T D L R I R L L R P A V G E I F V D E L H L A R Y F |
| 881 | TACGCGATCTCAGACATAAAGGTGCGAGGAAGGTGCAAGTGTAATCTCCATGCCACTGTATGTGTGTATGACAACAGCAA |
| | Y A I S D I K V R G R C K C N L H A T V C V Y D N S K |
| 961 | ATTGACATGCGAATGTGAGCACAACACTACAGGTCCAGACTGTGGGAAATGCAAGAAGAATTATCAGGGCCGACCTTGGA |
| | L T C E C E H N T T G P D C G K C K K N Y Q G R P W S |
| 1041 | GTCCAGGCTCCTATCTCCCCATCCCCAAAGGCACTGCAAATACCTGTATCCCCAGTATTTCCAGTATTGGTAATCCTCCA |
| | P G S Y L P I P K G T A N T C I P S I S S I G H P P |
| 1121 | AAGTTTAATAGGATATGGCCGAATATTTCTTCCCTTGAGGTTTCTAACCCAAAACAAGTTGCTCCCAAATTAGCTTTGTC |
| | K F N R I W P N I S S L E V S N P K Q V A P K L A L S |
| 1201 | AACAGTTTCTTCTGTTCAAGTTGCAAACCACAAGAGAGCGAATGTCTGCGACAACGAGCTCCTGCACTGCCAGAACGGAG |
| | T V S S V Q V A N H K R A N V C D N E L L H C Q N G G |
| 1281 | GGACGTGCCACAACAACGTGCGCTGCCTGTGCCCGGCCGCATACACGGGCATCCTCTGCGAGAAGCTGCGGTGCGAGGAG |
| | T C H N N V R C L C P A A Y T G I L C E K L R C E E |
| 1361 | GCTGGCAGCTGCGGCTCCGACTCTGGCCAGGGCGCGCCCCCGCACGGCTCCCCAGCGCTGCTGCTGCTGACCACGCTGCT |
| | A G S C G S D S G Q G A P P H G S P A L L L L T T L L |
| 1441 | GGGAACCGCCAGCCCCCTGGTGTTCAAGGGTGGGCGCGCC |
| | G T A S P L V F K G G R A |

### Example 11

CG107126-01 Type IIIa Membrane Protein-Like Proteins.

A novel protein encoded by a cDNA and/or by genomic DNA and proteins similar to it, namely, new proteins bearing sequence similarity to Type IIIa Membrane Protein, nucleic acids that encode these proteins or fragments thereof, and antibodies that bind immunospecifically to a protein of the invention, are given in this example.

The sequence of Acc. No. CG107126-01 was derived by laboratory cloning of cDNA fragments, by in silico prediction of the sequence. cDNA fragments covering either the full length of the DNA sequence, or part of the sequence, or both, were cloned. In silico prediction was based on sequences available in Curagen's proprietary sequence databases or in the public human sequence databases, and provided either the full length DNA sequence, or some portion thereof.

The laboratory cloning was performed using one or more of the methods summarized below:

cDNA libraries were derived from various human samples representing multiple tissue types, normal and diseased states, physiological states, and developmental states from different donors. Samples were obtained as whole tissue, primary cells or tissue cultured primary cells or cell lines. Cells and cell lines may have been treated with biological or chemical agents that regulate gene expression, for example, growth factors, chemokines or steroids. The cDNA thus derived was then directionally cloned into the appropriate two-hybrid vector (Gal4-activation domain (Gal4-AD) fusion). Such cDNA libraries as well as commercially available cDNA libraries from Clontech (Palo Alto, Calif.) were then transferred from E.coli into a CuraGen Corporation proprietary yeast strain (disclosed in U.S. Pat. Nos. 6,057,101 and 6,083,693, incorporated herein by reference in their entireties).

Gal4-binding domain (Gal4-BD) fusions of a CuraGen Corporation proprietary library of human sequences was used to screen multiple Gal4-AD fusion cDNA libraries resulting in the selection of yeast hybrid diploids in each of which the Gal4-AD fusion contains an individual cDNA. Each sample was amplified using the polymerase chain reaction (PCR) using non specific primers at the cDNA insert boundaries. Such PCR product was sequenced; sequence traces were evaluated manually and edited for corrections if appropriate. cDNA sequences from all samples were assembled together, sometimes including public human sequences, using bioinformatic programs to produce a consensus sequence for each assembly. Each assembly is included in CuraGen Corporation's database. Sequences were included as components for assembly when the extent of identity with another component was at least 95% over 50 bp. Each assembly represents a gene or portion thereof and includes information on variants, such as splice forms single nucleotide polymorphisms (SNPs), insertions, deletions and other sequence variations.

Exon Linking: The cDNA coding for the CG107126-01 sequence was cloned by the polymerase chain reaction (PCR) using the primers: 5′ AATTGGGAGAAGACTCACTGG 3′ (Seq ID NO: 82) and 5′ TGTGAGGTATTTATTTTGCAGC 3′ (SEQ ID NO: 83). Primers were designed based on in silico predictions of the full length or some portion (one or more exons) of the cDNA/protein sequence of the invention. These primers were used to amplify a cDNA from a pool containing expressed human sequences derived from the following tissues: adrenal gland, bone marrow, brain-amygdala, brain-cerebellum, brain-hippocampus, brain-substantia nigra, brain-thalamus, brain-whole, fetal brain, fetal kidney, fetal liver, fetal lung, heart, kidney, lymphoma-Raji, mammary gland, pancreas, pituitary gland, placenta, prostate, salivary gland, skeletal muscle, small intestine, spinal cord, spleen, stomach, testis, thyroid, trachea and uterus.

Multiple clones were sequenced and these fragments were assembled together, sometimes including public human sequences, using bioinformatic programs to produce a consensus sequence for each assembly. Each assembly is included in CuraGen Corporation's database. Sequences were included as components for assembly when the extent of identity with another component was at least 95% over 50 bp. Each assembly represents a gene or portion thereof and includes information on variants, such as splice forms single nucleotide polymorphisms (SNPs), insertions, deletions and other sequence variations.

Physical clone: the cDNA fragment derived by the screening procedure, covering the entire open reading frame is, as a recombinant DNA, cloned into pACT2 plasmid (Clontech) used to make the cDNA library. The recombinant plasmid is inserted into the host and selected by the yeast hybrid diploid generated during the screening procedure by the mating of both CuraGen Corporation proprietary yeast strains N106′ and YULH (U.S. Pat. Nos 6,057,101 and 6,083,693) to provide clone 58524::3440544_{—}0_{—}87.698034.B9. Physical clone: The PCR product derived by exon linking, covering the entire open reading frame, was cloned into the pCR2.1 vector from Invitrogen to provide clone 58524::3440544_{—}0_{—}87.698034.B9.

Variant sequences are also included in this application. A variant sequence can include a single nucleotide polymorphism (SNP). A SNP can, in some instances, be referred to as a “cSNP” to denote that the nucleotide sequence containing the SNP originates as a cDNA. A SNP can arise in several ways. For example, a SNP may be due to a substitution of one nucleotide for another at the polymorphic site. Such a substitution can be either a transition or a transversion. A SNP can also arise from a deletion of a nucleotide or an insertion of a nucleotide, relative to a reference allele. In this case, the polymorphic site is a site at which one allele bears a gap with respect to a particular nucleotide in another allele. SNPs occurring within genes may result in an alteration of the amino acid encoded by the gene at the position of the SNP. Intragenic SNPs may also be silent, when a codon including a SNP encodes the same amino acid as a result of the redundancy of the genetic code. SNPs occurring outside the region of a gene, or in an intron within a gene, do not result in changes in any amino acid sequence of a protein but may result in altered regulation of the expression pattern. Examples include alteration in temporal expression, physiological response regulation, cell type expression regulation, intensity of expression, and stability of transcribed message.

The DNA sequence and protein sequence for a novel Type IIIa Membrane Protein-like gene were obtained by exon linking and are reported here as CuraGen Acc. No. CG107126-01.

Results

The novel nucleic acid of 1569 nucleotides (designated CuraGen Acc. No. CG107126-01) encoding a novel Type IIIa Membrane Protein-like protein is shown in Table 17. An open reading frame was identified beginning at nucleotides 53-55 and ending at nucleotides 971-973. This polypeptide represents a novel functional Type IIIa Membrane Protein-like protein. The start and stop codons of the open reading frame are highlighted in bold type. Putative untranslated regions (underlined), if any, are found upstream from the initiation codon and downstream from the termination codon. The encoded protein having 306 amino acid residues is presented using the one-letter code in Table 18.

Similarities

In a search of sequence databases, it was found, for example, that the nucleic acid sequence of this invention has 980 of 991 bases (98%) identical to a gb:GENBANK-ID:AX050254|acc:AX050254.1 mRNA from Homo sapiens (Sequence 7 from Patent WO0070046) (Table 19). The full amino acid sequence of the protein of the invention was found to have 304 of 306 amino acid residues (99%) identical to, and 304 of 306 amino acid residues (99%) similar to, the 306 amino acid residue ptnr:REMTREMBL-ACC:CAC21787 protein from Homo sapiens (Human) (Sequence 7 from Patent WO0070046) (Table 20).

A multiple sequence alignment is given in (Table 22), with the protein of the invention being shown on the first line in a ClustalW analysis comparing the protein of the invention with related protein sequences.

Please see Table 22 to note the following amino acid differences between publicly available gene, a mouse homolog, a filing for patent originating from Curagen Corporation and the invention, which are structurally variable and possibly functionally variable:

| Position | Amino Acid difference |
|---|---|
| 32 | V → D |
| 92 | L → Q |

Please Table 23 noting the amino acid differences between CG107126-01 and the invention which are structurally variable and possibly functionally variable:

The presence of identifiable domains in the protein disclosed herein was determined by searches versus domain databases such as Pfam, PROSITE, ProDom, Blocks or Prints and then identified by the Interpro domain accession number. Significant domains are summarized in Table 24.

No Relevant Pfam Hits.

This indicates that the sequence of the invention has properties similar to those of other proteins known to contain this/these domain(s) and similar to the properties of these domains.

Chromosomal Information

The Type IIIa Membrane Protein-like gene disclosed in this invention maps to chromosome 1p32.1-p33. This assignment was made using mapping information associated with genomic clones, public genes and ESTs sharing sequence identity with the disclosed sequence and CuraGen Corporation's Electronic Northern bioinformatic tool.

Tissue Expression

The Type IIIa Membrane Protein-like gene disclosed in this invention is expressed in at least the following tissues: Pancreas, Lymph node, Synovium/Synovial membrane, Brain, Uterus, Cervix, Lung, Skin, Eye, Stomach, Liver, Spleen. Expression information was derived from the tissue sources of the sequences that were included in the derivation of the sequence of CuraGen Acc. No. CG107126-01.

Cellular Localization and Sorting

The PSORT, SignalP and hydropathy profile for the Type IIIa Membrane Protein-like protein are shown in FIG. 5. The results predict that this sequence has a signal peptide and is likely to be localized at the plasma membrane with a certainty of 0.6000. The signal peptide is predicted by SignalP to be cleaved at amino acid none given by Psort.

Functional Variants and Homologs

The novel nucleic acid of the invention encoding a Type IIIa Membrane Protein-like protein includes the nucleic acid whose sequence is provided in Table 17, or a fragment thereof. The invention also includes a mutant or variant nucleic acid any of whose bases may be changed from the corresponding base shown Table 17 while still encoding a protein that maintains its Type IIIa Membrane Protein-like activities and physiological functions, or a fragment of such a nucleic acid. The invention further includes nucleic acids whose sequences are complementary to the sequence of CuraGen Acc. No. CG107126-01, including nucleic acid fragments that are complementary to any of the nucleic acids just described. The invention additionally includes nucleic acids or nucleic acid fragments, or complements thereto, whose structures include chemical modifications. Such modifications include, by way of non-limiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 2% of the bases may be so changed.

The novel protein of the invention includes the Type IIIa Membrane Protein-like protein whose sequence is provided in Table 18. The invention also includes a mutant or variant protein any of whose residues may be changed from the corresponding residue shown Table 18, while still encoding a protein that maintains its Type IIIa Membrane Protein-like activities and physiological functions, or a functional fragment thereof. In the mutant or variant protein, up to about 1% of the amino acid residues may be so changed.

Chimeric and Fusion Proteins

The present invention includes chimeric or fusion proteins of the Type IIIa Membrane Protein-like protein, in which the Type IIIa Membrane Protein-like protein of the present invention is joined to a second polypeptide or protein that is not substantially homologous to the present novel protein. The second polypeptide can be fused to either the amino-terminus or carboxyl-terminus of the present CG107126-01 polypeptide. In certain embodiments a third nonhomologous polypeptide or protein may also be fused to the novel Type IIIa Membrane Protein-like protein such that the second nonhomologous polypeptide or protein is joined at the amino terminus, and the third nonhomologous polypeptide or protein is joined at the carboxyl terminus, of the CG107126-01 polypeptide. Examples of nonhomologous sequences that may be incorporated as either a second or third polypeptide or protein include glutathione S-transferase, a heterologous signal sequence fused at the amino terminus of the Type IIIa Membrane Protein-like protein, an immunoglobulin sequence or domain, a serum protein or domain thereof (such as a serum albumin), an antigenic epitope, and a specificity motif such as (His)₆.

The invention further includes nucleic acids encoding any of the chimeric or fusion proteins described in the preceding paragraph.

Antibodies

The invention further encompasses antibodies and antibody fragments, such as Fab, (Fab)₂ or single chain FV constructs, that bind immunospecifically to any of the proteins of the invention. Also encompassed within the invention are peptides and polypeptides comprising sequences having high binding affinity for any of the proteins of the invention, including such peptides and polypeptides that are fused to any carrier particle (or biologically expressed on the surface of a carrier) such as a bacteriophage particle.

Methods of Use for the Compositions of the Invention

The protein similarity information, expression pattern, cellular localization, and map location for the protein and nucleic acid disclosed herein suggest that this Type IIIa Membrane Protein-like protein may have important structural and/or physiological functions characteristic of the Type IIIa Membrane Protein family. Therefore, the nucleic acids and proteins of the invention are useful in potential diagnostic and therapeutic applications and as a research tool. These include serving as a specific or selective nucleic acid or protein diagnostic and/or prognostic marker, wherein the presence or amount of the nucleic acid or the protein are to be assessed. These also include potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), (v) an agent promoting tissue regeneration in vitro and in vivo, and (vi) a biological defense weapon.

The nucleic acids and proteins of the invention have applications in the diagnosis and/or treatment of various diseases and disorders. For example, the compositions of the present invention will have efficacy for the treatment of patients suffering from: Anterior segment mesenchymal dysgenesis; Basal cell carcinoma; Breast cancer, invasive intraductal; C8 deficiency, type I; C8 deficiency, type II; CPT deficiency, hepatic, type II; Ceroid lipofuscinosis, neuronal, variant juvenile type, with granular osmiophilic deposits; Colon adenocarcinoma; Leukemia-1, T-cell acute lymphocytic; Lymphoma, non-Hodgkin; Medulloblastoma; Myopathy due to CPT II deficiency; Asthma, susceptibility to; Epiphyseal dysplasia, multiple, type 2; Intervertebral disc disease; Von Hippel-Lindau (VHL) syndrome, Alzheimer's disease, Stroke, Tuberous sclerosis, hypercalceimia, Parkinson's disease, Huntington's disease, Cerebral palsy, Epilepsy, Lesch-Nyhan syndrome, Multiple sclerosis, Ataxia-telangiectasia, Leukodystrophies, Behavioral disorders, Addiction, Anxiety, Pain, Neuroprotection; diabetes, obesity, cancer as well as other diseases, disorders and conditions.

These materials are further useful in the generation of antibodies that bind immunospecifically to the novel substances of the invention for use in diagnostic and/or therapeutic methods.

TABLE 16

Nucleotide sequence encoding the Type IIIa
Membrane Protein-like protein, CG107126-01.

| | | |
|---|---|---|
| >CG107126_01 | | |
| GACAACCCTGCAATTTTCTTCCTCATAATTGGGAGAAGACTCACTGGCCGAATGGCAGC | 60 | (SEQ ID NO:84) |
| AGTAGATGACTTCCAATTTGAAGAATTTGGCAATGCAGCCACTTCTCTGACAGCAAACCC | 120 | |
| AGATGCCACCACAGTAAACATTGAGGATCCTGGTGAAACCCCAAAACATCAGCCAGGATC | 180 | |
| CCCAAGAGGCTCAGGAAGAGAAGAAGATGATGAGTTACTCGGAAATGATGACTCTGACAA | 240 | |
| AACTGAGTTACTTGCTGGACAGAAGAAAAGCTCCCCCTTCTGGACATTTGAATACTACCA | 300 | |
| AACATTCTTTGATGTGGACACCTACCAGGTCTTTGACAGAATTAAAGGATCTCTTTTGCC | 360 | |
| AATACCCGCGAAAAACTTTGTGAGGTTATATATCCGCAGCAATCCAGATCTCTATGGCCC | 420 | |
| CTTTTGGATATGTGCCACGTTGGTCTTTGCCATAGCAATTAGTGGGAATCTTTCCAACTT | 480 | |
| CTTGATCCATCTGGGAGAGAAGACGTACCATTATGTGCCCGAATTCCGAAAAGTGTCCAT | 540 | |
| AGCAGCTACCATCATCTATGCCTATGCCTGGCTGGTTCCTCTTGCACTCTGGCGTTTCCT | 600 | |
| CATGTGCAGAAACAGCAAAGTTATCAACATCGTCTCCTATTCATTTCTGGAGATTGTGTG | 660 | |
| TGTCTATGCATATTCCCTCTTCATTTATATCCCCACCGCAATACTGTGGATTATCCCCCA | 720 | |
| GAAAGCTGTTCGTTGCATTCTAGTCATGATTGCCCTGGGCATCTCAGGATCTCTCTTGGC | 780 | |
| AATGACATTTTGGCCAGCTGTTCGTGAGGATAACCGACGCGTTGCATTGGCCACAATTGT | 840 | |
| GACAATTGTGTTGCTCCATATGCTGCTTTCTGTGGGCTGCTTGGCATACTTTTTTGATGC | 900 | |
| ACCAGAGATGGACCATCTCCCAACAACTACACCTACTCCAAACCAAACAGTTGCTCCAGC | 960 | |
| CAAGTCCAGCTAATGAGGAAAGACTCACTTGAGATACCCTCTCCTTGCTGAAGTTTTTCT | 1020 | |
| TGACTTCTCCAGTTCTCTTTTGTTTTTTGGAGCATGGTTCTTTGGGAAGTGGCATCCACT | 1080 | |
| GCAGGAAAGCAGAATGAGCAGAGCCAGCAGAACTGATGGAGTGGCACAAATTCCCAGTGT | 1140 | |
| CTGGATGGTGCCACACTGGCGCCTAATCACCCGTTTAACAAGCAGAAATTAAATGTTGCT | 1200 | |
| CAGCACATGTGTCTTTCAGCTCTTCCTTTTCACCCATGCATGATCATTGCGAGCATCCGC | 1260 | |
| TGATTGGACTCAAATGCCGGGGAATAGGTTAGGCATCCTCACTGCCGTCCCTTTGCCACC | 1320 | |
| ACAGTCAAATGACATGCTTCACTGTGGTACCTTAATACCTGAAATAGAACCATGGAAAAT | 1380 | |
| TCTGATGTCCTCTCTCTGAATTATGTACAGACTACCTGGGGGATCCTCTTCTCTCCAAAT | 1440 | |
| GTTAGCCATCCTGAAGTAGCCGAACAGTAGAAACTTTGGTGGGGATTAACCGGGAGCTTG | 1500 | |
| AAAATTTGTCTTTGGTAACCTGATACTGGACAGCTGAACTGAATGGCTGCAAAATAAATA | 1560 | |
| CCTCACATG 1569 | | |

TABLE 17
Protein sequence encoded by the nucleotide sequence for CG107126-01.

| | | |
|---|---|---|
| >CG107126_01 | | |
| MAAVDDLQFEEFGNAATSLTANPDATTVNIEDPGETPKHQPGSPRGSGREEDDELLGNDD | 60 | (SEQ ID NO:85) |
| SDKTELLAGQKKSSPFWTFEYYQTFFDVDTYQVFDRIKGSLLPIPGKNFVRLYIRSNPDL | 120 | |
| YGPFWICATLVFAIAISGNLSNFLIHLGEKTYHYVPEFRKVSIAATIIYAYAWLVPLALW | 180 | |
| GFLMWRNSKVNNIVSYSFLEIVCVYGYSLFIYIPTAILWIIPQKAVRWILVMIALCISGS | 240 | |
| LLAMTFWPAVREDNRRVALATIVTIVLLHMLLSVGCLAYFFDAPENDHLPTTTATPNQTV | 300 | |
| AAAKSS 306 | | |

TABLE 18
BLASTN search using CuraGen Ace. No. CG107126-01.

>gb:GENBANK-1D:AX050254|acc:AX050254.1 Sequence 7 from Patent WO0070046-Homo sapiens,
1597 bp. Length = 1597
Plus Strand HSPs:
Score = 4856 (728.6 bits), Expect = 0.0, Sum P(2) = 0.0
Identities = 980/991 (98%), Positives = 980/991 (98%), Strand = Plus/Plus

| | | | | |
|---|---|---|---|---|
| Query: | 1 | GAGAACCCTGCAAATTTTCTTCCTCATAATTGGGAGAAGACTCACTGGCCGAATGGCAGC | 60 | (SEQ ID NO:86) |
| | | \|\|\|\| \|\|\|\|\|\|\|\|\|\|\|\|\| \|\|\|\|\|\|\|\|\|\|\|\|\|\|\|\|\|\|\|\|\|\|\|\|\|\|\|\|\|\|\|\|\|\|\|\|\|\|\|\|\| | | |
| Sbjct: | 70 | GAGACCCCTGCAAATTTTTTTCCTCATAATTGGCAGAAGACTCACTGGCCGAATGGCAGC | 129 | (SEQ ID NO:87) |
| Query: | 61 | AGTAGATGACTTGCAATTTGAAGAATTTGGCAATGCAGCCACTTCTCTGACAGCAAACCC | 120 | |
| | | \|\|\|\|\|\|\|\|\| \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| Sbjct: | 130 | AGTAGATGATTTGCAATTTGAAGAATTTGGCAATGCAGCCACTTCTCTGACAGCAAACCC | 189 | |
| Query: | 121 | AGATGCCACCACAGTAAACATTCAGCATCCTGGTGAAACCCCAAAACATCAGCCAGGATC | 180 | |
| | | \|\|\|\|\|\|\|\|\|\|\|\|\|\|\|\|\|\|\|\|\|\|\|\|\|\| \|\|\|\|\|\|\|\|\|\|\|\|\|\|\|\|\|\|\|\|\|\|\|\|\|\|\|\|\|\| \|\| | | |
| Sbjct: | 190 | AGATGCCACCACAGTAAACATTGAGGTTCCTGGTGAAACCCCAAAACATCAGCCACGTTC | 249 | |
| Query: | 181 | CCCAACAGGCTCAGGAAGAGAAGAAGATGATGAGTTACTGGGAAATGATGACTCTGACAA | 240 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| Sbjct: | 250 | CCCAAGACGCTCAGGAAGAGAAGAAGATCATGAGTTACTGGGAAATGATGACTCTGACAA | 309 | |
| Query: | 241 | AACTGAGTTACTTCCTGGACAGAAGAAAAGCTCCCCCTTCTGGACATTTGAATACTACCA | 300 | |
| | | \|\|\|\|\|\|\|\|\|\|\|\|\|\|\|\|\|\|\|\|\|\|\|\|\|\|\|\|\|\|\|\|\|\|\|\|\|\|\| \|\|\|\|\|\|\|\|\|\|\|\|\|\|\|\|\|\|\|\| | | |
| Sbjct: | 310 | AACTGAGTTACTTGCTGGACAGAAGAAAAGCTCCCCCTTTTGGACATTTGAATACTACCA | 369 | |
| Query: | 301 | AACATTCTTTGATGTGGACACCTACCAGGTCTTTGACACAATTAAAGGATCTCTTTTGCC | 4360 | |
| | | \|\|\|\|\|\|\|\|\|\|\|\|\|\|\|\|\|\|\|\|\|\|\|\|\| \|\|\|\|\|\|\|\|\|\|\|\|\|\|\|\|\|\|\|\|\|\|\|\|\|\|\|\|\|\|\|\|\|\| | | |
| Sbjct: | 370 | AACATTCTTTGATGTGGACACCTACCTGGTCTTTGACAGAATTAAAGGATCTCTTTTGCC | 429 | |
| Query: | 361 | AATACCCGGGAAAAACTTTGTGAGGTTATATATCCGCAGCAATCCAGATCTCTATGGCCC | 420 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| Sbjct: | 430 | AATACCCCGGAAAAACTTTGTGAGGTTATATATCCGCAGCAATCCAGATCTCTATGGCCC | 489 | |
| Query: | 421 | CTTTTGGATATGTGCCACGTTGGTCTTTGCCATAGCAATTAGTGGGAATCTTTCCAACTT | 480 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| Sbjct: | 490 | CTTTTGGATATGTGCCACGTTGGTCTTTGCCATAGCAATTAGTCGGAATCTTTCCAACTT | 549 | |
| Query: | 481 | CTTGATCCATCTGGGAGAGAAGACGTACCATTATGTGCCCGAATTCCGAAAAGTGTCCAT | 540 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| Sbjct: | 550 | CTTGATCCATCTGGGAGAGAAGACCTACCATTATGTGCCCGAATTCCGAAAAGTGTCCAT | 609 | |
| Query: | 541 | AGCAGCTACCATCATCTATGCCTATGCCTGGCTGGTTCCTCTTCCACTCTGGGGTTTCCT | 600 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| Sbjct: | 610 | AGCAGCTACCATCATCTATGCCTATGCCTGGCTGGTTCCTCTTGCACTCTGGGGTTTCCT | 669 | |
| Query: | 601 | CATGTGGAGAAACAGCAAAGTTATGAACATCGTCTCCTATTCATTTCTGGAGATTGTGIG | 660 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| Sbjct: | 670 | CATGTCGAGAAACAGCAAACTTATGAACATCGTCTCCTATTCATTTCTGCAGATTGTGTG | 729 | |
| Query: | 661 | TGTCTATCGATATTCCCTCTTCATTTATATCCCCACCGCAATACTGTGGATTATCCCCCA | 720 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| Sbjct: | 730 | TGTCTATGGATATTCCCTCTTCATTTATATCCCCACCGCAATACTGTGGATTATCCCCCA | 789 | |
| Query: | 721 | GAAAGCTGTTCGTTGGATTCTAGTCATCATTCCCCTGGGCATCTCAGGATCTCTCTTGGC | 780 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| Sbjct: | 790 | GAAAGCTGTTCGTTGGATTCTAGTCATCATTCCCCTGGGCATCTCAGGATCTCTCTTGGC | 849 | |
| Query: | 781 | AATGACATTTTGGCCAGCTGTTCGTGAGCATAACCGACGCGTTGCATTGGCCACAATTGT | 840 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| Sbjct: | 850 | AATGACATTTTGGCCAGCTGTTCCTGAGCATAACCGACGCGTTCCATTGGCCACAATTGT | 909 | |
| Query: | 841 | GACAATTGTGTTGCTCCATATGCTGCTTTCTGTGGGCTGCTTGGCATACTTTTTTGATGC | 900 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| Sbjct: | 910 | GACAATTGTGTTGCTCCATATGCTGCTTTCTCTGGGCTCCTTCGCATACTTTTTTGATGC | 969 | |
| Query: | 901 | ACCAGAGATGGACCATCTCCCAACAACTACAGCTACTCCAAACCAAACAGTTGCTGCAGC | 960 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| Sbjct: | 970 | ACCAGAGATGGACCATCTCCCAACAACTACAGCTACTCCAAACCAAACAGTTGCTGCAGC | 1029 | |
| Query: | 961 | CAAGTCCAGCTAATGAGGAAAGACTCACTTG 991 | | |
| | | \|\|\|\|\|\|\|\|\|\|\|\|\|\|\|\|\|\|\|\|\| \|\|\| \|\|\| | | |
| Sbjct: | 1030 | CAAGTCCACCTAATGAGGAAATTCTCTTTTG 1060 | | |

Score = 2686 (403.0 bits), Expect = 0.0, Sum P(2) = 0.0
Identities = 538/539 (99%), Positives = 538/539 (99%), Strand = Plus/Plus

| | | | |
|---|---|---|---|
| Query: | 1031 | AGTTCTCTTTTGTTTTTTGGAGCATGGTTCTTTGGGAAGTGGCATCCACTGCAGGAAAGC | 1090 |
| | | \| \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: | 1049 | AATTCTCTTTTGTTTTTTGGAGCATGGTTCTTTGGGAAGTGGCATCCACTGCAGGAAAGC | 1108 |
| Query: | 1091 | AGAATGAGCAGAGCCAGCAGAACTGATGGACTGGCACAAATTCCCAGTGTCTGGATGGTG | 1150 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: | 1109 | AGAATGAGCAGAGCCAGCAGAACTGATGGAGTGGCACAAATTCCCAGTGTCTGGATGGTG | 1168 |
| Query: | 1151 | CCACACTGGCGCCTAATCACCCGTTTAACAAGCAGAAATTAAATGTTGCTCAGCACATGT | 1210 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: | 1169 | CCACACTGGCGCCTAATCACCCGTrTAACAAGCACAAATTAAATGTTGCTCAGCACATGT | 1228 |
| Query: | 1211 | GTCTTTCACCTCTTCCTTTTCACCCATGGATGATCATTGCGAGCATGCGCTGATTGGACT | 1270 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: | 1229 | GTCTTTCAGCTCTTCCTTTTCACCCATCGATGATCATTGCGAGCATGCGCTGATTGGACT | 1288 |
| Query: | 1271 | GAAATGCCGCGGAATAGGTTAGGCATGCTCAGTGCCGTCCCTTTGCCACCACAGTCAAAT | 1330 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: | 1289 | GAAATGCCGGGGAATAGGTTAGGCATGCTCAGTGCCGTCCCTTTGCCACCACAGTCAAAT | 1348 |
| Query: | 1331 | GACATGCTTCACTGTGGTACCTTAATACCTGAAATAGAACCATGGAAAATTCTGATGTCC | 1390 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: | 1349 | GACATGCTTCACTGTGGTACCTTAATACCTGAAATAGAACCATGGAAAATTCTGATGTCC | 1408 |
| Query: | 1391 | TCTCTCTGAATTATGTACAGACTACCTGGGGGATCCTCTTCTCTCCAAATGTTAGCCATC | 1450 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: | 1409 | TCTCTCTGAATTATGTACAGACTACCTGGGGGATCCTCTTCTCTCCAAATGTTAGCCATC | 1468 |
| Query: | 1451 | CTGAAGTACCCGAACAGTAGAAACTTTGGTGGGGATTAACCGGGAGCTTGAAAATTTGTC | 1510 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: | 1469 | CTGAAGTAGCCGAACAGTAGAAACTTTGGTGGGGATTAACCGGGAGCTTGAAAATTTGTC | 1528 |
| Query: | 1511 | TTTCGTAACCTGATACTGGACAGCTGAACTCAATGGCTGCAAAATAAATACCTCACATG | 1569 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: | 1529 | TTTCGTAACCTGATACTGGACAGCTGAACTGAATGGCTGCAAAATAAATACCTCACATG | 1587 |

(Query: SEQ ID NO:88; Subject: SEQ ID NO:89)

TABLE 19
BLASTP search using the protein of CuraGen ACC. No. CG107126-01.

>ptnr:REMTREMBL_ACC:CAC21787 Sequence 7 from Patent WO0070046

Homo sapiens
(Human), 306 aa.
Length = 306
Score = 1592 (560.4 bits), Expect = 2.7e−163, P = 2.7e−163
Identities = 304/306 (99%), Positives = 304/306 (99%)

| | | | | |
|---|---|---|---|---|
| Query: | 1 | MAAVDDLQFEEFGNAATSLTANPDATTVNIEDPGETPKHQPCSPRGSGREEDDELLGNDD | 60 | (SEQ ID NO:85) |
| | | MAAVDDLQFEEFGNAATSLTANPDATTVNIE PGETPKHQPGSPRGSGREEDDELLGNDD | | (SEQ ID NO:91) |
| Sbjct: | 1 | MAAVDDLQFEEFGNAATSLTANPDATTVNIEVPGETPKHQPGSPRGSGREEDDELLGNDD | 60 | (SEQ ID NO:90) |
| Query: | 61 | SDKTELLAGQKKSSPFWTFEYYQTFFDVDTYQVFDRIKGSLLPIPGKNFVRLYIRSNPDL | 120 | |
| | | SDKTELLAGQKKSSPFWTFEYYQTFFDVDTY VFDRIKGSLLPIPGKNFVRLYIRSNPDL | | |
| Sbjct: | 61 | SDRTELLAGQKKSSPFWTFEYYQTFFDVDTYLVFDRIKGSLLPIPGKNFVRLYIRSNPDL | 120 | |
| Query: | 121 | YGPFWICATLVFAIAISGNLSNFLIELGEKTYHYVPEFRKVSIAATIIYAYAWLVPLALW | 180 | |
| | | YGPFWICATLVFAIAISGNLSNFLIHLGEKTYHYVPEFRKVSIAATIIYAYAWLVPLALW | | |
| Sbjct: | 121 | YGPFWICATLVFAIAISGNLSNFLIHLGEKTYHYVPEFRKVSIAATIIYAYAWLVPLALW | 180 | |
| Query: | 181 | GFLMWRNSKVNNIVSYSFLEIVCVYGYSLFIYIPTAILWIIPQKAVRWILVMIALGISGS | 240 | |
| | | GFLMWRNSKVMNIVSYSFLEIVCVYGYSLFIYIPTAILWIIPQKAVRWILVMIALGISGS | | |
| Sbjct: | 181 | GFLNWRNSKVMNIVSYSFLEIVCVYGYSLPIYIPTAILWIIPQKAVRWILVMTAIGISGS | 240 | |
| Query: | 241 | LLAMTFWPAVREDNRRVALATIVTIVLLHNLLSVGCLAYFFDAPEMDHLPTTTATPNQTV | 300 | |
| | | LLAMTFWPAVREDNRRVALATIVTIVLLHMLLSVGCLAYFFDAPEMDHLPTTTATPNQTV | | |
| Sbjct: | 241 | LLAMTFWPAVREDNRRVALATIVTIVLLHMLLSVGCLAYFFDAPENDHLPTTTATPNQTV | 300 | |
| Query: | 301 | AAAKSS 306 | | |
| | | AAAKSS | | |
| Sbjct: | 301 | AAAKSS 306 | | |

TABLE 20
BLASTN search using the protein of CuraGen ACe. No. CG107126-01.

>s3aq:171722308, 2894 bp.
Length = 2894
Plus Strand HSPs:
Score = 4919 (738.0 bits), Expect = 0.0, Sum P(2) = 0.0
Identities = 987/991 (99%), Positives = 987/991 (99%), Strand = Plus/Plus

| | | | | |
|---|---|---|---|---|
| Query: | 1 | GAGAACCCTGCAATTTTCTTCCTCATAATTGGGAGAAGACTCACTGGCCGAATGGCAGC | 60 | (SEQ ID NO:86) |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| Sbjct: | 1341 | GAGAACCCTGCAATTTTCTTCCTCATAATTGGGAGAAGACTCACTCGCCGAATGGCAGC | 1400 | (SEQ ID NO:92) |
| Query: | 61 | AGTAGATGACTTGCAATTTGAAGAATTTGGCAATGCAGCCACTTCTCTGACAGCAAACCC | 120 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| Sbjct: | 1401 | AGTAGATGACTTGCAATTTGAAGAATTTGGCAATGCAGCCACTTCTCTGACACCAAACCC | 1460 | |
| Query: | 121 | AGATGCCACCACAGTAAACATTGAGGATCCTGGTGAAACCCCAAAACATCAGCCAGGATC | 180 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| Sbjct: | 1461 | AGATGCCACCACAGTAAACATTGAGGATCCTGGTGAAACCCCAAAACATCAGCCAGGATC | 1520 | |
| Query: | 181 | CCCAAGAGGCTCAGGAAGAGAAGAAGATGATGAGTTACTGGGAAATGATGACTCTGACAA | 240 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| Sbjct: | 1521 | CCCAAGAGGCTCAGGAAGAGAAGAAGATGATGAGTTACTGGGAAATGATGACTCTGACAA | 1580 | |
| Query: | 241 | AACTGAGTTACTTGCTGGACAGAAGAAAACCTCCCCCTTCTGCACATTTGAATACTACCA | 300 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| Sbjct: | 1581 | AACTGAGTTACTTGCTGGACAGAAGAAAACCTCCCCCTTCTGGACATTTCAATACTACCA | 1640 | |
| Query: | 301 | AACATTCTTTGATGTCCACACCTACCAGGTCTTTGACAGAATTAAAGCATCTCTTTTCCC | 360 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| Sbjct: | 1641 | AACATTCTTTGATGTGCACACCTACCACGTCTTTGACACAATTAAAGGATCTCTTTTGCC | 1700 | |
| Query: | 361 | AATACCCGCGAAAAACTTTGTCAGGTTATATATCCGCACCAATCCACATCTCTATGCCCC | 420 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| Sbjct: | 1701 | AATACCCGCGAAAAACTTTGTGAGGTTATATATCCCCACCAATCCAGATCTCTATGGCCC | 1760 | |
| Query: | 421 | CTTTTGCATATGTGCCACCTTGCTCTTTCCCATAGCAATTAGTGCGAATCTTTCCAACTT | 480 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| Sbjct: | 1761 | CTTTTCGATATGTGCCACGTTGCTCTTTGCCATACCAATTAGTGGCAATCTTTCCAACTT | 1820 | |
| Query: | 481 | CTTGATCCATCTGGGAGAGAAGACGTACCATTATGTGCCCGAATTCCGAAAACTGTCCAT | 540 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| Sbjct: | 1821 | CTTGATCCATCTCGCAGACAAGACGTACCATTATGTGCCCGAATTCCGAAAAGTGTCCAT | 1880 | |
| Query: | 541 | AGCAGCTACCATCATCTATGCCTATGCCTGGCTGCTTCCTCTTCCACTCTGCGGTTTCCT | 600 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| Sbjct: | 1881 | AGCAGCTACCATCATCTATCCCTATGCCTCCCTGCTTCCTCTTGCACTCTGGGGTTTCCT | 1940 | |
| Query: | 601 | CATCTGCACAAACACCAAAGTTATCAACATCGPCTCCTATTCATTTCTGCAGATTGTGTG | 660 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| Sbjct: | 1941 | CATGTGGAGAAACAGCAAACTTATGAACATCCTCTCCTATTCATTTCTCGAGATTGTGTG | 2000 | |
| Query: | 661 | TGTCTATCGATATTCCCTCTTCATTTATATCCCCACCGCAATACTGTCGATTATCCCCCA | 720 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| Sbjct: | 2001 | TGTCTATCCATATTCCCTCTTCATTTATATCCCCACCGCAATACTGTGGATTATCCCCCA | 2060 | |
| Query: | 721 | CAAAGCTCTTCGTTGGATTCTAGTCATGATTGCCCTGGGCATCTCAGGATCTCTCTTGCC | 780 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| Sbjct: | 2061 | GAAACCTCTTCCTTGGATTCTAGTCATGATTGCCCTGGCCATCTCACCATCTCTCTTCGC | 2120 | |
| Query: | 781 | AATCACATTTTGGCCAGCTGTTCGTGACCATAACCGACCCGTTGCATTGGCCACAATTGT | 840 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| Sbjct: | 2121 | AATGACATTTTGGCCACCTGTTCCTGAGGATAACCCACCCGTTGCATTGGCCACAATTGT | 2180 | |
| Query: | 841 | GACAATTGTCTTGCTCCATATGCTGCTTTCTGTCGGCTCCTTGGCATACTTTTTTGATCC | 900 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| Sbjct: | 2181 | GACAATTCTGTTGCTCCATATGCTGCTTTCTCTGGCCTGCTTCCCATACTTTTTTGATGC | 2240 | |
| Query: | 901 | ACCAGACATCGACCATCTCCCAACAACTACAGCTACTCCAAACCAAACAGTTCCTCCACC | 960 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| Sbjct: | 2241 | ACCAGACATGGACCATCTCCCAACAACTACAGCTACTCCAAACCAAACAGTTCCTCCACC | 2300 | |
| Query: | 961 | CAAGTCCACCTAATCACGAAAGACTCACTTG 991 | | |
| | | \|\|\|\|\|\|\|\|\|\|\|\|\|\|\|\|\|\|\|\|\| \|\|\| \|\|\| | | |
| Sbjct: | 2301 | CAACTCCACCTAATGAGGAAATTCTCTTTTC 2331 | | |

Score = 2686 (403.0 bits), Expect = 0.0, Sum P(2) = 0.0
Identities = 538/539 (99%), Positives = 538/539 (99%), Strand Plus/Plus

(Query: SEQ ID NO:88; Subject: SEQ ID NO:93)

| | | | |
|---|---|---|---|
| Query: | 1031 | AGTTCTCTTTTGTTTTTTGGAGCATGGTTCTTTGGGAAGTGGCATCCACTGCAGGAAAGC | 1090 |
| | | \| \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: | 2320 | AATTCTCTTTTGTTTTTTGGAGCATGGTTCTTTCGGAAGTGGCATCCACTGCAGGAAAGC | 2379 |
| Query: | 1091 | AGAATGAGCAGACCCAGCAGAACTGATGGAGTGGCACAAATTCCCAGTGTCTGCATGGTG | 1150 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: | 2380 | AGAATGAGCAGACCCAGCAGAACTGATGGAGTGGCACAAATTCCCAGTGTCTGGATGGTG | 2439 |
| Query: | 1151 | CCACACTGGCCCCTAATCACCCGTTTAACAAGCAGAAATTAAATGTTGCTCAQCACATGT | 1210 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: | 2440 | CCACACTGGCGCCTAATCACCCGTTTAACAACCAGAAATTAAATGTTGCTCACCACATGT | 2499 |
| Query: | 1211 | GTCTTTCAGCTCTTCCTTTTCACCCATGGATGATCATTGCGAGCATGCGCTGATTGCACT | 1270 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: | 2500 | GTCTTTCAGCTCTTCCTTTTCACCCATGGATGATCATTGCGAGCATGCGCTGATTGGACT | 2559 |
| Query: | 1271 | GAAATCCCGGGGAATAGGTTACGCATGCTCAGTGCCCTCCCTTTGCCACCACAGTCAAAT | 1330 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: | 2560 | GAAATGCCGGGGAATAGGTTAGGCATGCTCAGTGCCCTCCCTTTGCCACCACAGTCAAAT | 2619 |
| Query: | 1331 | GACATGCTTCACTGTGGTACCTTAATACCTGAAATAGAACCATGGAAAATTCTCATGTCC | 1390 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: | 2620 | GACATCCTTCACTGTGGTACCTTAATACCTGAAATAGAACCATGGAAAATPCTCATGTCC | 2679 |
| Query: | 1391 | TCTCTCTCAATTATGTACAGACTACCTGGCGGATCCTCTTCTCTCCAAATGTTAGCCATC | 1450 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: | 2680 | TCTCTCTGAATTATGTACACACTACCTGGGGGATCCTCTTCTCTCCAAATGTTAGCCATC | 2739 |
| Query: | 1451 | CTGAAGTAGCCGAACAGTAGAAACTTTGGTGGGGATTAACCGGGAGCTTGAAAATTTGTC | 1510 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: | 2740 | CTGAAGTAGCCCAACAGTAGAAACTTTGGTGGGGATTAACCGGCAGCTTGAAAATTTGTC | 2799 |
| Query: | 1511 | TTTGGTAACCTGATACTGGACAGCTGAACTGAATGGCTGCAAAATAAATACCTCACATG | 1569 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: | 2800 | TTTGGTAACCTGATACTGGACAGCTGAACTGAATGGCTGCAAAATAAATACCTCACATG | 2858 |

Minus Strand HSPs:
Score = 6019 (903.1 bits), Expect = 4.2e−267, P = 4.2e−267
Identities = 1207/1211 (99%), Positives 1207/1211 (99%), Strand = Minus/Plus

| | | | | |
|---|---|---|---|---|
| Query: | 1569 | CATGTGACGTATTTATTTTCCAGCCATTCAGTTCACCTGTCCACTATCAGGTTACCAAAG | 1510 | (SEQ ID NO:94) |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| Sbjct: | 56 | CATGTGAGCTATTTATTTTGCAGCCATTCACTTCAGCTGTCCAGTATCAGGTTACCAAAG | 115 | (SEQ ID NO:95) |
| Query: | 1509 | ACAAATTTTCAAGCTCCCCGTTAATCCCCACCAAAGTTTCTACTGTTCGCCTACTTCAGG | 1450 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| Sbjct: | 116 | ACAAATTTTCAAGCTCCCGGTTAATCCCCACCAAAGTTTCTACTGTTCGGCTACTTCAGG | 175 | |
| Query: | 1449 | ATGGCTAACATTTGGAGAGAAGAGGATCCCCCAGGTAGTCTGTACATAATTCAGAGAGAG | 1390 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| Sbjct: | 176 | ATGGCTAACATTTGGAGAGAAGAGCATCCCCCACGTAGTCTGTACATAATTCAGAGAGAG | 235 | |
| Query: | 1389 | GACATCAGAATTTTCCATGGTTCTATTTCAGGTATTAAGGTACCACAGTGAAGCATGTCA | 1330 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| Sbjct: | 236 | GACATCAGAATTTTCCATGGTTCTATTTCAGCTATTAACGTACCACAGTGAAGCATCTCA | 295 | |
| Query: | 1329 | TTTGACTGTGGTGGCAAAGGGACGGCACTGAGCATGCCTAACCTATTCCCCGGCATTTCA | 1270 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| Sbjct: | 296 | TTTGACTGTGGTGGCAAAGGGACGGCACTGAGCATGCCTAACCTATTCCCCGCCATTTCA | 355 | |
| Query: | 1269 | GTCCAATCAGCGCATGCTCGCAATGATCATCCATGGGTGAAAAGGAAGAGCTGAAAGACA | 1210 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| Sbjct: | 356 | GTCCAATCAGCGCATGCTCGCAATGATCATCCATGGGTGAAAAGGAAGAGCTGAAAGACA | 415 | |
| Query: | 1209 | CATGTCCTGAGCAACATTTAATTTCTGCTTGTTAAACGGGTGATTAGGCGCCAGTGTGCC | 1150 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| Sbjct: | 416 | CATGTGCTGAGCAACATTTAATTTCTGCTTGTTAAACCGGTGATTAGGCGCCAGTGTGGC | 475 | |
| Query: | 1149 | ACCATCCAGACACTGGGAATTTGTGCCACTCCATCAGTTCTCCTGGCTCTGCTCATTCTG | 1090 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| Sbjct: | 476 | ACCATCCAGACACTGCGAATTTGTGCCACTCCATCAGTTCTGCTGGCTCTGCTCATTCTG | 535 | |
| Query: | 1089 | CTTTCCTCCAGTGGATGCCACTTCCCAAAGAACCATGCTCCAAAAAACAAAAGACAACTG | 1030 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| Sbjct: | 536 | CTTTCCTGCAGTGGATGCCACTTCCCAAAGAACCATGCTCCAAAAAACAAAAGAGAACTG | 595 | |
| Query: | 1029 | GAGAAGTCAAGAAAAACTTCAGCAAGCAGAGGCTATCTCAAGTGAGTCTTTCCTCATTAG | 970 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| Sbjct: | 596 | GAGAAGTCAAGAAAAACTTCAGCAAGGAGAGGGTATCTCAAGTGAGTCTTTCCTCATTAG | 655 | |
| Query: | 969 | CTGGACTTGGCTGCAGCAACTGTTTGGTTTGGAGTAGCTCTACTTGTTGGGAGATGGTCC | 910 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| Sbjct: | 656 | CTCCACTTGCCTGCAGCAACTGTTTGGTTTGGAGTAGCTGTAGTTGTTGGGAGATCGTCC | 715 | |
| Query: | 909 | ATCTCTGGTGCATCAAAAAAGTATGCCAAGCAGCCCACAGAAAGCAGCATATGGACCAAC | 850 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| Sbjct: | 716 | ATCTCTGGTGCATCAAAAAAGTATGCCAACCAGCCCACAGAAAGCACCATATGGAGCAAC | 775 | |
| Query: | 849 | ACAATTGTCACAATTGTGGCCAATGCAACGCGTCGGTTATCCTCACCAACAGCTGGCCAA | 790 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| Sbjct: | 776 | ACAATTGTCACAATTGTGGCCAATGCAACGCGTCGCTTATCCTCACGAACAGCTGGCCAA | 835 | |
| Query: | 789 | AATGTCATTGCCAAGAGAGATCCTCACATGCCCACGGCAATCATCACTAGAATCCAACGA | 730 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| Sbjct: | 836 | AATGTCATTGCCAAGAGAGATCCTGAGATGCCCAGGGCAATCATGACTAGAATCCAACGA | 895 | |
| Query: | 729 | ACAGCTTTCTGGGGGATAATCCACAGTATTGCGCTGGGGATATAAATGAAGAGGGAATAT | 670 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| Sbjct: | 896 | ACAGCTTTCTGGGGGATAATCCACAGTATTGCGGTGGGCATATAAATGAAGAGGGAATAT | 955 | |
| Query: | 669 | CCATAGACACACACAATCTCCAGAAATGAATAGGAGACGATGTTCATAACTTTGCTGTTT | 610 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| Sbjct: | 956 | CCATAGACACACACAATCTCCAGAAATGAATAGGAGACGATGTTCATAACTTTGCTGTTT | 1015 | |
| Query: | 609 | CTCCACATGAGGAAACCCCACACTGCAAGAGGAACCAGCCAGGCATAGGCATAGATGATG | 550 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| Sbjct: | 1016 | CTCCACATGACGAAACCCCAGAGTGCAAGAGGAACCACCCAGCCATACGCATACATGATG | 1075 | |
| Query: | 549 | GTAGCTGCTATCGACACTTTTCGGAATTCGGGCACATAATCGTACGTCTTCTCTCCCAGA | 490 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| Sbjct: | 1076 | GTAGCTGCTATCGACACTTTTCGGAATTCGGCCACATAATGGTACGTCTTCTCTCCCAGA | 1135 | |
| Query: | 489 | TGGATCAAGAAGTTGGAAAGATTCCCACTAATTGCTATGGCAAAGACCAACGTGGCACAT | 430 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| Sbjct: | 1136 | TGGATCAAGAACTTGGAAAGATTCCCACTAATTGCTATGGCAAAGACCAACGTGGCACAT | 1195 | |
| Query: | 429 | ATCCAAAAGGGGCCATAGAGATCTGGATTGCTGCGGATATATAACCTCACAAAGTTTTTC | 370 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| Sbjct: | 1196 | ATCCAAAAGGGGCCATAGAGATCTGGATTGCTGCGGATATATAACCTCACAAAGTTTTTT | 1255 | |
| Query: | 369 | CCGGGTATTGG 359 | | |
| | | \| \|\| \|\|\|\|\| | | |
| Sbjct: | 1256 | CGGGCAATTCG 1266 | | |

>s3aq:163994413, 1380 bp.
Length = 1380
Plus Strand HSPs:
Score = 1419 (212.9 bits), Expect = 8.7e−137, Sum P(3) = 8.7e−137
Identities = 287/291 (98%), Positives = 287/291 (98%), Strand = Plus/Plus

(Query: SEQ ID NO:96; Subject: SEQ ID NO:97)

| | | | |
|---|---|---|---|
| Query: | 413 | TATGGCCCCTTTTGGATATGTGCCACGTTGGTCTTTGCCATAGCAATTAGTGGGAATCTT | 472 |
| | | \|\|\|\|\|\|\|\|\|\|\|\|\|\|\|\|\|\|\|\|\|\|\|\|\|\|\|\|\|\|\|\|\|\|\|\|\|\|\|\|\|\| \|\| \|\|\|\| \|\|\|\|\|\|\|\|\| | |
| Sbjct: | 602 | TATGGCCCCTTTTGGATATGTCCCACGTTCGTCTTTGCCATAACATTTAGGGGGAATCTT | 661 |
| Query: | 473 | TCCAACTTCTTGATCCATCTGGGAGACAAGACGTACCATTATGTGCCCGAATTCCCAAAA | 532 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: | 662 | TCCAACTTCTTGATCCATCTGGGAGAGAACACGTACCATTATGTGCCCGAATTCCCAAAA | 721 |
| Query: | 533 | GTGTCCATAGCAGCTACCATCATCTATGCCTATGCCTCGCTGGTTCCTCTTGCACTCTGG | 592 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: | 722 | GTGTCCATAGCAGCTACCATCATCTATGCCTATGCCTGGCTGGTTCCTCTTGCACTCTGC | 781 |
| Query: | 593 | GGTTTCCTCATGTGGAGAAACAGCAAAGTTATGAACATCCTCTCCTATTCATTTCTGGAG | 652 |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: | 782 | GGTTTCCTCATCTGGAGAAACAGCAAAGTTATGAACATCGTCTCCTATTCATTTCTGGAG | 841 |
| Query: | 653 | ATTGTGTGTGTCTATGGATATTCCCTCTTCATTTATATCCCCACCGCAATA 703 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| \|\| | |
| Sbjct: | 842 | ATTGTGTGTGTCTATGCATATTCCCTCTTCATTTATATCCCCACCGCAGTA 892 | |

Score = 1371 (205.7 bits), Expect = 1.2e−134, Sum P(3) = 1.2e−134
Identities = 329/377 (87%), Positives = 329/377 (87%), Strand = Plus/Plus

| | | | | |
|---|---|---|---|---|
| Query: | 220 | GGGAAATGATG-ACTCTGACAAA-ACTGAG-TTACTTGCTCGACAGAAGAAAAGCTCCCC | 276 | (SEQ ID NO:98) |
| | | GCGAAA A G A C GAC A ACT TTACTTGCTCCACAGAAGAAAAGCTCCCC | | (SEQ ID NO:99) |
| Sbjct: | 308 | CGCAAAC-AGGCAAACAGACCATTACTTTCCTTACTTGCTGGACACAAGAAAAGCTCCCC | 366 | (SEQ ID NO:100) |
| Query: | 277 | CTTCTGGACATTTGAATACTACCAAACATTCTTTGATGTGGACACCTACCAGGTCTTTGA | 336 | |
| | | CTTCTCGACATTTGAATACTACCAAACATTCTTTGATGTGGACACCTACCAGGTCTTTGA | | |
| Sbjct: | 367 | CTTCTGGACATTTGAATACTACCAAACATTCTTTGATGTGGACACCTACCAGGTCTTTGA | 426 | |
| Query: | 337 | CAGAATTAAAGGATCTCTTTTGCCAATACCCGGGAAAAACTTTGTGAGGTTATATATCCG | 396 | |
| | | CAGAATTAAAGGATCTCTTTTGCCAATACCCGGGAAAAACTTTGTGAGGTTATATATCCG | | |
| Sbjct: | 427 | CAGAATTAAAGGATCTCTTTTGCCAATACCCGGGAAAAACTTTGTGAGGTTATATATCCG | 486 | |
| Query: | 397 | CAGCAATCCAGATCTCTATGGCCCCTTTTGGATATGTGCCACGTTGGTCTTTGCCATAGC | 456 | |
| | | CAGCAATCCAGATCTCTATGGCCCCTTTTGGATATGTGCCACGTTGGTCTTTGCCATAGC | | |
| Sbjct: | 487 | CAGCAATCCAGATCTCTATGGCCCCTTTTGGATATGTGCCACGTTGGTCTTTGCCATAGC | 546 | |
| Query: | 457 | AATTAGTGGGAATCTTTCCAACTTCTTGATCCATCTGGGAGAGAAGACGTACCATTATGT | 516 | |
| | | AATTAGTGGGAATCTTTCCAACTTCTTGATCCATCTGGGAGAGAAGACGTACCATTATG | | |
| Sbjct: | 547 | AATTAGTGGGAATCTTTCCAACTTCTTGATCCATCTGGGAGAGAAGACGTACCATTATG- | 605 | |
| Query: | 517 | GCCCGAATTCCGAAAAGTGTC-CATAGCAGCTA-CCAT--CATCTATGCCTATGCCTGGC | 572 | |
| | | GCCC TT GA A GTG C C T G T CCAT CAT TA G A C T C | | |
| Sbjct: | 606 | GCCCCTTTTG-GATATGTGCCACGTTGGTCTTTGCCATAACATTTAGCGGGAATCTTTCC | 664 | |
| Query: | 573 | TCGTTCCTCTTGCACTCTGGG 593 | | |
| | | TTC T T CA TCTGGG | | |
| Sbjct: | 665 | AACTTCTTGATCCA-TCTGGG 684 | | |

Score = 1245 (186.8 bits), Expect = 8.7e−137, Sum P(3) = 8.7e−137
Identities = 265/273 (97%), Positives = 265/273 (97%), Strand Plus/Plus

| | | | | |
|---|---|---|---|---|
| Query: | 1 | GAGAACCCTGCAAATTTTCTT-CCTCATAATTGGGAGAAGACTCACTGGCCGAATGGCAG | 59 | (SEQ ID NO:101) |
| | | GAGAACCCTGCAAATTTTCTT CCTCATAATTGGGAGAAGACTCACTGGCCGAATCGCAG | | (SEQ ID NO:102) |
| Sbjct: | 1 | GAGAACCCTGCAAATTTTCTTTCCTCATAATTGGGAGAAGACTCACTGGCCGAATGGCAG | 60 | (SEQ ID NO:103) |
| Query: | 60 | CAGTAGATGACTTGCAATTTGAAGAATTTCGCAATGCAGCCACTTCTCTGACAGCAAACC | 119 | |
| | | CAGTAGATGACTTGCAATTTGAAGAATTTGGCAATGCAGCCACTTCTCTCACAGCAAACC | | |
| Sbjct: | 61 | CAGTAGATGACTTGCAATTTGAAGAATTTGGCAATGCAGCCACTTCTCTGACAGCAAACC | 120 | |
| Query: | 120 | CAGATGCCACCACAGTAAACATTGAGGATCCTGGTGAAACCCCAAAACATCAGCCAGGAT | 179 | |
| | | CAGATGCCACCACAGTAAACATTGAGGATCCTGGTCAAACCCCAAAACATCAGCCAGGAT | | |
| Sbjct: | 121 | CAGATGCCACCACAGTAAACATTGAGGATCCTGGTGAAACCCCAAAACATCAGCCAGCAT | 180 | |
| Query: | 180 | CCCCAAGAGGCTCAGGAAGACAAGAAGATGATGAGTTACTGGGAAATGATGACTCTCACA | 239 | |
| | | CCCCAAGAGGCTCACGAAGAGAAGAAGATGATGAGTTACTGGGAAATGATGACTCTGACA | | |
| Sbjct: | 181 | CCCCAAGAGGCTCAGGAAGAGAAGAAGATGATGAGTTACTGGGAAATGATGACTCTGACA | 240 | |
| Query: | 240 | AAACTGAGTTACT-TG--CTGGACAGAAGAAAAGCTC 273 | | |
| | | AAACTGAG TAC TG C GG CA A GA A GCTC | | |
| Sbjct: | 241 | AAACTGACGTACAGTGTCAGG-CA--ATGACA-GCTC 273 | | |

Score = 561 (84.2 bits), Expect = 8.7e−137, Sum P(3) = 8.7e−137
Identities = 129/143 (90%), Positives = 129/143 (90%), Strand = Plus/Plus

| | | | | |
|---|---|---|---|---|
| Query: | 675 | CCCTCTTCATTTATATCCCCACCGCA-ATACTGTGGATTATCCCCCAGAAAGCTCTTCGT | 733 | (SEQ ID NO:104) |
| | | C CTC TC TTT T TC C CA ATACTGTGGATTATCCCCCAGAAAGCTGTTCGT | | (SEQ ID NO:105) |
| Sbjct: | 1240 | CGCTCCTCCTTT-TCTCTTCTTT-CAGATACTGTGGATTATCCCCCAGAAAGCTCTTCGT | 1297 | (SEQ ID NO:106) |
| Query: | 734 | TGGATTCTAGTCATGATTGCCCTCGCCATCTCAGGATCTCTCTTGGCAATGACATTTTGG | 793 | |
| | | TGCATTCTAGTCATGATTGCCCTGGGCATCTCAGCATCTCTCTTGGCAATGACATTTTGG | | |
| Sbjct: | 1298 | TGGATTCTAGTCATGATTGCCCTGGGCATCTCAGGATCTCTCTTGGCAATGACATTTTGG | 1357 | |
| Query: | 794 | CCAGCTGTTCGTGAGGATAACCGA 817 | | |
| | | C GCTGTTC TGAGGATAACCGA | | |
| Sbjct: | 1358 | CGCGCTGTTC-TGAGGATAACCGA 1380 | | |

>s3aq:163994407 Category D:, 630 bp.
Length = 630
Plus Strand HSPs:
Score = 2530 (379.6 bits), Expect = 1.2e−134, Sum P(2) 1.2e−134
Identities 506/506 (100%), Positives = 506/506 (100%), Strand = Plus/Plus

| | | | | |
|---|---|---|---|---|
| Query: | 28 | AATTGGGAGAAGACTCACTGGCCGAATGCCAGCAGTAGATGACTTGCAATTTGAAGAATT | 87 | (SEQ ID NO:107) |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| Sbjct: | 1 | AATTCGGAGAAGACTCACTGGCCGAATGGCAGCAGTAGATGACTTGCAATTTGAAGAATT | 60 | (SEQ ID NO:108) |
| Query: | 88 | TGGCAATGCACCCACTTCTCTGACAGCAAACCCAGATGCCACCACAGTAAACATTCAGGA | 147 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| Sbjbt: | 61 | TGGCAATGCAGCCACTTCTCTGACAGCAAACCCAGATGCCACCACAGTAAACATTGAGGA | 120 | |
| Query: | 148 | TCCTGGTGAAACCCCAAAACATCAGCCAGGATCCCCAAGAGGCTCAGGAAGACAAGAAGA | 207 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| Sbjct: | 121 | TCCTCCTCAAACCCCAAAACATCACCCAGCATCCCCAAGAGGCTCAGGAAGAGAACAAGA | 180 | |
| Query: | 208 | TGATGAGTTACTGGGAAATGATGACTCTGACAAAACTGAGTTACTTGCTGGACAGAAGAA | 267 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| Sbjct: | 181 | TGATGAGTTACTGGGAAATGATGACTCTGACAAAACTGAGTTACTTGCTGGACAGAAGAA | 240 | |
| Query: | 268 | AAGCTCCCCCTTCTGGACATTTCAATACTACCAAACATTCTTTGATGTGGACACCTACCA | 327 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| Sbjct: | 241 | AAGCTCCCCCTTCTGGACATTTGAATACTACCAAACATTCTTTGATGTGGACACCTACCA | 300 | |
| Query: | 328 | GGTCTTTGACACAATTAAAGGATCTCTTTTGCCAATACCCCGGAAAAACTTTGTGAGGTT | 387 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| Sbjct: | 301 | GGTCTTTGACAGAATTAAAGGATCTCTTTTGCCAATACCCGGCAAAAACTTTGTGAGGTT | 360 | |
| Query: | 388 | ATATATCCGCAGCAATCCAGATCTCTATGGCCCCTTTTGGATATGTCCCACGTTGGTCTT | 447 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| Sbjct: | 361 | ATATATCCGCAGCAATCCAGATCTCTATCGCCCCTTTTGGATATGTCCCACGTTCGTCTT | 420 | |
| Query: | 448 | TGCCATAGCAATTAGTGGGAATCTTTCCAACTTCTTGATCCATCTGGCAGAGAAGACGTA | 507 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| Sbjct: | 421 | TGCCATACCAATTAGTGGGAATCTTTCCAACTTCTTGATCCATCTGGGAGAGAAGACGTA | 480 | |
| Query: | 508 | CCATTATGTCCCCCAATTCCGAAAAG 533 | | |
| | | \|\|\|\|\|\|\|\|\|\|\|\|\|\|\|\|\|\|\|\|\|\|\|\|\|\| | | |
| Sbjct: | 481 | CCATTATGTGCCCCAATTCCGAAAAG 506 | | |

Score = 620 (93.0 bits), Expect = L2e−134, Sum P(2) = 1.2e−134
Identities = 124/124 (100%), Positives = 124/124 (100%), Strand = Plus/Plus

| | | | | |
|---|---|---|---|---|
| Query: | 701 | ATACTGTGGATTATCCCCCAGAAACCTGTTCGTTGGATTCTAGTCATCATTGCCCTGGGC | 760 | (SEQ ID NO:109) |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| Sbjct: | 507 | ATACTGTGGATTATCCCCCAGAAAGCTGTTCGTTGGATTCTAGTCATGATTGCCCTGGGC | 566 | (SEQ ID NO:110) |
| Query: | 761 | ATCTCACGATCTCTCTTGGCAATGACATTTTGGCCAGCTGTTCGTGAGGATAACCGACGC | 820 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| Sbjct: | 567 | ATCTCAGGATCTCTCTTGGCAATGACATTTTGGCCAGCTGTTCGTCAGGATAACCGACGC | 626 | |
| Query: | 821 | GTTG 824 | | |
| | | \|\|\|\| | | |
| Sbjct: | 627 | GTTG 630 | | |

>s3aq:162591670, 577 bp.
Length = 577
Plus Strand HSPs:
Score = 1316 (197.5 bits), Expect = 5.5e−54, P = 5.5e−54
Identities = 266/268 (99%), Positives = 266/268 (99%), Strand Plus/Plus

| | | | | |
|---|---|---|---|---|
| Query: | 1234 | CCATGGATGATCATTGCGAGCATGCGCTCATTGGACTGAAATGCCGGGGAATAGGTTAGG | 1293 | (SEQ ID NO:111) |
| | | C ATGGATGATCATTGCGAGCATGCGCTGATTGGACTGAAATGCCGGGGAATAGGTTAGG | | (SEQ ID NO:112) |
| Sbjct: | 311 | CTATGGATGATCATTGCGAGCATGCGCTGATTGGACTCAAATGCCGGGGAATAGGTTAGG | 370 | (SEQ ID NO:113) |
| Query: | 1294 | CATGCTCAGTGCCGTCCCTTTGCCACCACAGTCAAATGACATGCTTCACTGTGGTACCTT | 1353 | |
| | | CATGCTCAGTGCCGTCCCTTTCCCACCACAGTCAAATGACATGCTTCACTGTCGTACCTT | | |
| Sbjct: | 371 | CATGCTCAGTGCCGTCCCTTTGCCACCACAGTCAAATGACATGCTTCACTGTGGTACCTT | 430 | |
| Query: | 1354 | AATACCTGAAATAGAACCATGGAAAATTCTGATGTCCTCTCTCTGAATTATGTACAGACT | 1413 | |
| | | AATACCTGAAATAGAACCATGGAAAATTCTGATGTCCTCTCTCTGAATTATGTACAGACT | | |
| Sbjct: | 431 | AATACCTGAAATAGAACCATGCAAAATTCTGATGTCCTCTCTCTCAATTATGTACAGACT | 490 | |
| Query: | 1414 | ACCTGGGGGATCCTCTTCTCTCCAAATCTTAGCCATCCTCAAGTACCCGAACAGTAGAAA | 1473 | |
| | | ACCTGGGGGATCCTCTTCTCTCCAAATGTTAGCCATCCTGAAGTAGCCGAACAGTAGAAA | | |
| Sbjct: | 491 | ACCTGGGGGATCCTCTTCVCTCCAAATGTTAGCCATCCTGAAGTAGCCGAACAGTAGAAA | 550 | |
| Query: | 1474 | CTTTGGTGGGGATTAACCGGCAGCTTGA 1501 | | |
| | | CTTTGGTGCGGAT AACCCGGAGCTTGA | | |
| Sbjct: | 551 | CTTTGGTGGGGAT-AACCGGCACCTTGA 577 | | |

[image] TABLE 22
Information for the ClustalW proteins:

| Accno | Common Name | Length |
|---|---|---|
| CG107126_01 | novel Type IIIa Membrane Protein-like protein | 306 |
| AAH09080 | SIMILAR TO HYPOTHETICAL PROTEIN. | 306 |
| CAC21787 | Sequence 7 from Patent WO0070046. | 306 |
| Q9Y548 | DJ167A19.1 (NOVEL PROTEIN). | 306 |

In the alignment shown above, black outlined amino acid residues indicate residues identically conserved between sequences (i.e., residues that may be required to preserve structural or functional properties); amino acid residues with a gray background are similar to one another between sequences, possessing comparable physical and/or chemical properties without altering protein structure or function (e.g. the group L,V, I, and M may be considered similar); and amino acid residues with a white background are neither conserved nor similar between sequences.

[image]

### Example 12

CG51051-03, Novel Netrin-Like Protein.

Netrins are a conserved family of proteins that are involved in guiding growing axons and controlling neuronal cell migration. These functions are mediated by interactions with receptor complexes that can be either attractive or repellent in nature. There are a number of known netrin receptors that include DCC (deleted in colorectal cancer), neogenin and UNC5 related proteins. The DCC and neogenin receptors are involved in axon attraction and the UNC5 related proteins will form repellent receptors. In addition to axon guidance, the netrin-DCC signaling interaction may be involved in neuronal cell death regulation during nervous system development (Livesey, F. J. (1999). Cell Mol. Life Sci. 56(1-2):62-8). Netrin-G1 is a member of the UNC6/netrin family. There are multiple isoforms of netrin-G1, most of which are anchored to the plasma membrane through glycosyl-phosphatidyl-inositol linkages. The netrin-G1 proteins do not show a strong affinity for the known netrin receptors and display a lack of attraction for circumferentially growing axons from the cerebellar plate. The netrin-G1 proteins will most likely have functions in nervous system development that are novel from those of the other netrin proteins (Nakashiba, T., T. Ikeda, S. Nishimura, K. Tashiro, T. Honjo J. G. Culotti and S. Itohara (2000). J. Neurosci. 20(17):6540-50.

The sequence of Acc. No. CG51051-03 was derived by laboratory cloning of cDNA fragments, by in silico prediction of the sequence. cDNA fragments covering either the full length of the DNA sequence, or part of the sequence, or both, were cloned. In silico prediction was based on sequences available in Curagen's proprietary sequence databases or in the public human sequence databases, and provided either the full length DNA sequence, or some portion thereof.

The laboratory cloning was performed using one or more of the methods summarized below:

SeqCalling™ Technology: cDNA was derived from various human samples representing multiple tissue types, normal and diseased states, physiological states, and developmental states from different donors. Samples were obtained as whole tissue, primary cells or tissue cultured primary cells or cell lines. Cells and cell lines may have been treated with biological or chemical agents that regulate gene expression, for example, growth factors, chemokines or steroids. The cDNA thus derived was then sequenced using CuraGen's proprietary SeqCalling technology. Sequence traces were evaluated manually and edited for corrections if appropriate. cDNA sequences from all samples were assembled together, sometimes including public human sequences, using bioinformatic programs to produce a consensus sequence for each assembly. Each assembly is included in CuraGen Corporation's database. Sequences were included as components for assembly when the extent of identity with another component was at least 95% over 50 bp. Each assembly represents a gene or portion thereof and includes information on variants, such as splice forms single nucleotide polymorphisms (SNPs), insertions, deletions and other sequence variations.

Exon Linking: The cDNA coding for the CG51051-03 sequence was cloned by the polymerase chain reaction (PCR). Primers were designed based on in silico predictions of the full length or some portion (one or more exons) of the cDNA/protein sequence of the invention. These primers were used to amplify a cDNA from a pool containing expressed human sequences derived from the following tissues: adrenal gland, bone marrow, brain-amygdala, brain-cerebellum, brain-hippocampus, brain-substantia nigra, brain-thalamus, brain-whole, fetal brain, fetal kidney, fetal liver, fetal lung, heart, kidney, lymphoma-Raji, mammary gland, pancreas, pituitary gland, placenta, prostate, salivary gland, skeletal muscle, small intestine, spinal cord, spleen, stomach, testis, thyroid, trachea and uterus.

Multiple clones were sequenced and these fragments were assembled together, sometimes including public human sequences, using bioinformatic programs to produce a consensus sequence for each assembly. Each assembly is included in CuraGen Corporation's database. Sequences were included as components for assembly when the extent of identity with another component was at least 95% over 50 bp. Each assembly represents a gene or portion thereof and includes information on variants, such as splice forms single nucleotide polymorphisms (SNPs), insertions, deletions and other sequence variations.

Variant sequences are also included in this application. A variant sequence can include a single nucleotide polymorphism (SNP). A SNP can, in some instances, be referred to as a “cSNP” to denote that the nucleotide sequence containing the SNP originates as a cDNA. A SNP can arise in several ways. For example, a SNP may be due to a substitution of one nucleotide for another at the polymorphic site. Such a substitution can be either a transition or a transversion. A SNP can also arise from a deletion of a nucleotide or an insertion of a nucleotide, relative to a reference allele. In this case, the polymorphic site is a site at which one allele bears a gap with respect to a particular nucleotide in another allele. SNPs occurring within genes may result in an alteration of the amino acid encoded by the gene at the position of the SNP. Intragenic SNPs may also be silent, when a codon including a SNP encodes the same amino acid as a result of the redundancy of the genetic code. SNPs occurring outside the region of a gene, or in an intron within a gene, do not result in changes in any amino acid sequence of a protein but may result in altered regulation of the expression pattern. Examples include alteration in temporal expression, physiological response regulation, cell type expression regulation, intensity of expression, and stability of transcribed message.

The DNA sequence and protein sequence for a novel Netrin-like gene were obtained by exon linking and are reported here as CuraGen Ace. No. CG51051-03.

Results

The novel nucleic acid of 1544 nucleotides (designated CuraGen Ace. No. CG51051-03) encoding a novel Netrin-like protein is shown in FIG. 1. An open reading frame was identified beginning at nucleotides 230 and ending at nucleotides 1517. This polypeptide represents a novel functional Netrin-like protein. The start and stop codons of the open reading frame are highlighted in bold type. Putative untranslated regions (underlined), if any, are found upstream from the initiation codon and downstream from the termination codon. The encoded protein having 429 amino acid residues is presented using the one-letter code in FIG. 2.

Similarities

In a search of sequence databases, it was found, for example, that the nucleic acid sequence of this invention has 1090 of 1286 bases (84%) identical to a gb:GENBANK-ID:ABO38663|acc:AB038663.1 mRNA from Mus musculus (Mus musculus mRNA for Netrin-Gle, complete cds) (FIG. 3A). The full amino acid sequence of the protein of the invention was found to have 341 of 353 amino acid residues (96%) identical to, and 346 of 353 amino acid residues (98%) similar to, the 460 amino acid residue ptnr:SPTREMBL-ACC:Q9ESR7 protein from Mus musculus (Mouse) (NETRIN-G1E)(FIG. 3B).

A multiple sequence alignment is given in FIG. 4, with the protein of the invention being shown on the first line in a ClustalW analysis comparing the protein of the invention with related protein sequences. Please note this sequence represents a splice form of Netrin as indicated in positions 353 to 404

The presence of identifiable domains in the protein disclosed herein was determined by searches versus domain databases such as Pfam, PROSITE, ProDom, Blocks or Prints and then identified by the Interpro domain accession number. Significant domains are summarized in Table 1.

| | Do- | | | hmm- | | | |
|---|---|---|---|---|---|---|---|
| Mode 1 | main | seq-f | seq-t | f | hmm-t | score | E-value |
| laminin_{—} | 1/1 | 50 | 295 . . . | 1 | 264 [] | 31.4 | 4.3e − 12 |
| Nterm | | | | | | | |
| EGF | 1/2 | 299 | 326 . . . | 1 | 45 [] | 3.9 | 9.9 |
| laminin_{—} | 1/1 | 297 | 341 . . . | 1 | 59 [] | 31.8 | 1.5e − 05 |
| EGF | | | | | | | |
| squash | 1/1 | 355 | 384 . . . | 1 | 30 [] | 4.2 | 1.3 |
| EGF | 2/2 | 357 | 387 . . . | 1 | 45 [] | 19.1 | 0.11 |

IPR002049

Laminin-Type EGF-Like (LE) Domain

Laminins are the major noncollagenous components of basement membranes that mediate cell adhesion, growth migration, and differentiation. They are composed of distinct but related alpha, beta and gamma chains. The three chains form a cross-shaped molecule that consist of a long arm and three short globular arms. The long arm consists of a coiled coil structure contributed by all three chains and cross-linked by interchain disulfide bonds. Beside different types of globular domains each subunit contains, in its first half, consecutive repeats of about 60 amino acids in length that include eight conserved cysteines. The tertiary structure of this domain is remotely similar in its N-terminal to that of the EGF-like module (see PROSITEDOC PDOC00021). It is known as a ‘LE’ or ‘laminin-type EGF-like’ domain. The number of copies of the LE domain in the different forms of laminins is highly variable; from 3 up to 22 copies have been found.

IPR001886

Laminin N-Terminal (Domain VI)

Lamin is thought to mediate the attachment, migration and organisation of cells into tissues during embryonic development by interacting with other extracellular matrix components.

This indicates that the sequence of the invention has properties similar to those of other proteins known to contain this/these domain(s) and similar to the properties of these domains.

Chromosomal Information

The Netrin-like gene disclosed in this invention maps to chromosome 1. This assignment was made using mapping information associated with genomic clones, public genes and ESTs sharing sequence identity with the disclosed sequence and CuraGen Corporation's Electronic Northern bioinformatic tool.

Tissue Expression

The Netrin-like gene disclosed in this invention is expressed in at least the following tissues: Foreskin, Hypothalamus, and Thalamus. Expression information was derived from the tissue sources of the sequences that were included in the derivation of the sequence of CuraGen Acc. No. CG51051-03.The sequence is predicted to be expressed in the following tissues because of the expression pattern of (GENBANK-ID: gb:GENBANK-ID:ABO38663|acc:AB038663.1) a closely related Mus musculus mRNA for Netrin-G1e, complete cds homolog in species Mus musculus:Brain.

Cellular Localization and Sorting

The PSORT, SignalP and hydropathy profile for the Netrin-like protein are shown in FIG. 5. The best PSORT score indicates a localization in the endoplasmic reticulum, but related proteins have been localized to the plasma membrane. Therefore, this protein is likely to be localized at the plasma membrane with a certainty of 0.4960. The signal peptide is predicted by SignalP to be cleaved at amino acid 18 and 19: VSS-VM.

Functional Variants and Homologs

The novel nucleic acid of the invention encoding a Netrin-like protein includes the nucleic acid whose sequence is provided in FIG. 1, or a fragment thereof. The invention also includes a mutant or variant nucleic acid any of whose bases may be changed from the corresponding base shown in FIG. 1 while still encoding a protein that maintains its Netrin-like activities and physiological functions, or a fragment of such a nucleic acid. The invention further includes nucleic acids whose sequences are complementary to the sequence of CuraGen Acc. No. CG51051-03, including nucleic acid fragments that are complementary to any of the nucleic acids just described. The invention additionally includes nucleic acids or nucleic acid fragments, or complements thereto, whose structures include chemical modifications. Such modifications include, by way of non-limiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 16% of the bases may be so changed.

The novel protein of the invention includes the Netrin-like protein whose sequence is provided in FIG. 2. The invention also includes a mutant or variant protein any of whose residues may be changed from the corresponding residue shown in FIG. 2 while still encoding a protein that maintains its Netrin-like activities and physiological functions, or a functional fragment thereof. In the mutant or variant protein, up to about 4% of the amino acid residues may be so changed.

Antibodies

The invention further encompasses antibodies and antibody fragments, such as Fab, (Fab)₂ or single chain FV constructs, that bind immunospecifically to any of the proteins of the invention. Also encompassed within the invention are peptides and polypeptides comprising sequences having high binding affinity for any of the proteins of the invention, including such peptides and polypeptides that are fused to any carrier particle (or biologically expressed on the surface of a carrier) such as a bacteriophage particle.

Methods of Use for the Compositions of the Invention

The protein similarity information, expression pattern, cellular localization, and map location for the protein and nucleic acid disclosed herein suggest that this Netrin-like protein may have important structural and/or physiological functions characteristic of the Laminin family. Therefore, the nucleic acids and proteins of the invention are useful in potential diagnostic and therapeutic applications and as a research tool. These include serving as a specific or selective nucleic acid or protein diagnostic and/or prognostic marker, wherein the presence or amount of the nucleic acid or the protein are to be assessed. These also include potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), (v) an agent promoting tissue regeneration in vitro and in vivo, and (vi) a biological defense weapon.

The nucleic acids and proteins of the invention have applications in the diagnosis and/or treatment of various diseases and disorders. For example, the compositions of the present invention will have efficacy for the treatment of patients suffering from: Von Hippel-Lindau (VHL) syndrome, Alzheimer's disease, stroke, tuberous sclerosis, hypercalceimia, Parkinson's disease, Huntington's disease, cerebral palsy, epilepsy, Lesch-Nyhan syndrome, multiple sclerosis, ataxia-telangiectasia, leukodystrophies, behavioral disorders, addiction, anxiety, pain, neurodegeneration as well as other diseases, disorders and conditions.

These materials are further useful in the generation of antibodies that bind immunospecifically to the novel substances of the invention for use in diagnostic and/or therapeutic methods.

TABLE 24
Nucleotide sequence encoding the Netrin-like protein of the invention.

(SEQ ID NO:117)

>CG51051-03
GGCTTCCACCAAAGTCCTCAATATACCTGAATACGCACAATATCTTAACTCTTCATATTTGGTTTTGGGA
TCTCCTTTGAGGTCCCATCTTCATTTAAAAAAAAATACAGAGACCTACCTACCCGTACGCATACATACAT
ATGTGTATATATATGTAAACTAGACAAAGATCGCAGATCATAAAGCAAGCTCTGCTTTAGTTTCCAAGAA
GATTACAAAGAATTTAGAGATGTATTTGTCAAGATTCCTGTCCATTCATGCCCTTTGGGTTACGGTGTCC
TCAGTGATGCAGCCCTACCCTTTGGTTTGGGGACATTATGATTTGTGTAAGACTCAGATTTACACGGAAG
AAGGGAAAGTTTGGGATTACATGGCCTGCCAGCCGGAATCCACGGACATGACAAAATATCTGAAAGTGAA
ACTCGATCCTCCGGATATTACCTGTGGAGACCCTCCTGAGACGTTCTGTGCAATGGGCAATCCCTACATG
TGCAATAATGAGTGTGATGCGAGTACCCCTGAGCTGGCACACCCCCCTGAGCTGATGTTTGATTTTGAAG
GAAGACATCCCTCCACATTTTGGCAGTCTGCCACTTGGAAGGAGTATCCCAAGCCTCTCCAGGTTAACAT
CACTCTCTCTTGGAGCAAAACCATTGAGCTAACAGACAACATAGTTATTACCTTTGAATCTGGGCCTCCA
GACCAAATGATCCTGGAGAAGTCTCTCCATTATGGACGAACATGGCAGCCCTATCAGTATTATGCCACAG
ACTGCTTAGATGCTTTTCACATGGATCCTAAATCCGTGAAGGATTTATCACAGCATACGGTCTTAGAAAT
CATTTGCACAGAAGAGTACTCAACAGGGTATACAACAAATAGCAAAATAATCCACTTTGAAATCAAAGAC
AGGTTCCCGTTTTTTGCTGGACCTCGCCTACGCAATATCGCTTCCCTCTACGGACAGCTCGATACAACCA
AGAAACTCAGAGATTTCTTTACAGTCACAGACCTGAGGATAAGGCTGTTAAGACCAGCCGTTGGGGAAAT
ATTTGTAGATGAGCTACACTTGGCACGCTACTTTTACCCGATCTCACACATAAAGGCGCGAGGAAGGTGC
AAGTGTAATCTCCATGCCACTCTATGTGTGTATGACAACAGCAAATTGACATGCGAATGTGACCACAACA
CTACAGCTCCAGACTGTGGGAAATGCAAGAAGAATTATCAGGGCCGACCTTGGAGTCCAGGCTCCTATCT
CCCCATCCCCAAAGGCACTGCAAATACCTCGAATGTCTGCGACAACGAGCTCCTGCACTGCCAGAACGGA
GGGACGTGCCACAACAACGTCCGCTGCCTGTGCCCGCCCGCATACACGGGCATCCTCTGCGAGAAGCTGC
GGTGCGAGGAGGCTGGCAGCTGCGGCTCCGACTCTGGTCAGGGCGCGCCCCCGCACGGCTCCCCAGCGCT
GCTCCTGCTGACCACGCTGCTGGGAACCGCCAGCCCCCTGGTCTTTTAGGTGTCACCTCCAGCCACACCG
GACG

TABLE 25
Protein sequence for CG51051-03.

| | | |
|---|---|---|
| >CG51051-03 | | |
| MYLSRFLSIHALWVTVSSVMQPYPLVWGHYDLCKTQIYTEEGKVWDYNACQPESTDMTKY | 60 | (SEQ ID NO:118) |
| LKVKLDPPDITCGDPPETFCAMGNPYMCNNECDASTPELAHPPELMFDFEGRHPSTFWQS | 120 | |
| ATWKEYPKPLQVNITLSWSKTIELTDNIVITFESGRPDQNILEKSLDYGRTWQPYQYYAT | 180 | |
| DCLDAFHMDPKSVKDLSQHTVLEIICTEEYSTGYTTNSKIIHFEIKDRFAFPAGPRLRNM | 240 | |
| ASLYCQLDTTKKLRDFFTVTDLRIRLLRPAVGEIFVDELHLARYFYAISDIKARGRCKCN | 300 | |
| LHATVCVYDNSKLTCECEHNTTGPDCGKCKKNYQGRPWSPGSYLPIPKGTANTSNVCNDE | 360 | |
| LLHCQNGGTCHNNVRCLCPAAYTGILCEKLRCEEAGSCGSDSCQGAPPHCSPALLLLTTL | 420 | |
| LGTASPLVF 429 | | |

TABLE 26
BLASTN search using CuraGen ACC. No. CG51051-03.

>gb:GENBANK-ID:AB038663|acc:AB038663.1 Mus musculus in RNA for Netrin-Gle,
complete cds - Mus musculus, 1383 bp.
Length = 1383
Plus Strand HSPs:
Score = 4508 (676.4 bits), Expect = 9.4e−198, P = 9.4e−198
Identities = 1090/1286 (84%), Positives = 1090/1286 (84%), Strand Plus/Plus

| | | | | |
|---|---|---|---|---|
| Query: | 230 | ATGTATTTGTCAAGATTCCTGTCGATTCATGCCCTTTCGGTTACGGTGTCCTCAGTGATG | 289 | (SEQ ID NO:119) |
| | | ATGTATTTGTCAAGATTCCTGTCGAT CATGCCCT TGGGT AC GTGTCCTC CTGATG | | (SEQ ID NO:120) |
| Sbjct: | 1 | ATGTATTTGTCAACATTCCTGTCGATCCATGCCCTGTGGGTGACAGTGTCCTCTGTGATG | 60 | (SEQ ID NO:121) |
| Query: | 290 | CAGCCCTACCCTTTGGTTTGGGGACATTATGATTTGTGTAAGACTCAGATTTACACGGAA | 349 | |
| | | CAGCCCTACC TTT CT TGGGGACATTATGAT T TCTAAGA C GATTTACAC GAA | | |
| Sbjct: | 61 | CAGCCCTACCTTTTCGTGTGGGGACATTATGATGTATGTAACAGCCTGATTTACACAGAA | 120 | |
| Query: | 350 | GAAGGGAAAGTTTGGGATTACATGGCCTGCCAGCCGGAATCCACGGACATGACAAAATAT | 409 | |
| | | GAAGG AAAGTTTGGGATTACA GCCTGCCAGCCGGAATCCACGGACATGAC AA TAT | | |
| Sbjct: | 121 | GAAGGCAAAGTTTGGGATTACACAGCCTGCCACCCGGAATCCACGGACATGACCAAGTAT | 180 | |
| Query: | 410 | CTGAAAGTGAAACTCGATCCTCCGGATATTACCTGTCGAGACCCTCCTGAGACGTTCTGT | 469 | |
| | | CTGAAAGTGAAACT GA CCTCCGGATATTACCTGTGGAGACCCTCC GAG C TTCTGT | | |
| Sbjct: | 181 | CTGAAAGTGAAACTGGACCCTCCGGATATTACCTGTGGAGACCCTCCACAGTCCTTCTGT | 240 | |
| Query: | 470 | GCAATGGGCAATCCCTACATGTCCAATAATGAGTGTCATGCGAGTACCCCTGAGCTCGCA | 529 | |
| | | GCAATGGGCAA CC TACATGTGCAATAATGAGTGTCATGCCAGTACCCCTGA CTGGCA | | |
| Sbjct: | 241 | GCAATGGGCAACCCTTACATGTCCAATAATGAGTGTGATGCGAGTACCCCTGAACTGGCA | 300 | |
| Query: | 530 | CACCCCCCTGAGCTGATGTTTGATTTTGAAGGAAGACATCCCTCCACATTTTGGCAGTCT | 589 | |
| | | CACCC CCTGAGCTGATGTTTGATTTTGAAGCAAGACATCCCTCCACATTTTCGCAGTCT | | |
| Sbjct: | 301 | CACCCTCCTGAGCTGATGTTTGATTTTGAAGCAAGACATCCCTCCACATTTTGGCAGTCT | 360 | |
| Query: | 590 | GCCACTTGCAAGGAGTATCCCAAGCCTCTCCAGGTTAACATCACTCTGTCTTGGAGCAAA | 649 | |
| | | GC ACTTGGAAGGAGTA CCCAA CCTCTCCAGGTTAACATCACTCTGTCTTGGAGCAAA | | |
| Sbjct: | 361 | CCTACTTGGAACGAGTACCCCAAACCTCTCCAGGTTAACATCACTCTGTCTTGGACCAAA | 420 | |
| Query: | 650 | ACCATTGAGCTAACAGACAACATAGTTATTACCTTTGAATCTGGGCGTCCAGACCAAATG | 709 | |
| | | ACCATTGA CT ACAGACAACATAGTTATTACCTTTCAATC GGGCGTCCACACCAAATC | | |
| Sbjct: | 421 | ACCATTCAACTCACAGACAACATAGTTATTACCTTTGAATCGGGGCGTCCAGACCAAATG | 480 | |
| Query: | 710 | ATCCTGGACAAGTCTCTCGATTATGGACGAACATGGCAGCCCTATCAGTATTATGCCACA | 769 | |
| | | ATCCT GACAA TCTCTCGA TA GGACGAACATGGCAGCCCTATCAGTATTATGCCACA | | |
| Sbjct: | 481 | ATCCTAGAGAAATCTCTCGACTACGGACGAACATGGCAGCCCTATCAGTATTATGCCACA | 540 | |
| Query: | 770 | CACTCCTTAGATGCTTTTCACATGGATCCTAAATCCGTGAAGGATTTATCACACCATACG | 829 | |
| | | GACTGC T ATGC TT CACATGGA CC AAATCCGTGAAGGATTTATC CACCA ACG | | |
| Sbjct: | 541 | GACTGCCTCCATGCATTCCACATCGACCCGAAATCCGTGAAGGATTTATCTCAGCACACG | 600 | |
| Query: | 830 | GTCTTAGAAATCATTTGCACAGAAGAGTACTCAACAGGGTATACAACAAATAGCAAAATA | 889 | |
| | | GTCTT GAAATCATTTGCAC GAAGAGTACTC AC GGGTA C AC AATAGCAAAATA | | |
| Sbjct: | 601 | GTCTTGGAAATCATTTGCACGGAAGAGTACTCCACTGCGTACTCCACCAATACCAAAATA | 660 | |
| Query: | 890 | ATCCACTTTGAAATCAAAGACAGGTTCGCGTTTTTTGCTGGACCTCGCCTACGCAATATG | 949 | |
| | | ATCCACTT GA ATCAAAGACAGGTT GCGTTTTT GCTGGACCTCG CTACG AATATG | | |
| Sbjct: | 661 | ATCCACTTCCAGATCAAAGACAGGTTTGCGTTTTTCGCTGGACCTCGGCTACGAAATATG | 720 | |
| Query: | 950 | GCTTCCCTCTACGGACAGCTGGATACAACCAAGAAACTCAGAGATTTCTTTACACTCACA | 1009 | |
| | | GCTTCCCTCTA GGACAGCTGGATACAACCAAGAAACTCAGAGATTTCTT AC GTCACA | | |
| Sbjct: | 721 | GCTTCCCTCTATGGACAGCTGGATACAACCAAGAAACTCAGAGATTTCTTCACTGTCACA | 780 | |
| Query: | 1010 | GACCTGAGGATAAGGCTGTTAAGACCAGCCGTTCGCCAAATATTTGTAGATGAGCTACAC | 1069 | |
| | | CACCTGAGGAT AGGCTGTT AGACC GCCGTTGGGGAAATATTTGTAGATGA CTACA | | |
| Sbjct: | 781 | GACCTGAGGATCAGGCTGTTGAGACCCGCCGTTGGGGAAATATTTGTAGATGAACTACAT | 840 | |
| Query: | 1070 | TTGGCACGCTACTTTTACGCGATCTCAGACATAAAGGCGCGAGGAAGGTGCAAGTGTAAT | 1129 | |
| | | TTGGCACG TACTTTTA GCGATCTCAGACATAA-ACG GCGAGGAAGGTGCAAGTG AA | | |
| Sbjct: | 841 | TTGGCACGTTACTTTTATGCGATCTCAGACATAAAGGTGCGAGGAAGGTGCAAGTGCAAC | 900 | |
| Query: | 1130 | CTCCATGCCACTGTATGTGTGTATGACAACAGCAAATTGACATGCGAATGTGAGCACAAC | 1189 | |
| | | CT CATGCCACT TGT TGTATGACAACAGCAAA TGACATG GAATGTGAGCACAAC | | |
| Sbjct: | 901 | CTGCATGCCACTTCGTGTTTGTATGACAACAGCAAACTGACATGTGAATGTGAGCACAAC | 960 | |
| Query: | 1190 | ACTACAGGTCCAGACTGTGGGAAATGCAAGAAGAATTATCAGGGCCGACCTTGGAGTCCA | 1249 | |
| | | ACTACAGGTCC GACTGTGGGAAATGCAAGAAGAA TA CAGGGCCGACCTTGGAG CC | | |
| Sbjct: | 961 | ACTACAGGTCCCGACTGTGGGAAATGCAAGAAGAACTACCAGGGCCGACCTTGGAGCCCC | 1020 | |
| Query: | 1250 | GGCTCCTATCTCCCCATCCCCAAAGGCACTGCAAATACCTCGAATGTCT-GCGACAACGA | 1308 | |
| | | GGCTC TA CTCCCCATCCCCAAAGGCAC GCAAA ACCT G AT C GC A C A | | |
| Sbjct: | 1021 | GGCTCATACCTCCCCATCCCCAAAGGCACCGCAAACACCT-GTATCCCCAGC-ATTTCCA | 1078 | |
| Query: | 1309 | GCTCCTGCACTGCCAGAACGG--AG--GGACGTGCCACAACAACG-TGCGCTGCCTGTGC | 1363 | |
| | | G C G ACT C AA G A GGA TG C AA A T C CT GT | | |
| Sbjct: | 1079 | GTATCGGTACTCCTCCAAAGTTTAATAGGATATGGCCGAATATTTCTTCCCTTGAGGTTT | 1138 | |
| Query: | 1364 | CCGGCCGCATACACGGGCATCCTCTGCGAGAAGCTGCGG-TGCGAGG---AGGCTGGCAGC | 1420 | |
| | | C CC A ACA G AT TCTGCGA AA GC TGC C AG TGG G | | |
| Sbjct: | 1139 | CTAACCCAAAACAAGCTAATG-TCTGCGACAATGAGCTCCTGCACTGCCAGAATGGAGGG | 1197 | |
| Query: | 1421 | TGCGGCTCCGA-CTCTGGTCAGGGCGCGCCCCCGCACGGCTCCCCAGCGC-TGCTGCTGC | 1478 | |
| | | C GC C GA C TG C GCGCG CCC ACG CT C C G GC T CT CTG | | |
| Sbjct: | 1198 | ACCTGC-CAGAACAATGTGCGCTGCGCGTGCCCAGACGCCTACACCG-GCATCCT-CTG- | 1253 | |
| Query: | 1479 | TGACCACGCTGCTG-G--GAACCGCCAGCCCCCTG 1510 | | |
| | | TGA A GCT C G G GAA G C G C CTG | | |
| Sbjct: | 1254 | TGAGAA-GCTACGGTGCGAAGAGGCGGGCAGCTG 1286 | | |

Score = 873 (131.0 bits), Expect = 1.4e−33, P = 1.4e−33
Identities = 205/239 (85%), Positives = 205/239 (85%), Strand = Plus/Plus

(QUERY: SEQ ID NO:122; Consensus: SEQ ID NO:123; Subject: SEQ ID NO:124)

| | | | |
|---|---|---|---|
| Query: | 1281 | CAAATACCT-CGAATGTCTGCGACAACGAGCTCCTGCACTGCCAGAACGGAGGGACGTGC | 1339 |
| | | CAAA AC C AATGTCTGCGACAA GAGCTCCTGCACTGCCAGAA GGAGGGAC TGC | |
| Sbjct: | 1145 | CAAA-ACAAGCTAATGTCTGCGACAATGAGCTCCTGCACTGCCAGAATGGAGGGACCTGC | 1203 |
| Query: | 1340 | CACAACAACGTGCGCTGCCTGTGCCCGGCCGCATACACGGGCATCCTCTGCGAGAAGCTG | 1399 |
| | | CA AACAA GTGCGCTGC GTGCCC G CGC TACAC GGCATCCTCTG GAGAAGCT | |
| Sbjct: | 1204 | CAGAACAATGTGCGCTGCGCGTGCCCAGACGCCTACACCGGCATCCTCTGTGAGAAGCTA | 1263 |
| Query: | 1400 | CGGTGCGAGGAGGCTGGCAGCTGCGGCTCCGACTCTGGTCAGGGCGCGCCCCCGCACGGC | 1459 |
| | | CGGTGCGA GAGGC GGCAGCTG GGCTCCGA TC GG CAGGG GC CCCCCGC GGC | |
| Sbjct: | 1264 | CGGTGCGAAGAGGCGGGCAGCTGTGGCTCCGAATCCGGCCAGGGAGCACCCCCGCGGGGC | 1323 |
| Query: | 1460 | TCCCCAGCGCTGCTGCTGCTGACCACGCTGCTCGGAACCGCCAGCCCCCTGGTGTTTTAG | 1519 |
| | | TCCCCAGC CTGCTGCTGCTGACCA GCTGCTGGG AC GCC G CCCCTGGTGTT TAG | |
| Sbjct: | 1324 | TCCCCAGCACTGCTGCTGCTGACCATGCTGCTGGGGACTGCCGGTCCCCTGGTGTTCTAG | 1383 |

TABLE 27
BLASTP search using the protein of CuraGen ACC. No. CG51051-03.

>ptnr:SPTREMBL-ACC:Q9ESR7 NETRIN-G1E-Mus musculus (Mouse), 460 aa.
Length = 460
Score = 1896 (667.4 bits), Expect = 2.9e−233, Sum P(2) = 2.9e−233
Identities = 341/353 (96%), Positives = 346/353 (98%)

(QUERY: SEQ ID NO:125; Consensus: SEQ ID NO: 126; Subject: SEQ ID NO:127)

| | | | |
|---|---|---|---|
| Query: | 1 | MYLSRFLSIHALWVTVSSVMQPYPLVWGHYDLCKTQIYTEEGKVWDYMACQPESTDMTKY | 60 |
| | | MYLSRFLSIHALWVTVSSVMQPY VWGHYD+CK+ IYTEEGKVWDY ACQPESTDMTKY | |
| Sbjct: | 1 | MYLSRFLSIHALWVTVSSVMQPYLFVWGHYDVCKSLIYTEEGKVWDYTACQPESTDMTKY | 60 |
| Query: | 61 | LKVKLDPPDITCGDPPETFCAMGNPYMCNNECDASTPELAHPPELMFDFEGRHPSTFWQS | 120 |
| | | LKVKLDPPDITCGDPPE+FCAMGNPYMCNNECDASTPELAHPPELMFDFEGRHPSTFWQS | |
| Sbjct: | 61 | LKVKLDPPDITCGDPPESFCAMGNPYMCNNECDASTPELAHPPELMFDFEGRHPSTFWQS | 120 |
| Query: | 121 | ATWKEYPKPLQVNITLSWSKTIELTDNIVITFESGRPDQMILEKSLDYGRTWQPYQYYAT | 180 |
| | | ATWKEYPKPLQVNITLSWSKTIELTDNIVITFESGRPDQMILEKSLDYGRTWQPYQYYAT | |
| Sbjct: | 121 | ATWKEYPKPLQVNITLSWSKTIELTDNIVITFESGRPDQMILEKSLDYGRTWQPYQYYAT | 180 |
| Query: | 181 | DCLDAFHMDPKSVKDLSQHTVLEIICTEEYSTGYTTNSKIIHFEIKDRFAFFAGPRLRNM | 240 |
| | | DCL AFHMDPKSVKDLSQHTVLEILCTEEYSTGY+TNSKIIHFEIKDRFAFFAGPRLPNM | |
| Sbjct: | 181 | DCLHAFHMDPKSVKDLSQHTVLEIICTEEYSTGYSTNSKIIHFEIKDRFAFFAGPRLRNM | 240 |
| Query: | 241 | ASLYGQLDTTKKLRDFFTVTDLRIRLLRPAVGEIFVDELHLARFPYAISDIKARGRCKCN | 300 |
| | | ASLYGQLDTTKKLRDFFTVTDLRIRLLRPAVGEIFVDELHLARYFYAISDIK RGRCKCN | |
| Sbjct: | 241 | ASLYGQLDTTKKLRDFFTVTDLRIRLLRPAVGEIFVDELHLARYFYAISDIKVRGRCKCN | 300 |
| Query: | 301 | LHATVCVYDNSKLTCECEHNTTGPDCGKCKKNYQGRPWSPGSYLPIPKGTANT 353 | |
| | | LHAT C+YDNSKLTCECEHNTTGPDCGKCKKNYQCRPWSPGSYLPIPKGTANT | |
| Sbjct: | 301 | LHATSCLYDNSKLTCECEHNTTGPDCGKCKKNYQGRPWSPGSYLPIPKGTANT 353 | |

Score = 379 (133.4 bits), Expect = 2.9e−233, Sum P(2) = 2.9e−233
Identities = 72/94 (76%), Positives = 75/94 (79%)

| | | | | |
|---|---|---|---|---|
| Query: | 336 | RPWSPGSYLPIPKGTANTSNVCDNELLHCQNGGTCHNNVRCLCPAAYTGILCEKLRCEEA | 395 | (SEQ ID NO:128) |
| | | R W S L + +NVCDNELLHCQNGGTC NNVRC CP AYTGILCEKLRCEEA | | (SEQ ID NO:129) |
| Sbjct: | 369 | RIWPNISSLEV--SNPKQANVCDNELLHCQNGGTCQNNVRCACPDAYTGILCEKLRCEEA | 426 | (SEQ ID NO:130) |
| Query: | 396 | GSCGSDSGQGAPPHGSPALLLLTTLLGTASPLVF 429 | | |
| | | GSCGS+SGQGAPP GSPALLLLT LLGTA PLVF | | |
| Sbjct: | 427 | GSCGSESGQGAPPRGSPAILLLTMLLGTAGPLVF 460 | | |

TABLE 28

BLASTN identity search of CuraGen Corporation's Human SeqCalling
database using CuraGen Acc. No. CG51051-03.

>s3aq:158621742 Category E:, 4711 bp.
Length = 4711
Plus Strand HSPs:
Score = 6437 (965.8 bits), Expect = 3.7e−286, P = 3.7e−286
Identities = 1291/1294 (99%), Positives 1291/1294 (99%), Strand = Plus/Plus

| | | | | |
|---|---|---|---|---|
| Query: | 1 | GGCTTCCACCAAAGTCCTCAATATACCTGAATACGCACAATATCTTAACTCTTCATATTT | 60 | (SEQ ID NO:131) |
| | | GGCTTCCACCAAAGTCCTCAATATACCTGAATACGCACAATATCTTAACTCTTCATATTT | | (SEQ ID NO:132) |
| Sbjct: | 518 | GGCTTCCACCAAAGTCCTCAATATACCTGAATACGCACAATATCTTAACTCTTCATATTT | 577 | (SEQ ID NO:133) |
| Query: | 61 | GGTTTTGGGATCTGCTTTGAGGTCCCATCTTCATTTAAAAAAAAATACAGAGACCTACCT | 120 | |
| | | GGTTTTGGGATCTGCTTTGAGGTCCCATCTTCATTTAAAAAAAAATACAGAGACCTACCT | | |
| Sbjct: | 578 | GGTTTTGGGATCTGCTTTGAGCTCCCATCTTCATTTAAAAAAAAATACAGAGACCTACCT | 637 | |
| Query: | 121 | ACCCGTACGCATACATACATATGTGTATATATATGTAAACTAGACAAAGATCGCAGATCA | 180 | |
| | | ACCCGTACGCATACATACATATGTGTATATATATGTAAACTAGACAAAGATCGCAGATCA | | |
| Sbjct: | 638 | ACCCGTACGCATACATACATATGTGTATATATATGTAAACTAGACAAAGATCGCAGATCA | 697 | |
| Query: | 181 | TAAAGCAAGCTCTGCTTTAGTTTCCAAGAAGATTACAAAGAATTTAGAGATGTATTTGTC | 240 | |
| | | TAAAGCAAGCTCTGCTTTAGTTTCCAAGAAGATTACAAAGAATTTAGAGATGTATTTGTC | | |
| Sbjct: | 698 | TAAAGCAAGCTCTGCTTTAGTTTCCAAGAAGATTACAAAGAATTTAGAGATGTATTTGTC | 757 | |
| Query: | 241 | AAGATTCCTGTCGATTCATGCCCTTTCGGTTACCGTCTCCTCAGTGATGCAGCCCTACCC | 300 | |
| | | AAGATTCCTGTCGATTCATGCCCTTTGGGTTACGGTGTCCTCAGTGATGCAGCCCTACCC | | |
| Sbjct: | 758 | AAGATTCCTGTCGATTCATGCCCTTTGGGTTACGGTGTCCTCAGTGATGCAGCCCTACCC | 817 | |
| Query: | 301 | TTTGGTTTGGGGACATTATGATTTGTGTAAGACTCAGATTTACACGGAAGAAGGGAAAGT | 360 | |
| | | TTTGGTTTGGGGACATTATGATTTGTGTAAGACTCAGATTTACACGGAAGAAGGGAAAGT | | |
| Sbjct: | 818 | TTTGGTTTGGGGACATTATGATTTGTGTAAGACTCAGATTTACACGGAGAAAGGGAAAGT | 877 | |
| Query: | 361 | TTGGGATTACATGGCCTGCCAGCCGGAATCCACGGACATGACAAAATATCTGAAAGTGAA | 420 | |
| | | TTGGGATTACATGGCCTGCCAGCCGGAATCCACGGACATGACAAAATATCTGAAAGTGAA | | |
| Sbjct: | 878 | TTGGGATTACATGGCCTCCCAGCCGCAATCCACGGACATGACAAAATATCTGAAAGTGAA | 937 | |
| Query: | 421 | ACTCGATCCTCCGGATATTACCTGTGGAGACCCTCCTGAGACGTTCTGTGCAATGGGCAA | 480 | |
| | | ACTCGATCCTCCGGATATTACCTGTCGAGACCCTCCTCAGACGTTCTCTGCAATGGGCAA | | |
| Sbjct: | 938 | ACTCGATCCTCCCGATATTACCTGTGGAGACCCTCCTGAGACGTTCTGTGCAATGGGCAA | 997 | |
| Query: | 481 | TCCCTACATGTGCAATAATGAGTGTGATGCGAGTACCCCTGAGCTGGCACACCCCCCTGA | 540 | |
| | | TCCCTACATGTGCAATAATGAGTGTGATGCGAGTACCCCTGACCTGGCACACCCCCCTGA | | |
| Sbjct: | 998 | TCCCTACATGTGCAATAATGAGTGTGATGCGAGTACCCCTGAGCTGGCACACCCCCCTGA | 1057 | |
| Query: | 541 | GCTGATCTTTGATTTTGAACGAAGACATCCCTCCACATTTTGGCAGTCTGCCACTTGCAA | 600 | |
| | | GCTGATGTTTCATTTTGAAGCAAGACATCCCTCCACATTTTGGCAGTCTGCCACTTGGAA | | |
| Sbjct: | 1058 | CCTGATGTTTGATTTTGAAGGAAGACATCCCTCCACATTTTGGCAGTCTGCCACTTGGAA | 1117 | |
| Query: | 601 | GGAGTATCCCAAGCCTCTCCAGGTTAACATCACTCTGTCTTGGAGCAAAACCATTGAGCT | 660 | |
| | | GGAGTATCCCAAGCCTCTCCAGGTTAACATCACTCTGTCTTGGAGCAAAACCATTGACCT | | |
| Sbjct: | 1118 | GGAGTATCCCAAGCCTCTCCAGGTTAACATCACTCTGTCTTCGAGCAAAACCATTGAGCT | 1177 | |
| Query: | 661 | AACAGACAACATAGTTATTACCTTTGAATCTGGGCGTCCAGACCAAATGATCCTGGAGAA | 720 | |
| | | AACAGACAACATAGTTATTACCTTTGAATCTGGGCGTCCAGACCAATGATCCTGGAGAA | | |
| Sbjct: | 1178 | ACAGACAACATAGTTATTACCTTTCAATCTGCGCGTCCAGACCAAATGATCCTGGAGAA | 1237 | |
| Query: | 721 | GTCTCTCGATTATGGACGAACATGGCAGCCCTATCAGTATTATGCCACAGACTGCTTAGA | 780 | |
| | | GTCTCTCGATTATGGACGAACATGGCAGCCCTATCAGTATTATCCCACAGACTGCTTAGA | | |
| Sbjct: | 1238 | GTCTCTCCATTATGGACGAACATGGCAGCCCTATCAGTATTATGCCACAGACTGCTTAGA | 1297 | |
| Query: | 781 | TGCTTTTCACATGGATCCTAAATCCGTGAAGGATTTATCACACCATACGCTCTTAGAT | 840 | |
| | | TGCTTTTCACATGGATCCTAAATCCGTGAAGGATTTATCACAGCATACGGTCTTAGAAAT | | |
| Sbjct: | 1298 | TGCTTTTCACATGGATCCTAAATCCGTGAAGGATTTATCACAGCATACGGTCTTAGAAAT | 1357 | |
| Query: | 841 | CATTTGCACAGAACAGTACTCAACAGGCTATACAACATACCAAAATAATCCACTTTGA | 900 | |
| | | CATTTGCACAGAAGAGTACTCAACAGGGTATACAACAAATAGCAAAATAATCCACTTTGA | | |
| Sbjct: | 1358 | CATTTGCACAGAACAGTACTCAACAGGGTATACAACAAATAGCAAAATAATCCACTTTCA | 1417 | |
| Query: | 901 | AATCAAAGACAGGTTCGCGTTTTTTGCTGGACCTCGCCTACCCAATATGGCTTCCCTCTA | 960 | |
| | | ATCAAAGACAGGTTCGCGTTTTTTGCTGGACCTCGCCTACGCAATATGGCTTCCCTCTA | | |
| Sbjct: | 1418 | AATCAAAGACAGGTTCCCGTTTTTTGCTGCACCTCGCCTACCCAATATCGCTTCCCTCTA | 1477 | |
| Query: | 961 | CGGACAGCTCCATACAACCAAGAAACTCAGAGATTTCTTTACAGTCACAGACCTGAGGAT | 1020 | |
| | | CGGACAGCTGGATACAACCAAGAAACTCAGAGATTTCTTTACAGTCACAGACCTGAGGAT | | |
| Sbjct: | 1478 | CGGACAGCTGGATACAACCAAGAAACTCAGAGATTTCTTTACAGTCACAGACCTGAGGAT | 1537 | |
| Query: | 1021 | AAGGCTGTTAACACCAGCCGTTGGGGAAATATTTGTAGATGAGCTACACTTGGCACGCTA | 1080 | |
| | | AAGGCTGTTAAGACCAGCCGTTGGGGAAATATTTGTAGATGAGCTACACTTGGCACGCTA | | |
| Sbjct: | 1538 | AAGGCTGTTAAGACCAGCCGTTGGGGAAATATTTGTAGATGAGCTACACTTGGCACGCTA | 1597 | |
| Query: | 1081 | CTTTTACGCGATCTCAGACATAAAGGCGCGAGGAAGGTGCAAGTGTAATCTCCATGCCAC | 1140 | |
| | | CTTTTACGCGATCTCAGACATAAAGGGCGAGGAAGGTGCAAGTGTAATCTCCATGCCAC | | |
| Sbjct: | 1598 | CTTTTACGCGATCTCAGACATAAAGGTGCGAGGAAGGTGCAAGTGTAATCTCCATGCCAC | 1657 | |
| Query: | 1141 | TGTATGTGTGTATGACAACAGCAAATTGACATGCGAATGTGAGCACAACACTACAGGTCC | 1200 | |
| | | TGTATGTGTGTATGACAACAGCAAATTGACATGCGAATGTGAGCACAACACTACAGGTCC | | |
| Sbjct: | 1658 | TGTATGTGTGTATGACAACAGCAAATTGACATGCGAATGTGAGCACAACACTACAGGTCC | 1717 | |
| Query: | 1201 | AGACTGTGGGAAATGCAAGAAGAATTATCAGGGCCGACCTTGGAGTCCAGGCTCCTATCT | 1260 | |
| | | AGACTGTGGGAAATGCAAGAAGAATTATCAGGGCCGACCTTGGAGTCCAGGCTCCTATCT | | |
| Sbjct: | 1718 | AGACTGTGGGAAATGCAAGAAGAATTATCAGGGCCGACCTTGGAGTCCAGGCTCCTATCT | 1777 | |
| Query: | 1261 | CCCCATCCCCAAAGGCACTGCAAATACCTCGAAT 1294 | | |
| | | CCCCATCCCCAAAGGCACTGCAAATACCT G AT | | |
| Sbjct: | 1778 | CCCCATCCCCAAAGGCACTGCAAATACCT-GTAT 1810 | | |

>s3aq: 158627382, 794 bp.
Length = 794
Minus Strand HSPs:
Score = 1552 (232.9 bits), Expect = 2.3e−119, Sum P(2) 2.3e−119
Identities = 314/317 (99%), Positives = 314/317 (99%) , Strand = Minus/Plus

| | | | | |
|---|---|---|---|---|
| Query: | 1294 | ATTCGAGGTATTTGCAGTGCCTTTGGGGATGGGGAGATAGGAGCCTGGACTCCAAGGTCG | 1235 | (SEQ ID NO:134) |
| | | AT C AGGTATTTGCAGTGCCTTTGGGGATGGGGAGATAGGACCCTGGACTCCAAGGTCG | | (SEQ ID NO:135) |
| Sbjct: | 479 | ATAC-AGGTATTTGCAGTGCCTTTGGGGATGGGGACATAGGACCCTGGACTCCAAGGTCG | 537 | (SEQ ID NO:136) |
| Query: | 1234 | GCCCTGATAATTCTTCTTGCATTTCCCACAGTCTGGACCTCTAGTCTTCTGCTCACATTC | 1175 | |
| | | CCCCTGATAATTCTTCTTGCATTTCCCACAGTCTGGACCTGTAGTGTTGTGCTCACATTC | | |
| Sbjct: | 538 | GCCCTGATAATTCTTCTTGCATTTCCCACAGTCTGGACCTGTAGTGTTCTGCTCACATTC | 597 | |
| Query: | 1174 | GCATGTCAATTTGCTGTTGTCATACACACATACAGTGGCATGGAGATTACACTTGCACCT | 1115 | |
| | | GCATGTCAATTTGCTGTTGTCATACACACATACAGTGCCATGGACATTACACTTCCACCT | | |
| Sbjct: | 598 | GCATGTCAATTTGCTGTTGTCATACACACATACAGTGGCATGGAGATTACACTTGCACCT | 657 | |
| Query: | 1114 | TCCTCGCGCCTTTATGTCTGAGATCGCGTAAAAGTAGCGTGCCAAGTGTAGCTCATCTAC | 1055 | |
| | | TCCTCGCCCTTTATGTCTGAGATCGCGTAAAAGTAGCGTGCCAAGTGTACCTCATCTAC | | |
| Sbjct: | 658 | TCCTCGCACCTTTATGTCTGAGATCGCGTAAAAGTAGCGTGCCAACTCTAGCTCATCTAC | 717 | |
| Query: | 1054 | AAATATTTCCCCAACGGCTCGTCTTAACAGCCTTATCCTCACGTCTGTGACTGTAAAGAA | 995 | |
| | | AAATATTTCCCCAACCGCTGGTCTTAACAGCCTTATCCTCACGTCTGTCACTGTAAACAA | | |
| Sbjct: | 718 | AAATATTTCCCCAACGGCTGGTCTTAACAGCCTTATCCTCAGGTCTGTGACTGTAAAGAA | 777 | |
| Query: | 994 | ATCTCTGAGTTTCTTCG 978 | | |
| | | ATCTCTGAGTTTCTTGG | | |
| Sbjct: | 778 | ATCTCTGAGTTTCTTGG 794 | | |

Score = 1257 (188.6 bits), Expect = 2.3e−119, Sum P(2) 2.3e−119
Identities = 253/255 (99%), Positives = 253/255 (99%), Strand = Minus/Plus

| | | | | |
|---|---|---|---|---|
| Query: | 1544 | CGTCCGGTGTGGCTGGAGGTGACACCTAAAACACCAGGGGCCTGGCGGTTCCCAGCAGCG | 1485 | (SEQ ID NO:137) |
| | | \|\|\|\|\|\|\|\|\|\|\|\|\|\|\|\|\|\|\|\|\|\|\|\|\|\|\|\| \|\|\|\|\|\|\|\|\|\|\|\|\|\|\|\|\|\|\|\|\|\|\|\|\|\|\|\|\|\|\| | | |
| Sbjct: | 201 | CGTCCGGTGTGGCTGGAGGTGACACCTAGAACACCACGCGGCTGGCGGTTCCCAGCAGCG | 260 | (SEQ ID NO:138) |
| Query: | 1484 | TGGTCAGCAGCAGCAGCGCTGGGCAGCCGTGCGGGGGCGCGCCCTGACCAGAGTCGGAGC | 1425 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| \|\|\|\|\|\|\|\|\|\|\|\|\| | | |
| Sbjct: | 261 | TGGTCAGCAGCAGCAGCGCTGGGGAGCCGTGCGGGGGCGCGCCCTGGCCAGAGTCGGAGC | 320 | |
| Query: | 1424 | CGCAGCTGCCAGCCTCCTCGCACCGCAGCTTCTCGCAGAGGATGCCCGTGTATGCGGCCG | 1365 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| Sbjct: | 321 | CGCAGCTGCCAGCCTCCTCGCACCGCAGCTTCTCGCAGAGGATGCCCGTGTATGCGGCCG | 380 | |
| Query: | 1364 | GGCACAGGCAGCGCACGTTGTTGTGGCACGTCCCTCCGTTCTGGCAGTGCAGGAGCTCGT | 1305 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| Sbjct: | 381 | GGCACAGGCAGCGCACGTTCTTGTGGCACGTCCCTCCGTTCTGGCAGTGCACGAGCTCGT | 440 | |
| Query: | 1304 | TGTCGCAGACATTCG 1290 | | |
| | | \|\|\|\|\|\|\|\|\|\|\|\|\|\|\| | | |
| Sbjct: | 441 | TCTCGCAGACATTCG 455 | | |

>s3aq: 158627363, 487 bp.
Length = 487
Minus Strand HSPs:
Score = 1377 (206.6 bits), Expect = 2.8e−101, Sum P(2) = 2.8e−101
Identities = 277/279 (99%), Positives = 277/279 (99%), Strand Minus/Plus

| | | | | |
|---|---|---|---|---|
| Query: | 1116 | CTTCCTCGCGCCTTTATGTCTGAGATCGCGTAAAAGTAGCGTGCCAAGTGTAGCTCATCT | 1057 | (SEQ ID NO:139) |
| | | \|\|\|\|\|\|\|\|\| \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| Sbjct: | 209 | CTTCCTCGCACCTTTATGTCTGAGATCGCGTAAAAGTAGCGTGCCAAGTGTAGCTCATCT | 268 | (SEQ ID NO:140) |
| Query: | 1056 | ACAAATATTTCCCCAACGGCTGGTCTTAACAGCCTTATCCTCAGGTCTCTGACTGTAAAG | 997 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| Sbjct: | 269 | ACAAATATTTCCCCAACGGCTGGTCTTAACAGCCTTATCCTCAGGTCTGTCACTCTAAAG | 328 | |
| Query: | 996 | AAATCTCTGAGTTTCTTGGTTGTATCCAGCTCTCCGTAGAGGGAAGCCATATTGCGTAGG | 937 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| Sbjct: | 329 | AAATCTCTGAGTTTCTTGGTTGTATCCAGCTGTCCGTAGAGGGAAGCCATATTGCGTAGG | 388 | |
| Query: | 936 | CGAGGTCCAGCAAAAAACGCGAACCTGTCTTTGATTTCAAAGTGGATTATTTTGCTATTT | 877 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| Sbjct: | 389 | CGAGGTCCAGCAAAAAACGCGAACCTGTCTTTGATTTCAAAGTGGATTATTTTGCTATTT | 448 | |
| Query: | 876 | GTTGTATACCCTGTTGAGTACTCTTCTGTGCAAATGATT 838 | | |
| | | \|\|\|\|\|\|\|\|\|\|\|\|\|\|\|\|\|\|\|\|\|\| \|\|\|\|\|\|\|\|\|\|\|\|\|\|\|\| | | |
| Sbjct: | 449 | GTTCTATACCCTGTTGAGTACTTTTCTGTGCAAATGATT 487 | | |

Score = 1035 (155.3 bits), Expect = 2.8e−101, Sum P(2) = 2.8e−101
Identities = 229/251 (91%), Positives = 229/251 (91%), Strand Minus/Plus

| | | | | |
|---|---|---|---|---|
| Query: | 1497 | GTTCCCAGCAGCGTGGTCAGCAGCAGCAGCGCTGGGGAGCCGTGCGGGGGCGCGCCCTGA | 1438 | (SEQ ID NO:141) |
| | | GTTCCCAGCAGCGTCGTCAGCAGCAGCAGCGCTGGGGAGCCGTGCGGGGGCGCGCCCTG | | (SEQ ID NO:142) |
| Sbjct: | 1 | GTTCCCAGCAGCGTGGTCAGCAGCAGCAGCGCTGGGGAGCCGTGCGGGGGCGCGCCCTGG | 60 | (SEQ ID NO:143) |
| Query: | 1437 | CCAGAGTCGCAGCCGCAGCTGCCAGCCTCCTCGCACCGCAGCTTCTCGCAGAGGATGCCC | 1378 | |
| | | CCAGAGTCGGAGCCGCAGCTGCCAGCCTCCTCGCACCGCAGCTTCTCGCAGAGGATGCCC | | |
| Sbjct: | 61 | CCAGAGTCGGAGCCGCAGCTGCCAGCCTCCTCGCACCGCAGCTTCTCGCAGAGGATGCCC | 120 | |
| Query: | 1377 | GTGTATGCGGCCGGGCACAGGCAGCGCACGTTGTTGTGGCACGTCCCTCCGTTCTGGCAG | 1318 | |
| | | GTGTATGCGGCCGGGCACAGGCAGCGCACGTTGTTGTGGCACGTCCCTCCGTTCTGGCAG | | |
| Sbjct: | 121 | GTGTATGCGGCCGGGCACAGGCAGCGCACGTTGTTGTGGCACGTCCCTCCGTTCTGGCAG | 180 | |
| Query: | 1317 | TGCAGGAGCTCGTTGTCGCAGACATTCGAGGTATTTGCAG-TGCCTTTGGG-GATGGGGA | 1260 | |
| | | TGCAGGAGCTCGTTGTCGCAGACATTCG T T GCA T T T G GAT G G | | |
| Sbjct: | 181 | TGCAGGAGCTCGTTGTCGCAGACATTCGCT-TCCTCGCACCTTTATGTCTGAGATCGCGT | 239 | |
| Query: | 1259 | GATAGGAGCCTC 1248 | | |
| | | A AG AGC TG | | |
| Sbjct: | 240 | AAAAGTAGCCTC 251 | | |

>s3aq: 158627368, 570 bp.
Length = 570
Minus Strand HSPs:
Score = 1192 (178.8 bits), Expect = 2.2e−98, Sum P(2) = 2.2e−98
Identities = 242/245 (98%), Positives = 242/245 (98%), Strand = Minus/Plus

| | | | | |
|---|---|---|---|---|
| Query: | 1294 | ATTCGAGGTATTTGCAGTGCCTTTGGGGATGGGGAGATACGAGCCTCGACTCCAAGGTCG | 1235 | (SEQ ID NO:144) |
| | | AT C AGGTATTTGCAGTGCCTTTGGGGATGGGGAGATAGGAGCCTGGACTCCAAGGTCG | | (SEQ ID NO:145) |
| Sbjct: | 327 | ATAC-AGGTATTTGCAGTGCCTTTGGGGATGGGGAGATAGGAGCCTGCACTCCAAGGTCG | 385 | (SEQ ID NO:146) |
| Query: | 1234 | GCCCTGATAATTCTTCTTGCATTTCCCACAGTCTGGACCTGTACTGTTGTGCTCACATTC | 1175 | |
| | | GCCCTGATAATTCTTCTTGCATTTCCCACAGTCTGGACCTGTAGTGTTGTGCTCACATTC | | |
| Sbjct: | 386 | GCCCTGATAATTCTTCTTGCATTTCCCACAGTCTGGACCTGTAGTGTTGTGCTCACATTC | 445 | |
| Query: | 1174 | GCATGTCAATTTGCTGTTGTCATACACACATACAGTGCCATGGAGATTACACTTGCACCT | 1115 | |
| | | GCATGTCAATTTGCTGTTGTCATACACACATACACTGCCATGGACATTACACTTGCACCT | | |
| Sbjct: | 446 | GCATGTCAATTTGCTGTTGTCATACACACATACAGTGCCATGCAGATTACACTTGCACCT | 505 | |
| Query: | 1114 | TCCTCGCGCCTTTATGTCTGAGATCGCGTAAAAGTAGCGTCCCAAGTGTAGCTCATCTAC | 1055 | |
| | | TCCTCGC CCTTTATGTCTGAGATCGCGTAAAAGTAGCGTGCCAAGTGTAGCTCATCTAC | | |
| Sbjct: | 506 | TCCTCCCACCTTTATCTCTGAGATCGCGTAAAAGTAGCGTGCCAAGTGTAGCTCATCTAC | 565 | |
| Query: | 1054 | AAATA 1050 | | |
| | | AAATA | | |
| Sbjct: | 566 | AAATA 570 | | |

Score = 1154 (173.1 bits), Expect = 2.2e−98, Sum P(2) = 2.2e−98
Identities = 240/246 (97%), Positives = 240/246 (97%), Strand = Minus/Plus

| | | | | |
|---|---|---|---|---|
| Query: | 1526 | GTGACACCTAAAACACCAGGGGGCTGGCGGTTCCCAGCAGCGTGGTCAGCAGCAGCAGCG | 1467 | (SEQ ID NO:147) |
| | | GTGACACCTA AACACCAGGGG CTGGCGGTTCCCAGCAGCGTGGTCAGCAGCAGCAGCG | | (SEQ ID NO:148) |
| Sbjct: | 2 | GTGACACCTAGAACACCAGGGG-CTGGCGGTTCCCAGCAGCGPGGTCAGCAGCAGCAGCG | 60 | (SEQ ID NO:149) |
| Query: | 1466 | CTGGGGAGCCGTGCCGGGGCGCGCCCTGACCAGAGTCGGAGCCGCAGCTGCCAGCCTCCT | 1407 | |
| | | CTGCGGAGCCGTGCGGGGGCGCGCCCTG CCAGAGTCGGAGCCGCAGCTGCCAGCCTCCT | | |
| Sbjct: | 61 | CTGGGGAGCCGTGCGGGGGCGCGCCCTGGCCAGAGTCGGAGCCGCAGCTGCCAGCCTCCT | 120 | |
| Query: | 1406 | CGCACCGCAGCTTCTCGCAGAGGATGCCCGTGTATGCGGCCGGGCACAGGCAGCGCACGT | 1347 | |
| | | CGCACCGCAGCTTCTCGCAGAGGATGCCCGTGTATGCGGCCGGGCACAGGCAGCGCACGT | | |
| Sbjct: | 121 | CGCACCGCAGCTTCTCGCACAGGATGCCCGTGTATGCGGCCGGGCACAGGCAGCGCACGT | 180 | |
| Query: | 1346 | TGTTGTGGCACGTCCCTCCGTTCTGGCAGTGCAGGAGCTCGTTGTCGCAGACATTCGAG- | 1288 | |
| | | TGTTGTGGCACGTCCCTCCGTTCTGGCAGTGCAGGAGCTCGTTGTCGCAGACATTCG | | |
| Sbjct: | 181 | TGTTGTGGCACGTCCCTCCGTTCTGGCAGTGCAGGAGCTCGTTGTCGCAGACATTCGCTT | 240 | |
| Query: | 1287 | GTATTTG 1281 | | |
| | | GT TTTG | | |
| Sbjct: | 241 | GT-TTTG 246 | | |

>s3aq: 158627375, 592 bp.
Length = 592
Minus Strand HSPs:
Score = 1160 (174.0 bits), Expect = 8.8e−88, Sum P(2) = 8.8e−88
Identities = 242/253 (95%), Positives = 242/253 (95%), Strand Minus/Plus

| | | | | |
|---|---|---|---|---|
| Query: | 1526 | GTGACACCTAAAACACCAGGGGGCTGCCGGTTCCCAGCAGCGTGGTCAGCAGCAGCAGCG | 1467 | (SEQ ID NO:150) |
| | | GTGACACCTA AACACCAGGGCGCTGGCCGTTCCCAGCAGCGTGGTC GCAGCAGCAGCG | | (SEQ ID NO:151) |
| Sbjct: | 2 | GTGACACCTACAACACCACGGGGCTGCCGGTTCCCAGCAGCGTGGTCGGCAGCACCAGCG | 61 | (SEQ ID NO:152) |
| Query: | 1466 | CTGGGGAGCCGTGCGGGGGCGCGCCCTGACCAGAGTCGCAGCCGCAGCTGCCAGCCTCCT | 1407 | |
| | | CTGGGGAGCCGTGCGGGGGCGCGCCCTG CCAGAGTCGGAGCCGCAGCTGCCAGCCTCCT | | |
| Sbjct: | 62 | CTGCGGAGCCGTGCGGGGCCGCGCCCTGGCCACAGTCGGAGCCGCAGCTGCCAGCCTCCT | 121 | |
| Query: | 1406 | CGCACCGCAGCTTCTCGCAGAGGATCCCCGTCTATGCGGCCGGGCACAGGCAGCGCACGT | 1347 | |
| | | CGCACCGCAGCTTCTCGCAGAGGATGCCCGTGTATGCGGCCCGGCACAGGCAGCGCACGT | | |
| Sbjct: | 122 | CGCACCGCAGCTTCTCCCAGAGGATGCCCGTCTATGCGGCCCGGCACAGGCAGCGCACGT | 181 | |
| Query: | 1346 | TGTTGTGGCACGTCCCTCCGTTCTGGCAGTGCACGAGCTCGTTGTCGCAGACATTCGAGC | 1287 | |
| | | TGTTGTGGCACGTCCCTCCGTTCTGGCAGTGCAGGAGCTCGTTGTCGCAGACATTCG | | |
| Sbjct: | 182 | TGTTGTGGCACGTCCCTCCGTTCTCGCAGTGCAGGACCTCGTTGTCGCAGACATTCGCTC | 241 | |
| Query: | 1286 | TATT-TGCAGTGC 1275 | | |
| | | T TT TG TGC | | |
| Sbjct: | 242 | TCTTGTGGTTTGC 254 | | |

Score = 950 (142.5 bits), Expect = 8.8e−88, Sum P(2) = 8.8e−88
Identities = 200/208 (96%), Positives = 200/208 (96%), Strand = Minus/Plus

| | | | | |
|---|---|---|---|---|
| Query: | 1294 | ATTCGAGGTATTTGCAGTGCCTTTCGGGATCGGCACATAGGAGCCTGGACTCCAAGGTCG | 1235 | (SEQ ID NO:153) |
| | | AT C AGGTATTTGCAGTGCCTTTGGGGATGCGGAGATAGGAGCCTGGACTCCAAGGTCG | | (SEQ ID NO:154) |
| Sbjct: | 388 | ATAC-AGGTATTTGCAGTGCCTTTGGGATGGGGAGATAGGAGCCTGGACTCCAAGGTCG | 446 | (SEQ ID NO:155) |
| Query: | 1234 | GCCCTGATAATTCTTCTTGCATTTCCCACAGTCTGGACCTGTAGTGTTGTGCTCACATTC | 1175 | |
| | | GCCCTGATAATTCTTCTT CATTTCCCACAGTCTGGACC GTAGTGTTGTGCTCACATTC | | |
| Sbjct: | 447 | GCCCTGATAATTCTTCTTACATTTCCCACAGTCTGGACCCGTAGTGTTGTGCTCACATTC | 506 | |
| Query: | 1174 | GCATGTCAATTTGCTGTTGTCATACACACATACAGTGGCATGGAGATTACACTTGCACCT | 1115 | |
| | | GCATGTCAATTTGCTGTTGTCATACACACATACAGTGGCATGGAGATTACACT GCACCT | | |
| Sbjct: | 507 | GCATGTCAATTTGCTGTTGTCATACACACATACAGTGGCATGGAGATTACACT-GCACCT | 565 | |
| Query: | 1114 | TCCTCGCGCCTTTATGTCTGAGATCGCG 1087 | | |
| | | TCCTCGC C TTTATGTCTGAGATCG G | | |
| Sbjct: | 566 | TCCTCGCAC-TTTATGTCTGAGATCGGG 592 | | |

>s3aq: 126401933 Category D:, 556 bp.
Length = 556
Plus Strand HSPs:
Score = 925 (138.8 bits), Expect = 2.8e−36, P = 2.8e−36
Identities = 185/185 (100%), Positives = 185/185 (100%), Strand = Plus/Plus

| | | | | |
|---|---|---|---|---|
| Query: | 1 | GGCTTCCACCAAAGTCCTCAATATACCTGAATACGCACAATATCTTAACTCTTCATATTT | 60 | (SEQ ID NO:156) |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| Sbjct: | 372 | GCCTTCCACCAAGTCCTCAATATACCTGAATACGCACAATATCTTAACTCTTCATATTT | 431 | (SEQ ID NO:157) |
| Query: | 61 | GGTTTTGCGATCTGCTTTGAGGTCCCATCTTCATTTAAAAAAAAATACAGAGACCTACCT | 120 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| Sbjct: | 432 | GGTTTTGGGATCTGCTTTGAGGTCCCATCTTCATTTAAAAAAAAATACAGACACCTACCT | 491 | |
| Query: | 121 | ACCCGTACGCATACATACATATGTGTATATATATGTAAACTAGACAAAGATCGCAGATCA | 180 | |
| | | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | | |
| Sbjct: | 492 | ACCCGTACGCATACATACATATGTGTATATATATCTAAACTAGACAAAGATCGCAGATCA | 551 | |
| Query: | 181 | TAAAG 185 | | |
| | | \|\|\|\|\| | | |
| Sbjct: | 552 | TAAAG 556 | | |

[image]

[image]

In the alignment shown above, black outlined amino acid residues indicate residues identically conserved between sequences (i.e., residues that may be required to preserve structural or functional properties); amino acid residues with a gray background are similar to one another between sequences, possessing comparable physical and/or chemical properties without altering protein structure or function (e.g. the group L, V, I, and M may be considered similar); and amino acid residues with a white background are neither conserved nor similar between sequences.

TABLE 30

```
PSORT, SignalP and hydropathy results
for CuraGen Acc. No. CG51051-03.
endoplasmic reticulum (membrane)—Certainty =
0.6400 (Affirmative) <succ>
                                  plasma membrane—Certainty =
0.4960 (Affirmative) <succ>
                                            Golgi body—Certainty =
0.1900 (Affirmative) <succ>
                    microbody (peroxisome)—Certainty =
0.1301 (Affirmative) <succ>
INTEGRAL Likelihood = −2.87 Transmembrane 413−429
(412−429)
Seems to be a Type II (Ncyt Cexo) membrane protein
Is the sequence a signal peptide?
```

| | | | | |
|---|---|---|---|---|
| # Measure | Position | Value | Cutoff | Conclusion |

| | | | | |
|---|---|---|---|---|
| max. C | 19 | 0.369 | 0.37 | NO |
| max. Y | 19 | 0.507 | 0.34 | YES |
| max. S | 3 | 0.943 | 0.88 | YES |
| mean S | 1-18 | 0.870 | 0.48 | YES |

# Most likely cleavage site between pos. 18 and 19: VSS-VM

### Example 13

CG51051-07, Netrin/Laminin-Like Protein; Assembly: 228549906.

Example 13 shares the same background information as found for Example 12.

TABLE 31
Nucleotide Sequence for CG51051-07: FL_480

(SEQ ID NO:54)

>C051051-07
CAAGCTCTGCTTTAGTTTCCAAGAAGATTACAAAGAATTTAGAGATGTATTTGTCAAGATTCCTGTCGAT
TCATGCCCTTTGGGTTACGCTGTCCTCAGTGATGCAGCCCTACCCTTTGGTTTGGGGACATTATGATTTG
TGTAAGACTCAGATTTACACGGAAGAAGGGAAAGTTTGGGATTACATGCCCTGCCAGCCGGAATCCACGG
ACATGACAAAATATCTCAAAGTGAAACTCGATCCTCCGGATATTACCTCTGGAGACCCPCCTGAGACGTT
CTGTGCAATGGGCAATCCCTACATGTGCAATAATGACTGTGATGCGAGTACCCCTGAGCTGGCACACCCC
CCTGAGCTGATGTTTGATTTTGAAGGAAGACATCCCTCCACATTTTGGCACTCTGCCACTTGGAAGCAGT
ATCCCAACCCTCTCCAGCTTAACATCACTCTGTCTTGGAGCAAAACCATTCAGCTAACAGACAACATAGT
TATTACCTTTGAATCTGGCCGTCCAGACCAAATGATCCTGGAGAAGTCTCTCGATTATGGACGAACATCG
CAGCCCTATCACTATTATCCCACAGACTGCTTAGATGCTTTTCACATGCATCCTAAATCCGTGAAGGATT
TATCACAGCATACCGTCTTAGAAATCATTTGCACAGAAGAGTACTCAACAGGGTATACAACAAATAGCAA
AATAATCCACTTTGAAATCAAAGACAGGTTCGCGTTTTTTGCTGGACCTCGCCTACGCAATATGGCTTCC
CTCTACGGACAGCTGGATACAACCAAGAAACTCAGAGATTTCTTTACAGTCACAGACCTGAGGATAAGGC
TGTTAAGACCACCCGTTGGGGAAATATTTGTAGATGAGCTACACTTGGCACGCTACTTTTACGCGATCTC
AGACATAAAGGTGCGAGGAACCTGCAAGTGTAATCTCCATGCCACTGTATGTGTGTATGACAACAGCAAA
TTGACATGCGAATGTGAGCACAACACTACACGTCCAGACTGTGGGAAATGCAAGAAGAATTATCAGGGCC
GACCTTGGAGTCCACGCTCCTATCTCCCCATCCCCAAAGGCACTGCAAATACCTGTATCCCCAGTATTTC
CAGTATTGGTAATCCTCCAAAGTTTAATAGGATATGGCCGAATATTTCTTCCCTTGAGCTTTCTAACCCA
AAACAAGTTGCTCCCAAATTAGCTTTGTCAACACTTTCTTCTGTTCAAGTTGCAAACCACAAGAGAGCGA
ATGTCTGCGACAACGAGCTCCTGCACTGCCAGAACGGAGGGACGTGCCACAACAACGTGCGCTGCCTGTG
CCCGGCCGCATACACGGGCATCCTCTGCGAGAAGCTGCGGTGCGAGGAGGCTGGCAGCTGCGGCTCCGAC
TCTCGCCAGGGCGCGCCCCCGCACGGCTCCCCAGCGCTGCTGCTGCTGACCACGCTGCTGGGAACCGCCA
GCCCCCTGGTGTTCTAGGTGTCAC

The cDNA coding for a domain of CG51051-07 from residue 50 to 295 was targeted for “in-frame” cloning by PCR. The PCR template is based on the previously identified plasmid.

Two PCR reactions were set up using a total of 1-5 ng of the plasmid that contains the insert for CG51051-07.

The reaction mixtures contained 2 microliters of each of the primers (original concentration: 5 pmol/ul), 1 microliter of 10 mM dNTP (Clontech Laboratories, Palo Alto Calif.) and 1 microliter of 50× Advantage-HF 2 polymerase (Clontech Laboratories) in 50 microliter-reaction volume. The following reaction conditions were used:

PCR condition 1:

| | | |
|---|---|---|
| a) | 96° C. | 3 minutes |
| b) | 96° C. | 30 seconds denaturation |
| c) | 60° C. | 30 seconds, primer annealing |
| d) | 72° C. | 6 minutes extension |

Repeat steps b-d 15 times

| | | |
|---|---|---|
| e) | 96° C. | 15 seconds denaturation |
| f) | 60° C. | 30 seconds, primer annealing |
| g) | 72° C. | 6 minutes extension |

Repeat steps e-g 29 times

| | | |
|---|---|---|
| e) | 72° C. | 10 minutes final extension |

PCR condition 2:

| | | |
|---|---|---|
| a) | 96° C. | 3 minutes |
| b) | 96° C. | 15 seconds denaturation |
| c) | 76° C. | 30 seconds, reducing the temperature by 1° C. per cycle |
| d) | 72° C. | 4 minutes extension |

Repeat steps b-d 34 times

| | | |
|---|---|---|
| e) | 72° C. | 10 minutes final extension. |

An amplified product was detected by agarose gel electrophoresis. The fragment was gel-purified and ligated into the pCR2.1 vector (Invitrogen, Carlsbad, Calif.) following the manufacturer's recommendation. Twelve clones per PCR reaction were picked and sequenced. The inserts were sequenced using vector-specific M13 Forward and M13 Reverse primers and the following gene-specific primers:

| | | |
|---|---|---|
| SF1: | TCTGTCTTGGAGCAAAACCATTGAG | (SEQ ID NO:159) |
| SF2: | CGGTCTTAGAAATCATTTCCACAGAAGAG | (SEQ ID NO:160) |
| SR1: | AGTGATGTTAACCTGGAGAGGCTTG | (SEQ ID NO:161) |
| SR2: | CGTATGCTGTGATAAATCCTTCACG | (SEQ ID NO:162) |

Results:

The insert assembly 228549906 was found to encode an open reading frame between residues 50 and 295 of the target sequence CG51051-07. The cloned insert is 100% identical to the original sequence. The alignment with CG51051-07 is displayed in a ClustalW below. Note that differing amino acids have a white or grey background, and deleted/inserted amino acids can be detected by a dashed line in the sequence that does not code at that position.

[image]

TABLE 33

DNA Sequence Analysis, Including Protein Sequence,
for Assembly No. 228549906
(SEQ ID NO:163—Nucleotide; SEQ ID NO:164—Protein Sequence)
View DNA Sequence Analysis of 228549906
Translated Protein - Frame: - 2 - Nucleotide 2 to 778

The protein associated with 228549906 is encoded in a negative reading frame. The
sequence shown below has been reverse-complemented and renumbered to allow reading
of the protein in the expected N to C direction.
Printed 60 characters to a line.

| | |
|---|---|
| 1 | AGGCTCCGCGGCCGCCCCCTTCACCTGCCAGCCGGAATCCACGGACATGACAAAATATCTGAAAGTGAAACTCGATCCTC |
| | G S A A A P F T C Q P E S T D M T K Y L K V K L D P P |
| 81 | CGGATATTACCTGTGGAGACCCTCCTGAGACGTTCTGTGCAATGGGCAATCCCTACATGTGCAATAATGAGTGTGATGCG |
| | D I T C G D P P E T F C A M G N P Y M C N N E C D A |
| 161 | AGTACCCCTGAGCTGGCACACCCCCCTGAGCTGATGTTTGATTTTGAAGGAAGACATCCCTCCACATTTTGGCAGTCGC |
| | S T P E L A H P P E L M F D F E G R H P S T F W Q S A |
| 241 | CACTTGGAAGGAGTATCCCAAGCCTCTCCAGGTTAACATCACTCTGTCTTGGAGCAAAACCATTGAGCTAACAGACAACA |
| | T W K E Y P K P L Q V N I T L S W S K T I E L T D N I |
| 321 | TAGTTATTACCTTTGAATCTGGGCGTCCAGACCAAATGATCCTGGAGAAGTCTCTCGATTATGGACGAACATGGCAGCCC |
| | V I T F E S G R P D Q M I L E K S L D Y G R T W Q P |
| 401 | TATCAGTATTATGCCACAGACTGCTTAGATGCTTTTCACATGGATCCTAAATCCGTGAAGGATTTATCACAGCATACGGT |
| | Y Q Y Y A T D C L D A FH M D P K S V K D L S Q H T V |
| 481 | CTTAGAAATCATTTGCACAGAAGAGTACTCAACAGGGTATACAACAAATAGCAAAATAATCCACTTTGAAATCAAAGACA |
| | L E I I C T E E Y S T G Y T T N S K I I H F E I K D R |
| 561 | GGTTCGCGTTTTTTGCTGGACCTCGCCTACGCAATATGGCTTCCCTCTACGGACAGCTGGATACAACCAAGAAACTAGA |
| | F A F F A G P R L R N M A S L Y G Q L D T T K K L R |
| 641 | GATTTCTTTACAGTCACAGACCTGAGGATAAGGCTGTTAAGACCAGCCGTTGGGGAAATATTTGTACATGAGCTACACTT |
| | D F F T V T D L R I R L L RP A V G E I F V D E L H L |
| 721 | GGCACGCTACTTTTACGCGATCTCAGACATAAAGGTGCGAGGAAAGGGTGGGCGCGCC |
| | A R Y F Y A I S D I K V R G K G G R A |

TABLE 34
Nucleotide Sequence for Assembly No. 228549906.

(SEQ ID NO:165)

>228549906
GGCGCGCCCACCCTTTCCTCGCACCTTTATGTCTGAGATCGCGTAAAAGTAGCGTGCCAAGTGTAGCTCATCTACAAAT
ATTTCCCCAACGGCTGGTCTTAACAGCCTTATCCTCAGGTCTGTGACTGTAAAGAAATCTCTGAGTTTCTTGGTTGTATC
CAGCTGTCCGTAGAGGGAAGCCATATTGCGTAGGCGAGGTCCAGCAAAAAACGCGAACCTGTCTTTGATTTCAAAGTG
GATTATTTTGCTATTTGTTGTATACCCTGTTGAGTACTCTTCTGTGCAAATGATTTCTAAGACCGTATGCTGTGATAAATC
CTTCACGGATTTAGGATCCATGTGAAAAGCATCTAAGCAGTCTGTGGCATAATACTGATAGGGCTGCCATGTTCGTCCA
TAATCGAGAGACTTCTCCAGGATCATTTGGTCTGGACGCCCAGATTCAAAGGTAATAACTATGTTGTCTGTTAGCTCAA
TGGTFJTGCTCCAAGACAGAGTGATG1TAACCTGGAGAGGCYJGGGATACTCCTFCCAAGTGGCAGACTGCCAAAATGT
GGAGGGATGTCTTCCTTCAAAATCAAACATCAGCTCAGGGGGGTGTGCCAGCTCACGGGTACTCGCATCACACTCATTA
TTGCACATGTAGGGATTGCCCATTGCACAGAACGTCTCAGGAGGGTCTCCACAGGTAATATCCGGAGGATCGAGTTTCA
CTTTCAGATATTTTGTCATGTCCGTGGATTCCGGCTGGCAGGTGAAGGGGGCGGCCGCGGAGCCT

### Example 14

RTQ-PCR Quantitative Expression Analysis for CG53018-01, Also Known As No. 8484782 and TEN-M3-Like Protein.

Homology

AA Identity: extension of ODZ3, KIAA1455 and Hypothetical protein FLJ10474; 99% murine Ten-m3

Protein Size 1249 aa

InterPro Domains

7 EGF-Like Domain

NUL repeat (Peptidyl-alpha-hydroxyglycine alpha-amidating lyase (PAL) activity)

Glycosyl hydrolases family 38

Plexin repeat

Trypsin inhibitor like cysteine rich domain

Predicted Subcellular Location

Plasma Membrane, Type I Membrane Protein

Many ESTs have been isolated from lung tumors, some from kidney tumors. Indication of onco-fetal pattern (fetal lung, whole embryo). The taqman analysis support this hypothesis, strong expression in Kidney and lung tumor and tumor cell lines. Also in brain tumor cell lines and bladder and gastric tumors. Overall up-regulation in metastasis

Potential Role(s) of CG53018-01 (SC8484782) in Tumorgenesis:

cell migration and invasion, metastatic potential.

Impact of Therapeutic Targeting of CG53018-01 (SC8484782):

Therapeutic targeting of CG53018-01 (SC8484782) with a monoclonal antibody is anticipated to limit or block the extent of tumor cell migration and invasion and tumor metastasis, preferably in kidney and lung bladder and gastric tumors.

[image]

[image]

[image]

[image]

### Example 11

CG107126-01 Type IIIa Membrane Protein-Like Proteins

The quantitative expression of various clones was assessed using microtiter plates containing RNA samples from a variety of normal and pathology-derived cells, cell lines and tissues using real time quantitative PCR (RTQ PCR). RTQ PCR was performed on an Applied Biosystems ABI PRISM® 7700 or an ABI PRISM® 7900 HT Sequence Detection System. Various collections of samples are assembled on the plates, and referred to as Panel 1 (containing normal tissues and cancer cell lines), Panel 2 (containing samples derived from tissues from normal and cancer sources), Panel 3 (containing cancer cell lines), Panel 4 (containing cells and cell lines from normal tissues and cells related to inflammatory conditions), Panel 5D/51 (containing human tissues and cell lines with an emphasis on metabolic diseases), AI_comprehensive_panel (containing normal tissue and samples from autoimmune diseases), Panel CNSD.01 (containing central nervous system samples from normal and diseased brains) and CNS_neurodegeneration_panel (containing samples from normal and Alzheimer's diseased brains).

RNA integrity from all samples is controlled for quality by visual assessment of agarose gel electropherograms using 28S and 18S ribosomal RNA staining intensity ratio as a guide (2:1 to 2.5:1 28s:18s) and the absence of low molecular weight RNAs that would be indicative of degradation products. Samples are controlled against genomic DNA contamination by RTQ PCR reactions run in the absence of reverse transcriptase using probe and primer sets designed to amplify across the span of a single exon.

First, the RNA samples were normalized to reference nucleic acids such as constitutively expressed genes (for example, β-actin and GAPDH). Normalized RNA (5 ul) was converted to cDNA and analyzed by RTQ-PCR using One Step RT-PCR Master Mix Reagents (Applied Biosystems; Catalog No. 4309169) and gene-specific primers according to the manufacturer's instructions.

In other cases, non-normalized RNA samples were converted to single strand cDNA (sscDNA) using Superscript II (Invitrogen Corporation; Catalog No. 18064-147) and random hexamers according to the manufacturer's instructions. Reactions containing up to 10 μg of total RNA were performed in a volume of 20 μl and incubated for 60 minutes at 42° C. This reaction can be scaled up to 50 μg of total RNA in a final volume of 100 μl. sscDNA samples are then normalized to reference nucleic acids as described previously, using 1× TaqMan® Universal Master mix (Applied Biosystems; catalog No. 4324020), following the manufacturer's instructions.

Probes and primers were designed for each assay according to Applied Biosystems Primer Express Software package (version I for Apple Computer's Macintosh Power PC) or a similar algorithm using the target sequence as input. Default settings were used for reaction conditions and the following parameters were set before selecting primers: primer concentration=250 nM, primer melting temperature (Tm) range=58°-60° C., primer optimal Tm=59° C., maximum primer difference=2° C., probe does not have 5′G, probe Tm must be 10° C. greater than primer Tm, amplicon size 75 bp to 100 bp. The probes and primers selected (see below) were synthesized by Synthegen (Houston, Tex., USA). Probes were double purified by HPLC to remove uncoupled dye and evaluated by mass spectroscopy to verify coupling of reporter and quencher dyes to the 5′ and 3′ ends of the probe, respectively. Their final concentrations were: forward and reverse primers, 900 nM each, and probe, 200 nM.

PCR conditions: When working with RNA samples, normalized RNA from each tissue and each cell line was spotted in each well of either a 96 well or a 384-well PCR plate (Applied Biosystems). PCR cocktails included either a single gene specific probe and primers set, or two multiplexed probe and primers sets (a set specific for the target clone and another gene-specific set multiplexed with the target probe). PCR reactions were set up using TaqMan® One-Step RT-PCR Master Mix (Applied Biosystems, Catalog No. 4313803) following manufacturer's instructions. Reverse transcription was performed at 48° C. for 30 minutes followed by amplification/PCR cycles as follows: 95° C. 10 min, then 40 cycles of 95° C. for 15 seconds, 60° C. for 1 minute. Results were recorded as CT values (cycle at which a given sample crosses a threshold level of fluorescence) using a log scale, with the difference in RNA concentration between a given sample and the sample with the lowest CT value being represented as 2 to the power of delta CT. The percent relative expression is then obtained by taking the reciprocal of this RNA difference and multiplying by 100.

When working with sscDNA samples, normalized sscDNA was used as described previously for RNA samples. PCR reactions containing one or two sets of probe and primers were set up as described previously, using 1× TaqMan® Universal Master mix (Applied Biosystems; catalog No. 4324020), following the manufacturer's instructions. PCR amplification was performed as follows: 95° C. 10 min, then 40 cycles of 95° C. for 15 seconds, 60° C. for 1 minute. Results were analyzed and processed as described previously.

Panels 1, 1.1, 1.2, and 1.3D

The plates for Panels 1, 1.1, 1.2 and 1.3D include 2 control wells (genomic DNA control and chemistry control) and 94 wells containing cDNA from various samples. The samples in these panels are broken into 2 classes: samples derived from cultured cell lines and samples derived from primary normal tissues. The cell lines are derived from cancers of the following types: lung cancer, breast cancer, melanoma, colon cancer, prostate cancer, CNS cancer, squamous cell carcinoma, ovarian cancer, liver cancer, renal cancer, gastric cancer and pancreatic cancer. Cell lines used in these panels are widely available through the American Type Culture Collection (ATCC), a repository for cultured cell lines, and were cultured using the conditions recommended by the ATCC. The normal tissues found on these panels are comprised of samples derived from all major organ systems from single adult individuals or fetuses. These samples are derived from the following organs: adult skeletal muscle, fetal skeletal muscle, adult heart, fetal heart, adult kidney, fetal kidney, adult liver, fetal liver, adult lung, fetal lung, various regions of the brain, the spleen, bone marrow, lymph node, pancreas, salivary gland, pituitary gland, adrenal gland, spinal cord, thymus, stomach, small intestine, colon, bladder, trachea, breast, ovary, uterus, placenta, prostate, testis and adipose.

In the results for Panels 1, 1.1, 1.2 and 1.3D, the following abbreviations are used:

ca.=carcinoma,

*=established from metastasis,

met=metastasis,

s cell var=small cell variant,

non-s=non-sm=non-small,

squam=squamous,

pl. eff=pl effusion=pleural effusion,

glio=glioma,

astro=astrocytoma, and

neuro=neuroblastoma.

General_Screening_Panel_v1.4

The plates for Panel 1.4 include 2 control wells (genomic DNA control and chemistry control) and 94 wells containing cDNA from various samples. The samples in Panel 1.4 are broken into 2 classes: samples derived from cultured cell lines and samples derived from primary normal tissues. The cell lines are derived from cancers of the following types: lung cancer, breast cancer, melanoma, colon cancer, prostate cancer, CNS cancer, squamous cell carcinoma, ovarian cancer, liver cancer, renal cancer, gastric cancer and pancreatic cancer. Cell lines used in Panel 1.4 are widely available through the American Type Culture Collection (ATCC), a repository for cultured cell lines, and were cultured using the conditions recommended by the ATCC. The normal tissues found on Panel 1.4 are comprised of pools of samples derived from all major organ systems from 2 to 5 different adult individuals or fetuses. These samples are derived from the following organs: adult skeletal muscle, fetal skeletal muscle, adult heart, fetal heart, adult kidney, fetal kidney, adult liver, fetal liver, adult lung, fetal lung, various regions of the brain, the spleen, bone marrow, lymph node, pancreas, salivary gland, pituitary gland, adrenal gland, spinal cord, thymus, stomach, small intestine, colon, bladder, trachea, breast, ovary, uterus, placenta, prostate, testis and adipose. Abbreviations are as described for Panels 1, 1.1, 1.2, and 1.3D.

Panels 2D and 2.2

The plates for Panels 2D and 2.2 generally include 2 control wells and 94 test samples composed of RNA or cDNA isolated from human tissue procured by surgeons working in close cooperation with the National Cancer Institute's Cooperative Human Tissue Network (CHTN) or the National Disease Research Initiative (NDRI). The tissues are derived from human malignancies and in cases where indicated many malignant tissues have “matched margins” obtained from noncancerous tissue just adjacent to the tumor. These are termed normal adjacent tissues and are denoted “NAT” in the results below. The tumor tissue and the “matched margins” are evaluated by two independent pathologists (the surgical pathologists and again by a pathologist at NDRI or CHTN). This analysis provides a gross histopathological assessment of tumor differentiation grade. Moreover, most samples include the original surgical pathology report that provides information regarding the clinical stage of the patient. These matched margins are taken from the tissue surrounding (i.e. immediately proximal) to the zone of surgery (designated “NAT”, for normal adjacent tissue, in Table RR). In addition, RNA and cDNA samples were obtained from various human tissues derived from autopsies performed on elderly people or sudden death victims (accidents, etc.). These tissues were ascertained to be free of disease and were purchased from various commercial sources such as Clontech (Palo Alto, Calif.), Research Genetics, and Invitrogen.

Panel 3D

The plates of Panel 3D are comprised of 94 cDNA samples and two control samples. Specifically, 92 of these samples are derived from cultured human cancer cell lines, 2 samples of human primary cerebellar tissue and 2 controls. The human cell lines are generally obtained from ATCC (American Type Culture Collection), NCI or the German tumor cell bank and fall into the following tissue groups: Squamous cell carcinoma of the tongue, breast cancer, prostate cancer, melanoma, epidermoid carcinoma, sarcomas, bladder carcinomas, pancreatic cancers, kidney cancers, leukemias/lymphomas, ovarian/uterine/cervical, gastric, colon, lung and CNS cancer cell lines. In addition, there are two independent samples of cerebellum. These cells are all cultured under standard recommended conditions and RNA extracted using the standard procedures. The cell lines in panel 3D and 1.3D are of the most common cell lines used in the scientific literature.

Panels 4D, 4R, and 4.1D

Panel 4 includes samples on a 96 well plate (2 control wells, 94 test samples) composed of RNA (Panel 4R) or cDNA (Panels 4D/4.1D) isolated from various human cell lines or tissues related to inflammatory conditions. Total RNA from control normal tissues such as colon and lung (Stratagene, La Jolla, Calif.) and thymus and kidney (Clontech) was employed. Total RNA from liver tissue from cirrhosis patients and kidney from lupus patients was obtained from BioChain (Biochain Institute, Inc., Hayward, Calif.). Intestinal tissue for RNA preparation from patients diagnosed as having Crohn's disease and ulcerative colitis was obtained from the National Disease Research Interchange (NDRI) (Philadelphia, Pa.).

Astrocytes, lung fibroblasts, dermal fibroblasts, coronary artery smooth muscle cells, small airway epithelium, bronchial epithelium, microvascular dermal endothelial cells, microvascular lung endothelial cells, human pulmonary aortic endothelial cells, human umbilical vein endothelial cells were all purchased from Clonetics (Walkersville, Md.) and grown in the media supplied for these cell types by Clonetics. These primary cell types were activated with various cytokines or combinations of cytokines for 6 and/or 12-14 hours, as indicated. The following cytokines were used; IL-1 beta at approximately 1-5 ng/ml, TNF alpha at approximately 5-10 ng/ml, IFN gamma at approximately 20-50 ng/ml, IL-4 at approximately 5-10 ng/ml, IL-9 at approximately 5-10 ng/ml, IL-13 at approximately 5-10 ng/ml. Endothelial cells were sometimes starved for various times by culture in the basal media from Clonetics with 0.1% serum.

Mononuclear cells were prepared from blood of employees at CuraGen Corporation, using Ficoll. LAK cells were prepared from these cells by culture in DMEM 5% FCS (Hyclone), 100 μM non essential amuno acids (Gibco/Life Technologies, Rockville, Md.), 1 mM sodium pyruvate (Gibco), mercaptoethanol 5.5×10⁻⁵M (Gibco), and 10 mM Hepes (Gibco) and Interleukin 2 for 4-6 days. Cells were then either activated with 10-20 ng/ml PMA and 1-21 g/ml ionomycin, IL-12 at 5-10 ng/ml, IFN gamma at 20-50 ng/ml and IL-18 at 5-10 ng/ml for 6 hours. In some cases, mononuclear cells were cultured for 4-5 days in DMEM 5% FCS (Hyclone), 100 μM non essential amino acids (Gibco), 1 mM sodium pyruvate (Gibco), mercaptoethanol 5.5×10⁻⁵M (Gibco), and 10 mM Hepes (Gibco) with PHA (phytohemagglutinin) or PWM (pokeweed mitogen) at approximately 5 μg/ml. Samples were taken at 24, 48 and 72 hours for RNA preparation. MLR (mixed lymphocyte reaction) samples were obtained by taking blood from two donors, isolating the mononuclear cells using Ficoll and mixing the isolated mononuclear cells 1:1 at a final concentration of approximately 2×10⁶cells/ml in DMEM 5% FCS (Hyclone), 100 μM non essential amino acids (Gibco), 1 mM sodium pyruvate (Gibco), mercaptoethanol (5.5×10⁻⁵M) (Gibco), and 10 mM Hepes (Gibco). The MLR was cultured and samples taken at various time points ranging from 1-7 days for RNA preparation.

Monocytes were isolated from mononuclear cells using CD14 Miltenyi Beads, +ve VS selection columns and a Vario Magnet according to the manufacturer's instructions. Monocytes were differentiated into dendritic cells by culture in DMEM 5% fetal calf serum (FCS) (Hyclone, Logan, Utah), 100 μM non essential amino acids (Gibco), 1 mM sodium pyruvate (Gibco), mercaptoethanol 5.5×10⁻⁵M (Gibco), and 10 mM Hepes (Gibco), 50 ng/ml GMCSF and 5 ng/ml IL-4 for 5-7 days. Macrophages were prepared by culture of monocytes for 5-7 days in DMEM 5% FCS (Hyclone), 100 μM non essential amino acids (Gibco), 1 mM sodium pyruvate (Gibco), mercaptoethanol 5.5×10⁻⁵M (Gibco), 10 mM Hepes (Gibco) and 10% AB Human Serum or MCSF at approximately 50 ng/ml. Monocytes, macrophages and dendritic cells were stimulated for 6 and 12-14 hours with lipopolysaccharide (LPS) at 10 ng/ml. Dendritic cells were also stimulated with anti-CD40 monoclonal antibody (Pharmingen) at 10 μg/ml for 6 and 12-14 hours.

CD4 lymphocytes, CD8 lymphocytes and NK cells were also isolated from mononuclear cells using CD4, CD8 and CD56 Miltenyi beads, positive VS selection columns and a Vario Magnet according to the manufacturer's instructions. CD45RA and CD45RO CD4 lymphocytes were isolated by depleting mononuclear cells of CD8, CD56, CD14 and CD19 cells using CD8, CD56, CD14 and CD19 Miltenyi beads and positive selection. CD45RO beads were then used to isolate the CD45RO CD4 lymphocytes with the remaining cells being CD45RA CD4 lymphocytes. CD45RA CD4, CD45RO CD4 and CD8 lymphocytes were placed in DMEM 5% FCS (Hyclone), 100 μM non essential amino acids (Gibco), 1 mM sodium pyruvate (Gibco), mercaptoethanol 5.5×10⁻⁵M (Gibco), and 10 mM Hepes (Gibco) and plated at 10⁶cells/ml onto Falcon 6 well tissue culture plates that had been coated overnight with 0.51 g/ml anti-CD28 (Pharmingen) and 3 ug/ml anti-CD3 (OKT3, ATCC) in PBS. After 6 and 24 hours, the cells were harvested for RNA preparation. To prepare chronically activated CD8 lymphocytes, we activated the isolated CD8 lymphocytes for 4 days on anti-CD28 and anti-CD3 coated plates and then harvested the cells and expanded them in DMEM 5% FCS (Hyclone), 100 μM non essential amino acids (Gibco), 1 mM sodium pyruvate (Gibco), mercaptoethanol 5.5×10⁻⁵M (Gibco), and 10 mM Hepes (Gibco) and IL-2. The expanded CD8 cells were then activated again with plate bound anti-CD3 and anti-CD28 for 4 days and expanded as before. RNA was isolated 6 and 24 hours after the second activation and after 4 days of the second expansion culture. The isolated NK cells were cultured in DMEM 5% FCS (Hyclone), 100 μM non essential amino acids (Gibco), 1 mM sodium pyruvate (Gibco), mercaptoethanol 5.5×10⁻⁵M (Gibco), and 10 mM Hepes (Gibco) and IL-2 for 4-6 days before RNA was prepared.

To obtain B cells, tonsils were procured from NDRI. The tonsil was cut up with sterile dissecting scissors and then passed through a sieve. Tonsil cells were then spun down and resupended at 10⁶cells/ml in DMEM 5% FCS (Hyclone), 100 μM non essential amino acids (Gibco), 1 mM sodium pyruvate (Gibco), mercaptoethanol 5.5×10⁻⁵M (Gibco), and 10 mM Hepes (Gibco). To activate the cells, we used PWM at 5 μg/ml or anti-CD40 (Pharmingen) at approximately 10 μg/ml and IL-4 at 5-10 ng/ml. Cells were harvested for RNA preparation at 24,48 and 72 hours.

To prepare the primary and secondary Th1/Th2 and Tr1 cells, six-well Falcon plates were coated overnight with 10 μg/ml anti-CD28 (Pharmingen) and 2 μg/ml OKT3 (ATCC), and then washed twice with PBS. Umbilical cord blood CD4 lymphocytes (Poietic Systems, German Town, Md.) were cultured at 10⁵-10⁶ cells/ml in DMEM 5% FCS (Hyclone), 100 μM non essential amino acids (Gibco), 1 mM sodium pyruvate (Gibco), mercaptoethanol 5.5×10⁵M (Gibco), 10 mM Hepes (Gibco) and IL-2 (4 ng/ml). IL-12 (5 ng/ml) and anti-IL4 (1 μg/ml) were used to direct to Th1, while IL-4 (5 ng/ml) and anti-IFN gamma (1 μg/ml) were used to direct to Th2 and IL-10 at 5 ng/ml was used to direct to Tr1. After 4-5 days, the activated Th1, Th2 and Tr1 lymphocytes were washed once in DMEM and expanded for 4-7 days in DMEM 5% FCS (Hyclone), 100 μM non essential amino acids (Gibco), 1 mM sodium pyruvate (Gibco), mercaptoethanol 5.5×10⁻⁵M (Gibco), 10 mM Hepes (Gibco) and IL-2 (1 ng/ml). Following this, the activated Th1, Th2 and Tr1 lymphocytes were re-stimulated for 5 days with anti-CD28/OKT3 and cytokines as described above, but with the addition of anti-CD95L (1 μg/ml) to prevent apoptosis. After 4-5 days, the Th1, Th2 and Tr1 lymphocytes were washed and then expanded again with IL-2 for 4-7 days. Activated Th1 and Th2 lymphocytes were maintained in this way for a maximum of three cycles. RNA was prepared from primary and secondary Th1, Th2 and Tr1 after 6 and 24 hours following the second and third activations with plate bound anti-CD3 and anti-CD28 mAbs and 4 days into the second and third expansion cultures in Interleukin 2.

The following leukocyte cells lines were obtained from the ATCC: Ramos, EOL-1, KU-812. EOL cells were further differentiated by culture in 0.1 mM dbcAMP at 5×10⁵cells/ml for 8 days, changing the media every 3 days and adjusting the cell concentration to 5×10⁵cells/ml. For the culture of these cells, we used DMEM or RPMI (as recommended by the ATCC), with the addition of 5% FCS (Hyclone), 100 μM non essential amino acids (Gibco), 1 mM sodium pyruvate (Gibco), mercaptoethanol 5.5×10⁻⁵M (Gibco), 10 mM Hepes (Gibco). RNA was either prepared from resting cells or cells activated with PMA at 10 ng/ml and ionomycin at 1 μg/ml for 6 and 14 hours. Keratinocyte line CCD106 and an airway epithelial tumor line NCI-H292 were also obtained from the ATCC. Both were cultured in DMEM 5% FCS (Hyclone), 100 μM non essential amino acids (Gibco), 1 mM sodium pyruvate (Gibco), mercaptoethanol 5.5×10⁻⁵M (Gibco), and 10 mM Hepes (Gibco). CCD1106 cells were activated for 6 and 14 hours with approximately 5 ng/ml TNF alpha and 1 ng/ml IL-1 beta, while NCI-H292 cells were activated for 6 and 14 hours with the following cytokines: 5 ng/ml IL-4, 5 ng/ml IL-9, 5 ng/ml IL-13 and 25 ng/ml IFN gamma.

For these cell lines and blood cells, RNA was prepared by lysing approximately 10⁷ cells/ml using Trizol (Gibco BRL). Briefly, 1/10 volume of bromochloropropane (Molecular Research Corporation) was added to the RNA sample, vortexed and after 10 minutes at room temperature, the tubes were spun at 14,000 rpm in a Sorvall SS34 rotor. The aqueous phase was removed and placed in a 15 ml Falcon Tube. An equal volume of isopropanol was added and left at −20° C. overnight. The precipitated RNA was spun down at 9,000 rpm for 15 min in a Sorvall SS34 rotor and washed in 70% ethanol. The pellet was redissolved in 300 μl of RNAse-free water and 35 μl buffer (Promega) 5 μl DTT, 7 μl RNAsin and 8 μl DNAse were added. The tube was incubated at 37° C. for 30 minutes to remove contaminating genomic DNA, extracted once with phenol chloroform and re-precipitated with 1/10 volume of 3M sodium acetate and 2 volumes of 100% ethanol. The RNA was spun down and placed in RNAse free water. RNA was stored at −80° C.

AI_Comprehensive_Panel_v1.0

The plates for AI_comprehensive_panel_v1.0 include two control wells and 89 test samples comprised of cDNA isolated from surgical and postmortem human tissues obtained from the Backus Hospital and Clinomics (Frederick, Md.). Total RNA was extracted from tissue samples from the Backus Hospital in the Facility at CuraGen. Total RNA from other tissues was obtained from Clinomics.

Joint tissues including synovial fluid, synovium, bone and cartilage were obtained from patients undergoing total knee or hip replacement surgery at the Backus Hospital. Tissue samples were immediately snap frozen in liquid nitrogen to ensure that isolated RNA was of optimal quality and not degraded. Additional samples of osteoarthritis and rheumatoid arthritis joint tissues were obtained from Clinomics. Normal control tissues were supplied by Clinomics and were obtained during autopsy of trauma victims.

Surgical specimens of psoriatic tissues and adjacent matched tissues were provided as total RNA by Clinomics. Two male and two female patients were selected between the ages of 25 and 47. None of the patients were taking prescription drugs at the time samples were isolated.

Surgical specimens of diseased colon from patients with ulcerative colitis and Crohns disease and adjacent matched tissues were obtained from Clinomics. Bowel tissue from three female and three male Crohn's patients between the ages of 41-69 were used. Two patients were not on prescription medication while the others were taking dexamethasone, phenobarbital, or tylenol. Ulcerative colitis tissue was from three male and four female patients. Four of the patients were taking lebvid and two were on phenobarbital.

Total RNA from post mortem lung tissue from trauma victims with no disease or with emphysema, asthma or COPD was purchased from Clinomics. Emphysema patients ranged in age from 40-70 and all were smokers, this age range was chosen to focus on patients with cigarette-linked emphysema and to avoid those patients with alpha-lanti-trypsin deficiencies. Asthma patients ranged in age from 36-75, and excluded smokers to prevent those patients that could also have COPD. COPD patients ranged in age from 35-80 and included both smokers and non-smokers. Most patients were taking corticosteroids, and bronchodilators.

In the labels employed to identify tissues in the AI_comprehensive_panel_v1.0 panel, the following abbreviations are used:

AI=Autoimmunity

Syn=Synovial

Normal=No apparent disease

Rep22 /Rep20=individual patients

RA=Rheumatoid arthritis

Backus=From Backus Hospital

OA=Osteoarthritis

(SS) (BA) (MF)=Individual patients

Adj=Adjacent tissue

Match control=adjacent tissues

-M=Male

-F=Female

COPD=Chronic obstructive pulmonary disease

Panels 5D and 5I

The plates for Panel 5D and 5I include two control wells and a variety of cDNAs isolated from human tissues and cell lines with an emphasis on metabolic diseases. Metabolic tissues were obtained from patients enrolled in the Gestational Diabetes study. Cells were obtained during different stages in the differentiation of adipocytes from human mesenchymal stem cells. Human pancreatic islets were also obtained.

In the Gestational Diabetes study subjects are young (18-40 years), otherwise healthy women with and without gestational diabetes undergoing routine (elective) Caesarean section. After delivery of the infant, when the surgical incisions were being repaired/closed, the obstetrician removed a small sample (<1 cc) of the exposed metabolic tissues during the closure of each surgical level. The biopsy material was rinsed in sterile saline, blotted and fast frozen within 5 minutes from the time of removal. The tissue was then flash frozen in liquid nitrogen and stored, individually, in sterile screw-top tubes and kept on dry ice for shipment to or to be picked up by CuraGen. The metabolic tissues of interest include uterine wall (smooth muscle), visceral adipose, skeletal muscle (rectus) and subcutaneous adipose. Patient descriptions are as follows:

Patient 2: Diabetic Hispanic, overweight, not on insulin

Patient 7-9: Nondiabetic Caucasian and obese (BMI>30)

Patient 10: Diabetic Hispanic, overweight, on insulin

Patient 11: Nondiabetic African American and overweight

Patient 12: Diabetic Hispanic on insulin

Adipocyte differentiation was induced in donor progenitor cells obtained from Osirus (a division of Clonetics/BioWhittaker) in triplicate, except for Donor 3U which had only two replicates. Scientists at Clonetics isolated, grew and differentiated human mesenchymal stem cells (HuMSCs) for CuraGen based on the published protocol found in Mark F. Pittenger, et al., Multilineage Potential of Adult Human Mesenchymal Stem Cells Science Apr 2 1999: 143-147. Clonetics provided Trizol lysates or frozen pellets suitable for mRNA isolation and ds cDNA production. A general description of each donor is as follows:

Donor 2 and 3 U: Mesenchymal Stem cells, Undifferentiated Adipose

Donor 2 and 3 AM: Adipose, AdiposeMidway Differentiated

Donor 2 and 3 AD: Adipose, Adipose Differentiated

Human cell lines were generally obtained from ATCC (American Type Culture Collection), NCI or the German tumor cell bank and fall into the following tissue groups: kidney proximal convoluted tubule, uterine smooth muscle cells, small intestine, liver HepG2 cancer cells, heart primary stromal cells, and adrenal cortical adenoma cells.

These cells are all cultured under standard recommended conditions and RNA extracted using the standard procedures. All samples were processed at CuraGen to produce single stranded cDNA.

Panel 5I contains all samples previously described with the addition of pancreatic islets from a 58 year old female patient obtained from the Diabetes Research Institute at the University of Miami School of Medicine. Islet tissue was processed to total RNA at an outside source and delivered to CuraGen for addition to panel 5I.

In the labels employed to identify tissues in the 5D and 5I panels, the following abbreviations are used:

GO Adipose=Greater Omentum Adipose

SK=Skeletal Muscle

UT=Uterus

PL=Placenta

AD=Adipose Differentiated

AM=Adipose Midway Differentiated

U=Undifferentiated Stem Cells

Panel CNSD.01

The plates for Panel CNSD.01 include two control wells and 94 test samples comprised of cDNA isolated from postmortem human brain tissue obtained from the Harvard Brain Tissue Resource Center. Brains are removed from calvaria of donors between 4 and 24 hours after death, sectioned by neuroanatomists, and frozen at −80° C. in liquid nitrogen vapor. All brains are sectioned and examined by neuropathologists to confirm diagnoses with clear associated neuropathology.

Disease diagnoses are taken from patient records. The panel contains two brains from each of the following diagnoses: Alzheimer's disease, Parkinson's disease, Huntington's disease, Progressive Supernuclear Palsy, Depression, and “Normal controls”. Within each of these brains, the following regions are represented: cingulate gyrus, temporal pole, globus palladus, substantia nigra, Brodman Area 4 (primary motor strip), Brodman Area 7 (parietal cortex), Brodman Area 9 (prefrontal cortex), and Brodman area 17 (occipital cortex). Not all brain regions are represented in all cases; e.g., Huntington's disease is characterized in part by neurodegeneration in the globus palladus, thus this region is impossible to obtain from confirmed Huntington's cases. Likewise Parkinson's disease is characterized by degeneration of the substantia nigra making this region more difficult to obtain. Normal control brains were examined for neuropathology and found to be free of any pathology consistent with neurodegeneration.

In the labels employed to identify tissues in the CNS panel, the following abbreviations are used:

PSP=Progressive supranuclear palsy

Sub Nigra=Substantia nigra

Glob Palladus=Globus palladus

Temp Pole=Temporal pole

Cing Gyr=Cingulate gyrus

BA 4=Brodman Area 4

Panel CNS_Neurodegeneration_V1.0

The plates for Panel CNS_Neurodegeneration_V1.0 include two control wells and 47 test samples comprised of cDNA isolated from postmortem human brain tissue obtained from the Harvard Brain Tissue Resource Center (McLean Hospital) and the Human Brain and Spinal Fluid Resource Center (VA Greater Los Angeles Healthcare System). Brains are removed from calvaria of donors between 4 and 24 hours after death, sectioned by neuroanatomists, and frozen at −80° C. in liquid nitrogen vapor. All brains are sectioned and examined by neuropathologists to confirm diagnoses with clear associated neuropathology.

Disease diagnoses are taken from patient records. The panel contains six brains from Alzheimer's disease (AD) patients, and eight brains from “Normal controls” who showed no evidence of dementia prior to death. The eight normal control brains are divided into two categories: Controls with no dementia and no Alzheimer's like pathology (Controls) and controls with no dementia but evidence of severe Alzheimer's like pathology, (specifically senile plaque load rated as level 3 on a scale of 0-3; 0=no evidence of plaques, 3=severe AD senile plaque load). Within each of these brains, the following regions are represented: hippocampus, temporal cortex (Brodman Area 21), parietal cortex (Brodman area 7), and occipital cortex (Brodman area 17). These regions were chosen to encompass all levels of neurodegeneration in AD. The hippocampus is a region of early and severe neuronal loss in AD; the temporal cortex is known to show neurodegeneration in AD after the hippocampus; the parietal cortex shows moderate neuronal death in the late stages of the disease; the occipital cortex is spared in AD and therefore acts as a “control” region within AD patients. Not all brain regions are represented in all cases.

In the labels employed to identify tissues in the CNS_Neurodegeneration_V1.0 panel, the following abbreviations are used:

AD=Alzheimer's disease brain; patient was demented and showed AD-like pathology upon autopsy

Control=Control brains; patient not demented, showing no neuropathology

Control (Path)=Control brains; pateint not demented but showing sever AD-like pathology

SupTemporal Ctx=Superior Temporal Cortex

Inf Temporal Ctx=Inferior Temporal Cortex

A. CG53018-01/8484782: TEN-M3-Like Protein

Expression of gene CG53018-01 was assessed using the primer-probe sets Ag018b, Ag18, Ag690 and Ag2820, described in Tables AA, AB, AC and AD. Results of the RTQ-PCR runs are shown in Tables AE, AF, AG, AH, AI, and AJ.

TABLE AA

Probe Name Ag018b

| | | | | |
|---|---|---|---|---|
| Primers | Sequences | Length | Start Position | SEQ ID NO. |

| | | | | |
|---|---|---|---|---|
| Forward | 5′-atacccctgcttggcaactct-3′ | 21 | 670 | 166 |
| Probe | TET-5′-ctctctcaattttgcggcagtttgctcat-3′-TAMRA | 29 | 692 | 167 |
| Reverse | 5′-gagcaggaaatcaaaaaggtgtg-3′ | 23 | 723 | 168 |

TABLE AB

Probe Name Ag18

| | | | | |
|---|---|---|---|---|
| Primers | Sequences | Length | Start Position | SEQ ID NO. |

| | | | | |
|---|---|---|---|---|
| Forward | 5′-acccgctgtgtttgctgac-3′ | 19 | 420 | 169 |
| Probe | TET-5′-aacctaccctggagttccggagcg-3′-TAMRA | 24 | 443 | 170 |
| Reverse | 5′-ttttctaccgctccccagtct-3′ | 21 | 478 | 171 |

TABLE AC

Probe Name Ag690

| | | | | |
|---|---|---|---|---|
| Primers | Sequences | Length | Start Position | SEQ ID NO. |

| | | | | |
|---|---|---|---|---|
| Forward | 5′-ctgggtggccactatcctt-3′ | 19 | 366 | 172 |
| Probe | TET-5′-acaacccgctgtgtttgctgacag-3′-TAMRA | 24 | 417 | 173 |
| Reverse | 5′-cggaactccagggtaggtt-3′ | 19 | 443 | 174 |

TABLE AD

Probe Name Ag2820

| | | | | |
|---|---|---|---|---|
| Primers | Sequences | Length | Start Position | SEQ ID NO. |

| | | | | |
|---|---|---|---|---|
| Forward | 5′-cagagaagcagacgagttcact-3′ | 22 | 1093 | 175 |
| Probe | TET-5′-caaggacagaattttaccctaaggca-3′-TAMRA | 26 | 1118 | 176 |
| Reverse | 5′-gttgctggttcacaaactccta-3′ | 22 | 1146 | 177 |

TABLE 35
CNS_neurodegeneration_v1.0

| | Rel. Exp.(%) | | |
|---|---|---|---|
| | Ag018b, | | Rel. Exp.(%) |
| | Run | | Ag018b, |
| Tissue Name | 206989716 | Tissue Name | Run 206989716 |
| AD 1 Hippo | 10.2 | Control (Path) 3 | 6.6 |
| | | Temporal Ctx | |
| AD 2 Hippo | 27.5 | Control (Path) 4 | 38.7 |
| | | Temporal Ctx | |
| AD 3 Hippo | 9.4 | AD 1 Occipital | 14.1 |
| | | Ctx | |
| AD 4 Hippo | 9.7 | AD 2 Occipital | 0.0 |
| | | Ctx (Missing) | |
| AD 5 hippo | 79.6 | AD 3 Occipital | 5.6 |
| | | Ctx | |
| AD 6 Hippo | 46.3 | AD 4 Occipital | 22.1 |
| | | Ctx | |
| Control 2 Hippo | 47.3 | AD 5 Occipital | 19.5 |
| | | Ctx | |
| Control 4 Hippo | 5.8 | AD 6 Occipital | 57.0 |
| | | Ctx | |
| Control (Path) 3 | 5.6 | Control 1 | 3.6 |
| Hippo | | Occipital Ctx | |
| AD 1 Temporal Ctx | 13.3 | Control 2 | 63.7 |
| | | Occipital Ctx | |
| AD 2 Temporal Ctx | 30.8 | Control 3 | 17.6 |
| | | Occipital Ctx | |
| AD 3 Temporal Ctx | 7.6 | Control 4 | 5.9 |
| | | Occipital Ctx | |
| AD 4 Temporal Ctx | 21.8 | Control (Path) 1 | 95.9 |
| | | Occipital Ctx | |
| AD 5 Inf Temporal | 65.1 | Control (Path) 2 | 12.4 |
| Ctx | | Occipital Ctx | |
| AD 5 Sup Temporal | 43.8 | Control (Path) 3 | 3.3 |
| Ctx | | Occipital Ctx | |
| AD 6 Inf Temporal | 36.6 | Control (Path) 4 | 22.5 |
| Ctx | | Occipital Ctx | |
| AD 6 Sup Temporal | 36.6 | Control 1 Parietal | 7.3 |
| Ctx | | Ctx | |
| Control 1 Temporal | 7.2 | Control 2 Parietal | 38.7 |
| Ctx | | Ctx | |
| Control 2 Temporal | 53.2 | Control 3 Parietal | 13.8 |
| Ctx | | Ctx | |
| Control 3 Temporal | 15.9 | Control (Path) 1 | 88.9 |
| Ctx | | Parietal Ctx | |
| Control 4 Temporal | 11.7 | Control (Path) 2 | 24.5 |
| Ctx | | Parietal Ctx | |
| Control (Path) 1 | 100.0 | Control (Path) 3 | 3.9 |
| Temporal Ctx | | Parietal Ctx | |
| Control (Path) 2 | 46.0 | Control (Path) 4 | 45.4 |
| Temporal Ctx | | Parietal Ctx | |

TABLE 36
Panel 1

| | Rel. | Rel. | | Rel. | Rel. |
|---|---|---|---|---|---|
| | Exp.(%) | Exp.(%) | | Exp.(%) | Exp.(%) |
| | Ag018b, Run | Ag18, Run | | Ag018b, Run | Ag18, Run |
| Tissue Name | 109657241 | 87352731 | Tissue Name | 109657241 | 87352731 |
| Endothelial cells | 0.0 | 0.0 | Renal ca. 786- | 0.0 | 0.0 |
| | | | 0 | | |
| Endothelial cells | 0.0 | 0.0 | Renal ca. | 0.0 | 0.0 |
| (treated) | | | A498 | | |
| Pancreas | 0.0 | 0.0 | Renal ca. RXF | 0.0 | 0.0 |
| | | | 393 | | |
| Pancreatic ca. | 0.0 | 0.0 | Renal ca. | 0.0 | 0.0 |
| CAPAN 2 | | | ACHN | | |
| Adrenal gland | 0.0 | 100.0 | Renal ca. UO- | 0.0 | 0.0 |
| | | | 31 | | |
| Thyroid | 0.0 | 0.0 | Renal ca. TK- | 0.0 | 0.0 |
| | | | 10 | | |
| Salivary gland | 0.0 | 0.0 | Liver | 0.0 | 0.0 |
| Pituitary gland | 0.0 | 0.0 | Liver (fetal) | 0.0 | 0.0 |
| Brian (fetal) | 0.0 | 4.4 | Liver ca. | 0.0 | 0.0 |
| | | | (hepatoblast) | | |
| | | | HepG2 | | |
| Brain (whole) | 0.0 | 31.4 | Lung | 0.0 | 0.0 |
| Brain (amygdala) | 0.0 | 1.7 | Lung (fetal) | 0.0 | 0.0 |
| Brain | | 23.3 | Lung ca. | 0.0 | 0.0 |
| (cerebellum) | | | (small cell) | | |
| | | | LX-1 | | |
| Brain | 0.0 | 5.8 | Lung ca. | 0.0 | 0.1 |
| (hippocampus) | | | (small cell) | | |
| | | | NCI-H69 | | |
| Brain (substantia | 0.0 | 0.3 | Lung ca. | 0.0 | 0.0 |
| nigra) | | | (s.cell var.) | | |
| | | | SHP-77 | | |
| Brain (thalamus) | 0.0 | 0.1 | Lung ca. | 100.0 | 0.0 |
| | | | (large | | |
| | | | cell)NCI-H460 | | |
| Brain | 0.0 | 0.5 | Lung ca. (non- | 0.0 | 0.0 |
| (hypothalamus) | | | sm.cell) A549 | | |
| Spinal cord | 0.0 | 0.0 | Lung ca. (non- | 0.0 | 0.0 |
| | | | s.cell) NCI- | | |
| | | | H23 | | |
| glio/astro U87- | 0.0 | 0.0 | Lung ca. (non- | 0.0 | 0.0 |
| MG | | | s.cell) HOP-62 | | |
| glio/astro U-118- | 0.0 | 0.0 | Lung ca. (non | 0.0 | 0.0 |
| MG | | | s.cl) NCI- | | |
| | | | H522 | | |
| astrocytoma | 0.0 | 0.0 | Lung ca. | 0.0 | 0.0 |
| SW1783 | | | (squam.) SW | | |
| | | | 900 | | |
| neuro*; met SK- | 0.0 | 0.0 | Lung ca. | 0.0 | 3.6 |
| N-AS | | | (squam.) NCI- | | |
| | | | H596 | | |
| astrocytoma SF- | 0.0 | 0.0 | Mammary | 0.0 | 0.0 |
| 539 | | | gland | | |
| astrocytoma | 0.0 | 0.0 | Breast ca.* | 0.0 | 0.0 |
| SNB-75 | | | (pl.ef) MCF-7 | | |
| glioma SNB-19 | 0.0 | 6.0 | Breast ca.* | 0.0 | 0.0 |
| | | | (pl.ef) MDA- | | |
| | | | MB-231 | | |
| glioma U251 | 0.0 | 0.0 | Breast ca.*(pl. | 0.0 | 0.0 |
| | | | ef) T47D | | |
| glioma SF-295 | 0.0 | 0.0 | Breast ca. BT- | 0.0 | 0.0 |
| | | | 549 | | |
| Heart | 0.0 | 0.0 | Breast ca. | 0.0 | 0.0 |
| | | | MDA-N | | |
| Skeletal muscle | 0.0 | 0.0 | Ovary | 0.0 | 0.0 |
| Bone marrow | 0.0 | 0.0 | Ovarian ca. | 0.0 | 0.0 |
| | | | OVCAR-3 | | |
| Thymus | 0.0 | 0.0 | Ovarian ca. | 0.0 | 0.0 |
| | | | OVCAR-4 | | |
| Spleen | 0.0 | 0.0 | Ovarian ca. | 0.0 | 0.0 |
| | | | OVCAR-5 | | |
| Lymph node | 0.0 | 0.0 | Ovarian ca. | 0.0 | 0.0 |
| | | | OVCAR-8 | | |
| Colon | 0.0 | 0.0 | Ovarian ca. | 0.0 | 0.0 |
| (ascending) | | | IGROV-1 | | |
| Stomach | 0.0 | 0.0 | Ovarian ca. | 0.0 | 0.0 |
| | | | (ascites) SK- | | |
| | | | OV-3 | | |
| Small intestine | 0.0 | 0.0 | Uterus | 0.0 | 0.0 |
| Colon ca. | 0.0 | 0.0 | Placenta | 0.0 | 00 |
| SW480 | | | | | |
| Colon ca.* | 0.0 | 0.0 | Prostate | 0.0 | 0.0 |
| SW620 (SW480 | | | | | |
| met) | | | | | |
| Colon ca. HT29 | 0.0 | 0.0 | Prostate ca.* | 0.0 | 0.0 |
| | | | (bone met) | | |
| | | | PC-3 | | |
| Colon ca. HCT- | 0.0 | 0.0 | Testis | 0.0 | 0.0 |
| 116 | | | | | |
| Colon ca. CaCo- | 0.0 | 0.0 | Melanoma | 0.0 | 0.0 |
| 2 | | | Hs688(A).T | | |
| Colon ca. HCT- | 0.0 | 0.0 | Melanoma* | 0.0 | 0.0 |
| 15 | | | (met) | | |
| | | | Hs688(B).T | | |
| Colon ca. HCC- | 0.0 | 0.0 | Melanoma* | 0.0 | 0.0 |
| 2998 | | | UACC-62 | | |
| Gastric ca. * | 0.0 | 0.0 | Melanoma | 0.0 | 0.0 |
| (liver met) NCI- | | | M14 | | |
| N87 | | | | | |
| Bladder | 0.0 | 0.0 | Melanoma | 0.0 | 0.0 |
| | | | LOX IMVI | | |
| Trachea | 0.0 | 0.0 | Melanoma* | 0.0 | 0.0 |
| | | | (met) SK- | | |
| | | | MEL-5 | | |
| Kidney | 0.0 | 0.0 | Melanoma | 0.0 | 0.0 |
| | | | SK-MEL-28 | | |
| Kidney (fetal) | 0.0 | 0.0 | | | |

TABLE 37
Panel 1.2

| | Rel. | Rel. | | Rel. | Rel. |
|---|---|---|---|---|---|
| | Exp.(%) | Exp.(%) | | Exp.(%) | Exp.(%) |
| | Ag690, Run | Ag690, Run | | Ag690, Run | Ag690, Run |
| Tissue Name | 114253492 | 116349889 | Tissue Name | 114253492 | 116349889 |
| Endothelial | 0.0 | 0.0 | Renal ca. 786- | 0.0 | 0.0 |
| cells | | | 0 | | |
| Heart (Fetal) | 0.6 | 0.2 | Renal ca. | 0.1 | 0.0 |
| | | | A498 | | |
| Pancreas | 0.3 | 0.0 | Renal ca. RXF | 0.0 | 0.0 |
| | | | 393 | | |
| Pancreatic ca. | 0.0 | 0.0 | Renal ca. | 0.0 | 0.0 |
| CAPAN 2 | | | ACHN | | |
| Adrenal Gland | 81.2 | 100.0 | Renal ca. UO- | 0.1 | 0.0 |
| | | | 31 | | |
| Thyroid | 0.0 | 0.0 | Renal ca. TK- | 0.1 | 0.0 |
| | | | 10 | | |
| Salivary gland | 0.8 | 0.4 | Liver | 0.0 | 0.0 |
| Pituitary gland | 6.7 | 6.6 | Liver (fetal) | 0.0 | 0.0 |
| Brain (fetal) | 5.7 | 5.1 | Liver ca. | 0.0 | 0.0 |
| | | | (hepatoblast) | | |
| | | | HepG2 | | |
| Brain (whole) | 6.1 | 6.8 | Lung | 0.4 | 0.1 |
| Brain | 5.3 | 4.9 | Lung (fetal) | 0.6 | 0.2 |
| (amygdala) | | | | | |
| Brain | 1.9 | 1.0 | Lung ca. | 0.0 | 0.0 |
| (cerebellum) | | | (small cell) | | |
| | | | LX-1 | | |
| Brain | 5.6 | 4.4 | Lung ca. | 40.6 | 21.3 |
| (hippocampus) | | | (small cell) | | |
| | | | NCI-H69 | | |
| Brain | 4.0 | 3.7 | Lung ca. | 7.2 | 4.8 |
| (thalamus) | | | (s.cell var.) | | |
| | | | SHP-77 | | |
| Cerebral Cortex | 18.4 | 17.6 | Lung ca. | 1.6 | 0.9 |
| | | | (large cell) | | |
| | | | NCI-H460 | | |
| Spinal cord | 2.3 | 1.7 | Lung ca. (non- | 0.3 | 0.0 |
| | | | sm. cell) A549 | | |
| glio/astro U87- | 0.0 | 0.0 | Lung ca. (non | 0.0 | 0.0 |
| MG | | | s.cell) NCI- | | |
| | | | H23 | | |
| glio/astro U- | 0.2 | 0.0 | Lung ca. (non- | 1.0 | 0.3 |
| 118-MG | | | s.cell) HOP- | | |
| | | | 62 | | |
| astrocytoma | 0.0 | 0.0 | Lung ca. (non | 0.1 | 0.0 |
| SW1783 | | | s.cl) NCI- | | |
| | | | H522 | | |
| neuro*; met SK- | 1.8 | 0.6 | Lung ca. | 0.8 | 0.4 |
| N-AS | | | (squam.) SW | | |
| | | | 900 | | |
| astrocytoma SF- | 0.5 | 0.0 | Lung ca. | 100.0 | 71.2 |
| 539 | | | (squam.) NCI- | | |
| | | | H596 | | |
| astrocytoma | 0.0 | 0.0 | Mammary | 0.1 | 0.0 |
| SNB-75 | | | gland | | |
| glioma SNB-19 | 12.9 | 6.4 | Breast ca.* | 0.2 | 0.1 |
| | | | (pl.ef) MCF-7 | | |
| glioma U251 | 1.6 | 0.4 | Breast ca.* | 0.0 | 0.0 |
| | | | (pl.ef) MDA- | | |
| | | | MB-231 | | |
| glioma SF-295 | 0.1 | 0.1 | Breast ca.* | 0.4 | 0.1 |
| | | | (pl. ef) T47D | | |
| Heart | 3.2 | 3.1 | Breast ca. BT- | 0.1 | 0.0 |
| | | | 549 | | |
| Skeletal Muscle | 0.0 | 0.0 | Breast ca. | 0.2 | 0.0 |
| | | | MDA-N | | |
| Bone marrow | 0.0 | 0.0 | Ovary | 1.2 | 0.3 |
| Thymus | 0.0 | 0.0 | Ovarian ca. | 0.0 | 0.0 |
| | | | OVCAR-3 | | |
| Spleen | 0.0 | 0.00 | Ovarian ca. | 0.0 | 0.0 |
| | | | OVCAR-4 | | |
| Lymph node | 0.0 | 0.0 | Ovarian ca. | 0.3 | 0.0 |
| | | | OVCAR-5 | | |
| Colorectal | 0.1 | 0.0 | Ovarian ca. | 0.0 | 0.0 |
| Tissue | | | OVCAR-8 | | |
| Stomach | 0.0 | 0.0 | Ovarian ca. | 0.0 | 0.0 |
| | | | IGROV-1 | | |
| Small intestine | 0.1 | 0.0 | Ovarian ca. | 0.3 | 0.0 |
| | | | (ascites) SK- | | |
| | | | OV-3 | | |
| Colon ca. | 0.0 | 0.0 | Uterus | 0.0 | 0.0 |
| SW480 | | | | | |
| Colon ca. | 0.0 | 0.0 | Placenta | 0.0 | 0.0 |
| SW620 (SW480 | | | | | |
| met) | | | | | |
| Colon ca. HT29 | 0.1 | 0.0 | Prostate | 0.0 | 0.0 |
| Colon ca. HCT- | 0.0 | 0.0 | Prostate ca.* | 0.0 | 0.0 |
| 116 | | | (bone met) | | |
| | | | PC-3 | | |
| Colon Ca. | 0.0 | 0.0 | Testis | 2.1 | 1.3 |
| CaCo-2 | | | | | |
| Colon Ca. | 0.4 | 0.0 | Melanoma | 0.0 | 0.0 |
| Tissue | | | Hs688(A).T | | |
| (ODO3866) | | | | | |
| Colon ca. HCC | 0.0 | 0.0 | Melanoma* | 0.1 | 0.0 |
| 2998 | | | (met) | | |
| | | | Hs688(B).T | | |
| Gastric ca.* | 0.1 | 0.0 | Melanoma | 0.0 | 0.0 |
| (liver met) NCI- | | | UACC-62 | | |
| N87 | | | | | |
| Bladder | 2.3 | 1.0 | Melanoma | 0.7 | 0.0 |
| | | | M14 | | |
| Trachea | 0.1 | 0.0 | Melanoma | 0.0 | 0.0 |
| | | | LOX IMVI | | |
| Kidney | 0.6 | 0.0 | Melanoma* | 0.0 | 0.0 |
| | | | (met) SK- | | |
| | | | MEL-5 | | |
| Kidney (fetal) | 1.7 | 0.7 | | | |

TABLE 38
Panel 1.3D

| | Rel. | Rel. | | Rel. | Rel. |
|---|---|---|---|---|---|
| | Exp.(%) | Exp.(%) | | Exp.(%) | Exp.(%) |
| | Ag2820, Run | Ag2820, Run | | Ag2820, Run | Ag2820, Run |
| Tissue Name | 165527000 | 165544916 | Tissue Name | 165527000 | 165544916 |
| Liver | 17.2 | 8.2 | Kidney (fetal) | 2.5 | 1.3 |
| adenocarcinoma | | | | | |
| Pancreas | 0.0 | 0.1 | Renal ca. 786- | 19.9 | 9.5 |
| | | | 0 | | |
| Pancreatic ca. | 10.4 | 6.3 | Renal ca. | 13.2 | 7.2 |
| CAPAN 2 | | | A498 | | |
| Adrenal gland | 4.9 | 2.7 | Renal ca. | 21.3 | 26.1 |
| | | | RXF 393 | | |
| Thyroid | 0.6 | 0.2 | Renal ca. | 7.6 | 7.5 |
| | | | ACHN | | |
| Salivary gland | 0.0 | 0.1 | Renal ca. UO- | 13.8 | 9.5 |
| | | | 31 | | |
| Pituitary gland | 0.8 | 0.1 | Renal ca. TK- | 0.0 | 0.0 |
| | | | 10 | | |
| Brain (fetal) | 2.3 | 1.1 | Liver | 0.0 | 0.0 |
| Brain (whole) | 1.7 | 2.1 | Liver (fetal) | 0.0 | 0.0 |
| Brain (amygdala) | 2.0 | 2.0 | Liver ca. | 0.0 | 0.4 |
| | | | (hepatoblast) | | |
| | | | HepG2 | | |
| Brain | 0.3 | 0.3 | Lung | 0.2 | 0.0 |
| (cerebellum) | | | | | |
| Brain | 3.5 | 2.1 | Lung (fetal) | 0.0 | 0.7 |
| (hippocampus) | | | | | |
| Brain (substantia | 0.4 | 0.1 | Lung ca. | 0.0 | 0.0 |
| nigra) | | | (small cell) | | |
| | | | LX-1 | | |
| Brain (thalamus) | 2.2 | 3.2 | Lung ca. | 5.4 | 11.2 |
| | | | (small cell) | | |
| | | | NCI-H69 | | |
| Cerebral Cortex | 4.8 | 3.6 | Lung ca. | 0.0 | 0.0 |
| | | | (s.cell var.) | | |
| | | | SHP-77 | | |
| Spinal cord | 0.4 | 1.0 | Lung ca. | 26.1 | 12.9 |
| | | | (large | | |
| | | | cell)NCI- | | |
| | | | H460 | | |
| glio/astro U87- | 18.8 | 26.1 | Lung ca. | 0.6 | 0.2 |
| MG | | | (non-sm. cell) | | |
| | | | A549 | | |
| glio/astro U-118- | 100.0 | 100.0 | Lung ca. | 1.2 | 0.1 |
| MG | | | (non-s.cell) | | |
| | | | NCI-H23 | | |
| astrocytoma | 24.8 | 19.3 | Lung ca. | 16.0 | 6.8 |
| SW1783 | | | (non-s.cell) | | |
| | | | HOP-62 | | |
| neuro*; met SK- | 18.8 | 16.3 | Lung ca. | 15.3 | 5.8 |
| N-AS | | | (non-s.cl) | | |
| | | | NCI-H522 | | |
| astrocytoma SF- | 22.2 | 19.3 | Lung ca. | 0.2 | 0.1 |
| 539 | | | (squam.) SW | | |
| | | | 900 | | |
| astrocytoma | 27.2 | 15.7 | Lung ca. | 19.2 | 12.3 |
| SNB-75 | | | (squam.) | | |
| | | | NCI-H596 | | |
| glioma SNB-19 | 4.0 | 3.4 | Mammary | 0.5 | 0.2 |
| | | | gland | | |
| glioma U251 | 88.3 | 76.8 | Breast ca.* | 5.1 | 2.1 |
| | | | (pl.ef) MCF-7 | | |
| glioma SF-295 | 5.6 | 3.5 | Breast ca.* | 0.5 | 0.4 |
| | | | (pl.ef) MDA- | | |
| | | | MB-231 | | |
| Heart (fetal) | 0.3 | 0.3 | Breast ca.* | 1.9 | 1.1 |
| | | | (pl.ef) T47D | | |
| Heart | 0.0 | 0.0 | Breast ca. BT- | 0.0 | 0.0 |
| | | | 549 | | |
| Skeletal muscle | 2.3 | 1.3 | Breast ca. | 1.5 | 1.1 |
| (fetal) | | | MDA-N | | |
| Skeletal muscle | 0.0 | 0.2 | Ovary | 0.3 | 0.0 |
| Bone marrow | 0.0 | 0.0 | Ovarian ca. | 26.8 | 20.0 |
| | | | OVCAR-3 | | |
| Thymus | 0.5 | 0.6 | Ovarian ca. | 3.1 | 2.0 |
| | | | OVCAR-4 | | |
| Spleen | 1.0 | 0.9 | Ovarian ca. | 0.0 | 0.0 |
| | | | OVCAR-5 | | |
| Lymph node | 2.4 | 2.0 | Ovarian ca. | 1.7 | 2.8 |
| | | | OVCAR-8 | | |
| Colorectal | 0.5 | 0.1 | Ovarian ca. | 0.0 | 0.4 |
| | | | IGROV-1 | | |
| Stomach | 2.2 | 0.1 | Ovarian ca.* | 22.2 | 0.0 |
| | | | (ascites) SK- | | |
| | | | OV-3 | | |
| Small intestine | 1.3 | 0.7 | Uterus | 1.2 | 0.9 |
| Colon ca. SW480 | 2.4 | 2.0 | Placenta | 7.7 | 4.1 |
| Colon ca.* | 0.0 | 0.0 | Prostate | 0.0 | 0.0 |
| SW620(SW480 | | | | | |
| met) | | | | | |
| Colon ca. HT29 | 0.6 | 0.8 | Prostate ca.* | 0.0 | 0.0 |
| | | | (bone | | |
| | | | met)PC-3 | | |
| Colon ca. HCT- | 0.0 | 0.1 | Testis | 0.0 | 0.1 |
| 116 | | | | | |
| Colon ca. CaCo-2 | 9.7 | 7.4 | Melanoma | 12.8 | 7.5 |
| | | | Hs688(A).T | | |
| Colon ca. | 2.6 | 1.4 | Melanoma* | 12.0 | 4.2 |
| tissue(ODO3866) | | | (met) | | |
| | | | Hs688(B).T | | |
| Colon ca. HCC- | 2.4 | 1.2 | Melanoma | 0.4 | 0.3 |
| 2998 | | | UACC-62 | | |
| Gastric ca.* (liver | 2.4 | 0.6 | Melanoma | 14.4 | 7.8 |
| met) NCI-N87 | | | M14 | | |
| Bladder | 2.3 | 0.4 | Melanoma | 0.0 | 0.0 |
| | | | LOX IMVI | | |
| Trachea | 0.0 | 0.2 | Melanoma* | 3.8 | 1.8 |
| | | | (met) SK- | | |
| | | | MEL-5 | | |
| Kidney | 0.0 | 0.0 | Adipose | 12.9 | 0.6 |

TABLE 39
Panel 2D

| | Rel. Exp.(%) | Rel. Exp.(%) | Rel. Exp.(%) | Rel. Exp.(%) |
|---|---|---|---|---|
| | Ag2820, Run | Ag2820, Run | Ag690, Run | Ag690, Run |
| Tissue Name | 163578010 | 165910586 | 147633637 | 148015549 |
| Normal Colon | 12.4 | 15.7 | 4.8 | 5.4 |
| CC Well to Mod | 7.2 | 7.4 | 4.0 | 1.7 |
| Diff (ODO3866) | | | | |
| CC Margin | 0.8 | 0.4 | 7.5 | 2.8 |
| (ODO3866) | | | | |
| CC Gr.2 | 3.8 | 2.3 | 0.0 | 0.0 |
| rectosigmoid | | | | |
| (ODO3868) | | | | |
| CC Margin | 2.2 | 1.2 | 1.2 | 0.0 |
| (ODO3868) | | | | |
| CC Mod Diff | 0.7 | 0.0 | 0.0 | 0.0 |
| (ODO3920) | | | | |
| CC Margin | 1.6 | 1.4 | 1.2 | 1.3 |
| (ODO3920) | | | | |
| CC Gr.2 ascend | 2.9 | 3.6 | 1.3 | 1.4 |
| colon (ODO3921) | | | | |
| CC Margin | 1.3 | 0.0 | 0.0 | 0.0 |
| (ODO3921) | | | | |
| CC from Partial | 12.3 | 12.5 | 2.1 | 0.0 |
| Hepatectomy | | | | |
| (ODO4309) Mets | | | | |
| Liver Margin | 0.4 | 0.0 | 0.0 | 0.0 |
| (OD04309) | | | | |
| Colon mets to lung | 1.5 | 1.1 | 0.0 | 1.0 |
| (OD04451-01) | | | | |
| Lung Margin | 0.0 | 0.8 | 0.0 | 5.5 |
| (OD04451-02) | | | | |
| Normal Prostate | 6.3 | 2.0 | 0.0 | 0.0 |
| 6546-1 | | | | |
| Prostate Cancer | 4.9 | 4.1 | 1.2 | 0.0 |
| (OD04410) | | | | |
| Prostate Margin | 6.0 | 1.9 | 3.4 | 2.5 |
| (OD04410) | | | | |
| Prostate Cancer | 3.2 | 1.2 | 4.1 | 0.0 |
| (OD04720-01) | | | | |
| Prostate Margin | 8.0 | 3.9 | 0.0 | 0.0 |
| (OD04720-02) | | | | |
| Normal Lung | 13.4 | 11.8 | 9.5 | 15.3 |
| 0610101 | | | | |
| Lung Met to | 64.2 | 39.2 | 4.9 | 8.1 |
| Muscle (ODO4286) | | | | |
| Muscle Margin | 1.3 | 1.1 | 0.0 | 0.0 |
| (ODO4286) | | | | |
| Lung Malignant | 66.9 | 57.8 | 1.1 | 7.1 |
| Cancer (OD03126) | | | | |
| Lung Margin | 10.6 | 5.9 | 32.3 | 26.1 |
| (OD03126) | | | | |
| Lung Cancer | 10.4 | 11.6 | 3.9 | 0.0 |
| (OD04404) | | | | |
| Lung Margin | 10.7 | 14.4 | 24.8 | 22.7 |
| (OD04404) | | | | |
| Lung Cancer | 8.5 | 4.5 | 0.0 | 0.0 |
| (OD04565) | | | | |
| Lung Margin | 5.3 | 6.2 | 13.0 | 8.6 |
| (OD04565) | | | | |
| Lung Cancer | 13.6 | 4.5 | 0.0 | 5.7 |
| (OD04237-01) | | | | |
| Lung Margin | 5.3 | 3.8 | 10.6 | 10.5 |
| (OD04237-02) | | | | |
| Ocular Mel Met to | 0.0 | 0.0 | 0.0 | 0.0 |
| Liver (ODO4310) | | | | |
| Liver Margin | 0.0 | 0.3 | 0.0 | 0.0 |
| (ODO4310) | | | | |
| Melanoma Mets to | 57.4 | 31.6 | 1.3 | 0.0 |
| Lung (OD04321) | | | | |
| Lung Margin | 7.0 | 3.5 | 2.5 | 6.3 |
| (OD04321) | | | | |
| Normal Kidney | 18.9 | 14.4 | 30.1 | 30.4 |
| Kidney Ca, Nuclear | 5.6 | 2.9 | 0.0 | 2.6 |
| grade 2 (OD04338) | | | | |
| Kidney Margin | 10.8 | 9.0 | 3.6 | 0.0 |
| (OD04338) | | | | |
| Kidney Ca Nuclear | 82.4 | 67.8 | 0.0 | 0.0 |
| grade ½ | 82.4 | 67.8 | 0.0 | 0.0 |
| (OD04339) | | | | |
| Kidney Margin | 17.7 | 8.8 | 15.2 | 13.4 |
| (OD04339) | | | | |
| Kidney Ca, Clear | 0.0 | 0.3 | 0.0 | 0.0 |
| cell type | | | | |
| (OD04340) | | | | |
| Kidney Margin | 13.9 | 8.0 | 0.0 | 1.0 |
| (OD04340) | | | | |
| Kidney Ca, Nuclear | 84.7 | 58.2 | 0.0 | 0.0 |
| grade 3 (OD04348) | | | | |
| Kidney Margin | 4.6 | 11.1 | 4.3 | 2.6 |
| (OD04348) | | | | |
| Kidney Cancer | 0.0 | 4.6 | 0.0 | 0.0 |
| (OD04622-01) | | | | |
| Kidney Margin | 3.9 | 1.1 | 0.0 | 1.5 |
| (OD04622-03) | | | | |
| Kidney Cancer | 100.0 | 78.5 | 0.0 | 0.8 |
| (OD04450-01) | | | | |
| Kidney Margin | 12.0 | 6.9 | 5.3 | 7.0 |
| (OD04450-03) | | | | |
| Kidney Cancer | 4.9 | 4.2 | 0.0 | 0.0 |
| 8120607 | | | | |
| Kidney Margin | 1.7 | 3.7 | 1.3 | 0.0 |
| 8120608 | | | | |
| Kidney Cancer | 0.0 | 0.0 | 0.0 | 0.0 |
| 8120613 | | | | |
| Kidney Margin | 1.6 | 0.0 | 4.6 | 0.0 |
| 8120614 | | | | |
| Kidney Cancer | 39.5 | 36.1 | 100.0 | 100.0 |
| 9010320 | | | | |
| Kidney Margin | 22.5 | 11.6 | 14.5 | 5.1 |
| 9010321 | | | | |
| Normal Uterus | 4.1 | 7.0 | 0.0 | 0.4 |
| Uterus Cancer | 5.5 | 2.3 | 0.0 | 1.1 |
| 064011 | | | | |
| Normal Thyroid | 4.7 | 1.1 | 2.6 | 0.0 |
| Thyroid Cancer | 36.3 | 40.9 | 0.0 | 0.0 |
| 064010 | | | | |
| Thyroid Cancer | 5.8 | 2.8 | 0.0 | 0.0 |
| A302152 | | | | |
| Thyroid Margin | 10.0 | 7.2 | 0.0 | 3.0 |
| A302153 | | | | |
| Normal Breast | 9.5 | 11.3 | 13.6 | 11.1 |
| Breast Cancer | 0.7 | 0.8 | 0.0 | 0.0 |
| (OD04566) | | | | |
| Breast Cancer | 4.0 | 2.9 | 0.0 | 2.6 |
| (OD04590-01) | | | | |
| Breast Cancer Mets | 32.5 | 15.9 | 0.0 | 0.0 |
| (OD04590-03) | | | | |
| Breast Cancer | 12.2 | 2.9 | 2.5 | 1.3 |
| Metastasis | | | | |
| (OD04655-05) | | | | |
| Breast Cancer | 7.5 | 5.5 | 0.0 | 0.0 |
| 064006 | | | | |
| Breast Cancer 1024 | 1.8 | 1.3 | 2.6 | 1.3 |
| Breast Cancer | 3.5 | 1.2 | 0.0 | 2.6 |
| 9100266 | | | | |
| Breast Margin | 4.9 | 1.7 | 0.0 | 1.3 |
| 9100265 | | | | |
| Breast Cancer | 3.5 | 1.7 | 2.0 | 0.0 |
| A209073 | | | | |
| Breast Margin | 0.6 | 2.0 | 0.0 | 1.1 |
| A2090734 | | | | |
| Normal Liver | 1.2 | 0.3 | 0.0 | 0.0 |
| Liver Cancer | 0.0 | 0.0 | 0.0 | 0.0 |
| 064003 | | | | |
| Liver Cancer 1025 | 0.0 | 0.0 | 0.0 | 0.0 |
| Liver Cancer 1026 | 0.0 | 0.0 | 2.2 | 1.3 |
| Liver Cancer 6004- | 0.0 | 0.0 | 0.0 | 0.0 |
| T | | | | |
| Liver Tissue 6004- | 1.0 | 0.3 | 9.7 | 4.3 |
| N | | | | |
| Liver Cancer 6005- | 0.0 | 0.0 | 0.0 | 0.0 |
| T | | | | |
| Liver Tissue 6005- | 0.0 | 0.0 | 0.0 | 0.0 |
| N | | | | |
| Normal Bladder | 14.7 | 12.7 | 6.3 | 3.8 |
| Bladder Cancer | 9.2 | 2.0 | 0.0 | 0.6 |
| 1023 | | | | |
| Bladder Cancer | 3.9 | 2.3 | 1.5 | 1.5 |
| A302173 | | | | |
| Bladder Cancer | 89.5 | 82.4 | 0.0 | 0.0 |
| (OD04718-01) | | | | |
| Bladder Normal | 3.5 | 3.9 | 0.0 | 0.0 |
| Adjacent | | | | |
| (OD04718-03) | | | | |
| Normal Ovary | 2.1 | 0.6 | 4.4 | 3.7 |
| Ovarian Cancer | 36.3 | 100.0 | 16.8 | 13.1 |
| 064008 | | | | |
| Ovarian Cancer | 0.0 | 0.4 | 0.0 | 0.0 |
| (OD04768-07) | | | | |
| Ovary Margin | 6.9 | 4.4 | 1.3 | 0.0 |
| (OD04768-08) | | | | |
| Normal Stomach | 2.2 | 1.9 | 0.0 | 2.5 |
| Gastric Cancer | 3.0 | 2.8 | 1.3 | 0.0 |
| 9060358 | | | | |
| Stomach Margin | 0.7 | 1.5 | 1.1 | 0.0 |
| 9060359 | | | | |
| Gastric Cancer | 1.9 | 1.8 | 0.6 | 0.0 |
| 9060395 | | | | |
| Stomach Margin | 2.2 | 2.3 | 0.0 | 0.0 |
| 9060394 | | | | |
| Gastric Cancer | 22.7 | 28.5 | 0.0 | 1.4 |
| 9060397 | | | | |
| Stomach Margin | 0.7 | 0.0 | 1.1 | 0.0 |
| 9060396 | | | | |
| Gastric Cancer | 9.2 | 6.5 | 0.0 | 0.0 |
| 064005 | | | | |

TABLE 40
Panel 4D

| | Rel. | Rel. | Rel. | | Rel. | Rel. | Rel. |
|---|---|---|---|---|---|---|---|
| | Exp.(%) | Exp.(%) | Exp.(%) | | Exp.(%) | Exp.(%) | Exp.(%) |
| | Ag018b, Run | Ag2820, Run | Ag2820, Run | | Ag018b, Run | Ag2820, Run | Ag2820, Run |
| Tissue Name | 146087211 | 162350531 | 164329602 | Tissue Name | 146087211 | 162350531 | 164329602 |
| Secondary Th1 act | 0.0 | 0.0 | 0.0 | HUVEC IL- | 0.0 | 0.0 | 0.0 |
| | | | | 1beta | | | |
| Secondary Th2 act | 0.0 | 0.0 | 0.5 | HUVEC IFN | 0.0 | 0.0 | 0.3 |
| | | | | gamma | | | |
| Secondary Tr1 act | 0.0 | 0.0 | 0.3 | HUVEC TNF | 0.0 | 0.0 | 0.0 |
| | | | | alpha + IFN | | | |
| | | | | gamma | | | |
| Secondary Th1 | 0.0 | 0.0 | 0.0 | HUVEC TNF | 0.0 | 0.0 | 0.0 |
| rest | | | | alpha + IL4 | | | |
| Secondary Th2 | 0.0 | 0.0 | 0.0 | HUVEC IL-11 | 0.0 | 0.0 | 0.0 |
| rest | | | | | | | |
| Secondary Tr1 | 0.0 | 0.0 | 0.0 | Lung | 0.0 | 0.0 | 0.0 |
| rest | | | | Microvascular | | | |
| | | | | EC none | | | |
| Primary Th1 act | 0.0 | 0.0 | 0.0 | Lung | 0.0 | 0.0 | 0.2 |
| | | | | Microvascular | | | |
| | | | | EC TNFalpha + | | | |
| | | | | IL-1beta | | | |
| Primary Th2 act | 0.0 | 0.0 | 0.0 | Microvascular | 0.0 | 0.0 | 0.0 |
| | | | | Dermal EC none | | | |
| Primary Tr1 act | 0.0 | 0.0 | 0.0 | Microvascular | 0.0 | 0.0 | 0.0 |
| | | | | Dermal EC | | | |
| | | | | TNFalpha + IL- | | | |
| | | | | 1beta | | | |
| Primary Th1 rest | 0.0 | 0.0 | 0.0 | Bronchial | 0.0 | 2.4 | 19.9 |
| | | | | epithelium | | | |
| | | | | TNFalpha + | | | |
| | | | | IL1beta | | | |
| Primary Th2 rest | 0.0 | 0.0 | 0.0 | Small airway | 0.0 | 1.0 | 1.7 |
| | | | | epithelium none | | | |
| Primary Tr1 rest | 0.0 | 0.0 | 0.0 | Small airway | 0.0 | 0.0 | 0.0 |
| | | | | epithelium | | | |
| | | | | TNFalpha + IL- | | | |
| | | | | 1beta | | | |
| CD45RA CD4 | 0.0 | 1.6 | 0.8 | Coronery artery | 11.7 | 1.9 | 2.6 |
| lymphocyte act | | | | SMC rest | | | |
| CD45RO CD4 | 0.0 | 0.0 | 0.0 | Coronery artery | 0.0 | 3.0 | 1.2 |
| lymphocyte act | | | | SMC TNFalpha + | | | |
| | | | | IL-1beta | | | |
| CD8 lymphocyte | 0.0 | 0.0 | 0.0 | Astrocytes rest | 0.0 | 100.0 | 100.0 |
| act | | | | | | | |
| Secondary CD8 | 0.0 | 0.0 | 0.0 | Astrocytes | 0.0 | 71.7 | 65.5 |
| lymphocyte rest | | | | TNFalpha + | | | |
| | | | | IL-1beta | | | |
| Secondary CD8 | 0.0 | 0.0 | 0.0 | KU-812 | 0.0 | 0.0 | 0.0 |
| lymphocyte act | | | | (Basophil) rest | | | |
| CD4 lymphocyte | 0.0 | 0.0 | 0.0 | KU-812 | 0.0 | 0.0 | 0.0 |
| none | | | | (Basophil) | | | |
| | | | | PMA/ionomycin | | | |
| 2ry | 0.0 | 0.0 | 0.0 | CCD1106 | 12.9 | 35.6 | 70.2 |
| Th1/Th2/Tr1_anti- | 0.0 | 0.0 | 0.0 | (Keratinocytes) | | | |
| CD95 CH11 | | | | none | | | |
| LAK cells rest | 0.0 | 0.0 | 0.0 | CCD1106 | 0.0 | 13.4 | 29.3 |
| | | | | (Keratinocytes) | | | |
| | | | | TNFalpha + | | | |
| | | | | IL-1beta | | | |
| LAK cells IL-2 | 0.0 | 0.3 | 0.0 | Liver cirrhosis | 0.0 | 0.3 | 0.0 |
| LAK cells IL- | 0.0 | 0.0 | 0.7 | Lupus kidney | 0.0 | 6.2 | 8.1 |
| 2 + IL-12 | | | | | | | |
| LAK cells IL- | 0.0 | 0.0 | 0.0 | NCI-H292 none | 0.0 | 0.0 | 0.0 |
| 2 + IFN gamma | | | | | | | |
| LAK cells IL-2 + | 0.0 | 0.0 | 0.0 | NCI-H292 IL-4 | 0.0 | 0.3 | 0.4 |
| IL-18 | | | | | | | |
| LAK cells | 0.0 | 0.0 | 0.5 | NCI-H292 IL-9 | 0.0 | 0.0 | 0.0 |
| PMA/ionomycin | | | | | | | |
| NK Cells IL-2 rest | 0.0 | 0.0 | 0.0 | NCI-H292 IL- | 0.0 | 0.0 | 0.0 |
| | | | | 13 | | | |
| Two Way MLR 3 | 0.0 | 0.0 | 0.0 | NCI-H292 IFN | 0.0 | 0.0 | 0.0 |
| day | | | | gamma | | | |
| Two Way MLR 5 | 0.0 | 0.0 | 0.0 | HPAEC none | 0.0 | 0.0 | 0.0 |
| day | | | | | | | |
| Two Way MLR 7 | 0.0 | 0.0 | 0.0 | HPAEC TNF | 0.0 | 0.6 | 0.0 |
| day | | | | alpha + IL-1 | | | |
| | | | | beta | | | |
| PBMC rest | 0.0 | 0.0 | 0.0 | Lung fibroblast | 0.0 | 0.0 | 0.4 |
| | | | | none | | | |
| PBMC PWM | 0.0 | 0.0 | 0.0 | Lung fibroblast | 0.0 | 0.0 | 0.0 |
| | | | | TNF alpha + IL- | | | |
| | | | | 1 beta | | | |
| PBMC PHA-L | 0.0 | 0.0 | 0.0 | Lung fibroblast | 0.0 | 1.7 | 3.7 |
| | | | | IL-4 | | | |
| Ramos (B cell) | 0.0 | 0.0 | 0.0 | Lung fibroblast | 0.0 | 1.2 | 2.0 |
| none | | | | IL-9 | | | |
| Ramos (B cell) | 0.0 | 0.0 | 0.0 | Lung fibroblast | 0.0 | 1.6 | 3.3 |
| ionomycin | | | | IL-13 | | | |
| B lymphocytes | 0.0 | 0.3 | 2.5 | Lung fibroblast | 0.0 | 2.3 | 0.2 |
| PWM | | | | IFN gamma | | | |
| B lymphocytes | 0.0 | 0.0 | 0.0 | Dermal | 0.0 | 10.3 | 8.4 |
| CD40L and IL-4 | | | | fibroblast | | | |
| | | | | CCD1070 rest | | | |
| EOL-1 dbcAMP | 100.0 | 0.3 | 0.7 | Dermal | 0.0 | 10.0 | 11.3 |
| | | | | fibroblast | | | |
| | | | | CCD1070 TNF | | | |
| | | | | alpha | | | |
| EOL-1 dbcAMP | 27.0 | 0.9 | 0.0 | Dermal | 0.0 | 4.5 | 3.8 |
| PMA/ionomycin | | | | fibroblast | | | |
| | | | | CCD1070 IL-1 | | | |
| | | | | beta | | | |
| Dendritic cells | 0.0 | 0.0 | 0.0 | Dermal | 0.0 | 0.3 | 1.6 |
| none | | | | fibroblast IFN | | | |
| | | | | gamma | | | |
| Dendritic cells | 0.0 | 0.0 | 0.0 | Dermal | 0.0 | 12.1 | 13.4 |
| LPS | | | | fibroblast IL-4 | | | |
| Dendritic cells | 0.0 | 0.0 | 0.0 | IBD Colitis 2 | 0.0 | 0.3 | 0.0 |
| anti-CD40 | | | | | | | |
| Monocytes rest | 0.0 | 0.0 | 0.0 | IBD Crohn's | 0.0 | 0.8 | 3.7 |
| Monocytes LPS | 0.0 | 0.0 | 0.0 | Colon | 0.0 | 1.7 | 1.9 |
| Macrophages rest | 0.0 | 0.0 | 0.0 | Lung | 13.2 | 3.8 | 6.3 |
| Macrophages LPS | 0.0 | 0.0 | 0.0 | Thymus | 51.1 | 4.1 | 4.4 |
| HUVEC none | 0.0 | 0.0 | 0.0 | Kidney | 0.0 | 13.0 | 20.2 |
| HUVEC starved | 0.0 | 0.0 | 0.0 | | | | |

CNS_neurodegeneration_v1.0 Summary: Ag018b While no association between expression of the CG53018-01 gene and Alzheimer's disease is apparent in this panel, the profile here confirms expression of this gene in the brain. Please see Panel 1.3D for discussion of potential utility of this gene in the brain. Please note that one experiment with the probe primer set Ag2820 is not included because the amp plot indicates that there was a problem with this run.

Panel 1 Summary: Ag18/018b Two experiments show highest expression of this gene in a lung cancer cell line and the adrenal gland. This pattern is consistent with the results in Panel 1.2. Please see Panel 1.2 for discussion of utility of this gene in metabolic disease and cancer.

Panel 1.2 Summary: Ag690 The expression of the CG53018-01 gene was assessed in two independent runs in panel 1.2. Both runs show excellent concordance. The expression of this gene was highest in the adrenal gland and a lung cancer cell line. In addition, there is expression in other lung cancer cell lines as well as low but substantial expression in the brain. Thus, the expression of this gene could be used to distinguish these tissues from other tissues in the panel. Moreover, therapeutic modulation of this gene, through the use of small molecule drugs, antibodies or protein therapeutics might be of benefit in the treatment of lung cancer.

In addition to high levels of expression in the adrenal gland, this gene product is moderately expressed in pancreas, pituitary, and heart. Thus, this gene product may be an antibody target for the treatment of metabolic disease, including Types 1 and 2 diabetes and obesity. In addition, this TEN M3-like molecule may be an antibody target for the treatment of diseases of the adrenal gland, including Addison's disease and Cushings syndrome

Panel 1.3D Summary: Ag2820 The expression of the CG53018-01 gene was assessed in two independent runs in panel 1.3D, with good concordance between the different runs. Overall, the expression of this gene is highest in brain cancer cell lines. In addition, there is substantial expression in other samples derived from cancer cell lines, such as lung cancer and ovarian cancer. Thus, the expression of this gene could be used to distinguish these samples from other samples in the panel. Moreover, therapeutic modulation of this gene, through the use of small molecule drugs, antibodies or protein therapeutics might be of use in the treatment of brain cancer, lung cancer, or ovarian cancer.

Panel 2D Summary: Ag690/Ag2820 The expression of the CG53018-01 gene was assessed in three independent runs in panel 2D using two different probe/primer sets. The highest expression of this gene is generally associated with kidney cancers. Of particular note is the consistent absence of expression in normal kidney tissue adjacent to malignant kidney. In addition, there is substantial expression associated with ovarian cancer, bladder cancer and lung cancer. This is consistent with the expression seen in Panel 1.3D. Thus, the expression of this gene could be used to distinguish the above listed malignant tissue from other tissues in the panel. Particularly, the expression of this gene could be used to distinguish malignant kidney tissue from normal kidney. Moreover, therapeutic modulation of this gene, through the use of small molecule drugs, antibodies or protein therapeutics might be of benefit in the treatment of kidney cancer, ovarian cancer, bladder cancer or lung cancer.

Panel 3D Summary: Ag2820 Data from one experiment with this probe and primer set and the CG53018-01 gene is not included. The amp plot suggests that there were experimental difficulties with this run.

Panel 4D Summary: Ag018b/Ag2820 Two out of three experiments show highest expression of the CG53018-01 transcript is highest in astrocytes and microvascular dermal endothelial cells (CTs=29-30), with low but significant expression in keratinocytes, and dermal fibroblasts. Expression is not modulated by any treatment, suggesting that this protein may be important in normal homeostasis. Thus, this transcript or the protein it encodes could be used to identify the tissues and cells in which it is expressed.

### Example 15

FGF10-X

Construction of the mammalian expression vector pCEP4/Sec. The oligonucleotide primers, pSec-V5-His Forward (CTCGTC CTCGAG GGT AAG CCT ATC CCT AAC) (SEQ ID NO: 52) and the pSec-V5-His Reverse (CTCGTC GGGCCCCTGATCAGCGGGTTTAAAC) (SEQ ID NO: 53), were designed to amplify a fragment from the pcDNA3.1-V5His (Invitrogen, Carlsbad, Calif.) expression vector. The PCR product was digested with XhoI and ApaI and ligated into the XhoI/ApaI digested pSecTag2 B vector (Invitrogen, Carlsbad Calif.). The correct structure of the resulting vector, pSecV5His, was verified by DNA sequence analysis. The vector pSecV5His was digested with PmeI and NheI, and the PmeI-NheI fragment was ligated into the BamHI enow and NheI treated vector pCEP4 (Invitrogen, Carlsbad, Calif.). The resulting vector was named as pCEP4/Sec.

Expression of hFGF10-X in human embryonic kidney 293 cells. A 0.5 kb BglII-XhoI fragment containing the hFGF10-X sequence was isolated from pCR2.1-FGF10-X and subcloned into BamHI-XhoI digested pCEP4/Sec to generate expression vector pCEP4/Sec-FGF10-X. The pCEP4/Sec-FGF10-X vector was transfected into 293 cells using the LipofectaminePlus reagent following the manufacturer's instructions (Gibco/BRL). The cell pellet and supernatant were harvested 72 hours after transfection and examined for hFGF10-X expression by Western blotting (reducing conditions) with an anti-V5 antibody. FIG. 1 shows that hFGF10-X is expressed as a 27 kDa protein secreted by 293 cells.

Construction of Recombinant E. coli Expression Vector pETMY hFGF10-X.

The vector pRSETA (In Vitrogen Inc., Carlsbad, Calif.) was digested with XhoI and NcoI restriction enzymes. Oligonucleotide linkers CATGGTCAGCCTAC (SEQ ID NO: 178) and TCGAGTAGGCTGAC (SEQ ID NO: 179) were annealed at 37 degree Celsius and ligated into the XhoI-NcoI treated pRSETA. The resulting vector was confirmed by restriction analysis and sequencing and was named as pETMY. The BamHI-XhoI fragment (see above) was ligated into the pETMY that was digested with BamHI and XhoI restriction enzymes. The expression vector is named as pETMY-FGF10-X. In this vector, hFGF10-X was fused to the 6× His tag and T7 epitope at its N-terminus. The plasmid pETMY-FGF10-X was then transformed into the E. coli expression host BL21(DE3, pLys) (Novagen, Madison, Wis.) and the expression induction of protein FGF10-X was carried out according to the manufacturer's instructions. After induction, total cells were harvested, and proteins were analyzed by Western blotting using anti-HisGly antibody (Invitrogen, Carlsbad, Calif.). FIG. 2 shows hFGF10-X was expressed as a 29 kDa protein in E. coli cells.

Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. An isolated polypeptide comprising an amino acid sequence selected from the group consisting of:
a) a mature form of an amino acid sequence selected from the group consisting of SEQ ID NO: 2, 4, 6, 8, 10, 12, 14, 16, 18, 20, MM, AAA, CCC, EEE and LLL;
b) a variant of a mature form of an amino acid sequence selected from the group consisting of SEQ ID NO: 2, 4, 6, 8, 10, 12, 14, 16, 18, 20, MM, AAA, CCC, EEE and LLL, wherein one or more amino acid residues in said variant differs from the amino acid sequence of said mature form, provided that said variant differs in no more than 15% of the amino acid residues from the amino acid sequence of said mature form;
c) an amino acid sequence selected from the group consisting of SEQ ID NO: 2, 4, 6, 8, 10, 12, 14, 16, 18, 20, MM, AAA, CCC, EEE and LLL; and
d) a variant of an amino acid sequence selected from the group consisting of SEQ ID NO: 2, 4, 6, 8, 10, 12, 14, 16, 18, 20, MM, AAA, CCC, EEE and LLL wherein one or more amino acid residues in said variant differs from the amino acid sequence of said mature form, provided that said variant differs in no more than 15% of amino acid residues from said amino acid sequence.

2. The polypeptide of claim 1, wherein said polypeptide comprises the amino acid sequence of a naturally occurring allelic variant of an amino acid sequence selected from the group consisting of SEQ ID NO: 2, 4, 6, 8, 10, 12, 14, 16, 18, 20, MM, AAA, CCC, EEE and LLL.

3. The polypeptide of claim 2, wherein said allelic variant comprises an amino acid sequence that is the translation of a nucleic acid sequence differing by a single nucleotide from a nucleic acid sequence selected from the group consisting of SEQ ID NOs: 1, 3, 5, 7, 9, 11, 13, 15, 17, 19, AA, LL, ZZ, BBB, DDD, FFF, and KKK.

4. The polypeptide of claim 1, wherein the amino acid sequence of said variant comprises a conservative amino acid substitution.

5. An isolated nucleic acid molecule comprising a nucleic acid sequence encoding a polypeptide comprising an amino acid sequence selected from the group consisting of:
a) a mature form of an amino acid sequence selected from the group consisting of SEQ ID NO: 2, 4, 6, 8, 10, 12, 14, 16, 18, 20, MM, AAA, CCC, EEE and LLL;
b) a variant of a mature form of an amino acid sequence selected from the group consisting of SEQ ID NO: 2, 4, 6, 8, 10, 12, 14, 16, 18, 20, MM, AAA, CCC, EEE and LLL, wherein one or more amino acid residues in said variant differs from the amino acid sequence of said mature form, provided that said variant differs in no more than 15% of the amino acid residues from the amino acid sequence of said mature form;
c) an amino acid sequence selected from the group consisting of SEQ ID NO: 2, 4, 6, 8, 10, 12, 14, 16, 18, 20, MM, AAA, CCC, EEE and LLL;
d) a variant of an amino acid sequence selected from the group consisting of SEQ ID NO: 2, 4, 6, 8, 10, 12, 14, 16, 18, 20, MM, AAA, CCC, EEE and LLL, wherein one or more amino acid residues in said variant differs from the amino acid sequence of said mature form, provided that said variant differs in no more than 15% of amino acid residues from said amino acid sequence;
e) a nucleic acid fragment encoding at least a portion of a polypeptide comprising an amino acid sequence chosen from the group consisting of SEQ ID NO: 2, 4, 6, 8, 10, 12, 14, 16, 18, 20, MM, AAA, CCC, EEE and LLL, or a variant of said polypeptide, wherein one or more amino acid residues in said variant differs from the amino acid sequence of said mature form, provided that said variant differs in no more than 15% of amino acid residues from said amino acid sequence; and
f) a nucleic acid molecule comprising the complement of of a), b), c), d) or e).

6. The nucleic acid molecule of claim 5, wherein the nucleic acid molecule comprises the nucleotide sequence of a naturally occurring allelic nucleic acid variant

7. The nucleic acid molecule of claim 5, wherein the nucleic acid molecule encodes a polypeptide comprising the amino acid sequence of a naturally occurring polypeptide variant.

8. The nucleic acid molecule of claim 5, wherein the nucleic acid molecule differs by a single nucleotide from a nucleic acid sequence selected from the group consisting of SEQ ID NOs: 1, 3, 5, 7, 9, 11, 13, 15, 17, 19, AA, LL, ZZ, BBB, DDD, FFF, and KKK.

9. The nucleic acid molecule of claim 5, wherein said nucleic acid molecule comprises a nucleotide sequence selected from the group consisting of
a) a nucleotide sequence selected from the group consisting of SEQ ID NOs: 1, 3, 5, 7, 9, 11, 13, 15, 17, 19, AA, LL, ZZ, BBB, DDD, FFF, and KKK;
b) a nucleotide sequence differing by one or more nucleotides from a nucleotide sequence selected from the group consisting of SEQ ID NOs: 1, 3, 5, 7, 9, 11, 13, 15, 17, 19, AA, LL, ZZ, BBB, DDD, FFF, and KKK, provided that no more than 20% of the nucleotides differ from said nucleotide sequence;
c) a nucleic acid fragment of a); and
d) a nucleic acid fragment of b).

10. The nucleic acid molecule of claim 5, wherein said nucleic acid molecule hybridizes under stringent conditions to a nucleotide sequence chosen from the group consisting of SEQ ID NOs: 1, 3, 5, 7, 9, 11, 13, 15, 17, 19, AA, LL, ZZ, BBB, DDD, FFF, and KKK, or a complement of said nucleotide sequence.

11. The nucleic acid molecule of claim 5, wherein the nucleic acid molecule comprises a nucleotide sequence selected from the group consisting of
a) a first nucleotide sequence comprising a coding sequence differing by one or more nucleotide sequences from a coding sequence encoding said amino acid sequence, provided that no more than 20% of the nucleotides in the coding sequence in said first nucleotide sequence differ from said coding sequence;
b) an isolated second polynucleotide that is a complement of the first polynucleotide; and
c) a nucleic acid fragment of a) or b).

12. A vector comprising the nucleic acid molecule of claim 11.

13. The vector of claim 12, further comprising a promoter operably linked to said nucleic acid molecule.

14. A cell comprising the vector of claim 12.

15. An antibody that binds immunospecifically to the polypeptide of claim 1.

16. The antibody of claim 15, wherein said antibody is a monoclonal antibody.

17. The antibody of claim 15, wherein the antibody is a humanized antibody.

18. A method for determining the presence or amount of the polypeptide of claim 1 in a sample, the method comprising:
(a) providing the sample;
(b) contacting the sample with an antibody that binds immunospecifically to the polypeptide; and
(c) determining the presence or amount of antibody bound to said polypeptide,
thereby determining the presence or amount of polypeptide in said sample.

19. A method for determining the presence or amount of the nucleic acid molecule of claim 5 in a sample, the method comprising:
(a) providing the sample;
(b) contacting the sample with a probe that binds to said nucleic acid molecule; and
(c) determining the presence or amount of the probe bound to said nucleic acid molecule,
thereby determining the presence or amount of the nucleic acid molecule in said sample.

20. A method of identifying an agent that binds to a polypeptide of claim 1, the method comprising:
(a) contacting said polypeptide with said agent; and
(b) determining whether said agent binds to said polypeptide.

21. A method for identifying an agent that modulates the expression or activity of the polypeptide of claim 1, the method comprising:
(a) providing a cell expressing said polypeptide;
(c) contacting the cell with said agent, and
(d) determining whether the agent modulates expression or activity of said polypeptide,
whereby an alteration in expression or activity of said peptide indicates said agent modulates expression or activity of said polypeptide.

22. A method for modulating the activity of the polypeptide of claim 1, the method comprising contacting a cell sample expressing the polypeptide of said claim with a compound that binds to said polypeptide in an amount sufficient to modulate the activity of the polypeptide.

23. A method of treating or preventing a SECX-associated disorder, said method comprising administering to a subject in which such treatment or prevention is desired the polypeptide of claim 1 in an amount sufficient to treat or prevent said SECX-associated disorder in said subject.

24. The method of claim 23, wherein said subject is a human.

25. A method of treating or preventing a SECX-associated disorder, said method comprising administering to a subject in which such treatment or prevention is desired the nucleic acid of claim 5 in an amount sufficient to treat or prevent said SECX-associated disorder in said subject.

26. The method of claim 25, wherein said subject is a human.

27. A method of treating or preventing a SECX-associated disorder, said method comprising administering to a subject in which such treatment or prevention is desired the antibody of claim 15 in an amount sufficient to treat or prevent said SECX-associated disorder in said subject.

28. The method of claim 15, wherein the subject is a human.

29. A pharmaceutical composition comprising the polypeptide of claim 1 and a pharmaceutically acceptable carrier.

30. A pharmaceutical composition comprising the nucleic acid molecule of claim 5 and a pharmaceutically acceptable carrier.

31. A pharmaceutical composition comprising the antibody of claim 15 and a pharmaceutically acceptable carrier.

32. A kit comprising in one or more containers, the pharmaceutical composition of claim 29.

33. A kit comprising in one or more containers, the pharmaceutical composition of claim 30.

34. A kit comprising in one or more containers, the pharmaceutical composition of claim 31.

35. A method for determining the presence of or predisposition to a disease associated with altered levels of the polypeptide of claim 1 in a first mammalian subject, the method comprising:
a) measuring the level of expression of the polypeptide in a sample from the first mammalian subject; and
b) comparing the amount of said polypeptide in the sample of step (a) to the amount of the polypeptide present in a control sample from a second mammalian subject known not to have, or not to be predisposed to, said disease,
wherein an alteration in the expression level of the polypeptide in the first subject as compared to the control sample indicates the presence of or predisposition to said disease.

36. A method for determining the presence of or predisposition to a disease associated with altered levels of the nucleic acid molecule of claim 5 in a first mammalian subject, the method comprising:
a) measuring the amount of the nucleic acid in a sample from the first mammalian subject; and
b) comparing the amount of said nucleic acid in the sample of step (a) to the amount of the nucleic acid present in a control sample from a second mammalian subject known not to have or not be predisposed to, the disease;
wherein an alteration in the level of the nucleic acid in the first subject as compared to the control sample indicates the presence of or predisposition to the disease.

37. A method of treating a pathological state in a mammal, the method comprising administering to the mammal a polypeptide in an amount that is sufficient to alleviate the pathological state, wherein the polypeptide is a polypeptide having an amino acid sequence at least 95% identical to a polypeptide comprising an amino acid sequence of at least one of SEQ ID NOS: 2, 4, 6, 8, 10, 12, 14, 16, 18, 20, MM, AAA, CCC, EEE and LLL, or a biologically active fragment thereof.

38. A method of treating a pathological state in a mammal, the method comprising administering to the mammal the antibody of claim 15 in an amount sufficient to alleviate the pathological state
